SEQUENCE LISTING (264 sequences)

SEQ ID NO: 1 - protein, 575 aa, Unknown [sequence omitted]

SEQ ID NO: 2 - DNA, 1743 bp, Unknown
  Description of Unknown Phi29 polynucleotide
ggtaccatgaaacatatgccgcgcaaaatgtatagctgcgattttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgatggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaataataagagctc

SEQ ID NO: 3 - protein, 575 aa, Unknown
  Description of Unknown Phi29 polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 4 - DNA, 1725 bp, Unknown
  Description of Unknown Phi29 polynucleotide
atgaaacatatgccgcgcaaaatgtatagctgcgcctttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgccggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 5 - protein, 540 aa, Thermus aquaticus
ALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - DNA, 1623 bp, Thermus aquaticus
gccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 7 - protein, 540 aa, Thermus aquaticus
ALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMCGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 8 - DNA, 1622 bp, Thermus aquaticus
gccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgtgcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 9 - DNA, 33 bp, Artificial Sequence
  Description of Artificial Sequence Synthetic primer
ggcatggtgtttgatgcgaacagcctgtatccg

SEQ ID NO: 10 - DNA, 33 bp, Artificial Sequence
  Description of Artificial Sequence Synthetic primer
cggatacaggctgttcgcatcaaacaccatgcc

SEQ ID NO: 11 - DNA, 355 bp, Artificial Sequence
  Description of Artificial Sequence Synthetic polynucleotide
atatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgcgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaag

SEQ ID NO: 12 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgcgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 13 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgcgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgcaggcgcgattaaagcgctggccaaactgatgctgaatagcctgtacggcaaatttgcgagcaatccggatgtgaccggcaaagtgccgtatctgaaagaaaa

SEQ ID NO: 14 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgcgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctgcggcgcgattaaacagctggccaaactgatgctgaatagcctgtacggcaaatttgcgagcaatccggatgtgaccggcaaagtgccgtatctgaaagaaaa

SEQ ID NO: 15 - DNA, 33 bp, Artificial Sequence
  Description of Artificial Sequence Synthetic primer
ggcatggtgtttgatatcaacagcctgtatccg

SEQ ID NO: 16 - DNA, 33 bp, Artificial Sequence
  Description of Artificial Sequence Synthetic primer
cggatacaggctgttgatatcaaacaccatgcc

SEQ ID NO: 17 - DNA, 355 bp, Artificial Sequence
  Description of Artificial Sequence Synthetic polynucleotide
atatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatatcaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaag

SEQ ID NO: 18 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatatcaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 19 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatatcaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgcaggcgcgattaaagcgctggccaaactgatgctgaatagcctgtacggcaaatttgcgagcaatccggatgtgaccggcaaagtgccgtatctgaaagaaaa

SEQ ID NO: 20 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatatcaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctgcggcgcgattaaacagctggccaaactgatgctgaatagcctgtacggcaaatttgcgagcaatccggatgtgaccggcaaagtgccgtatctgaaagaaaa

SEQ ID NO: 21 - DNA, 39 bp, Artificial Sequence
  Description of Artificial Sequence Synthetic primer
gatcgtattatctactgcgaaaccgatagcattcatctg

SEQ ID NO: 22 - DNA, 39 bp, Artificial Sequence
  Description of Artificial Sequence Synthetic primer
cagatgaatgctatcggtttcgcagtagataatacgatc

SEQ ID NO: 23 - DNA, 385 bp, Artificial Sequence
  Description of Artificial Sequence Synthetic polynucleotide
ctgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgaaaccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggca

SEQ ID NO: 24 - DNA, 994 bp, Artificial Sequence
  Description of Artificial Sequence Synthetic polynucleotide
gattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgcgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgcaggcgcgattaaagcgctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgaaaccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtg

SEQ ID NO: 25 - DNA, 39 bp, Artificial Sequence
  Description of Artificial Sequence Synthetic primer
gatcgtattatctactgctcgaccgatagcattcatctg

SEQ ID NO: 26 - DNA, 39 bp, Artificial Sequence
  Description of Artificial Sequence Synthetic primer
cagatgaatgctatcggtcgagcagtagataatacgatc

SEQ ID NO: 27 - DNA, 385 bp, Artificial Sequence
  Description of Artificial Sequence Synthetic polynucleotide
ctgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgctcgaccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggca

SEQ ID NO: 28 - DNA, 33 bp, Artificial Sequence
  Description of Artificial Sequence Synthetic primer
ggcatggtgtttgattncaacagcctgtatccg

SEQ ID NO: 29 - DNA, 33 bp, Artificial Sequence
  Description of Artificial Sequence Synthetic primer
cggatacaggctgttgnaatcaaacaccatgcc

SEQ ID NO: 30 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatttcaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 31 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgattccaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 32 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgattacaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 33 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgattgcaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 34 - DNA, 33 bp, Artificial Sequence
  Description of Artificial Sequence Synthetic primer
ggcatggtgtttgatcngaacagcctgtatccg

SEQ ID NO: 35 - DNA, 33 bp, Artificial Sequence
  Description of Artificial Sequence Synthetic primer
cggatacaggctgttcngatcaaacaccatgcc

SEQ ID NO: 36 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatctgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 37 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatccgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 38 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatcagaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 39 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatcggaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 40 - DNA, 33 bp, Artificial Sequence
  Description of Artificial Sequence Synthetic primer
ggcatggtgtttgatangaacagcctgtatccg

SEQ ID NO: 41 - DNA, 33 bp, Artificial Sequence
  Description of Artificial Sequence Synthetic primer
cggatacaggctgttcntatcaaacaccatgcc

SEQ ID NO: 42 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatacgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 43 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgataagaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 44 - DNA, 33 bp, Artificial Sequence
  Description of Artificial Sequence Synthetic primer
ggcatggtgtttgatvacaacagcctgtatccg

SEQ ID NO: 45 - DNA, 33 bp, Artificial Sequence
  Description of Artificial Sequence Synthetic primer
cggatacaggctgttgtbatcaaacaccatgcc

SEQ ID NO: 46 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatcacaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 47 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgataacaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 48 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgacaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 49 - DNA, 33 bp, Artificial Sequence
  Description of Artificial Sequence Synthetic primer
ggcatggtgtttgatkggaacagcctgtatccg

SEQ ID NO: 50 - DNA, 33 bp, Artificial Sequence
  Description of Artificial Sequence Synthetic primer
cggatacaggctgttccmatcaaacaccatgcc

SEQ ID NO: 51 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgattggaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 52 - DNA, 770 bp, Artificial Sequence
  Description of Artificial Sequence Synthetic polynucleotide
ccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgggaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaa

SEQ ID NO: 53 - DNA, 44 bp, Artificial Sequence
  Description of Artificial Sequence Synthetic primer
cctacattaaaaccaccagctgcggcgcgattaaacagctggcc

SEQ ID NO: 54 - DNA, 44 bp, Artificial Sequence
  Description of Artificial Sequence Synthetic primer
ggccagctgtttaatcgcgccgcagctggtggttttaatgtagg

SEQ ID NO: 55 - DNA, 355 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctgcggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccgg

SEQ ID NO: 56 - DNA, 44 bp, Artificial Sequence
  Description of Combined DNA/RNA Molecule Synthetic primer
cctacattaaaaccaccagcuacggcgcgattaaacagctggcc

SEQ ID NO: 57 - DNA, 44 bp, Artificial Sequence
  Description of Artificial Sequence Synthetic primer
ggccagctgtttaatcgcgccgtagctggtggttttaatgtagg

SEQ ID NO: 58 - DNA, 355 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccgg

SEQ ID NO: 59 - DNA, 770 bp, Artificial Sequence
  Description of Artificial Sequence Synthetic polynucleotide
gattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctggttccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccg

SEQ ID NO: 60 - DNA, 385 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatggcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccg

SEQ ID NO: 61 - DNA, 807 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcgcactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 62 - DNA, 807 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctacggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcgcattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 63 - DNA, 50 bp, Artificial Sequence
  Description of Artificial Sequence Synthetic primer
cattaaaaccaccagcgcaggcgcgattaaagcgctggccaaactgatgc

SEQ ID NO: 64 - DNA, 50 bp, Artificial Sequence
  Description of Artificial Sequence Synthetic primer
gcatcagtttggccagcgctttaatcgcgcctgcgctggtggttttaatg

SEQ ID NO: 65 - DNA, 385 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgcaggcgcgattaaagcgctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccg

SEQ ID NO: 66 - DNA, 49 bp, Artificial Sequence
  Description of Artificial Sequence Synthetic primer
gctacgatcgtattatctactgcaataccgatagcattcatctgaccgg

SEQ ID NO: 67 - DNA, 49 bp, Artificial Sequence
  Description of Artificial Sequence Synthetic primer
ccggtcagatgaatgctatcggtattgcagtagataatacgatcgtagc

SEQ ID NO: 68 - DNA, 385 bp, Artificial Sequence
  Description of Artificial Sequence Synthetic polynucleotide
ctgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcaataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggca

SEQ ID NO: 69 - DNA, 45 bp, Artificial Sequence
  Description of Artificial Sequence Synthetic primer
cgtattatctactgcgataccaatagcattcatctgaccggcacc

SEQ ID NO: 70 - DNA, 45 bp, Artificial Sequence
  Description of Artificial Sequence Synthetic primer
ggtgccggtcagatgaatgctattggtatcgcagtagataatacg

SEQ ID NO: 71 - DNA, 385 bp, Artificial Sequence
  Description of Artificial Sequence Synthetic polynucleotide
ctgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccaatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggca

SEQ ID NO: 72 - DNA, 32 bp, Artificial Sequence
  Description of Artificial Sequence Synthetic primer
gattacaccgatatcgcattcagcgtgaaatg

SEQ ID NO: 73 - DNA, 32 bp, Artificial Sequence
  Description of Artificial Sequence Synthetic primer
catttcacgctgaatgcgatatcggtgtaatc

SEQ ID NO: 74 - DNA, 322 bp, Artificial Sequence
  Description of Artificial Sequence Synthetic polynucleotide
agatatcgtggatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcgcattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaataataagagctc

SEQ ID NO: 75 - DNA, 35 bp, Artificial Sequence
  Description of Artificial Sequence Synthetic primer
cattaaaaccaccagcgcaggcgcgattaaacagc

SEQ ID NO: 76 - DNA, 35 bp, Artificial Sequence
  Description of Artificial Sequence Synthetic primer
gctgtttaatcgcgcctgcgctggtggttttaatg

SEQ ID NO: 77 - DNA, 355 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgcaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccgg

SEQ ID NO: 78 - DNA, 36 bp, Artificial Sequence
  Description of Artificial Sequence Synthetic primer
gcgaaggcgcgattaaagcgctggccaaactgatgc

SEQ ID NO: 79 - DNA, 36 bp, Artificial Sequence
  Description of Artificial Sequence Synthetic primer
gcatcagtttggccagcgctttaatcgcgccttcgc

SEQ ID NO: 80 - DNA, 355 bp, Artificial Sequence
  Description of Artificial Sequence Synthetic polynucleotide
gagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaagcgctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcag

SEQ ID NO: 81 - DNA, 35 bp, Artificial Sequence
  Description of Artificial Sequence Synthetic primer
gatgtgaacagcctggttccggcgcagatgtatag

SEQ ID NO: 82 - DNA, 35 bp, Artificial Sequence
  Description of Artificial Sequence Synthetic primer
ctatacatctgcgccggaaccaggctgttcacatc

SEQ ID NO: 83 - DNA, 355 bp, Artificial Sequence
  Description of Artificial Sequence Synthetic polynucleotide
atatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctggttccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaag

SEQ ID NO: 84 - DNA, 39 bp, Artificial Sequence
  Description of Artificial Sequence Synthetic primer
gccaaactgatgctgaatggcctgtacggcaaatttgcg

SEQ ID NO: 85 - DNA, 39 bp, Artificial Sequence
  Description of Artificial Sequence Synthetic primer
cgcaaatttgccgtacaggccattcagcatcagtttggc

SEQ ID NO: 86 - DNA, 385 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatggcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccg

SEQ ID NO: 87 - DNA, 39 bp, Artificial Sequence
  Description of Artificial Sequence Synthetic primer
atgaaagaagtggacggcgcactggtggaaggcagcccg

SEQ ID NO: 88 - DNA, 39 bp, Artificial Sequence
  Description of Artificial Sequence Synthetic primer
cgggctgccttccaccagtgcgccgtccacttctttcat

SEQ ID NO: 89 - DNA, 355 bp, Artificial Sequence
  Description of Artificial Sequence Synthetic polynucleotide
tacgatcgtattatctactgcgataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcgcactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgc

SEQ ID NO: 90 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 91 - DNA, 1725 bp, Artificial Sequence
  Description of Artificial Sequence Synthetic polynucleotide
atgaaacatatgccgcgcaaaatgtatagctgcgattttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgatggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgctatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 92 - DNA, 45 bp, Artificial Sequence
  Description of Artificial Sequence Synthetic primer
gaatacgcgtatatcaaaaacgctatccagattattgcggaagcg

SEQ ID NO: 93 - DNA, 45 bp, Artificial Sequence
  Description of Artificial Sequence Synthetic primer
cgcttccgcaataatctggatagcgtttttgatatacgcgtattc

SEQ ID NO: 94 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgctatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 95 - DNA, 39 bp, Artificial Sequence
  Description of Artificial Sequence Synthetic primer
tatagctgcgattttgaaatcaccaccaaagtggaagat

SEQ ID NO: 96 - DNA, 39 bp, Artificial Sequence
  Description of Artificial Sequence Synthetic primer
atcttccactttggtggtgatttcaaaatcgcagctata

SEQ ID NO: 97 - DNA, 1725 bp, Artificial Sequence
  Description of Artificial Sequence Synthetic polynucleotide
atgaaacatatgccgcgcaaaatgtatagctgcgattttgaaatcaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgatggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 98 - DNA, 39 bp, Artificial Sequence
  Description of Artificial Sequence Synthetic primer
gcggatctgtattttcacgacctgaaatttgatggcgcg

SEQ ID NO: 99 - DNA, 39 bp, Artificial Sequence
  Description of Artificial Sequence Synthetic primer
cgcgccatcaaatttcaggtcgtgaaaatacagatccgc

SEQ ID NO: 100 - DNA, 1725 bp, Artificial Sequence
  Description of Artificial Sequence Synthetic polynucleotide
atgaaacatatgccgcgcaaaatgtatagctgcgattttgaaatcaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacgacctgaaatttgatggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 101 - DNA, 37 bp, Artificial Sequence
  Description of Artificial Sequence Synthetic primer
ccaccaaagtggaagattcccgtgtgtgggcgtatgg

SEQ ID NO: 102 - DNA, 37 bp, Artificial Sequence
  Description of Artificial Sequence Synthetic primer
ccatacgcccacacacgggaatcttccactttggtgg

SEQ ID NO: 103 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattcccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 104 - DNA, 42 bp, Artificial Sequence
  Description of Artificial Sequence Synthetic primer
catattcagcatattcgctccgaattcgagctgaaagaaggc

SEQ ID NO: 105 - DNA, 42 bp, Artificial Sequence
  Description of Artificial Sequence Synthetic primer
gccttctttcagctcgaattcggagcgaatatgctgaatatg

SEQ ID NO: 106 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctccgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 107 - DNA, 41 bp, Artificial Sequence
  Description of Artificial Sequence Synthetic primer
ctacgatcgtattatctactccgataccgatagcattcatc

SEQ ID NO: 108 - DNA, 41 bp, Artificial Sequence
  Description of Artificial Sequence Synthetic primer
gatgaatgctatcggtatcggagtagataatacgatcgtag

SEQ ID NO: 109 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactccgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 110 - DNA, 45 bp, Artificial Sequence
  Description of Artificial Sequence Synthetic primer
gatatcaaattcagcgtgaaatccgcgggcatgaccgataaaatc

SEQ ID NO: 111 - DNA, 45 bp, Artificial Sequence
  Description of Artificial Sequence Synthetic primer
gattttatcggtcatgcccgcggatttcacgctgaatttgatatc

SEQ ID NO: 112 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatccgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 113 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattgccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctccgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactccgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatccgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 114 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattcccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactccgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatccgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 115 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattcccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctccgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatccgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 116 - DNA, 1725 bp, Artificial Sequence
  Description of Artificial Sequence Synthetic polynucleotide
atgaaacatatgccgcgcaaaatgtatagctgcgcttttgaaaccaccaccaaagtggaagattcccgtgtgtgggcgtatggctatatgaacatcgaagatcacagcgaatataaaattggcaacagcctggatgaatttatggcgtgggtgctgaaagtgcaggcggatctgtattttcacaacctgaaatttgctggcgcgtttattattaactggctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtatccggcgcagatgtatagccgtctgctgccgtatggcgaaccgattgtgtttgaaggcaaatacgtgtgggatgaagattatccgctgcatattcagcatattcgctccgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactccgataccgatagcattcatctgaccggcaccgaaattccggatgtgatcaaagatatcgttgatccgaaaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 117 - DNA, 35 bp, Artificial Sequence
  Description of Artificial Sequence Synthetic primer
gatgtgaacagcctgtytccggcgcagatgtatag

SEQ ID NO: 118 - DNA, 35 bp, Artificial Sequence
  Description of Artificial Sequence Synthetic primer
ctatacatctgcgccggaracaggctgttcacatc

SEQ ID NO: 119 - DNA, 355 bp, Artificial Sequence
  Description of Artificial Sequence Synthetic polynucleotide
atatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtttccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaag

SEQ ID NO: 120 - DNA, 355 bp, Artificial Sequence
  Description of Artificial Sequence Synthetic polynucleotide
atatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtctccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaag

SEQ ID NO: 121 - DNA, 32 bp, Artificial Sequence
  Description of Artificial Sequence Synthetic primer
gtgaacagcctgtattcggcgcagatgtatag

SEQ ID NO: 122 - DNA, 32 bp, Artificial Sequence
  Description of Artificial Sequence Synthetic primer
ctatacatctgcgccgaatacaggctgttcac

SEQ ID NO: 123 - DNA, 355 bp, Artificial Sequence
  Description of Artificial Sequence Synthetic polynucleotide
atatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctgtattcggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaag

SEQ ID NO: 124 - DNA, 33 bp, Artificial Sequence
  Description of Artificial Sequence Synthetic primer
gaagtggacggcgcagcggtggaaggcagcccg

SEQ ID NO: 125 - DNA, 33 bp, Artificial Sequence
  Description of Artificial Sequence Synthetic primer
cgggctgccttccaccgctgcgccgtccacttc

SEQ ID NO: 126 - DNA, 355 bp, Artificial Sequence
  Description of Artificial Sequence Synthetic polynucleotide
tacgatcgtattatctactgcgataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcgcagcggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgc

SEQ ID NO: 127 - DNA, 39 bp, Artificial Sequence
  Description of Artificial Sequence Synthetic primer
gcgattaaacagctggcccgactgatgctgaatagcctg

SEQ ID NO: 128 - DNA, 39 bp, Artificial Sequence
  Description of Artificial Sequence Synthetic primer
caggctattcagcatcagtcgggccagctgtttaatcgc

SEQ ID NO: 129 - DNA, 355 bp, Artificial Sequence
  Description of Artificial Sequence Synthetic polynucleotide
gagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggcccgactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcag

SEQ ID NO: 130 - DNA, 39 bp, Artificial Sequence
  Description of Artificial Sequence Synthetic primer
gcgattaaacagctggccccactgatgctgaatagcctg

SEQ ID NO: 131 - DNA, 39 bp, Artificial Sequence
  Description of Artificial Sequence Synthetic primer
caggctattcagcatcagtggggccagctgtttaatcgc

SEQ ID NO: 132 - DNA, 355 bp, Artificial Sequence
  Description of Artificial Sequence Synthetic polynucleotide
gagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccccactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcag

SEQ ID NO: 133 - DNA, 39 bp, Artificial Sequence
  Description of Artificial Sequence Synthetic primer
gcgattaaacagctggccgcactgatgctgaatagcctg

SEQ ID NO: 134 - DNA, 39 bp, Artificial Sequence
  Description of Artificial Sequence Synthetic primer
caggctattcagcatcagtgcggccagctgtttaatcgc

SEQ ID NO: 135 - DNA, 355 bp, Artificial Sequence
  Description of Artificial Sequence Synthetic polynucleotide
gagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccgcactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcag

SEQ ID NO: 136 - DNA, 44 bp, Artificial Sequence
  Description of Artificial Sequence Synthetic primer
gatcgtattatctactgcaataccaatagcattcatctgaccgg

SEQ ID NO: 137 - DNA, 44 bp, Artificial Sequence
  Description of Artificial Sequence Synthetic primer
ccggtcagatgaatgctattggtattgcagtagataatacgatc

SEQ ID NO: 138 - DNA, 385 bp, Artificial Sequence
  Description of Artificial Sequence Synthetic polynucleotide
ctgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcaataccaatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggca

SEQ ID NO: 139 - DNA, 639 bp, Artificial Sequence
  source: Description of Artificial Sequence Synthetic polynucleotide
cagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcccgaaatttaaagcgaccaccggcctgtttaaagatttcatcgataaatggacctacattaaaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcaataccaatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcaaactggtggaa

SEQ ID NO: 140 - DNA, 40 bp, Artificial Sequence
  Description of Artificial Sequence Synthetic primer
ggccaaactgatgctggctagcctgtacggcaaatttgcg

SEQ ID NO: 141 - DNA, 40 bp, Artificial Sequence
  Description of Artificial Sequence Synthetic primer
cgcaaatttgccgtacaggctagccagcatcagtttggcc

SEQ ID NO: 142 - DNA, 355 bp, Artificial Sequence
  Description of Artificial Sequence Synthetic polynucleotide
cgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctggctagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgc

SEQ ID NO: 143 - DNA, 40 bp, Artificial Sequence
  Description of Artificial Sequence Synthetic primer
ggccaaactgatgctgtatagcctgtacggcaaatttgcg

SEQ ID NO: 144 - DNA, 40 bp, Artificial Sequence
  Description of Artificial Sequence Synthetic primer
cgcaaatttgccgtacaggctatacagcatcagtttggcc

SEQ ID NO: 145 - DNA, 355 bp, Artificial Sequence
  Description of Artificial Sequence Synthetic polynucleotide
cgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgtatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgc

SEQ ID NO: 146 - DNA, 32 bp, Artificial Sequence
  Description of Artificial Sequence Synthetic primer
ggcaaatttgcgagcgctccggatgtgaccgg

SEQ ID NO: 147 - DNA, 32 bp, Artificial Sequence
  Description of Artificial Sequence Synthetic primer
ccggtcacatccggagcgctcgcaaatttgcc

SEQ ID NO: 148 - DNA, 355 bp, Artificial Sequence
  Description of Artificial Sequence Synthetic polynucleotide
cgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaactgatgctgaatagcctgtacggcaaatttgcgagcgctccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgc

SEQ ID NO: 149 - DNA, 44 bp, Artificial Sequence
  Description of Artificial Sequence Synthetic primer
gcgattaaacagctggccaaaaggatgctgaatagcctgtacgg

SEQ ID NO: 150 - DNA, 44 bp, Artificial Sequence
  Description of Artificial Sequence Synthetic primer
ccgtacaggctattcagcatccttttggccagctgtttaatcgc

SEQ ID NO: 151 - DNA, 355 bp, Artificial Sequence
  Description of Artificial Sequence Synthetic polynucleotide
cgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattaaacagctggccaaaaagatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgc

SEQ ID NO: 152 - DNA, 37 bp, Artificial Sequence
  Description of Artificial Sequence Synthetic primer
gggctattgggcgcatgcaagcacctttaaacgcgcg

SEQ ID NO: 153 - DNA, 37 bp, Artificial Sequence
  Description of Artificial Sequence Synthetic primer
cgcgcgtttaaaggtgcttgcatgcgcccaatagccc

SEQ ID NO: 154 - DNA, 355 bp, Artificial Sequence
  Description of Artificial Sequence Synthetic polynucleotide
tgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccgaaaaaactgggctattgggcgcatgcaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtggg

SEQ ID NO: 155 - DNA, 37 bp, Artificial Sequence
  Description of Artificial Sequence Synthetic primer
gggctattgggcgcatgacagcacctttaaacgcgcg

SEQ ID NO: 156 - DNA, 37 bp, Artificial Sequence
  Description of Artificial Sequence Synthetic primer
cgcgcgtttaaaggtgctgtcatgcgcccaatagccc

SEQ ID NO: 157 - DNA, 355 bp, Artificial Sequence
  Description of Artificial Sequence Synthetic polynucleotide
tgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccgaaaaaactgggctattgggcgcatgacagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtggg

SEQ ID NO: 158 - DNA, 44 bp, Artificial Sequence
  Description of Combined DNA/RNA Molecule Synthetic primer
cctacattaaaaccaccagcuggggcgcgattaaacagctggcc

SEQ ID NO: 159 - DNA, 44 bp, Artificial Sequence
  Description of Artificial Sequence Synthetic primer
ggccagctgtttaatcgcgccccagctggtggttttaatgtagg

SEQ ID NO: 160 - DNA, 355 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagctggggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccgg

SEQ ID NO: 161 - DNA, 47 bp, Artificial Sequence
  Description of Artificial Sequence Synthetic primer
gatcaaagatatcgtggatccggcaaaactgggctattgggcgcatg

SEQ ID NO: 162 - DNA, 47 bp, Artificial Sequence
  Description of Artificial Sequence Synthetic primer
catgcgcccaatagcccagttttgccggatccacgatatctttgatc

SEQ ID NO: 163 - DNA, 355 bp, Artificial Sequence
  Description of Artificial Sequence Synthetic polynucleotide
tgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccggcaaaactgggctattgggcgcatgaaagcacctttaaacgcgcgaaatatctgcgtcagaaaacctatatccaggatatctatatgaaagaagtggacggcaaactggtggaaggcagcccggatgattacaccgatatcaaattcagcgtgaaatgcgcgggcatgaccgataaaatcaaaaaagaagtgaccttcgaaaactttaaagtggg

SEQ ID NO: 164 - DNA, 39 bp, Artificial Sequence
  Description of Artificial Sequence Synthetic primer
ctgccgtttccggtgaaagcaatcgcgaaagatttcaaa

SEQ ID NO: 165 - DNA, 39 bp, Artificial Sequence
  Description of Artificial Sequence Synthetic primer
tttgaaatctttcgcgattgctttcaccggaaacggcag

SEQ ID NO: 166 - DNA, 355 bp, Artificial Sequence
  Description of Artificial Sequence Synthetic polynucleotide
ctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaagcaatcgcgaaagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggca

SEQ ID NO: 167 - DNA, 42 bp, Artificial Sequence
  Description of Artificial Sequence Synthetic primer
ccggtgaaaaaaatcgcggcagatttcaaactgaccgtgctg

SEQ ID NO: 168 - DNA, 42 bp, Artificial Sequence
  Description of Artificial Sequence Synthetic primer
cagcacggtcagtttgaaatctgccgcgatttttttcaccgg

SEQ ID NO: 169 - DNA, 355 bp, Artificial Sequence
  Description of Artificial Sequence Synthetic polynucleotide
ctggaacgcaacggctttaaatggagcgcggatggcctgccgaacacctataacaccattattagccgtatgggccagtggtatatgattgatatctgcctgggctataaaggcaaacgcaaaatccataccgtgatctacgatagcctgaaaaaactgccgtttccggtgaaaaaaatcgcggcagatttcaaactgaccgtgctgaaaggcgatatcgattatcataaagaacgtccggtgggctataaaattaccccggaagaatacgcgtatatcaaaaacgatatccagattattgcggaagcgctgctgattcagtttaaacagggcctggatcgtatgaccgcgggca

SEQ ID NO: 170 - DNA, 39 bp, Artificial Sequence
  Description of Artificial Sequence Synthetic primer
gataaatggacctacattacaaccaccagcgaaggcgcg

SEQ ID NO: 171 - DNA, 39 bp, Artificial Sequence
  Description of Artificial Sequence Synthetic primer
cgcgccttcgctggtggttgtaatgtaggtccatttatc

SEQ ID NO: 172 - DNA, 355 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattacaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccgg

SEQ ID NO: 173 - DNA, 39 bp, Artificial Sequence
  Description of Artificial Sequence Synthetic primer
gataaatggacctacattgcaaccaccagcgaaggcgcg

SEQ ID NO: 174 - DNA, 39 bp, Artificial Sequence
  Description of Artificial Sequence Synthetic primer
cgcgccttcgctggtggttgcaatgtaggtccatttatc

SEQ ID NO: 175 - DNA, 355 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattgcaaccaccagcgaaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccgg

SEQ ID NO: 176 - DNA, 39 bp, Artificial Sequence
  Description of Artificial Sequence Synthetic primer
accagcgaaggcgcgattgcacagctggccaaactgatg

SEQ ID NO: 177 - DNA, 39 bp, Artificial Sequence
  Description of Artificial Sequence Synthetic primer
catcagtttggccagctgtgcaatcgcgccttcgctggt

SEQ ID NO: 178 - DNA, 284 bp, Artificial Sequence
  Description of Artificial Sequence Synthetic polynucleotide
cgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgcgattgcacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccgg

SEQ ID NO: 179 - DNA, 35 bp, Artificial Sequence; identical to SEQ ID NO: 75
  Description of Artificial Sequence Synthetic primer

SEQ ID NO: 180 - DNA, 35 bp, Artificial Sequence; identical to SEQ ID NO: 76
  Description of Artificial Sequence Synthetic primer

SEQ ID NO: 181 - DNA, 807 bp, Artificial Sequence
  Description of Artificial Sequence Synthetic polynucleotide
agccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgcaggcgcgattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtgtttattaccgcgtgggcgcgttataccaccattaccgcggcgcaggcgtgctacgatcgtattatctactgcgataccgatagcattcatctgaccggcaccgaaattccggatgtgatcaaagatatcgtggatccgaaaaaactgggctattgggcgcatgaaagcacctttaaacgcgcgaaatatctgcgtcagaaaacctatatccaggatatctatatgaaagaagtggacggcgcactggtggaaggcagcccggatgattacaccgatatcaaattcagcgtgaaatgcgcgggcatgaccgataaaatcaaaaaagaagtgaccttcgaaaactttaaagtgggcttcagccgtaaaatgaaaccgaaaccggtgcaggtgccgggtggtgtggtgctggttgatgataccttcaccatcaaa

SEQ ID NO: 182 - DNA, 760 bp, Artificial Sequence
  Description of Artificial Sequence Synthetic polynucleotide
gattcagtttaaacagggcctggatcgtatgaccgcgggcagcgacagtctgaaaggcttcaaagatatcatcaccaccaaaaaattcaaaaaagtgttcccgaccctgagcctgggcctggataaagaagtgcgttacgcgtatcgtggcggctttacctggctgaacgatcgcttcaaagaaaaagaaatcggcgaaggcatggtgtttgatgtgaacagcctggttccggcgcagatgtatagccgtctgctgccgtatggcgaaccgattgtgtttgaaggcaaatacgtgtgggatgaagattatccgctgcatattcagcatattcgctgcgaattcgagctgaaagaaggctatattccgaccattcagattaaacgcagccgcttttataaaggcaacgaatatctgaaaagcagcggcggtgaaattgcggatctgtggctgagcaacgtggatctggagctgatgaaagaacactatgatctgtataacgtggaatatatcagcggcctgaaatttaaagcgaccaccggcctgtttaaagatttcatcgataaatggacctacattaaaaccaccagcgaaggcgagattaaacagctggccaaactgatgctgaatagcctgtacggcaaatttgcgagcaatccggatgtgaccggcaaagtgccgtatctgaaagaaaacggcgcgctgggctttcgtctgggcgaagaagaaaccaaagatccggtgtataccccgatgggcgtg

SEQ ID NO: 183 - DNA, 10 bp, Artificial Sequence
  Description of Artificial Sequence Synthetic oligonucleotide
ccttctctcc

SEQ ID NO: 184 - DNA, 35 bp, Artificial Sequence
  Description of Artificial Sequence Synthetic oligonucleotide
actgtatacgtacgatgctatgcatcgattcgtac

SEQ ID NO: 185 - DNA, 35 bp, Artificial Sequence
  Description of Artificial Sequence Synthetic oligonucleotide
actgtatacgtacggtgctatgcatcgattcgtac

SEQ ID NO: 186 - DNA, 29 bp, Artificial Sequence
  Description of Artificial Sequence Synthetic oligonucleotide
catcgattcgtacgtgcctcgagtttctg

SEQ ID NO: 187 - DNA, 29 bp, Artificial Sequence
  Description of Artificial Sequence Synthetic oligonucleotide
catcgattcgtacgtgcctcgagcctctg

SEQ ID NO: 188 - DNA, 51 bp, Artificial Sequence
  Description of Artificial Sequence Synthetic oligonucleotide
actgtatacgtacgntgctatgcatcgattcgtacgtgcctcgagnntctg

SEQ ID NO: 189 - DNA, 51 bp, Artificial Sequence
  Description of Artificial Sequence Synthetic oligonucleotide
actgtatacgtacgatgctatgcatcgattcgtacgtgcctcgagtttctg

SEQ ID NO: 190 - DNA, 51 bp, Artificial Sequence
  Description of Artificial Sequence Synthetic oligonucleotide
actgtatacgtacgatgctatgcatcgattcgtacgtgcctcgagcctctg

SEQ ID NO: 191 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDANSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 192 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDANSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSAGAIKALAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 193 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDANSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSCGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 194 - protein, 575 aa, Artificial Sequence; identical to SEQ ID NO: 191
  Description of Artificial Sequence Synthetic polypeptide

SEQ ID NO: 195 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDINSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSAGAIKALAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 196 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDINSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSCGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 197 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDANSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 198 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDINSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 199 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSAGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 200 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSCGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 201 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 202 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSAGAIKALAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 203 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKALAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 204 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCNTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 205 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCETDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 206 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCSTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 207 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTNSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 208 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDANSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSAGAIKALAKLMLNSLYGKFASNPDVTGKVPYLKENGALGFRLGEEETKDPVYTPMGVFITAWARYTTITAAQACYDRIIYCETDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 209 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDLNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 210 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDPNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 211 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDQNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 212 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDRNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 213 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDYNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 214 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDFNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 215 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDSNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 216 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDCNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 217 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDTNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 218 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDKNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 219 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDHNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 220 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDNNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 221 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDDNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 222 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDGNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 223 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDWNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 224 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNGLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 225 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGALVEGSPDDYTDIKFSVKCAGMTDKIKKEVTFENFKVGFSRKMKPKPVQVPGGVVLVDDTFTIK

SEQ ID NO: 226 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIAFSVKCAGMTDKIKKEVTFENFKVGFSRKMKPKPVQVPGGVVLVDDTFTIK

SEQ ID NO: 227 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLVPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSYGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 228 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKAIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 229 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAALMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 230 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLARLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 231 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAPLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 232 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIATTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 233 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYITTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 234 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLFPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 235 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLVPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 236 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLSPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 237 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIAQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 238 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIAFSVKCAGMTDKIKKEVTFENFKVGFSRKMKPKPVQVPGGVVLVDDTFTIK

SEQ ID NO: 239 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAADFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 240 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYSAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 241 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNGLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 242 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGALVEGSPDDYTDIKFSVKCAGMTDKIKKEVTFENFKVGFSRKMKPKPVQVPGGVVLVDDTFTIK

SEQ ID NO: 243 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKRMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 244 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHASTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 245 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHDSTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 246 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPAKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 247 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSWGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 248 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLASLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 249 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLYSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 250 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDANSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSWGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 251 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGPKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCNTNSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 252 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLVPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGEIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 253 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCNTNSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 254 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNAIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 255 - protein, 575 aa, Artificial Sequence
  Description of Artificial Sequence Synthetic polypeptide
MKHMPRKMYSCAFEITTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 256 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHDLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 257 - protein, 575 aa, Artificial Sequence
  Description of Artificial Sequence Synthetic polypeptide
MKHMPRKMYSCAFETTTKVEDSRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 258 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRSEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 259 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQASYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 260 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKSAGMTDKIKKEVTFENFKVGFSRKMKPKPVQVPGGVVLVDDTFTIK

SEQ ID NO: 261 - protein, 575 aa, Artificial Sequence
  Description of Artificial Sequence Synthetic polypeptide
MKHMPRKMYSCAFETTTKVEDCRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRSEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQASYDRIIYCDTDSIHLTGTEIPDVIKDIVDPKKLGYWAHESTFKRAKYLRQKTYIQDIYMKEVDGKLVEGSPDDYTDIKFSVKSAGMTDKIKKEVTFENFKVGFSRKMKPKPVQVPGGVVLVDDTFTIK

SEQ ID NO: 262 - protein, 575 aa, Artificial Sequence
  Description of Artificial Sequence Synthetic polypeptide
MKHMPRKMYSCAFETTTKVEDSRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQASYDRIIYCDTDSIHLTGTEIPDVIKDIVDPKKLGYWAHESTFKRAKYLRQKTYIQDIYMKEVDGKLVEGSPDDYTDIKFSVKSAGMTDKIKKEVTFENFKVGFSRKMKPKPVQVPGGVVLVDDTFTIK

SEQ ID NO: 263 - protein, 575 aa, Artificial Sequence
  Description of Artificial Sequence Synthetic polypeptide
MKHMPRKMYSCAFETTTKVEDSRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRSEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKSAGMTDKIKKEVTFENFKVGFSRKMKPKPVQVPGGVVLVDDTFTIK

SEQ ID NO: 264 - protein, 575 aa, Artificial Sequence
  Description of Artificial Sequence Synthetic polypeptide
MKHMPRKMYSCAFETTTKVEDSRVWAYGYMNIEDHSEYKIGNSLDEFMAWVLKVQADLYFHNLKFAGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRSEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQASYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

(19) United States
(12) Patent Application Publication
(10) Pub. No.: US 2011/0165652 A1
(43) Pub. Date: Jul. 7, 2011

(54) COMPOSITIONS, METHODS AND SYSTEMS FOR SINGLE MOLECULE SEQUENCING

(75) Inventors: Susan H. Hardin, College Station, TX (US); Tommie Lloyd Lincecum, JR., Houston, TX (US); Norha Deluge, Pearland, TX (US); Hongyi Wang, Pearland, TX (US); Yuri Belosludtsev, The Woodlands, TX (US); Kristi Kincaid, Houston, TX (US); Anelia Kraltcheva, Houston, TX (US); Benjamin Stevens, Philadelphia, PA (US); Ming Fa, Pearland, TX (US); Amy Bryant, Fredericksburg, TX (US); Amy Castillo, Houston, TX (US); Hye Eun Kim, Fort Worth, TX (US); Uma Nagaswamy, Pearland, TX (US); Mitsu Sreedhar Reddy, Pearland, TX (US); Alok N. Bandekar, Pearland, TX (US); Ivan Pan, Houston, TX (US); Andrei Volkov, Houston, TX (US)

(73) Assignee: LIFE TECHNOLOGIES CORPORATION, Carlsbad, CA (US)

(21) Appl. No.: 12/812,952

(22) PCT Filed: Jan. 14, 2009
(86) PCT No.: PCT/US2009/031027

§ 371 Date: Jul. 14, 2010

Related U.S. Application Data
(60) Provisional application No. 61/020,995, filed on Jan. 14, 2008.

(51) Int. Cl. C12N 9/12

(52) U.S. Cl. 435/194

(57) ABSTRACT

Compositions, systems and methods of sequencing are disclosed, where the compositions and systems include polymerase enzymes that have been genetically modified to more efficiently incorporate nucleotides including labels having a detectable properties that are released during incorporation, to augment a rate of labeled nucleotide incorporation, to augment a rate of pyrophosphate release, or to augment two or more of these properties and rates. Also disclosed are terminally labeled and dual labeled nucleotides, and click-chemistry based methods of synthesizing the same.

This application claims priority to U.S. provisional application No. 61/020,995, filed on Jan. 14, 2008, which is hereby incorporated by reference in its entirety.

### BACKGROUND

1. Field of the Art

The present disclosure relates to compositions, methods and systems of nucleotide sequencing at the single molecule level using engineered polymerases and/or engineered nucleotides.

2. Description of the Related Art

Recently, several groups have made great strides in harnessing the ability to sequence DNA at the single molecule level. Although the approaches differ, most require the use of labeled nucleotides. Generally, such labeled nucleotides have low incorporation efficiencies or are not incorporated at all. To overcome these incorporation problems, these groups have sought polymerases that are capable of efficiently incorporating labeled nucleotides. This work is still on-going. Thus, there is a need in the art for new mutant polymerases that are capable of effectively and efficiently incorporating labeled nucleotides.

Additionally, current single molecule sequencing strategies are hampered by the need for a strategy that facilitates accurate assembly of the gathered sequence information. Thus, there remains a need for improved strategies that facilitate assembly of of multiple sequence reads into a single ordered sequence.

### SUMMARY OF THE DISCLOSURE

The present disclosure relates to methods, compositions and systems for nucleotide sequencing at the single molecule level using polymerases and nucleotides. More particularly, the present disclosure relates to methods, compositions and systems wherein the polymerase and/or nucleotides have been modified, engineered or otherwise adapted to facilitate the detection of one or more nucleotide incorporation events during a nucleotide polymerase reaction. Typically, this is accomplished by monitoring detectable signals emitted by labels operably linked or otherwise attached to various components of the nucleotide polymerase reaction. In some embodiments, the detectable signals are a result of Forster Resonance Energy Transfer (FRET) between a single FRET donor and a single FRET acceptor, wherein the donor and acceptor are attached to different components of the polymerase reaction.

The present disclosure also provides Phi29 polymerase variants exhibiting altered properties for nucleotide binding, altered rates of pyrophosphate (or polyphosphate) release, and/or altered rates of nucleotide incorporation.

Also provided herein are methods for modifying a polymerizing agent, for example a nucleotide polymerase, to obtain a polymerase exhibiting altered properties for nucleotide binding, altered rates of pyrophosphate (or polyphosphate) release, and/or altered rates of nucleotide incorporation.

The present disclosure also provides Phi29 polymerase mutants that are capable of effectively and efficiently incorporating modified nucleotides, where the modification comprises a non-persistent label having a detectable property and optionally a persistent label also optionally having a detectable property and where the mutants are selected from the group consisting of those set forth in Table 1 below. For example, provided herein are isolated variants of Phi-29 polymerase comprising one or more mutations selected from the group consisting of: V250A/E375Y, V250A/E375A/Q380A, V250A/E375C, V250A/E375Y, V250I/E375A/Q380A, V250I/E375C, V250A, V250I, E375A, E375C, E375Y, E375A/Q380A, Q380A, D456N, D456E, D456S, D458N, V250A/E375A/Q380A/D456E, E375Y/V250L, E375Y/V250P, E375Y/V250Q, E375Y/V250R, E375Y/V250Y, E375Y/V250F, E375YN250S, E375Y/V250C, E375Y/V250T, E375Y/V250K, E375Y/V250H, E375Y/V250N, E375Y/V250D, E375Y/V250G, E375Y/V250W, E375Y/S388G, E375Y/K512A, E375Y/K525A, Y254V/E375Y, K132A, K383A, K383R, K383P, K371A, K371T, Y254F, Y254V, Y254S, Y254V, Y254S, K379A, K525A, K135A, P255S, S388G, K512A, L384R, E486A, E486D, K478A, E375W, N387A, N387Y, V250A/E375W, D456N/D458N/L351P, Y254V/A377E, D456N/D458N, D169A, D12A/D66A/D169A, T151, N62D, C22S, C290S, C448S, C530S, C290S/C448S/C530S, C22S/C448S/C530S, C22S/C290S/C530S and C22S/C290S/C448S.

Also provided herein are isolated variants of a Phi-29 polymerase comprising the amino acid sequence shown in SEQ ID NO: 3, wherein the variant comprises an amino acid sequence that is at least 80% identical to SEQ ID NO: 3, and wherein the variant further comprises one or more mutations selected from the group consisting of: V250A/E375Y, V250A/E375A/Q380A, V250A/E375C, V250A/E375Y, V250I/E375A/Q380A, V2501/E375C, V250A, V2501, E375A, E375C, E375Y, E375A/Q380A, Q380A, D456N, D456E, D456S, D458N, V250A/E375A/Q380A/D456E, E375Y/V250L, E375Y/V250P, E375Y/V250Q, E375Y/V250R, E375Y/V250Y, E375Y/V250F, E375YN2505, E375Y/V250C, E375Y/V250T, E375Y/V250K, E375Y/V250H, E375Y/V250N, E375Y/V250D, E375Y/V250G, E375Y/V250W, E375Y/S388G, E375Y/K512A, E375Y/K525A, Y254V/E375Y, K132A, K383A, K383R, K383P, K371A, K371T, Y254F, Y254V, Y254S, Y254V, Y254S, K379A, K525A, K135A, P255S, S388G, K512A, L384R, E486A, E486D, K478A, E375W, N387A, N387Y, V250A/E375W, D456N/D458N/L351P, Y254V/A377E, D456N/D458N, D169A, D12A/D66A/D169A, T151, N62D, C22S, C290S, C448S, C530S, C290S/C448S/C530S, C22S/C448S/C530S, C22S/C290S/C530S and C22S/C290S/C448S.

The present disclosure also provides a method for detecting one or more nucleotide incorporation events using the mutant polymerases of this disclosure, comprising: conducting a nucleotide polymerase reaction in the presence of one or more detectably labeled nucleotides and a mutant polymerase of this disclosure, which reaction results the production of a detectable signal before, after or during a nucleotide incorporation event; and detecting the detectable signal, thereby determining if a nucleotide incorporation event has occurred. Optionally, the detectable label of the nucleotide is a FRET acceptor, and/or the detectable signal is a FRET signal. Optionally, the methods further comprise the step of analyzing the signal to determine the identity of the nucleobase of the incorporated nucleotide.

The present disclosure also provides a method for sequencing nucleic acid using the mutant polymerases of this disclosure. For example, disclosed herein is a method for determining a nucleotide sequence of a nucleic acid molecule, comprising conducting a nucleic acid polymerase reaction in the presence of at least one detectably labeled nucleotide and a mutant polymerase of this disclosure, which reaction results the production of a detectable signal before, after or during a nucleotide incorporation event; detecting a time sequence of incorporation events; and determining the identity of individual nucleotides incorporated during the polymerase reaction, thereby determining a nucleotide sequence of the nucleic acid molecule.

Also provided herein is an isolated variant of Taq polymerase comprising the mutation F647C.

Also provided herein is an isolated variant of Taq polymerase comprising the amino acid sequence shown in SEQ ID NO: 7, wherein the variant comprises an amino acid sequence that is at least 80% identical to SEQ ID NO: 7, and wherein the variant further comprises the mutation F647C.

The present disclosure also provides a nucleotide synthetic methodology for forming a terminally labeled nucleotide using so-called “click chemistry”. For example, disclosed herein is a method for synthesizing a detectably labeled nucleotide, comprising: (a) introducing a first click group onto a nucleotide; (b) introducing a second click group capable of specifically reacting with the first click group onto a detectable label; and (c) reacting the nucleotide with the detectable label, thereby forming a detectably labeled nucleotide.

Also disclosed herein is a method for synthesizing a terminally labeled nucleotide, comprising: (a) introducing a terminal alkyne group onto the terminal phosphate of a nucleotide; (b) reacting the nucleotide with a first compound comprising an azide group and a linking group, thereby forming a nucleotide comprising a linking group attached to the terminal phosphate; and (c) reacting the linking group with a second compound comprising a detectable label, thereby forming a terminally labeled nucleotide. Also disclosed herein is another method for synthesizing a terminally labeled nucleotide, comprising: (a) introducing an azide group onto the terminal phosphate of a nucleotide; (b) reacting the nucleotide with a first compound comprising a terminal alkyne group and a linking group, thereby forming a nucleotide comprising a linking group attached to the terminal phosphate; and (c) reacting the linking group with a second compound comprising a detectable label, thereby forming a terminally labeled nucleotide.

Also disclosed herein are dual labeled nucleotide compositions, comprising a first detectable label operably linked to the terminal phosphate and a second detectable label operably linked to the nucleobase, wherein the first and second detectable labels do not significantly quench each other. More particularly, disclosed herein are nucleotide having the formula: D1-P—(P)ₙ—S—B-D2, wherein P is phosphate (PO3) and derivatives thereof; n is 2 or greater; B is a nucleobase; S is an acyclic moiety, a carbocyclic moiety, or sugar moiety; D1 is a detectable label that is attached to the terminal phosphate; and D2 is a detectable label that is attached to nucleobase; and wherein D1 and D2 do not significantly quench each other.

The present disclosure also provides methods for synthesis of dual labeled nucleotides using click chemistry. For example, disclosed herein is a method for synthesizing a dual labeled nucleotide, comprising: (a) introducing a terminal alkyne group onto the nucleobase of the nucleotide to form an alkynyl nucleotide; (b) attaching a first detectable label to the terminal phosphate of the nucleotide to form a labeled alkynyl nucleotide; and (c) reacting the terminal alkyne group of the nucleobase with a labeled azide compound comprising an azide group and a second detectable label, thereby forming a nucleotide comprising a first detectable label attached to the terminal phosphate and a second detectable label attached to the nucleobase. Also disclosed herein is alternative method for synthesizing a dual labeled nucleotide, comprising: (a) introducing a terminal azide group onto the nucleobase of the nucleotide to form a nucleotide azide; (b) attaching a first detectable label to the terminal phosphate of the nucleotide to form a labeled nucleotide azide; and (c) reacting the azide group of the nucleobase with a labeled alkyne compound comprising a terminal alkyne group and a second detectable label, thereby forming a nucleotide comprising a first detectable label attached to the terminal phosphate and a second detectable label attached to the nucleobase.

Also disclosed herein are improved DNA sequencing methods and compositions that provide for enhancement of the FRET signals associated with incorporation of a detectably labeled nucleotide and decreased background noise, thereby improving the detectability of sequence information and reliability of the determined sequence data. The present disclosure also provides a set of methodologies adapted to either increase acceptor signal strength and/or duration, decrease background noise or a combination of both increasing acceptor signal strength and/or duration and decreasing background noise. For example, disclosed herein are methods, systems and compositions for increasing the signal associated with the detectable label by increasing the amount of energy transferred to the acceptor and/or by increasing the time during which the acceptor is in close proximity to the donor.

Also disclosed are methods, systems and compositions for decreasing the background signal by optimizing surface chemistry.

Also disclosed herein are methods for decreasing the background signal by reducing acceptor fluorophore concentration via the use of novel nucleotides termed ‘star’ nucleotides, wherein two or more nucleotides are operably linked to, or otherwise associated with, a single detectable label.

Also disclosed herein is an exemplary discrete and ordered read strategy with the potential to resolve sequence order along the length of a DNA strand—ideally a strand the length of a chromosome. This strategy, termed ‘donor replacement sequencing” or “intercalation sequencing”, can additionally facilitate identification of structure and copy number variation.

Also disclosed herein are methods of screening polymerases for the ability to utilize gamma or omega-modified nucleotides; determining the incorporation efficiency for any given modified nucleotide; and monitoring the purity of labeled nucleotide stocks using thin layer chromatography (TLC) and/or electrophoretic methods.

Also disclosed herein are methods and compositions for purifying detectably labeled nucleotides from contaminating natural nucleotides using phosphatase treatments.

More particularly, disclosed herein are dendrimer compounds comprising a branched molecular structure containing multiple instances of a first linking capable of attachment to a nucleotide. In some embodiments, the compound further comprises a single instance of a second linking group capable of attachment to a detectable label. Also disclosed herein are methods for synthesizing a branched and labeled nucleotide compound using a dendrimer compound, comprising: (a) attaching a single dye moiety to a branched dendrimer compound, and (b) attaching multiple nucleotides to the dendrimers.

### BRIEF DESCRIPTION OF THE DRAWINGS

FIG. 1 illustrates the incorporation of labeled nucleotides into a polymerase with the expected fluorescent nucleotides signature.

FIG. 2 is a table illustrating cost of sequencing, assuming different base incorporation rates and read lengths, for a haploid human genome.

FIG. 3 illustrates a γ-phosphate labeled nucleotide.

FIG. 4 illustrates exemplary γ-phosphate labeled nucleotide synthetic schemes.

FIG. 5 illustrates results of primer extension assays using γ-phosphate labeled nucleotides, as detected using thin layer chromatography (TLC).

FIG. 6 illustrates γ-phosphate labeled nucleotide incorporations using various polymerases.

FIG. 7A illustrates a system for performing single molecule FRET sequencing using γ-phosphate labeled nucleotide.

FIG. 7B illustrates images of quadrants representing different dye signatures for the dyes Cy5, Rox and Alexa Fluor 488.

FIG. 8 illustrates single-pair FRET using labeled duplex.

FIG. 9A illustrates single-pair FRET using labeled duplex and a single donor type (Alexa Fluor 488) with 10 different acceptor dyes.

FIG. 9B illustrates confidence values for four dyes in the red and orange channel

FIG. 10 illustrates software routine for analyzing single pair FRET.

FIG. 11 illustrates single pair FRET evidencing incorporation of base-labeled nucleotides.

FIG. 12A illustrates event duration versus start time.

FIG. 12B illustrates normalized percent events versus duration in bar graph representation.

FIG. 13 illustrates real-time, on surface detection of sequential FRET events.

FIG. 14 illustrates detectability of 1-6 frame events.

FIG. 15 illustrates quantum dot dynamics for FRET detection.

FIG. 16 illustrates 150 real-time FRET events detected between polymerase and nucleotides.

FIG. 17 illustrates intercalating sequencing.

FIG. 18 illustrates intercalating sequencing.

FIGS. 19A-C illustrate active site design of Phi29 slowing incorporation chemistry.

FIGS. 20-59 and 59′ illustrate extension data for specific variants of Table 1.

FIG. 60 illustrates molecular structure designed to have a plurality of γ-phosphate labeled nucleotides attached thereto.

FIG. 61 illustrates exemplary click chemistry for the preparation of γ-phosphate labeled nucleotides.

FIG. 62 illustrates extension data for exemplary click modified γ-phosphate labeled nucleotides.

FIG. 63 illustrates an exemplary set of nucleotides designed to produce dual labeled nucleotides.

FIG. 64 illustrates extension data for the nucleotides of FIG. 63.

FIG. 65 illustrates extension data for the nucleotides of FIG. 63.

FIG. 66 illustrates extension data for the dual labeled nucleotides of FIG. 63.

FIG. 67 illustrates duplex/polymerase complexes, where the duplex is anchored to the polymerase.

FIG. 68 illustrates a sequencing composition and method for replenishing donor using donor labeled polymerases in solution and in surface-bound or immobilized duplexes.

FIG. 69 illustrates the detection of binding events that occur using the methodology of FIG. 68.

FIGS. 70A-D illustrate characterization data for various Phi29 variants of Table 1.

FIGS. 71A-C illustrate fidelity and disassociation assay data for various Phi29 variants of Table 1.

FIGS. 72A-C illustrate characterization of Phi29 variants on the detection system.

FIGS. 73A-E illustrate characterization of signal attributes.

FIG. 74 illustrates a bar graph of signals detected over time-profile.

FIGS. 75A-C illustrate on surface real time incorporation of gamma and base nucleotides.

FIG. 76 illustrates a synthetic scheme for quantum dot fluorescent donor for use in DNA sequencing.

FIGS. 77A-D illustrates extension data for the nucleotides of FIG. 63.

FIGS. 78A&B illustrate average donor image and segmented lamda DNA and average Acceptor image and segmented lambda DNA.

FIGS. 79A&B illustrate normalized donor profile, normalized acceptor profile, DNA lamda selection and sample profile.

FIGS. 80A&B illustrate representative FRET signals using two different Phi-29 variants.

FIG. 81 illustrates a ribbon model of Taq DNA polymerase, with the residue F647 highlighted in white and shown in ball-and-stick format.

FIG. 82 illustrates an exemplary method of synthesis for the dual-labeled nucleotide Alx594-dU3P-2-Cy5.

FIG. 83 illustrates the structures of exemplary dendrimer molecules that can be used to synthesize “star” molecules.

FIG. 84 illustrates the excitation, i.e., absorption, spectrum (solid line) and the emission spectrum (dashed line) for Cy5 (top panel) and Alexa Fluor 594 (bottom panel), respectively, plus a composite overlay of these spectra (middle panel).

FIG. 85 illustrates the structures of two exemplary dual labeled nucleotides, AF647-dU3P-22-AF680 (top) and AF647-dU3P-2-AF680.

FIG. 86 illustrates an exemplary method of synthesis of the dual labeled nucleotide AF647-dU3P-2-AF680.

FIG. 87 illustrates an exemplary method of synthesis of the dual labeled nucleotide AF647-dU3P-22-AF680.

FIG. 88 illustrates an exemplary method of synthesis of the dual labeled nucleotide AF647-dU4P-2-AF680.

FIG. 89 illustrates the structures of the products of enzymatic digestion of the dual labeled nucleotide Alx594-dU3P-2-Cy5.

FIG. 90 illustrates the results of analysis of the products of enzymatic digestion of the dual labeled nucleotide Alx594-dU3P-2-Cy5 using electrophoresis (left panel) and thin layer chromatography (TLC).

FIG. 91 illustrates the excitation and emission spectra for preparations of purified dual labeled nucleotides and analysis of the enzymatic digestion products of both single-labeled and dual-labeled nucleotides, as well as a mixture of the two.

### DETAILED DESCRIPTION

Unless defined otherwise, all technical and scientific terms used herein have the same meaning as is commonly understood by one of ordinary skill in the art to which these inventions belong. All patents, patent applications, published applications, treatises and other publications referred to herein, both supra and infra, are incorporated by reference in their entirety. If a definition and/or description is set forth herein that is contrary to or otherwise inconsistent with any definition set forth in the patents, patent applications, published applications, and other publications that are herein incorporated by reference, the definition and/or description set forth herein prevails over the definition that is incorporated by reference.

The practice of the present disclosure will employ, unless otherwise indicated, conventional techniques of molecular biology, microbiology and recombinant DNA techniques, which are within the skill of the art. Such techniques are explained fully in the literature. See, for example, Sambrook, J., and Russell, D. W., 2001, Molecular Cloning: A Laboratory Manual, Third Edition; Ausubel, F. M., et al., eds., 2002, Short Protocols In Molecular Biology, Fifth Edition.

Unless indicated otherwise, the reagents and solvents were obtained from Aldrich and used without further treatment. Full names of chemical compounds are provided for the first time with the abbreviation, and the latter is subsequently used in the remaining text.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps.

As used herein, the terms “a,” “an,” and “the” and similar referents are to be construed to cover both the singular and the plural unless their usage in context indicates otherwise. Accordingly, the use of the word “a” or “an” when used in conjunction with the term “comprising” in the claims or specification may mean “one,” but it is also consistent with the meaning of “one or more,” “at least one,” and “one or more than one.”

As used herein, the term “label” and its variants refer to any moiety that can be detected using suitable means, including but not limited to detection of fluorescence, luminescence, color, mass tag, radiation, magnetic resonance, energy transfer, reduction/oxidation potential and the like.

As used herein, the terms “linked”, “operably linked” and “operably bound” and variants thereof mean, for purposes of the specification and claims, to refer to fusion, bond, adherence or association of sufficient stability to withstand conditions encountered in single molecule applications and/or the methods and systems disclosed herein, between a combination of different molecules such as, but not limited to: between a detectable label and nucleotide, between a detectable label and a linker, between a nucleotide and a linker, between a protein and a functionalized nanocrystal; between a linker and a protein; and the like. For example, in a labeled polymerase, the label is operably linked to the polymerase in such a way that the resultant labeled polymerase can readily participate in a polymerization reaction. See, for example, Hermanson, G., 2008, Bioconjugate Techniques, Second Edition. Such operable linkage or binding may comprise any sort of fusion, bond, adherence or association, including, but not limited to, covalent, ionic, hydrogen, hydrophilic, hydrophobic or affinity bonding, affinity bonding, van der Waals forces, mechanical bonding, etc.

The term “linker” and its variants, as used herein, include any compound or moiety that can act as a molecular bridge that operably links two different molecules.

As used herein, the terms “nucleotide” and “nucleotide analog” and their variants refer to any compounds that can be polymerized and/or incorporated into a newly synthesized strand by a naturally occurring, genetically modified or engineered nucleotide polymerase, or a functional fragment thereof. Typically but not necessarily, the nucleotide or nucleotide analog comprises a nucleobase or derivative thereof; a sugar, acyclic or carbocyclic moiety or derivative thereof, and a phosphate chain comprising three, four, five or more phosphate groups or derivatives thereof. Examples of nucleotide compounds that may be used in the disclosed methods and compositions include, but are not limited to, ribonucleotides, deoxyribonucleotides, modified ribonucleotides, modified deoxyribonucleotides, ribonucleotide triphosphates, deoxyribonucleotide triphosphates, ribonucleotide polyphosphates comprising four or more phosphates, deoxyribonucleotide polyphosphates comprising four or more phosphates, modified ribonucleotide polyphosphates, modified deoxyribonucleotide polyphosphates, nucleoside triphosphates, nucleoside polyphosphates, peptide nucleotides, modified peptide nucleotides, and modified phosphate-sugar backbone nucleotides, and any derivatives, analogs or variants of the foregoing.

As used herein, the term “alpha phosphate” or “α-phosphate” and its variants refer to any phosphate group that is directly linked to the sugar moiety of a nucleotide.

As used herein, the term “beta phosphate” or “β-phosphate” and its variants refer to any phosphate group that is directly linked to the alpha phosphate of a nucleotide.

As used herein, the term “gamma phosphate” or “γ-phosphate” and its variants refer to any phosphate group that is directly linked to the beta phosphate of a nucleotide and that is not an alpha phosphate.

As used herein, the term “terminal phosphate” and its variants refer to any phosphate group that is located at the end, i.e., most distally from the sugar moiety, of a nucleotide phosphate chain.

As used herein, the term “terminally labeled nucleotide” and its variants refer to any nucleotide comprising a detectable label that is operably linked to, or otherwise associated with the terminal phosphate.

As used herein, the terms “nucleotide base” and “nucleobase” and their variants mean a substituted or unsubstituted nitrogen-containing parent heteroaromatic ring of a type that is commonly found in nucleic acids, as well as any natural, substituted, modified, or engineered variants or analogs of the same. Typically, the nucleobase is capable of forming Watson-Crick and/or Hoogsteen hydrogen bonds with an appropriately complementary nucleobase. Exemplary nucleobases include, but are not limited to, purines such as 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; base (Y); etc. Additional exemplary nucleobases can be found in Fasman, 1989, Practical Handbook of Biochemistry and Molecular Biology, pp. 385-394, CRC Press, Boca Raton, Fla., and the references cited therein. Typical nucleobases are purines, 7-deazapurines and pyrimidines. Typical nucleobases are the normal nucleobases, defined infra, and their common analogs, e.g., 2ms6iA, 6iA, 7-deaza-A, D, 2dmG, 7-deaza-G, 7mG, hypoxanthine, 4sT, 4sU and Y.

As used herein, the term “polymerase” means any molecule or molecular assembly that can polymerize a set of monomers into a polymer. The term “nucleotide polymerase” and its variants, as used herein, refer to any polymerase capable of polymerizing nucleotides, as defined above, into polynucleotides, including, without limitation, naturally occurring polymerases or reverse transcriptases, mutated, modified or engineered versions of naturally occurring polymerases or reverse transcriptases, where the mutation can involve the replacement of one or more or many amino acids with other amino acids, the insertion or deletion of one or more or many amino acids from the polymerases or reverse transcriptases, or the conjugation of parts of one or more polymerases or reverse transcriptases, non-naturally occurring polymerases or reverse transcriptases. The term polymerase also embraces synthetic molecules or molecular assembly that can polymerize a polymer having a pre-determined sequence of monomers, or any other molecule or molecular assembly that may have additional sequence tags that facilitate purification and/or immobilization and/or molecular interaction of the tags, and that can polymerize a polymer from monomer subunits.

The terms “nucleic acid polymerization”, “DNA polymerization” and “RNA polymerization” and their variants, as used herein, refers to a series of multiple nucleotide incorporation events onto the terminal 3′OH of a single nucleotide strand by a polymerase. By way of a non-limiting example of polynucleotide polymerization, the steps or events of DNA polymerization are well known and comprise: (1) complementary base-pairing a target DNA molecule with a DNA primer molecule having a terminal 3′ OH (the terminal 3′ OH provides the polymerization initiation site for DNA polymerase); (2) binding the base-paired target DNA/primer with a DNA-dependent polymerase to form an complex (e.g., open complex); (3) a candidate nucleotide binds with the DNA polymerase which interrogates the candidate nucleotide to determine if it is complementary with the nucleotide on the target DNA molecule; (4) the DNA polymerase undergoes a conformational change (e.g., to a closed complex if the candidate nucleotide is complementary); (5) the terminal 3′ OH of the primer exerts a nucleophilic attack on the bond between the α and β phosphates of the candidate nucleotide to mediate a nucleotidyl transferase reaction resulting in phosphodiester bond formation between the terminal 3′ end of the primer and the candidate nucleotide (i.e., nucleotide incorporation in a template-dependent manner), and concomitant cleavage and liberation of a polyphosphate product.

A nucleotide incorporation event refers to the incorporation of a single nucleotide onto the terminal 3′OH of a newly synthesized nucleic acid molecule by a polymerase. Typically, the incorporation event involves covalent attachment of the nucleotide to the terminal 3′OH of the newly synthesized nucleic acid molecule. As used herein, the term “nucleotide incorporation event” further comprises events starting from binding the candidate nucleotide with the DNA polymerase (as part of the complex), and includes all events through and including phosphodiester bond formation, and concomitant cleavage and release of the polyphosphate product.

As used herein, the term “alkyne”, “alkynyl” and their variants refer to any compound or moiety comprising at least one triple bond between two carbon atoms.

As used herein, the term “azide”, “azido” and their variants refer to any moiety or compound comprising the monovalent group —N₃ or the monovalent ion —N₃.

As used herein, the term “carboxyl” and its variants refer to any moiety or compound comprising a carboxyl group, which has the formula —C(═O)OH, usually written —COOH or —CO₂H.

As used herein, the term “amine”, “amino” “amido” and their variants refer to any moiety or compound comprising a group derived from ammonia by replacing hydrogen atoms by univalent hydrocarbon radicals.

Disclosed herein are compositions, methods and systems for single molecule sequencing via detection of signals emitted by labels attached to various components of a nucleotide polymerase reaction. The term “nucleotide polymerase reaction”, as used herein, means any mixture comprising a polymerase and one or more nucleotides wherein the polymerase incorporates one or more nucleotides onto the 3′OH of a newly synthesized nucleic acid molecule. Typically, in a given nucleotide polymerase reaction the polymeric molecule of interest (referred to as the “template”) is contacted with a reaction mixture comprising a polymerase and individual nucleotides capable of polymerization by the polymerase. Signals emitted by one or more detectable labels linked or attached to one or more components of the nucleotide polymerase reaction are detected and analyzed to determine a time sequence of nucleotide incorporation events.

In some embodiments, one of the detectable labels is operably linked or otherwise attached to the nucleotide polymerase of the nucleotide polymerase reaction. Any detectable label is suitable for attachment to the polymerase may be used, such as a chromophore, luminophore, or fluorophore capable of acting as a FRET donor. In some embodiments, the polymerase is conjugated or otherwise operably linked to a semiconductor nanocrystal. The label may be operably linked to the polymerase using any suitable method that preserves the ability of the polymerase to catalyze a polymerization reaction.

In some embodiments, the signals emitted and monitored during the nucleotide polymerase reaction are the result of Forster Resonance Energy Transfer (FRET). FRET occurs when two appropriately labeled molecules or moieties are sufficiently proximal to each other to transfer energy. During a FRET reaction, a first, excited moiety, called a FRET donor, non-radiatively transfers energy to a second moiety, called a FRET acceptor, which may then emit a detectable signal, called a FRET signal. A FRET donor is any moiety that is capable of transferring energy via FRET with a suitable acceptor. A FRET acceptor is any moiety that is capable of receiving energy via FRET from a suitable FRET donor.

In some embodiments, sequencing is accomplished via monitoring single-pair Fërster resonance energy transfer (spFRET) between a FRET donor operably linked to or otherwise associated with the polymerase, the primer-template duplex or the immobilization matrix, and a FRET acceptor operably linked to or otherwise associated with any suitable component of the sequencing machinery, for example the nucleotide. Typically, the FRET donor is operably linked or otherwise attached to the polymerase, and the FRET acceptor is operably linked or otherwise attached to the incoming nucleotide. The donor-labeled polymerase molecule attaches to priming sites within the polymeric template, and then binds to an incoming nucleotide in a template-dependent fashion. When the polymerase binds to the incoming nucleotide, the FRET donor attached to the polymerase is brought into proximity with the FRET acceptor of the monomer and FRET occurs, resulting in localized and detectable FRET emission events that permit monitoring of each localized sequencing reaction in situ. As the polymerase extends the newly synthesized strand by adding labeled nucleotides to the free 3′ end of the strand in a template-dependent fashion, the identity of each successive incoming nucleotide bound and incorporated by the polymerase will be identifiable by the emission spectrum of the FRET acceptor attached to that particular nucleotide. Accordingly, the nucleotide can be identified by optical detection and characterization of the FRET signal, as described below.

Typically, the detectable label of the nucleotide is attached to a phosphate of the nucleotide monomer that is released upon incorporation into the primer strand, for example the gamma or terminal (omega) phosphate of the polyphosphate chain of the nucleotide, which upon polymerization by the polymerase releases a labeled polyphosphate into the surrounding environment. In certain embodiments, the label is attached to a portion of the nucleotide that is cleaved by the polymerase from the nucleotide before, during or after nucleotide incorporation, for example the β-phosphate, the γ-phosphate, or the terminal phosphate of the incoming nucleotide. Such labels are termed “non-persistent” because they do not become incorporated into the nascent nucleic acid molecule synthesized by the polymerase. Upon cleavage of the phosphate during nucleotide incorporation and consequent release of the label, the FRET signal between the quantum dot and the label ceases after the nucleotide is incorporated and the label diffuses away. Thus, in these embodiments, a FRET signal is generated as each incoming nucleotide hybridizes to a complementary nucleotide in the target nucleic acid molecule, and upon incorporation of the nucleotide into the elongating primer strand, the label is released and the FRET signal ends. By releasing the label upon incorporation, successive extensions can each be detected without interference from nucleotides previously incorporated into the complementary strand. Alternatively, the nucleotide may not terminally-labeled, but rather labeled with a “persistent” label on an internal phosphate, for example, the alpha phosphate or another internal phosphate. Such labels are termed “persistent” because they will become incorporated into the nascent nucleic acid molecule synthesized by the polymerase, thus producing a lasting signal that continues after nucleotide incorporation is completed.

One advantage of the use of detectably labeled nucleotides according to the present disclosure is the increased accuracy of the resulting sequence information as compared to information gathered through methods using unlabeled nucleotides. As described more fully in U.S. Pat. No. 7,211,414 and U.S. application Ser. No. 11/648,107, when fidelity of nucleotide incorporation is assayed by performing extensions with 4-color, detection-system-relevant γ-nucleotides, it can be determined that a slightly lower error rate is associated with the γ-modified nucleotide reactions (99.97% correct), relative to natural nucleotide reactions (99.92% correct). This fidelity analysis indicated that these γ-modified nucleotides were accurately incorporated and that modification of the γ-phosphate may be a general mechanism to affect the accuracy at which a nucleotide is incorporated.

In some embodiments, the labeled nucleotide has three, four or more phosphates.

In the single molecule applications disclosed herein, the polymeric molecule to be sequenced is typically a nucleic acid. Suitable nucleic acid molecules that can be sequenced according to the present disclosure include without limitation single-stranded DNA, double-stranded DNA, single stranded DNA hairpins, DNA/RNA hybrids, RNA with an appropriate polymerase recognition site, and RNA hairpins. In a typical embodiment, the polymer is DNA, the polymerase is a DNA polymerase or an RNA polymerase, and the labeled monomer is a nucleotide. In another embodiment, the polymer to be sequenced is RNA and the polymerase is reverse transcriptase.

The polymerase can be any suitable naturally occurring, mutated, modified or engineered polymerase, including any variants or fragments of the same, that is capable of polymerizing monomeric subunits into polymers. Typically, the polymerase is a nucleotide polymerase, i.e., a polymerase that can polymerize nucleotides. The polymerase may be an entire intact and native nucleotide polymerase; alternatively, it can be a fragment, fragment combination, mutant or other derivative of a polymerase that retains the ability to polymerize monomers. Typically, the polymerase will elongate a pre-existing polynucleotide strand, typically a primer, by polymerizing nucleotides on to the 3′ end of the strand. Exemplary polymerases include without limitation DNA polymerases, RNA polymerases and reverse transcriptases. Suitable nucleotide polymerases include, without limitation, any naturally occurring nucleotide polymerases as well as mutated, truncated, modified, genetically engineered or fusion variants of such polymerases. Known conventional naturally occurring DNA polymerases include without limitation bacterial DNA polymerases, eukaryotic DNA polymerases, archaeal DNA polymerases, viral DNA polymerases and phage DNA polymerases. Suitable bacterial DNA polymerase include without limitation E. coli DNA polymerases I, II and III, IV and V, the Klenow fragment of E. coli DNA polymerase. Suitable eukaryotic DNA polymerases include without limitation the DNA polymerases α, δ, ε, η, ζ, β, σ, λ, μ, τ, and κ, as well as the Rev1 polymerase (terminal deoxycytidyl transferase) and terminal deoxynucleotidyl transferase (TdT). Suitable viral DNA polymerases include without limitation T4 DNA polymerase, Phi29 DNA polymerase and T7 DNA polymerase. Suitable archaeal DNA polymerases include without limitation the thermostable and/or thermophilic DNA polymerases such as, for example, DNA polymerases isolated from Thermus aquaticus (Taq) DNA polymerase and the like or Vent DNA polymerase, Pyrococcus sp. GB-D polymerase, “Deep Vent” DNA polymerase, New England Biolabs) Similarly, suitable RNA polymerases include, without limitation, T7, T3 and SP6 RNA polymerases. Suitable reverse transcriptases include without limitation reverse transcriptases from HIV, HTLV-I, HTLV-II, FeLV, FIV, SIV, AMV, MMTV and MoMuLV, as well as the commercially available “Superscript” reverse transcriptases, (Invitrogen) and telomerases. In addition to naturally occurring polymerases, the polymerase peptides disclosed herein may also be derived from any subunits, mutated, modified, truncated, genetically engineered or fusion variants of naturally occurring polymerases (wherein the mutation involves the replacement of one or more or many amino acids with other amino acids, the insertion or deletion of one or more or many amino acids, or the conjugation of parts of one or more polymerases) non-naturally occurring polymerases, synthetic molecules or any molecular assembly that can polymerize a polymer having a pre-determined or specified or templated sequence of monomers may be used in the methods disclosed herein. For example, incorporation of gamma-labeled nucleotides has been achieved using HIV reverse transcriptase as well as modified versions of E. coli DNA polymerase I and Phi-29 polymerase to achieve processive DNA synthesis (data not shown).

Optionally, the FRET donor moiety is operably linked to the polymerase using any suitable methods that preserve polymerase activity and the ability of the donor to undergo FRET with an incoming acceptor-labeled nucleotide. For example, the polymerase may be selected to be deficient in solvent exposed cysteine residues. Alternatively, the polymerase may be engineered to contain an N-terminal tag to serve as the site for donor fluorophore attachment and/or immobilization. Sites for cysteine introduction and subsequent fluorophore labeling include positions that are within close proximity (less than 35 Å) of the gamma-phosphate on an incoming nucleotide and replace either serine or threonine to avoid significant alterations in the protein structure. Donor fluorophores are not placed within the polymerase's active site, as this may hinder enzyme function.

In some embodiments, at least one component of the nucleotide polymerase reaction, such as the polymerase, oligonucleotide primer, or template is immobilized. In one embodiment, the FRET donor is operably linked to, or otherwise associated with, an immobilized polymerase. This embodiment may yield more consistent spFRET signals than embodiments wherein the donor is linked to a specific site on the primer/template duplex (which increases the distance between the donor-acceptor with each nucleotide insertion and produces less consistent signals). A donor-labeled, immobilized polymerase maintains a constant distance between the donor and acceptor during nucleotide incorporation, producing high FRET with consistent intensity signatures, and positions the nanomachine within the illuminated volume at a relatively constant and higher energy position near the surface. Together, these consistencies minimize data analysis complexity and facilitate longer sequence reads. Alternatively, the FRET donor may be located on the primer-template duplex, thereby obviating the need to use a detectably labeled polymerase.

When conducting FRET-based sequencing according to the methods described herein, donor-acceptor pairs are typically selected such that there is sufficient overlap between the emission spectrum of the donor and excitation spectrum of the acceptor for detectable FRET to occur. Any suitable FRET donor:acceptor pair may be used in the disclosed methods and compositions, including but not limited to a fluorescein, cyanine, rhodamine, coumarin, acridine, Texas Red dye, BODIPY, Alexa Fluor, GFP, rhodol, ROX, Tokyo Green, resorufin or a derivative or modification of any of the foregoing. See, for example, U.S. Pub. No. 2008/0091995. This approach is directly influenced by distance—both for maximizing signal and minimizing background—because FRET efficiency (FE) is an inverse function of the 6th power of the distance between donor and acceptor fluorophores (Förster 1948; Stryer and Haugland 1967; Stryer 1978; Dale, Eisinger et al. 1979; Clegg, Murchie et al. 1993; Selvin 2000; Weiss 2000).

Although the energy transfer from the donor to the acceptor does not involve emission of light, it may be thought of in the following terms: excitation of the donor produces energy in its emission spectrum that is then picked up by the acceptor in its excitation spectrum, leading to the emission of light from the acceptor in its emission spectrum. In effect, excitation of the donor sets off a chain reaction, leading to emission from the acceptor when the two are sufficiently close to each other.

In addition to spectral overlap between the donor and acceptor, other factors affecting FRET efficiency include the quantum yield of the donor and the extinction coefficient of the acceptor. The FRET signal may be maximized by selecting high yielding donors and high absorbing acceptors, with the greatest possible spectral overlap between the two. Additional information on FRET and parameters affecting FRET efficiency and signal detection may be found in Piston, D. W., and Kremers, G. J., 2007, Trends Biochem. Sci., 32:407.

Typically, the sequencing reaction is initiated by the addition of a suitable polymerase and labeled nucleotides to a nucleic acid template molecule comprising one or more priming sites. Suitable temperatures and the addition of other components such as divalent metal ions can be determined and optimized based on the particular nucleotide polymerase and the target nucleic acid sequences. Illumination of the reaction site permits observation of the FRET reactions that mark the nucleotide incorporation.

Any suitable reaction conditions may be employed for the nucleotide polymerase reaction that permit binding of the polymerase to a nucleotide in a template-dependent fashion. In one example, reaction conditions for the Klenow fragment of DNA polymerase I typically include a buffer comprising 50 mM Tris HCl, 10 mM MgCl₂ and 50 mM NaCl at pH 8.0, incubated at room temperature to 37° C. See, for example, Sambrook, J., and Russell, D. W., 2001, Molecular Cloning: A Laboratory Manual, Third Edition, or Ausubel, F. M., et al., eds., 2002, Short Protocols In Molecular Biology, Fifth Edition.

The initiation site for sequencing can be created through any suitable means. In some embodiments, the polymer to be sequenced comprises, or is associated with, a polymerase priming site capable of extension via polymerization of monomers by the polymerase. The priming site may be generated, for example, by treatment of the polymer so as to produce nicks or cleavage sites. Alternatively, a priming site may be generated by any other suitable methods, such as, for example, by annealing the polymer to a complementary primer that can be extended by the polymerase. Yet another option is for the target polymer to undergo “hairpin” formation, either through annealing to a self-complementary region within the target sequence itself or through ligation to a self-complementary sequence, resulting in a structure that undergoes self-priming under suitable conditions.

In one embodiment, a suitable primer is included in the nucleic acid polymerase reaction. The primer length is typically determined by the specificity desired for binding the complementary template as well as the stringency of the annealing and reannealing conditions employed. The primer can comprise ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, and modified phosphate-sugar backbone nucleotides, and any analogs or variants of the foregoing compounds. The primer can be synthetic, or produced naturally by primases, RNA polymerases, or other oligonucleotide synthesizing enzymes. The primer may be any suitable length including at least 5 nucleotides, 5 to 10, 15, 20, 25, 50, 75, 100 nucleotides or longer in length. In a typical embodiment, the polymerase extends the primer by a plurality of nucleotides. Optionally, the primer is extended at least 50, 100, 250, 500, 1000, or at least 2000 nucleotide monomers.

According to the present disclosure, one, some or all of the components of the polymerase reaction can be operably linked to any suitable detectable label, including a donor-labeled polymerase or oligonucleotide primer, and acceptor-labeled nucleotides, using suitable methods. See, for example, Hermanson, G., 2008, Bioconjugate Techniques, Second Edition. Suitable linkers include, for example, any compound or moiety that can act as a molecular bridge to operably link two different molecules. Any suitable linker may be used to operably link suitable groups, moieties or molecules to form the sequencing compositions disclosed herein. Typically but not necessarily the linker will be covalently attached to one, some or all of the linked moieties. Exemplary linkers include, but are not limited to, chemical chains, chemical compounds (e.g., reagents), and the like. The linkers may include, but are not limited to, homobifunctional linkers and heterobifunctional linkers. For example, heterobifunctional linkers contain one end having a first reactive functionality to specifically link to a first molecule, and an opposite end having a second reactive functionality to specifically link to a second molecule. Depending on such factors as the molecules to be linked and the conditions in which the method of strand synthesis is performed, the linker may vary in length and composition for optimizing properties such as stability, length, FRET efficiency, resistance to certain chemicals and/or temperature parameters, and be of sufficient stereo-selectivity or size to operably link a nanocrystal or a label to a polymerase or nucleotide such that the resultant conjugate is useful in optimizing a polymerization reaction. Linkers can be employed using standard chemical techniques and include but not limited to, amine linkers for attaching labels to nucleotides (see, for example, U.S. Pat. No. 5,151,507); a linker typically contain a primary or secondary amine for operably linking a label to a nucleotide; and a rigid hydrocarbon arm added to a nucleotide base (see, for example, Science 282:1020-21, 1998).

In some embodiments, the linker comprises reactive groups suitable for forming attachments to the moieties to be linked. Exemplary reactive groups include without limitation hydroxyl, sulfhydryl, amino, haloalkyl, azido, propargyl, carboxyl and acetylene groups. In some embodiments, the attachments formed between the linker and the linked moiety may comprise alkyl, hydroxyl, sulfhydryl, amino, haloalkyl, azido, amido, propargyl, carboxyl, alkene and alkyne bonds. Some examples of suitable linkers are disclosed in Hardin et al., Ser. No. 11/007,794; Wang et al., Ser. No. 11/781,160; Wang et al., 60/891,029. These documents also describe linker variants and associated synthesis chemistries to attach a variety of appropriate acceptor fluorophores to the gamma- or terminal phosphate. Such linkers may be rationally designed to minimally impact polymerase function.

Typically, donor and acceptor fluorophores can be chosen with regard to enzyme compatibility and their spectral and photophysical properties. In some embodiments, the donor is a very stable, high quantum yield, blue-green fluorophore that does not interfere with enzyme activity. In some embodiments, the acceptor is a high quantum yield yellow-red fluorophores, with large molar extinction coefficients at wavelengths near the peak of the donor emission spectrum. Typically, the acceptor fluorophore is selected or modified to ensure that it does not display significant absorption at the excitation wavelength of the donor fluorophore, and that the emission spectra of the donor and acceptor fluorophores do not significantly overlap. In some embodiments, each of four different nucleotides is labeled with one of four different types of acceptor, and all four acceptor types undergo efficient FRET with the donor and can be unambiguously resolved via their emission properties.

In some embodiments, the polymerase and/or nucleotides are engineered to undergo maximum FRET (characterized by anti-correlated donor and acceptor signals) when the acceptor-labeled nucleotide docks within the polymerase active site. During nucleotide insertion, the 3′ end of the primer attacks the alpha phosphate within the nucleotide, cleaving the bond between the alpha- and beta-phosphates and also possibly changing the spectral properties of the FRET acceptor (which, if originally attached to a releasable portion of the incoming nucleotide, such as the gamma or terminal phosphate group of a nucleotide polyphosphate, remains attached to the released pyrophosphate (PP) or polyphosphate moiety, as the case may be). One advantage of using such non-persistent labels is, because the nucleotides are fluorescently modified at the gamma- or terminal phosphate and the label is released before, during or after nucleotide incorporation, a native DNA polymer is produced from the polymerization, rather than a highly modified polymer that could negatively impact polymerase activity.

Optionally, the sequence applications disclosed herein may incorporate suitable methods of minimizing sequencing errors arising from contamination of the detectably labeled nucleotide sample with natural, i.e., unlabeled, nucleotides. Such natural nucleotides may be present in trace amounts as a remnant of labeled nucleotide synthesis or as by-product of labeled nucleotide degradation during storage. Sequencing based on detection of spFRET events associated with nucleotide incorporation requires the use of detectably labeled nucleotides, whereas polymerases tend to preferentially incorporate natural (i.e., unlabeled) nucleotides. Incorporation of the contaminating unlabeled nucleotide will not produce a spFRET event, resulting in the loss of sequence information or, worse, an apparent deletion at that site. Optionally the labeled nucleotide stocks may be subjected to an enzymatic treatment prior to inclusion in the sequencing reaction to eliminate potential problems arising from the presence of contaminating natural nucleotides. For example, Hardin et al., U.S. application Ser. No. 11/007,794 discloses methods to treat such stocks with a phosphatase, such as calf intestinal alkaline phosphatase (CIAP) or shrimp alkaline phosphatase (SAP), that preferentially hydrolyzes natural nucleotides. Additionally, inclusion of phosphatase in the extension reaction destroys labeled pyrophosphate or polyphosphate produced during nucleotide incorporation, thereby minimizing pyrophosphorolysis (reverse polymerization) and improving sequence data accuracy.

In other embodiments, the label operably linked or attached to the nucleotide may be a quencher. Quenchers are useful as acceptors in FRET applications, because they produce a signal through the reduction or quenching of fluorescence from the donor fluorophore. As with conventional fluorescent labels, quenchers have an absorption spectrum and large extinction coefficients, however the quantum yield for quenchers is extremely reduced, such that the quencher emits little to no light upon excitation. For example, in a FRET detection system, illumination of the donor fluorophore excites the donor, and if an appropriate acceptor is not close enough to the donor, the donor emits light. This light signal is reduced or abolished when FRET occurs between the donor and a quencher acceptor, resulting in little or no light emission from the quencher. Thus, interaction or proximity between a donor and quencher-acceptor may be detected by the reduction or absence of donor light emission. For an example of the use of a quencher as an acceptor with a nanocrystal donor in a FRET system, see Medintz, I L et al. (2003) Nat. Mater. 2:630, herein incorporated by reference in its entirety. Examples of quenchers include the QSY dyes available from Molecular Probes (Eugene, Oreg.).

One exemplary method involves the use of quenchers in conjunction with fluorescent labels. In this strategy, certain nucleotides in the reaction mixture are labeled with a fluorescent label, while the remaining nucleotides are labeled with one or more quenchers. Alternatively, each of the nucleotides in the reaction mixture is labeled with one or more quenchers. Discrimination of the nucleotide bases is based on the wavelength and/or intensity of light emitted from the FRET acceptor, as well as the intensity of light emitted from the FRET donor. If no signal is detected from the FRET acceptor, a corresponding reduction in light emission from the FRET donor indicates incorporation of a nucleotide labeled with a quencher. The degree of intensity reduction may be used to distinguish between different quenchers.

Another exemplary method involves modulating FRET efficiency by varying the distance between the nanocrystal donor and the fluorescent label or quencher acceptor. In this strategy, the same type of fluorescent label or quencher may be used, however, the distance between the nanocrystal and the label is varied for each nucleotide to be identified, causing a modulation of FRET efficiency. The distance may be varied through the structure of the nucleotide itself, the position of the fluorescent label or quencher on the nucleotide, or the use of spacers or linkers during attachment of the fluorescent label or quencher to the nucleotide. Modulation of FRET efficiency results in a detectable modulation of emission intensity and/or quenching.

In another strategy, FRET efficiency may be modulated by varying the number of fluorescent labels or quenchers attached to each incoming nucleotide. In this strategy, differing numbers of the same fluorescent label or quencher are attached to each nucleotide. For example, one fluorescent label may be attached to A, two to T, three to G, and four to C. Increasing the number of acceptors relative to the nanocrystal donors increases FRET efficiency and quantum yield, such that base discrimination may be based on the intensity of light emission from the acceptor(s) or the reduction of light emission from the nanocrystal donor(s).

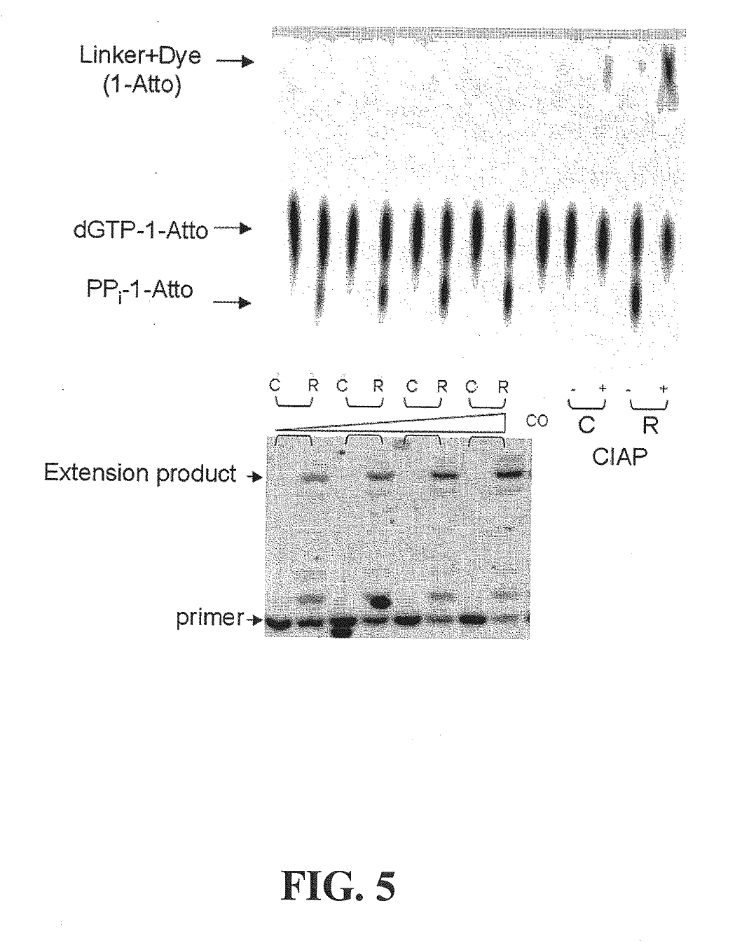

FIG. 5 depicts an exemplary method wherein the gamma labeled nucleotide dGTP-1-Atto was treated with SAP to remove natural nucleotides, following which SAP was heat inactivated. The SAP-treated nucleotide was then used in primer extension reactions with a homopolymeric C template for increasing amounts of time (indicated with a triangle). A portion of each reaction was examined via TLC (top) and denaturing gel electrophoresis (bottom). Reaction products are indicated at left. ‘C’ indicates that the reaction was assembled without polymerase (control). ‘R’ indicates a complete reaction. Note that the Atto-PPᵢ product is only observed in a complete reaction (R), and that its intensity increases with increased reaction time. Intensity of the 7-base extension products mirrors the accumulation of the Atto-PPᵢ. ‘CO’ indicates a co-spotting of a control reaction and a complete reaction using natural dGTP to show that the PPᵢ spot in the reaction lanes is not a TLC artifact. Following extension the reactions were treated with CIAP (+) to destroy the released PPᵢ, thereby confirming the identity of the new TLC spot.

Any suitable methods may be used to detect and analyse FRET signals to determine whether a nucleotide incorporation has taken place, and optionally to determine the nucleobase identity of the incoming nucleotide. For example, signals from a non-persistent acceptor attached to the nucleotide may be detected and analyzed to determine base identity. Donor fluorescence is equally informative, as it is anti-correlated with acceptor fluorescence throughout the incorporation reaction. After an spFRET event, the donor's emission returns to its original state and is ready to undergo a similar intensity oscillation cycle with the next acceptor-labeled nucleotide. In this way, the emissions from the donor fluorophore act as a punctuation mark between nucleotide incorporation events. As is demonstrated below, the increase in donor fluorescence between incorporations is especially important during analysis of homopolymeric sequences.

Additionally, in certain instances it is useful to perform reactions with reference controls, similar to microarray assays. Comparison of signal(s) between the reference sequence and the test sample are used to identify differences and similarities in sequences or sequence composition. Such reactions can be used for fast screening of DNA polymers to determine degrees of homology between the polymers, to determine polymorphisms in DNA polymers, or to identity pathogens.

In some embodiments, the method further comprises sequencing one or more additional nucleic acid molecules, for example a second nucleic acid, in parallel with sequencing the first nucleic acid. In other embodiments, the rate of nucleotide sequencing determination (based on a single read of a nucleic acid template) is equal to or greater than 1 nucleotide per second, 10 nucleotides per second, or 100 nucleotides per second.

Typically, the sequencing error rate will be equal to or less than 1 in 100,000 bases. In some embodiments, the error rate of nucleotide sequence determination is equal to or less than 1 in 10 bases, 1 in 20 bases, 3 in 100 bases, 1 in 100 bases, 1 in 1000 bases, and 1 in 10,000 bases. In another preferred embodiment, the test DNA will comprise a complete and intact chromosome. Optionally, the methods disclosed herein may be performed in a multiplex fashion (including in array format), such that additional nucleic acid molecules are sequenced in parallel with a first nucleic acid molecule.

The signals emitted by various components of the polymerase reaction mixture as the polymerase incorporates nucleotide(s) into an elongating strand in a template-directed fashion can be detected by means of any suitable system capable of detecting and/or monitoring such signals. Typically, the optical system will achieve these functions by first generating and transmitting an incident wavelength to the polynucleotides isolated within nanostructures, and then collecting and analyzing the emissions from the reactants.

The optical system applicable for the present invention comprises at least two elements, namely an excitation source and a detector. The excitation source generates and transmits incident radiation used to excite the reactants contained in the array. Depending on the intended application, the source of the incident light can be a laser, laser diode, a light-emitting diode (LED), a ultra-violet light bulb, and/or a white light source. Where desired, more than one source can be employed simultaneously. The use of multiple sources is particularly desirable in applications that employ multiple different reagent compounds having differing excitation spectra, consequently allowing detection of more than one fluorescent signal to track the interactions of more than one or one type of molecules simultaneously.

Any suitable detection strategies can be employed to determine the identity of the nitrogenous base of the incoming nucleotides, depending on the nature of the labeling strategy that is employed. Exemplary labeling and detection strategies include but are not limited to those disclosed in U.S. Pat. Nos. 6,423,551 and 6,864,626; U.S. Pub. Nos. 2005/0003464, 2006/0176479, 2006/0177495, 2007/0109536, 2007/0111350, 2007/0116868, 2007/0250274 and 2008/08825. Detection of emissions during the polymerization reaction permits the discrimination of independent interactions between uniquely labeled moieties, reactants or subunits. On exposure to suitable chemical, electrical, electromagnetic energy (potentially any light source, typically a laser) or upon resonance as in FRET, the label linked to the nucleotide undergoes a transition to an ‘excited state’ whereby it emits photons over a spectral range characterized by the identity of the emitting moiety. The donor moiety must be sufficiently excited in order for FRET to occur.

Emissions may be detected using any suitable device. A wide variety of detectors are available in the art. Representative detectors include but are not limited to optical readers, high-efficiency photon detection systems, photodiodes (e.g. avalanche photo diodes (APD); APD arrays, etc.), cameras, charge couple devices (CCD), electron-multiplying charge-coupled device (EMCCD), intensified charge coupled device (ICCD), photomultiplier tubes (PMT), a muti-anode PMT, and a confocal microscope equipped with any of the foregoing detectors. Where desired, the subject arrays contain various alignment aides or keys to facilitate a proper spatial placement of each spatially addressable array location and the excitation sources, the photon detectors, or the optical transmission element as described below.

Typically, characteristic signals from different independently labeled, nucleotides are simultaneously detected and resolved using a suitable detection method capable of discriminating between the respective labels. Typically, the characteristic signals from each nucleotide are distinguished by resolving the characteristic spectral properties of the different labels. See, for example, Lakowitz, J. R., 2006, Principles of Fluorescence Spectroscopy, Third Edition. Spectral detection may also optionally be combined and/or replaced by other detection methods capable of discriminating between chemically similar or different labels in parallel, including, but not limited to, polarization, lifetime, Raman, intensity, ratiometric, time-resolved anisotropy, fluorescence recovery after photobleaching (FRAP) and parallel multi-color imaging. See, for example, Lakowitz, supra. In the latter technique, use of an image splitter (such as, for example, a dichroic mirror, filter, grating, prism, etc.) to separate the spectral components characteristic of each label is preferred to allow the same detector, typically a CCD, to collect the images in parallel. Optionally, multiple cameras or detectors may be used to view the sample through optical elements (such as, for example, dichroic mirrors, filters, gratings, prisms, etc.) of different wavelength specificity. Other suitable methods to distinguish emission events include, but are not limited to, correlation/anti-correlation analysis, fluorescent lifetime measurements, anisotropy, time-resolved methods and polarization detection. Suitable imaging methodologies that may be implemented for detection of emissions include, but are not limited to, confocal laser scanning microscopy, Total Internal Reflection (TIR), Total Internal Reflection Fluorescence (TIRF), near-field scanning microscopy, far-field confocal microscopy, wide-field epi-illumination, light scattering, dark field microscopy, photoconversion, wide field fluorescence, single and/or multi-photon excitation, spectral wavelength discrimination, evanescent wave illumination, scanning two-photon, scanning wide field two-photon, Nipkow spinning disc, multi-foci multi-photon, and/or other forms of microscopy.

The detection system may optionally include one or more optical transmission elements that serve to collect and/or direct the incident wavelength to the reactant array; to transmit and/or direct the signals emitted from the reactants to the photon detector; and/or to select and modify the optical properties of the incident wavelengths or the emitted wavelengths from the reactants. Illustrative examples of suitable optical transmission elements and optical detection systems include but are not limited to diffraction gratings, arrayed wave guide gratings (AWG), optic fibers, optical switches, mirrors, lenses (including microlens and nanolens), collimators. Other examples include optical attenuators, polarization filters (e.g., dichroic filters), wavelength filters (low-pass, band-pass, or high-pass), wave-plates, and delay lines.

Typically, the detection system comprises optical transmission elements suitable for channeling light from one location to another in either an altered or unaltered state. Non-limiting examples of such optical transmission devices include optical fibers, diffraction gratings, arrayed waveguide gratings (AWG), optical switches, mirrors, (including dichroic mirrors), lenses (including microlens and nanolens), collimators, filters, prisms, and any other devices that guide the transmission of light through proper refractive indices and geometries.

In one embodiment, the detection system comprises an optical train that directs signals from an organized array onto different locations of an array-based detector to simultaneously detect multiple different optical signals from each of multiple different locations. In particular, the optical trains typically include optical gratings and/or wedge prisms to simultaneously direct and separate signals having differing spectral characteristics from each spatially addressable location in an array to different locations on an array-based detector, e.g., a CCD. By separately directing signals from each array location to different locations on a detector, and additionally separating the component signals from each array location, one can simultaneously monitor multiple signals from each array location.

In a preferred embodiment, detection is performed using multifluorescence imaging wherein each of the different types of nucleotide is operably linked to a label with different spectral properties from the rest, thereby permitting the simultaneous detection of incorporation of all different nucleotide types. For example, each of the different types of nucleotide may be operably linked to a FRET acceptor fluorophore, wherein each fluorophore has been selected such that the overlapping of the absorption and emission spectra between the different fluorophores, as well as the the overlapping between the absorption and emission maxima of the different fluorophores, is minimized. Detection of different nucleotide label is performed by observing two or more targets at the same time, wherein the emissions from each label are separated in the detection path. Such separation is typically accomplished through use of suitable filters, including but not limited to band pass filters, image splitting prisms, band cutoff filters, wavelength dispersion prisms and dichroic mirrors, hat can selectively detect specific emission wavelengths. Such filters may optionally be used in combination with suitable diffraction gratings.

Alternatively, multifluorescence studies involving differently labeled nucleotide types may be performed by observing each label separately, requiring section of special filter combinations for each excitation line and each emission band. In one embodiment, the detection system utilizes tunable excitation and/or tunable emission fluorescence imaging. For tunable excitation, light from a light source passes through a tuning section and condenser prior to irradiating the sample. For tunable emissions, emissions from the sample are imaged onto a detector after passing through imaging optics and a tuning section. The user may control the tuning sections to optimize performance of the system.

A number of labeling and detection strategies are available for base discrimination using the FRET technique. For example, different fluorescent labels may be used for each type of nucleotide present in the extension reaction with discrimination between the different labels based on the wavelength and/or the intensity of the light emitted from the fluorescent label.

A second strategy involves the use of fluorescent labels and quenchers. In this strategy, certain nucleotides in the reaction mixture are labeled with a fluorescent label, while the remaining nucleotides are labeled with one or more quenchers. Alternatively, each of the nucleotides in the reaction mixture is labeled with one or more quenchers. Discrimination of the nucleotide bases is based on the wavelength and/or intensity of light emitted from the FRET acceptor, as well as the intensity of light emitted from the FRET donor. If no signal is detected from the FRET acceptor, a corresponding reduction in light emission from the FRET donor indicates incorporation of a nucleotide labeled with a quencher. The degree of intensity reduction may be used to distinguish between different quenchers.

A third strategy involves modulating FRET efficiency by varying the distance between the nanocrystal donor and the fluorescent label or quencher acceptor. In this strategy, the same type of fluorescent label or quencher may be used, however, the distance between the nanocrystal and the label is varied for each nucleotide to be identified, causing a modulation of FRET efficiency. The distance may be varied through the structure of the nucleotide itself, the position of the label or quencher on the nucleotide, or the use of spacers or linkers during attachment of the fluorescent label or quencher to the nucleotide. Modulation of FRET efficiency results in a detectable modulation of emission intensity or quenching.

In another strategy, FRET efficiency may be modulated by varying the number of labels or quenchers attached to each incoming nucleotide. In this strategy, differing numbers of the same label or quencher are attached to each nucleotide. For example, one label may be attached to A, two to T, three to G, and four to C. Increasing the number of acceptors relative to the nanocrystal donors increases FRET efficiency and quantum yield, such that base discrimination may be based on the intensity of light emission from the acceptor(s) or the reduction of light emission from the nanocrystal donor(s).

Typically, the signal from the detector is converted into a digital signal with an A-D converter and an image of the sample is reconstructed on a monitor. The user can optionally select a composite image that combines the images derived at a number of different wavelengths into a single image. The user can also specify that an artificial color system is to be used in which particular probes are artificially associated with specific colors. In an alternate artificial color system the user can designate specific colors for specific emission intensities.

In one embodiment, a single molecule sequencing system of the present disclosure comprises a microscope capable of single molecule fluorescence microscopy, and uses Total Internal Reflection (TIR) to reduce the excitation volume. Donor and acceptor signals are collected by a high numerical aperture objective and then separated by color via dichroic mirrors (Chroma); fluorescence is also passed through bandpass filters to increase signal-to-noise ratio before forming an image on the camera. The cameras are back-illuminated to give 90% quantum yield, and provide on-chip amplification. Data analysis is conducted off-line with the FRETAN software (Volkov et al., Ser. No. 11/671,956).

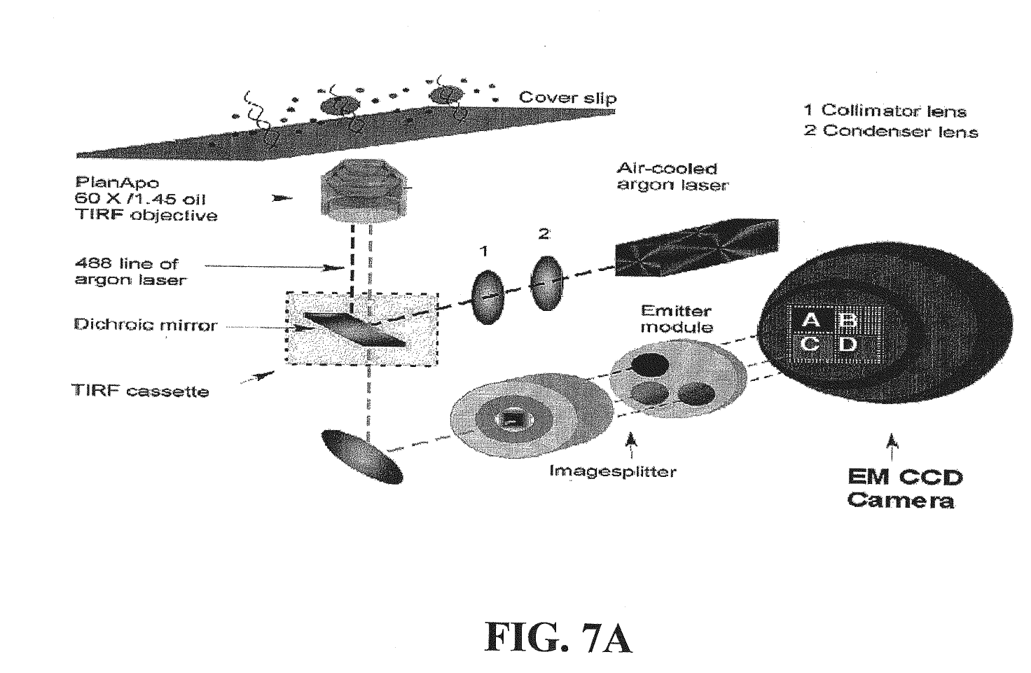

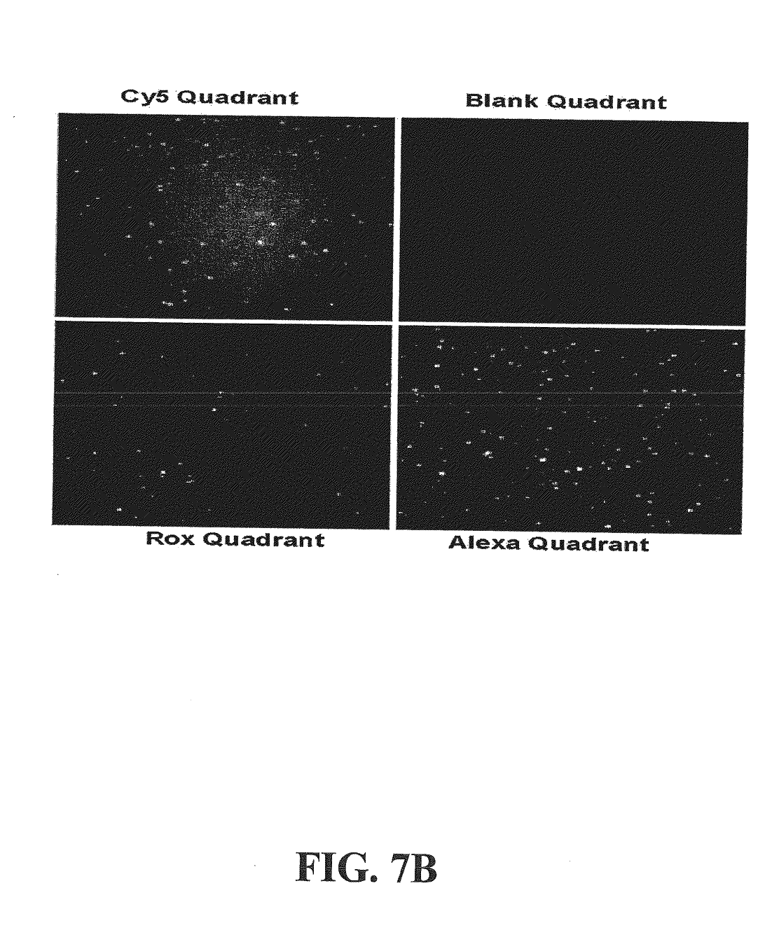

FIG. 7A (left) depicts a schematic of an exemplary single-molecule detection system. FIG. 7A (right) depicts a composite QuadView single molecule image after averaging and signal processing of each quadrant (independently). These exemplary detection systems used in are summarized in the following Table:

| Microscope | TIR type | Excitation lasers | Objective lens | L separation | Camera |
|---|---|---|---|---|---|
| Nikon | Objective | Argon ion tunable | Nikon 60x Plan | Quadview (MAG | Cascade II |
| Eclipse | (Nikon TIRF | (457, 488, 514 nm) | Apo TIRF 1.45 | Biosystems) | 512; |
| TE2000-U | illuminator) | | Oil | | MetaMorph |
| | | | | | software |
| Nikon | Objective | 488 nm Argon ion | Nikon 60x Plan | Quadview (MAG | Cascade |
| Eclipse | (Nikon TIRF | 594 nm HeNe | Apo TIRF 1.45 | Biosystems) | 512B: |
| TE2000-E | illuminator) | 633 nm HeNe | Oil | | MetaMorph |
| | | | | | software |
| Olympus | Prism (Fused | 488 nm Argon ion | Olympus 60x | Custom-built | iXon DU897 |
| IX71 | silica; CVI | 532 nm solid state | UPlanSApo 1.2 | | (Andor); |
| | Laser) | 594 633 nm HeNe | Water | | Solis |
| | | | | | software |

Any combination of the above described labeling and detection strategies may be employed together in the same sequencing reaction. Depending on the number of distinguishable labels and quenchers used in any of the above strategies, the identities of one, two, or four nucleotides may be determined in a single sequencing reaction. Multiple sequencing reactions may then be run, rotating the identities of the nucleotides determined in each reaction, to determine the identities of the remaining nucleotides. In some embodiments, these reactions may be run at the same time, in parallel, to allow for complete sequencing in a reduced amount of time.

The identities of the incorporated nucleotides may be determined rapidly, for example in real time or near real time, as extension of the primer strand occurs, through FRET interactions between a nanocrystal attached to the polymerase, typically at or near the reaction site and a FRET acceptor moiety attached to the incoming nucleotides as they are incorporated into the complementary strand.

Typically, the raw data generated by the detector represents between multiple time-dependent fluorescence data stream comprising wavelength and intensity information. Once the emissions are detected and gathered, the data may be analyzed using suitable methods to correlate the particular spectral characteristics of the emissions with the identity of the incorporated base. In some embodiments, such analysis is performed by means of a suitable information processing and control system. Preferably, the information processing and control system comprises a computer or microprocessor attached to or incorporating a data storage unit containing data collected from the detection system. The information processing and control system may maintain a database associating specific spectral emission characteristics with specific nucleotides. The information processing and control system may record the emissions detected by the detector and may correlate those emissions with incorporation of a particular nucleotide. The information processing and control system may also maintain a record of nucleotide incorporations that indicates the sequence of the template molecule. The information processing and control system may also perform standard procedures known in the art, such as subtraction of background signals.

An exemplary information processing and control system may incorporate a computer comprising a bus for communicating information and a processor for processing information. In one embodiment, the processor is selected from the Pentium®, Celeron®, Itanium®, or a Pentium Xeon® family of processors (Intel Corp., Santa Clara, Calif.). Alternatively, other processors may be used. The computer may further comprise a random access memory (RAM) or other dynamic storage device, a read only memory (ROM) and/or other static storage and a data storage device such as a magnetic disk or optical disc and its corresponding drive. The information processing and control system may also comprise other peripheral devices known in the art, such a display device (e.g., cathode ray tube or Liquid Crystal Display), an alphanumeric input device (e.g., keyboard), a cursor control device (e.g., mouse, trackball, or cursor direction keys) and a communication device (e.g., modem, network interface card, or interface device used for coupling to Ethernet, token ring, or other types of networks).

In particular embodiments, the detection system may also be coupled to the bus. Data from the detection unit may be processed by the processor and the data stored in the main memory. Data on emission profiles for standard nucleotides may also be stored in main memory or in ROM. The processor may compare the emission spectra from nucleotide in the polymerase reaction to identify the type of nucleotide precursor incorporated into the newly synthesized strand. The processor may analyze the data from the detection system to determine the sequence of the template nucleic acid.

It is appreciated that a differently equipped information processing and control system than the example described above may be used for certain implementations. Therefore, the configuration of the system may vary in different embodiments. It should also be noted that, while the processes described herein may be performed under the control of a programmed processor, in alternative embodiments, the processes may be fully or partially implemented by any programmable or hardcoded logic, such as Field Programmable Gate Arrays (FPGAs), TTL logic, or Application Specific Integrated Circuits (ASICs), for example. Additionally, the method may be performed by any combination of programmed general purpose computer components and/or custom hardware components.

Following the data gathering operation, the data will typically be reported to a data analysis operation. To facilitate the analysis operation, the data obtained by the detection system will typically be analyzed using a digital computer. Typically, the computer will be appropriately programmed for receipt and storage of the data from the detection system, as well as for analysis and reporting of the data gathered.

Any suitable base-calling algorithms may be employed. See, for example, U.S. Provisional App. No. 61/037,285. In certain embodiments, custom designed software packages may be used to analyze the data obtained from the detection system. In alternative embodiments, data analysis may be performed, using an information processing and control system and publicly available software packages. Non-limiting examples of available software for DNA sequence analysis include the PRISM™. DNA Sequencing Analysis Software (Applied Biosystems, Foster City, Calif.), the Sequencher™ package (Gene Codes, Ann Arbor, Mich.), and a variety of software packages available through the National Biotechnology Information Facility at website www.nbif.org/links/1.41.php. Data collection allows data to be assembled from partial information to obtain sequence information from multiple polymerase molecules in order to determine the overall sequence of the template or target molecule.

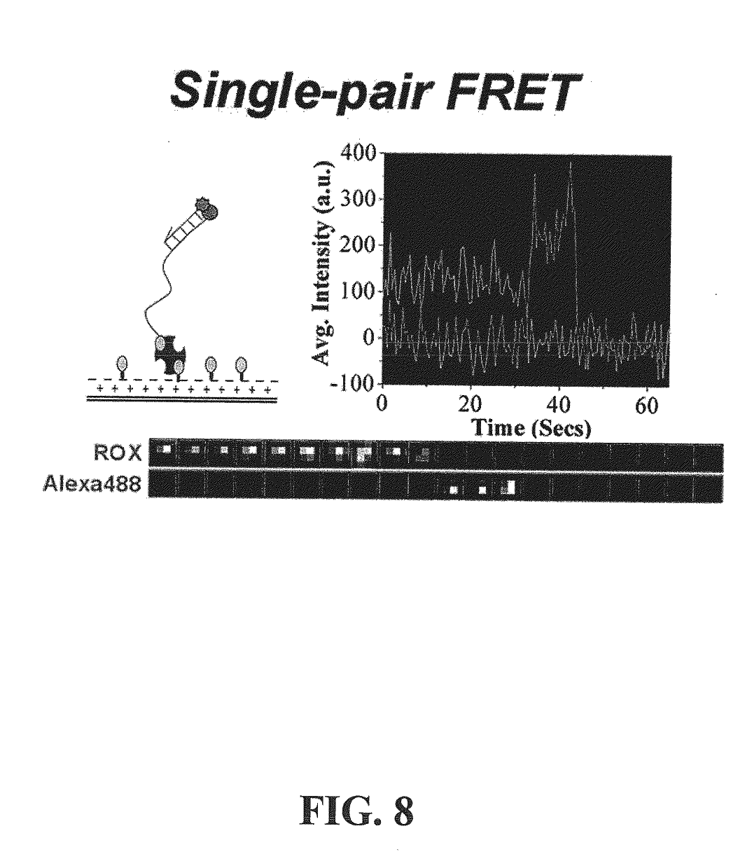

Typically, detection of spFRET events involves detection of anti-correlated changes in fluorescence at the donor and acceptor emission wavelengths during laser excitation at the donor excitation wavelength. In one exemplary embodiment, each fluorescence wavelength is monitored by a separate quadrant of the CCD imager. Registration of the quadrants is carried out prior to the experiment using images of multi-wavelength emitting microbeads (Molecular Probes). Fluorescence from a single molecule pair, a “spot”, is confined to ˜4 adjacent pixels and displays a characteristic single-step photobleaching profile. The intensities of fluorescence as a function of time at each wavelength (averaged over the 4 pixels) represent the signals of interest. In the exemplary embodiment of FIG. 8, the sample is excited at 488 nm. The acceptor fluorescence due to spFRET is high until the acceptor photobleaches (at 32 seconds). Immediately, donor fluorescence appears, as it can no longer transfer energy to an acceptor in close proximity. Donor fluorescence continues until photobleaching at 42 seconds. Automated analysis software, FRETAN, identifies each of the spots in the sample (taking into consideration noise thresholds), subtracts the background fluorescence, and identifies anti-correlated changes in the fluorescence of each donor/acceptor pair to identify spFRET (Volkov et al., Ser. No. 11/671,956).

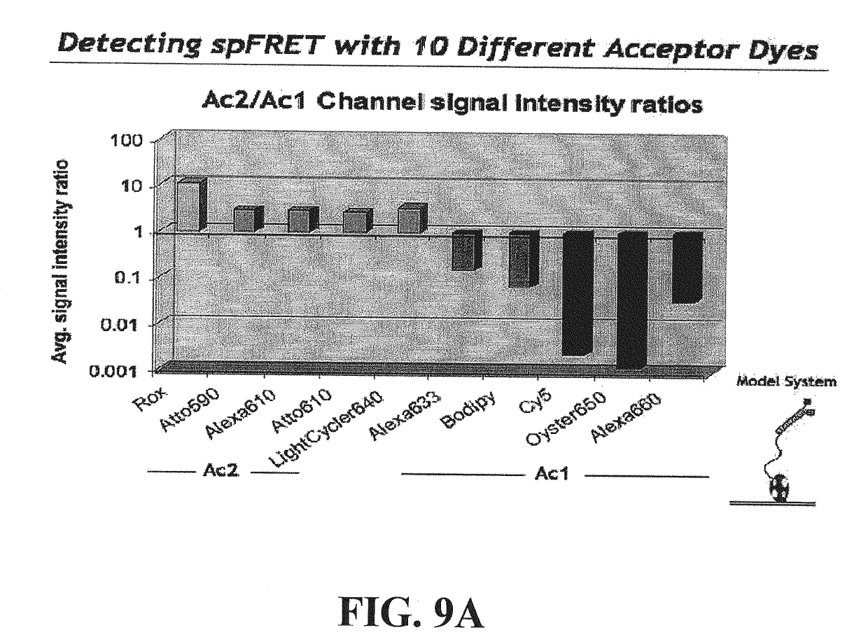

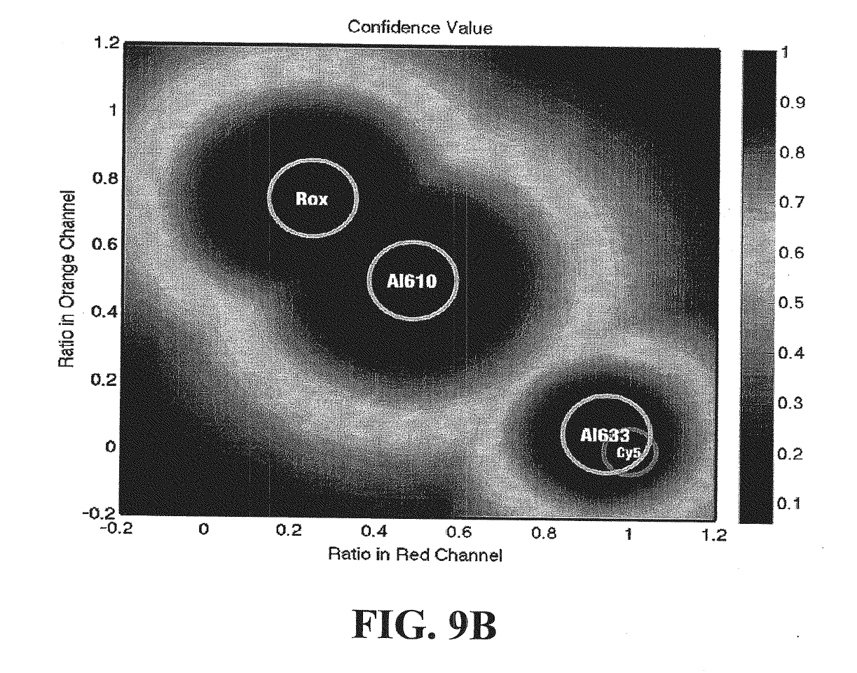

Typically, dye sets are chosen to maximize the efficiency of energy transfer between an acceptor labeled-nucleotide and the donor fluorophore. Optionally, before synthesizing a series of modified nucleotides with a candidate fluorophore, the candidate fluorophore may be screened for its ability to spFRET with the donor of choice. This may be accomplished using a static spFRET assay. In one study, fluorophore stability and FRET efficiency with the donor Alexa488 was determined for 30 different candidate acceptors in 3 spectral channels. Characteristic signal intensity ratios for each acceptor fluorophore among the 3 quadrants were evident, and these ratios were used to increase the confidence in acceptor color identification, similar to methods used to determine base calling and error probability of automated sequencer traces (Ewing and Green 1998; Ewing, Hillier et al., 1998). Assigning confidence values, CV, to individual base calls allows the objective evaluation of data quality and automate data analysis and assembly. Additionally, acceptor fluorophore characteristics (in the context of the intact nucleotide vs PPᵢ), nucleotide incorporation efficiency, and nucleotide synthesis/solubility/stability are considered before a nucleotide is chosen for use in a sequencing system according to the present disclosure. FIGS. 9A&B depict the results for 10 exemplary candidate acceptors analyzed via Ac1 and Ac2 intensity ratios during FRET. FIG. 9A: 10 Ac ratios. FIG. 9B: Confidence values for Rox, Alexa610 & 633, Cy5 in non-optimized detection system. This exemplary detection system was illustrates the feasibility of a single-molecule sequencing approach. As the above data illustrates, the fluorescence signatures of two groups of acceptor fluorophores can be distinguished with high confidence based on the intensity and distribution of signal among the three detector channels during spFRET in the presence of Cy5 and ROX γ-nucleotide at concentrations that promote polymerase activity.

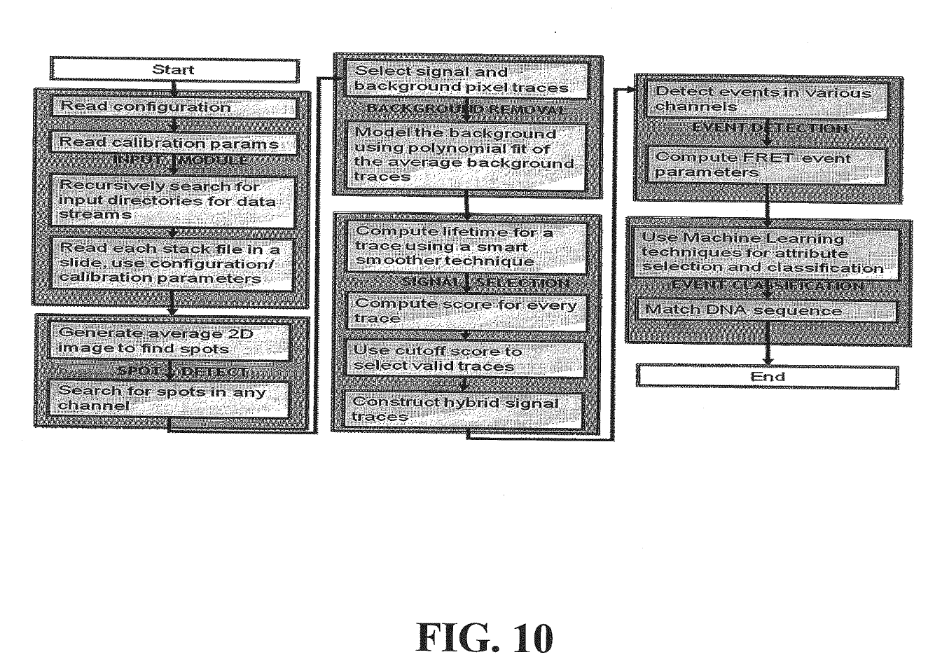

In some embodiments, detection and base calling involves the use of the FRETAN software (Volkov et al., Ser. No. 11/671,956), wherein approximately 50 attributes associated with each signal detected in the disclosed sequencing systems can be analyzed to determine the confidence value (CV) associated with each base call, and the CV for each call is evaluated to determine each base in the consensus sequence. The FRETAN software associates each base call with a particular confidence value, and will permanently associate this information about base quality with the determined consensus sequence. FIG. 10 depicts an overview of the FRETAN software, which consists of six modules. The input module reads acquired image data as well as the configuration/calibration parameters associated with the data. The spot detect module averages the stack of acquired images in the time (Z) plane and deploys a spot detection algorithm that employs thresholds to detect spots in a particular channel. In the background removal module nine signal pixel traces are selected in a 3×3 neighborhood of the detected spots and then eight darkest background pixels are selected in a 5×5 neighborhood around the detected spots. The average background trace is obtained from the eight background pixels and a local regression curve is fitted to the average background trace to model the background. In the signal selection module, the objective is to identify the most appropriate signals from the nine pixel traces initially selected. A lifetime for every trace is determined using a ‘smart smoother’ technique and a score is computed for every trace. A cutoff score, depending on the max score, is used to select the best signal traces, and a hybrid signal trace results from averaging the selected best signal traces. The event detection module detects FRET events in various channels and computes ˜50 attributes associated with FRET for that event. In the event classification module the goal is to classify FRET events into types (binding; incorporation—correct/incorrect; noise). Classification is done by machine learning techniques, and mainly consists of a training phase and a testing phase. In the training phase, attribute selection is performed using information gain ratio criteria to select the most important attributes from the pool of 50 FRET attributes and classifiers, which also provide high accuracy, true positive rate, and low false positive rate. In the testing phase, the selected attributes from the training phase are used and deploy chosen classifiers (e.g., support vector machine is one of the classifier used to classify the events). Finally, the DNA sequence is matched to the classified events.

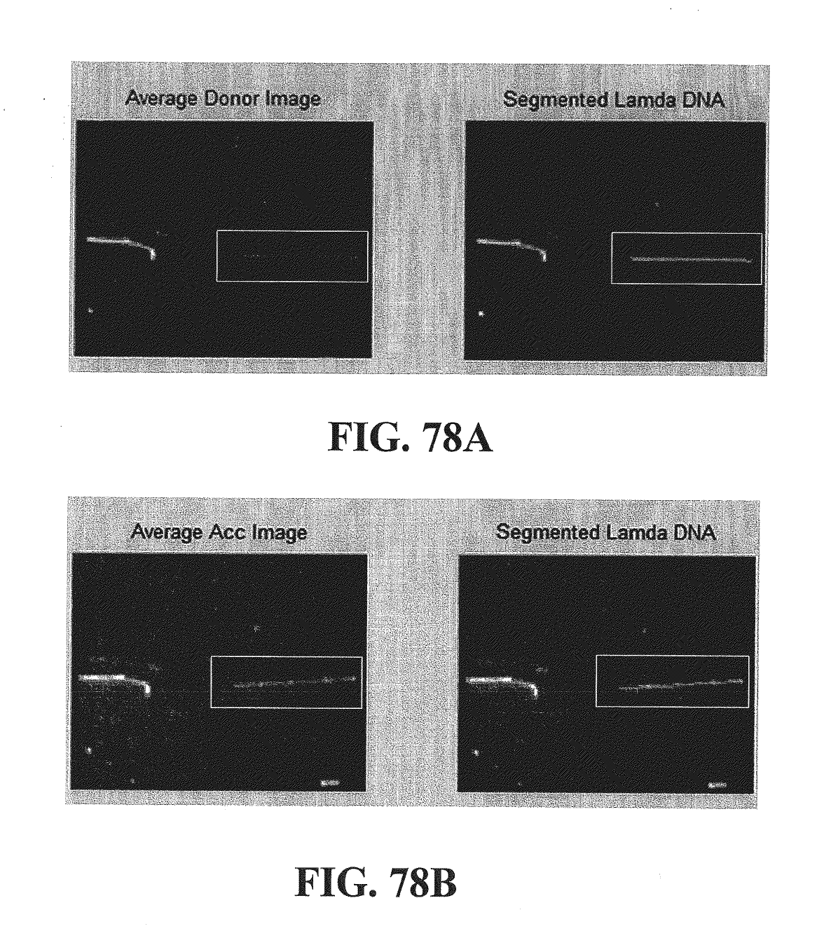

Frequently, it has been observed that donor bleed into the acceptor channel can mask detection of acceptor signals. This problem may optionally be addressed through use of analysis software that extracts acceptor signals. In one embodiment, experimentally determined thresholding followed by the largest connected component analysis method was used to segment the lambda DNA in the donor channel (FIG. 78A). Using the information about the spatial extent of the region of interest (ROI) in the donor channel, the lambda DNA in the acceptor channel is next segmented in a similar way (FIG. 78B).

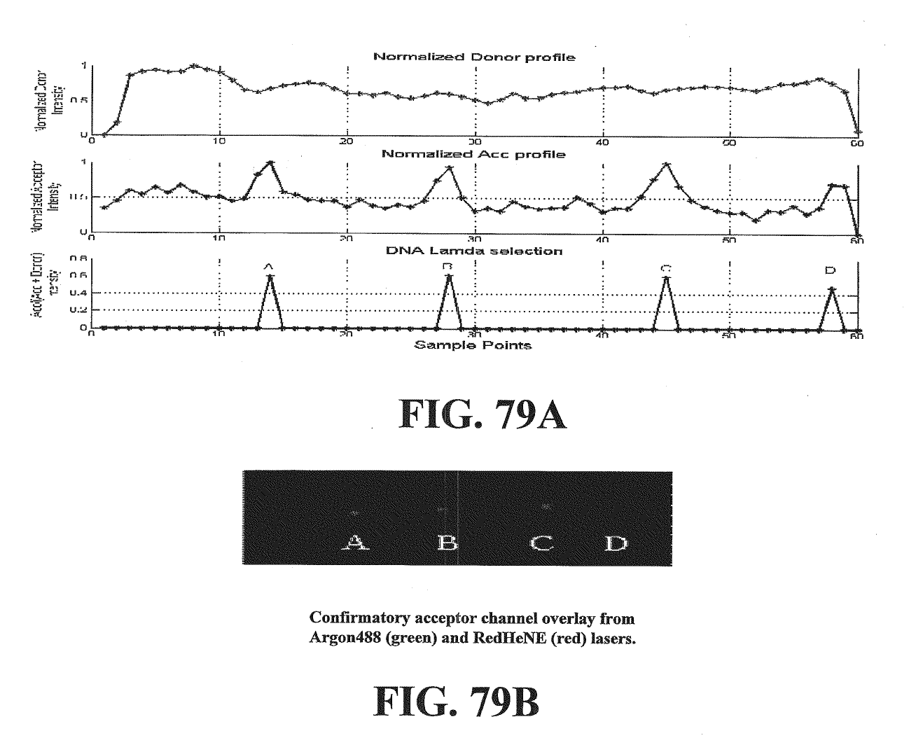

Registration of the segmented ROI was performed in both channels and then compared the normalized intensity of every spatially corresponding point in both donor and acceptor channels. Using a function of normalized acceptor and donor intensity, criteria were defined to accept certain spatial coordinates as incorporated acceptor labels. FIG. 79A shows four automatically detected DNA acceptors (A, B, C, D). Note that we observe higher intensity at those points only in the acceptor channel, compared to the donor channel. FIG. 79B shows co-localization of the detected points in the acceptor channel via Argon or RedHeNe excitation, confirming the accuracy—to the level of pixel registration—of the automated analysis.

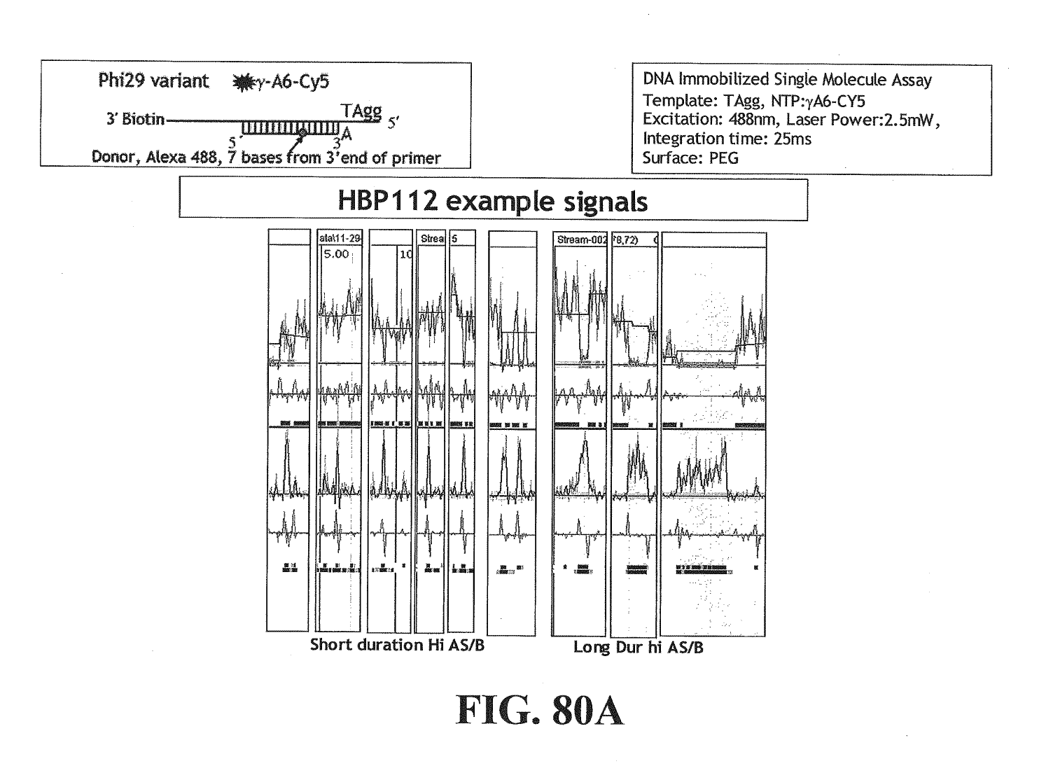

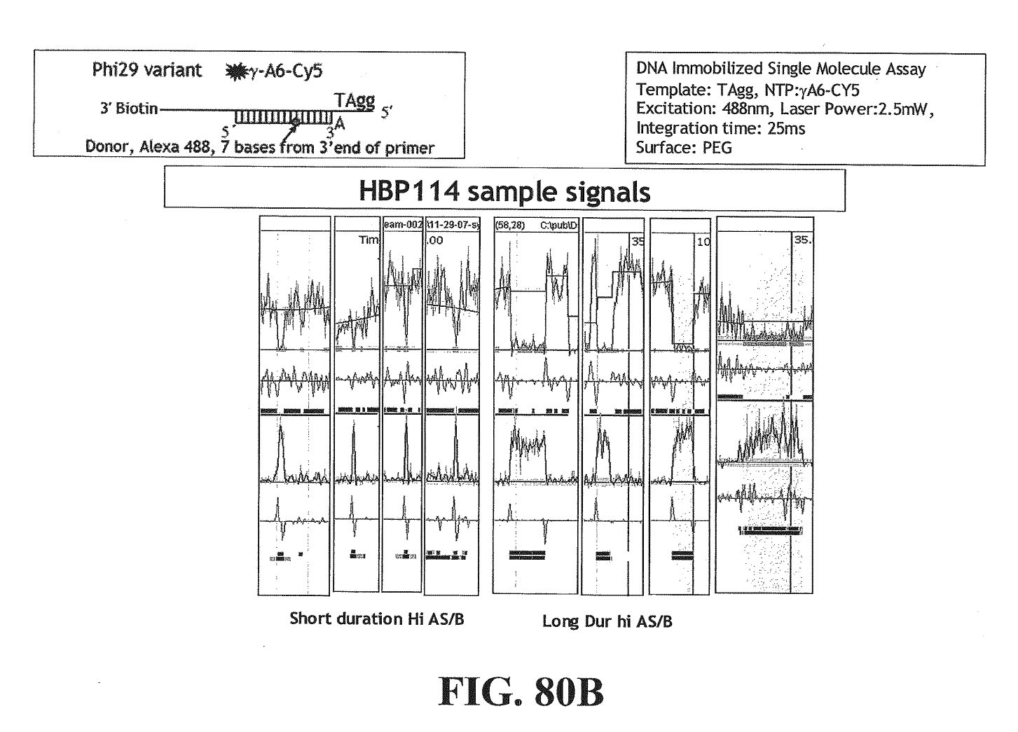

Referring to FIGS. 80A&B, extension traces are shown for two different Phi29 variants in a DNA immobilized single molecule assay using the donor Alexa 488 attached to the oligonucleotide primer (specifically, 7 nucleotides from the 3′ end of the oligonucleotide primer) and nucleotides labeled on the terminal phosphate with a Cy5 dye moiety. The short duration and long duration signals evidence the differences in incorporation dynamics of the two variants, suggesting variant optimization is possible to improve acceptor signal detection and noise reduction.

Some metal-ligand complexes (MLC), ie Ru(bpy) have fluorescence lifetimes on the order of 1 us, much longer than the nanosecond lifetime of organic fluorophores, making them amenable to be used as FRET donors while using time gating of the fluorescence to decrease acceptor background. In this scheme, a MLC is used as a FRET donor and is excited with a pulsed laser (˜10 ns pulse width). During excitation of the MLC, the camera (a CCD camera with an image intensifier) is gated off to prevent acquisition of photons from the acceptor molecules in solution. After a suitable time (˜1 us, after solution acceptor fluorescence has decayed), the camera is gated on and fluorescence is collected from acceptor molecules that are able to undergo energy transfer from the donor. Background signal could thus be almost entirely removed. With a pulsed laser and image intensified camera, the cycle of pump-wait-record would then be carried out fast enough to catch the transient signals of gamma-NTP incorporation (˜100 kHz).

The long fluorescence lifetime of MLC may also be used in a scheme in which the NTPs are labeled with MLC and the fast (<1 us) diffusion of these small molecules is used to decrease background. In this scheme, no donor is used, but the luminescence is collected directly from the MLC-NTP while it is in the binding pocket of the enzyme. Background is reduced because other excited MLC-NTP in solution will diffuse out of the detection volume quickly enough to not be detected with the time-gated camera.

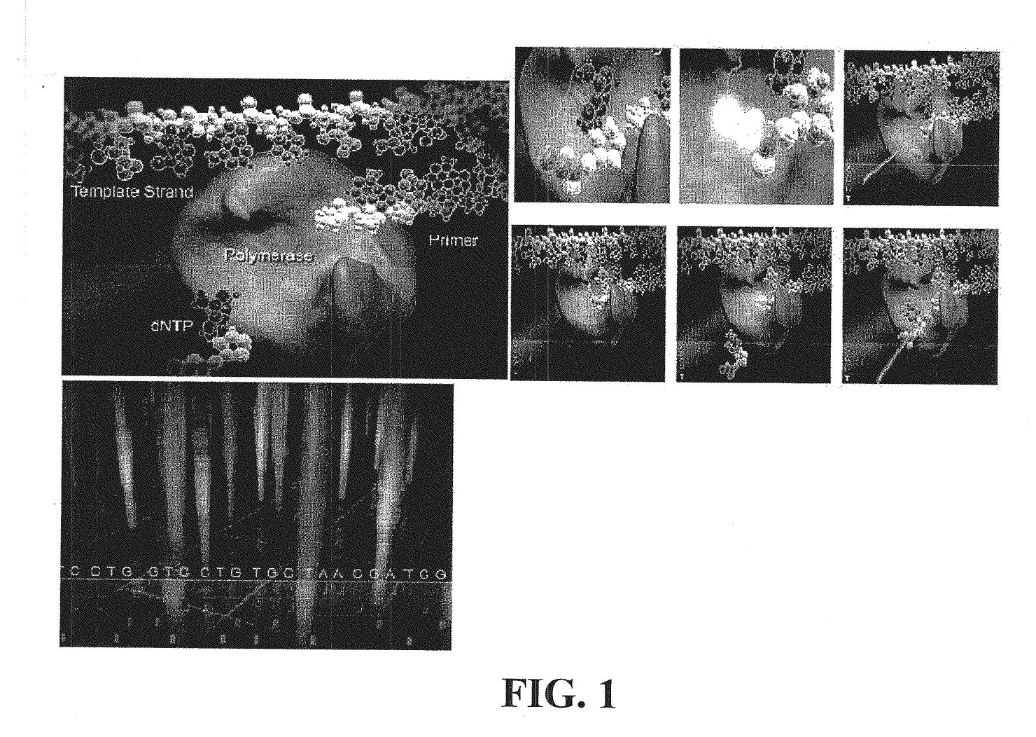

FIG. 1 depicts real-time detection of nucleotide incorporation in an exemplary system. Top, left. Reaction components of an exemplary sequencing system according to the present disclosure, comprising modified polymerase and nucleotide, primer, and template. Top, right. Energy transfers from a donor operably linked or otherwise attached to the polymerase to acceptor on gamma-labeled nucleotide triphosphates, stimulating acceptor emission and detection. Fluorescently-labeled pyrophosphate PP, leaves the sequencing complex, producing natural DNA. A non-cyclical approach enables rapid detection of subsequent incorporation events. Left. Arrays of nano-sequencing machines. The time-dependent fluorescence signals emitted from each asynchronous sequencing complex are monitored and analyzed to determine DNA sequence information. Massively parallel arrays enable ultra-high throughput (at least 1 million bases/second/machine).

The closed system design eliminates the need for extensive microfluidics and minimizes the volume of reagents needed per reaction. See, for example, Rea, U.S. Ser. No. 11/781,157. Because data is collected during DNA replication, a single reagent injection produces data, and there is no requirement for serial addition of reaction components, thereby minimizing reagent consumption (waste).

In one exemplary embodiment, single-molecule sequencing is performed using an immobilized sequencing complex. Optionally, detection of dynamic fluorescence is performed near the substrate-solution interface. Although acceptor fluorophores are selected to minimize direct excitation at the donor wavelength, some of the many millions of acceptor molecules in solution above the interface and in transient interactions with the interface will be sufficiently excited to fluoresce. This would result in unacceptably poor single-to-noise performance. Evanescent wave excitation by illumination during total internal reflection is an effective strategy for restricting illumination to within approximately 100 nm of the substrate-solution interface. When a collimated beam of light encounters an interface between two media of different refractive index (e.g., a glass-aqueous interface), a combination of refraction and reflection of the incident light will occur. When the medium with lower refractive index (i.e., aqueous) lies beyond the interface, more of the incident light will be reflected from the interface as the angle of incident light is increased relative to the normal. At the critical angle, given by Snell's Law as

$$θ = {sin}^{- 1} ( \frac{{n}_{1}}{{n}_{2}} ) \\ [ where {n}_{1} > {n}_{2} ; n = refractive index ]$$

all of the light is reflected by the interface. Some of the reflected light propagates parallel to the interface, resulting in the establishment of an electromagnetic field on the opposite side of the interface. This ‘evanescent wave’ displays the same wavelength as the incident light, but does not propagate into the solution. Rather, the field strength decays exponentially such that, for a glass-aqueous interface, only those fluorophores located within the first 100 nm of the interface will be excited. At the concentrations employed in our exemplary sequencing assays, less than 20 acceptor-labeled nucleotide molecules are present within the focal volume above a 4-pixel domain, greatly reducing noise from direct excitation of acceptor fluorophores.

As an example, an Alexa-488 donor was linked to the 3′ base of an oligonucleotide that is biotinylated at the 5′ end, and a ROX acceptor was linked to the 5′ base of the complementary strand of a duplex. These 5′ biotinylated oligonucleotides were stably immobilized onto a polyelectrolyte-biotin-neutravidin (PEBN) surface, resulting in a random distribution of single molecules, the density of which can be tuned by adjusting concentration of the biotinylated DNA. See, for example, Osborne, Barnes et al., 2001; Ha, Rasnik et al., 2002; Braslaysky, Hebert et al., 2003; Kartalov, Unger et al., 2003 (describing immobliization of molecules on surfaces). As shown in FIG. 8, sustained spFRET was observed using such a system. Analysis of duplex DNA lacking the donor fluorophore failed to detect fluorescence in the ROX channel, when illuminated with a 488 nm Argon ion laser, demonstrating that the acceptor signal is due to energy transfer from the donor. FIG. 8, Top left: This figure contains a cartoon depicting immobilized DNA duplex on a PEBN surface. Alexa488 (donor, shown as a circle) is attached to the 3′ of one strand, and ROX (acceptor, shown as a star) to the 5′ base of the complementary strand. Top right: Single molecule signals plotting intensities of Alexa488 (donor) and ROX (acceptor) during and after FRET via Argon 488 nm laser. Note that the decrease in the acceptor signal intensity coincides with the increase in donor intensity. Bottom: Mosaics created from the corresponding areas collected during single molecule detection.

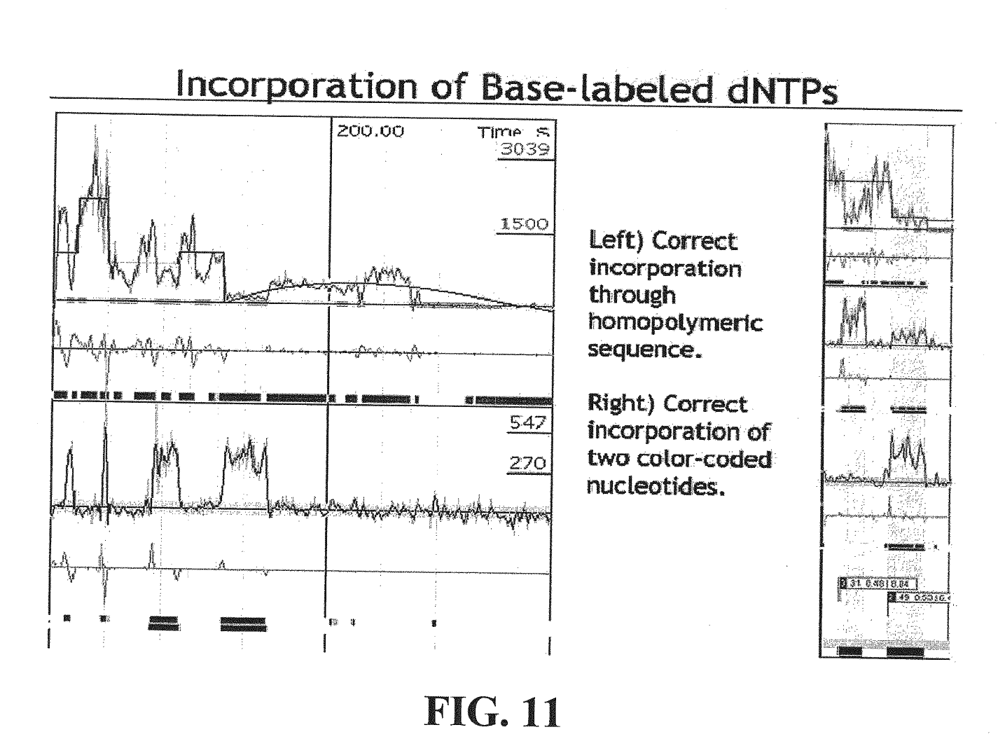

In another exemplary embodiment, the FRET donor is operably linked to the nucleotide base instead of a phosphate. Such a a BL-nucleotide can serve as a ‘punctuation mark’ to facilitate characterization of dynamic spFRET (FRET occurring transiently during γ-labeled nucleotide incorporation preceding the stable BL signal). In FIG. 11, on-surface incorporation of base-labeled nucleotides (BL-nucleotide) was monitored to detect a stable spFRET event with a donor-labeled primer. Two different DNA duplexes containing Alexa488-labeled primer were immobilized and incubated in the presence of reaction buffer containing 0.5 μM dUTP-Cy5 or dCTP-Cy5 and dUTP-Alexa594 (all base-labeled) and the polymerase enzyme Phi29 exo(−), which comprises the protein encoded by SEQ. ID: 3. This mutant protein, also referred to herein as “HBP1” or simply “HP1”, comprises the protein sequence of wild type Phi29 polymerase, as provided in SEQ. ID: 1, but additionally includes the mutations D12A and D66A, and exhibits reduced exonuclease activity as compared to its wild-type counterpart. The samples were excited using a 488 nm Argon laser at 400 uW and the data were collected at is or 300 ms integration times. The signals were separated using dichroics (560 nm, 640 nm) and band pass filters (525/50 nm, 620/60 nm and 700/75 nm). The traces show anti-correlated spFRET incorporation signals and acceptor photobleaching. At this integration time, the detected spFRET signals have a signal to noise ratio ranging from 3-14.

Note that this type of ‘donor-acceptor-donor’ fluorescence signal was observed in the presence of the enzyme, and that acceptor duration and single-step photobleaching kinetics are consistent with spFRET. These experiments are similar to the static spFRET experiments described above—with one significant difference: they visualize real-time incorporation of base-labeled nucleotides (additional analysis, below). Optionally, the acceptor fluorophore attached to the base may be removed, e.g., by photobleaching (as above) or chemical/photo-cleavage, before the next nucleotide is incorporated to improve detectability of subsequent nucleotides and incorporation efficiency. Reducing nucleotide concentration helps minimize background fluorescence due to acceptor excitation and can be used to control the rate of the polymerase reaction for real-time monitoring. A single, lower-wavelength excitation laser is used to achieve high selectivity. If a more stable donor is introduced at or near the 3′ end of the primer, real-time incorporation of 15 acceptor-labeled nucleotides may be detected.

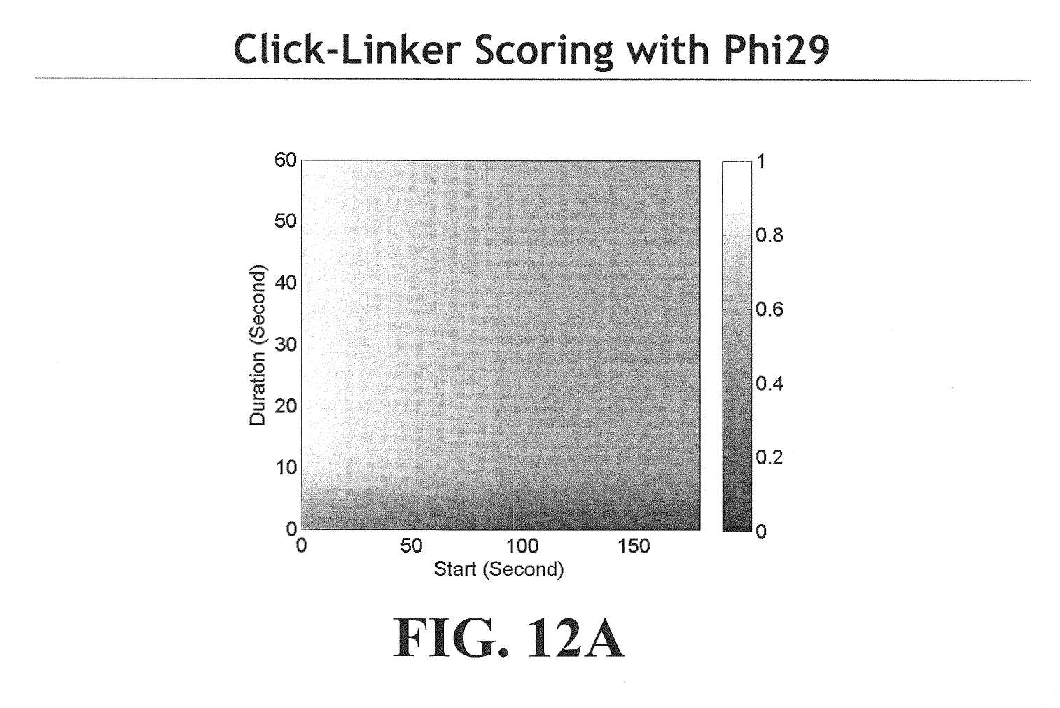

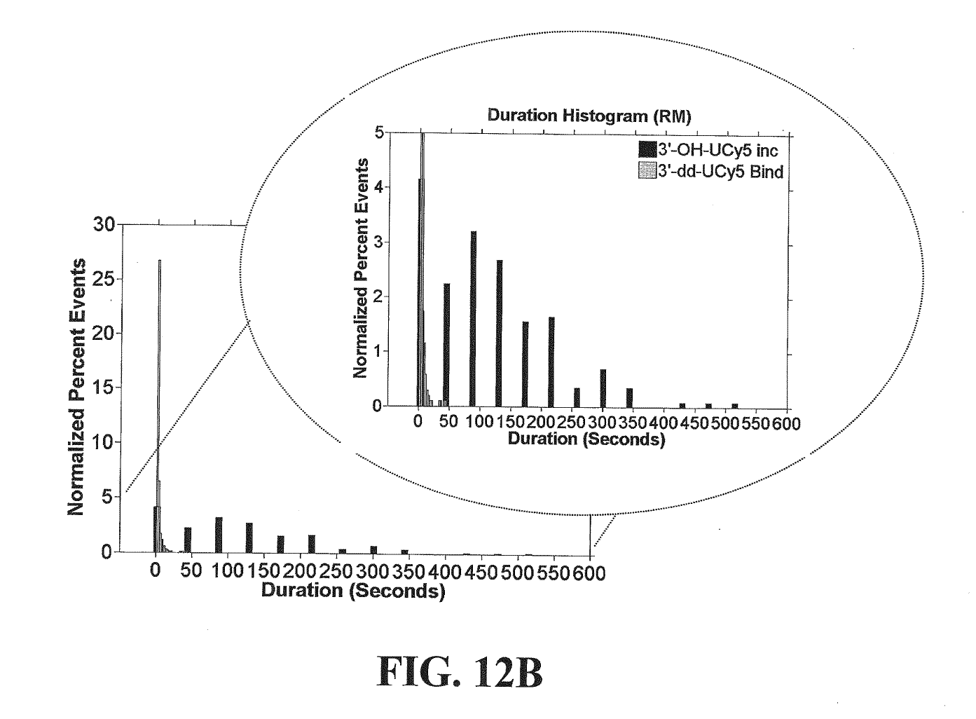

Optionally, sequencing applications involving the use of base-labeled nucleotides may include an analysis procedure to assign confidence values to BL-nucleotide events is relevant to γ-labeled nucleotide event characterization. To identify informative event attributes associated with incorporation (3′-OH-inc) vs binding (3′-dd duplex) vs mis-incorporation (3′-OH mis-match) of BL-nucleotides, reactions were performed using conditions similar to those described above substituting a template specifying incorporation of a single BL-nucleotide and a primer containing a donor at −7 position, such that the distance between the donor and acceptor was ˜27 Å, i.e, high FRET. FRETAN software was used to obtain donor and acceptor traces and define FRET attributes, which can be imported into a comprehensive database, named FRET_db, of single molecule events produced using the disclosed sequencing technology. FRET_db organizes data in a hierarchical fashion with data cascading across different nodes of information pertaining to donor and acceptor properties summarized in eight tables. The database provides an easy and quick way to analyze vast amounts of data based on different experimental conditions. In particular, the ˜50 attributes associated with each FRET event are stored in the database and can be extracted using smart SQL queries. The results are displayed in tab delimited text files that are utilized for downstream analysis (i.e., statistical analysis and graph generation). Custom-designed Perl and MATLAB scripts can also be used to extract, graph, and fit the FRET duration data to single exponential decays as shown in FIGS. 12A&B, below, right.

Furthermore, sequencing applications of the present disclosure allow signals arising from a binding reaction to be distinguished from signals arising from incorporation of a BL-nucleotide (oxygen scavenging system present). The mean duration for binding signals are shorter than the persistent signals associated with the incorporation of BL-nucleotides (80% of binding signals have a duration between 1-5 seconds, while 92.5% of incorporation signals have a duration longer than 5 seconds). Additionally, most of the events in the incorporation reaction occur within 20 seconds with an exponential distribution, whereas in the binding reaction the FRET signals are distributed randomly throughout the data collection, ending only when the donor photobleaches.

Signal frequencies, duration, and start time were identified as the most relevant attributes for distinguishing binding vs. incorporation, and these attributes were used to evaluate confidence values for the incorporation signals. The color map of the scatter plot (duration and start time) is shown in FIG. 12A, and visualizes the confidence values associated with the combination of attribute values at that data point. The confidence value color map is the scatter plot of the attribute data range (start, 0-180 seconds; duration, 0-60 seconds) and the confidence values are visualized in color. The incorporation signals with duration in the range of 10-60 seconds and a start time in the range of 0-20 seconds have very high confidence values (0.9-1) and outside of these attribute ranges, the confidence values progressively decline. Additionally, the frequencies of signals detected from one FOV for correct incorporation is 54/300, for 3′-dd reaction is 216/300, and for mis-match reaction is 5/300. The frequency of the detected events is the basis of the confidence value determination. The confidence value is calculated to reflect the frequency of the events in a given data range between the different reaction conditions (i.e., for the CV of the start attribute between 1 and 5 seconds).

Furthermore, this analysis demonstrates that the events detected in a mis-incorporation reaction are infrequent and typically of short duration, suggesting that the binding signals maybe of a shorter duration not discernable at the is integration time. To resolve signals shorter than 1 second, the 3′dd binding and control experiments were performed using a 1000-fold less active enzyme (termed “DOA” polymerase) at 100 ms integration time and 2 mW laser power. At this integration time, the mean duration for FRET signals with the 3′dd sample is 414 ms, and that for DOA is 257 ms, suggesting that the dd-terminated primer may hold the nucleotide in the binding pocket as compared to the DOA which binds the incoming correct nucleotide much faster, but with a significantly lower incorporation efficiency. Thus, by selecting appropriate integration times, binding and incorporation signals were distinguished.

Multiple spFRET interactions have been detected between 2 different acceptor-labeled gamma nucleotides and an immobilized, donor-labeled polymerase. The data demonstrate that sequential interactions with the same nucleotide type are detected, due to the reappearance of the donor between events, and that two different acceptor-labeled nucleotides are distinguished in this system.

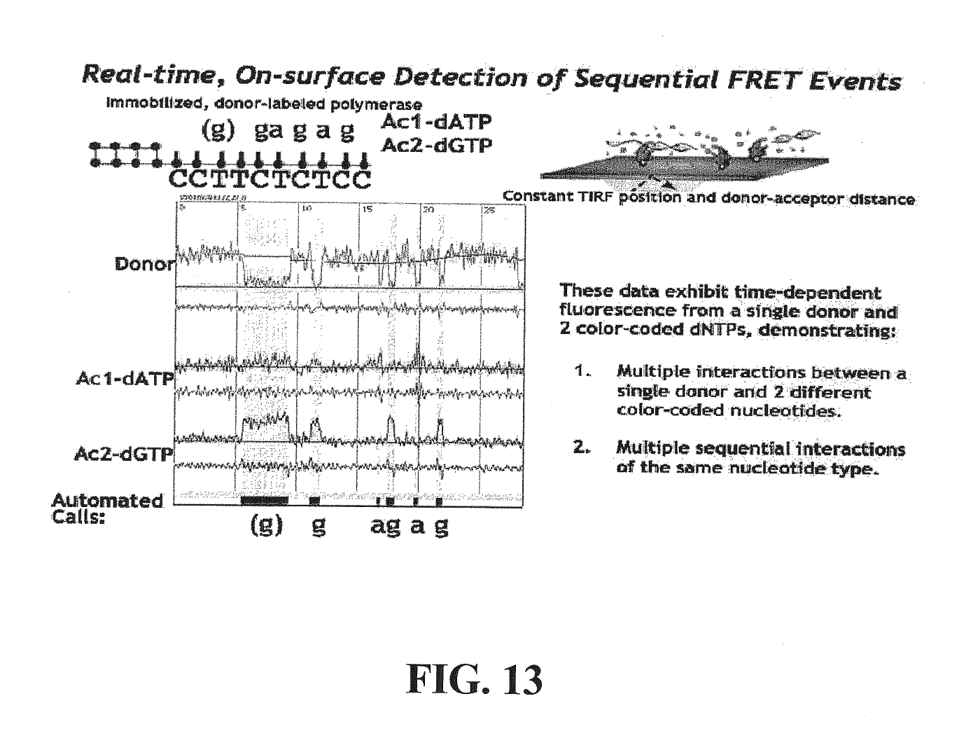

Referring now to FIG. 13, donor labeled enzyme was immobilized on a Ni—NTA-HRP conjugate coated glass surface. The prepared slide was mounted onto the detection system, and 300 μL of extension mix (0.5 μM γ-dGTP-2-Alexa610, 1 μM γ-dATP-2-Cy5, and primer/template duplex) were added. The sample was excited using an Argon laser and fluorescence was detected after separating the emitted light through a beam splitter, as described earlier. Data were collected in real-time with exposure times of 50 ms integration time. Each data set consists of 1000 frames collected FOV (360×360 pixels). Data analysis was performed using the FRETAN analysis program.

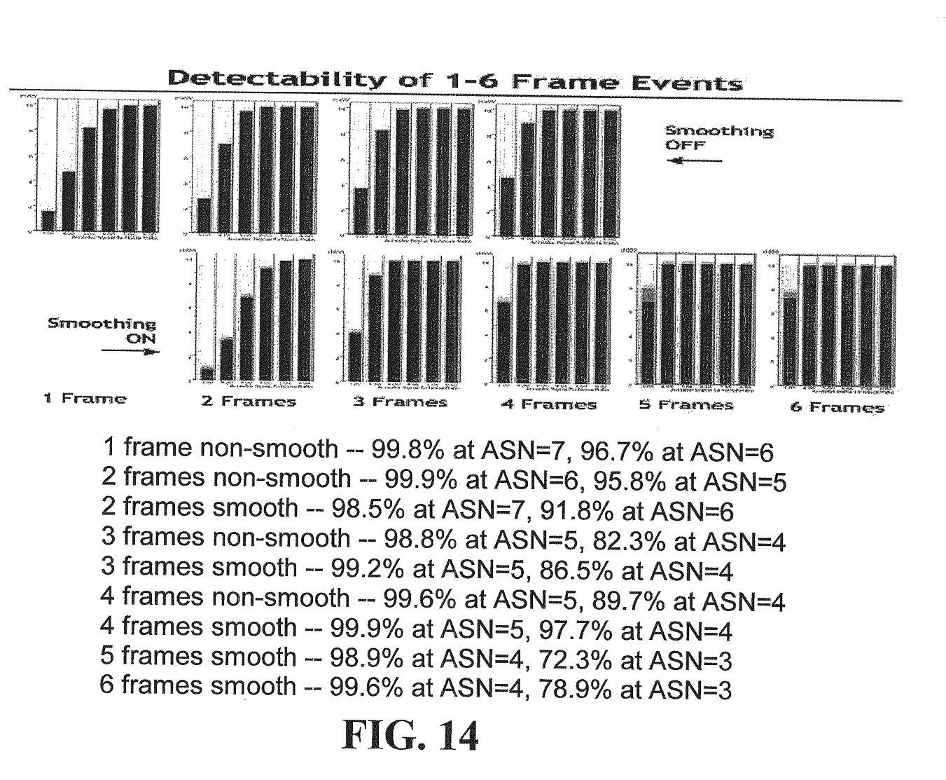

FIG. 14 depicts a comparison of simulated data analyzed by two FRETAN versions. The initial version performs trace smoothing, but the newer version does not. The version with smoothing has a higher detection efficiency with relatively long events, 4 frames or more, and the detection rate at acceptor signal to noise (hereinafter, “ASN”) of 4 is ˜98%. On the other hand, this version misses 1-3 frame events. Thus, because γ signals are short duration events, a separate version of FRETAN was developed that does not use smoothing and works reliably even with 1-frame events, with a detection rate of ˜97% starting at ASN=6 and 99.8% at ASN=7, underscoring the importance of increasing the acceptor ASN associated with γ-labeled nucleotide incorporation. The non-smoothing version works best with 1-2 frame events that are normally difficult to detect with the smoothing version. A similar approach (analyzing simulated data sets) can be taken to assign confidence values to detected signals based on their ASNs, durations, and the algorithm used as shown in FIG. 14.

In some embodiments, the acceptor fluorophore is located on a phosphate group of the nucleotide, typically the terminal phosphate, rather than on the base. This strategy is more demanding, with regard to detection, due to the short time that the acceptor and donor are in close proximity to produce spFRET. Onset of saturation of the acceptors would only begin at excitation intensities ˜1000 times higher than those that those used in the disclosed examples (˜50 W cm-2), which are typical intensities for wide-field single molecule fluorescence microscopy. At the utilized intensities, acceptors are not saturated. Improved detection and color identification of γ-labeled nucleotides will be accomplished by increasing the acceptor signal and reducing background, as described below. Higher excitation intensities will make single frame detection easily possible at high detection efficiency.

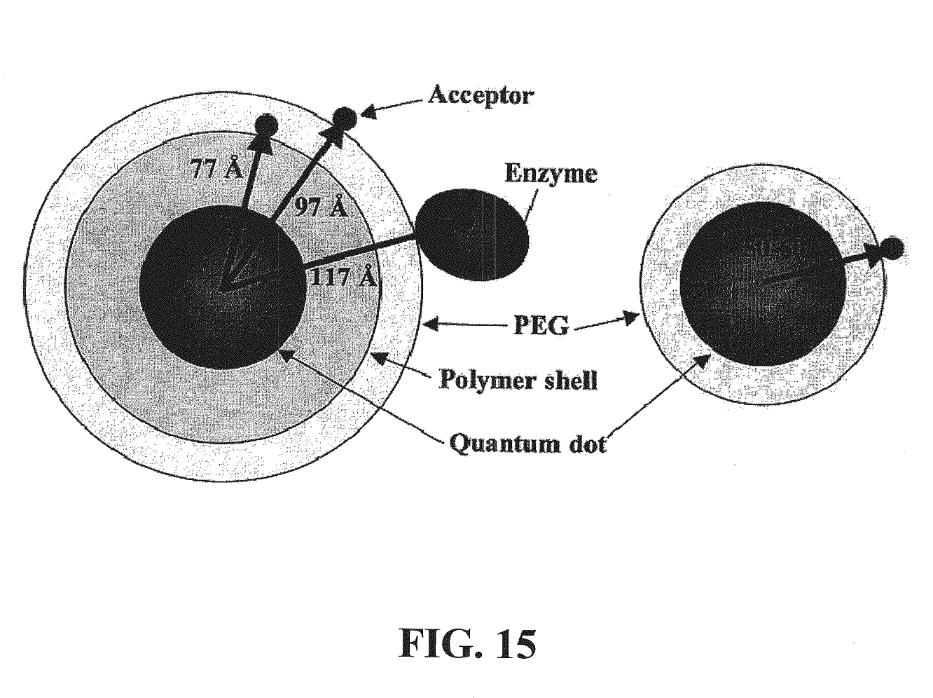

In one embodiment, the FRET donor comprises a nanocrystal, such as a quantum dot. Such nanocrystal-based donors will have several advantages, including the ability to increase donor duration and spFRET signal intensity. FIG. 15 depicts the structure of an exemplary quantum dot. Colloidal semiconductor nanocrystals, or quantum dots (Qdots), are a relatively new generation of fluorescent biological labels that may overcome the photostability issues of organic dyes and allow spFRET sequencing of extended DNA lengths. Our preliminary screening of commercially available Qdots indicated that they are not yet ready to replace organic fluorophores used in our sequencing approach. Another reason for pursuing the use of a Qdot donor is that such donors produce high intensity spFRET events.

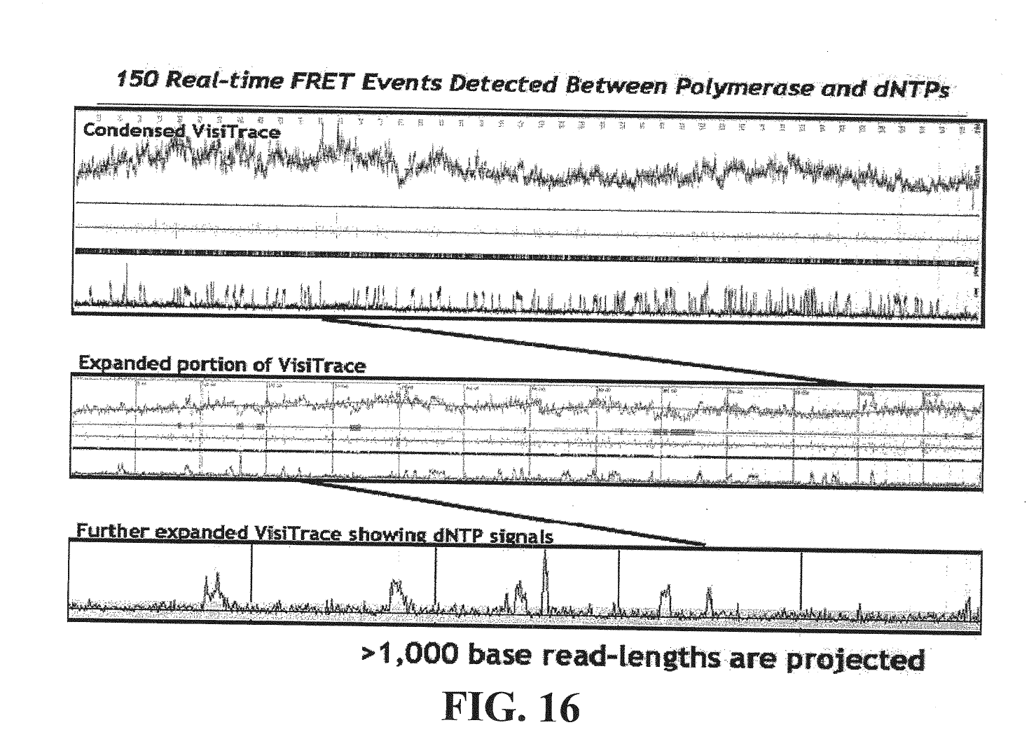

The trace in FIG. 16 illustrates the potential benefit of a novel, quantum dot-based donor (Qdot) by providing data showing 150 on-surface, real-time interactions between multiple γ-labeled nucleotides and immobilized, donor-labeled Phi29 DNA polymerase. Note that the Qdot did not photobleach during the 225 seconds of data collection using a 10ms integration time, and that interactions between the Qdot/immobilized_Phi29 polymerase and Oyster650-labeled γ-nucleotide produced exceptional ASN events (ranging from 1-26). These donors, which have a high extinction coefficient (˜20 times higher than organic donors) and exhibit high FRET, should give an ASN of 5 for modest excitation intensities (˜10 W cm-2) and high nucleotide concentrations (up to 10 mM) even at a 5 ms integration time. Higher excitation intensities will make single frame detection easily possible at high detection efficiency, especially using the non-smoothing version of FRETAN discussed above. In fact, the number of high FRET, single frame events detected in the trace of FIG. 16 doubled when the non-smoothing version was used to detect spFRET. Importantly, high intensity spFRET events have been detected between a single Qdot and two acceptor types (data not shown, but includes: Alexa 594 and Cy5; Alexa 610 and Alexa647; Alexa610 and Cy5; Alexa594 and Alexa647; Alexa595 and Alexa633; Cy5 and Alexa700).

As shown in FIG. 16, the 457 nm laser used to excite the Qdot donor additionally reduced direct excitation of acceptor-labeled nucleotides. In fact, the lower wavelength laser allows us to increase the concentration of acceptor-labeled nucleotides 5-fold, relative to excitation with a 488 laser. Note, too, that the acceptors signals are not (significantly) anti-correlated with the donor signal. The donor signal is about 2500 counts on average, while the acceptor signal bursts are about 680. However, most of the donor signal is purposefully not being collected because it is so bright (the detection system used a 635/25 filter, thereby removing 94% of the signal, which means the donor signal would otherwise be 39,700). Thus, the experimentally determined FRET efficiency (FE) is 3% as shown in the table below. The calculated FE is 5%, given the distance numbers for R₀ (71.5 Å for Oyster650), the polymer thickness (radius of 97 Å), and assuming another ˜20 Å for the distance to the acceptor in the polymerase active site. Fluctuations in the Qdot signal are larger than 5%, thus impeding the ability to detect donor dipping.

Because the quantum dots function as standard FRET donors, one expects that fluorophores closer to the center of the quantum dot will have higher FRET efficiencies. PEGylated Qdots of various sizes were prepared and then immobilized on a microscope slide and acceptor-labeled nucleotides were added to the solution bathing the immobilized quantum dots (See FIG. 15). The polymer coated Qdots, shown in the left hand side of FIG. 16, were obtained from Invitrogen Corp. (Qdot ITK series). For preparation of the non-polymer coated PEG-ylated quantum dots, shown in the right hand side of FIG. 15, 10 μL of a 6.7 μM solution of mercaptoundecanoic acid coated quantum dots (575 nm emission; catalog #CZW-Y-1, NN-Labs, Fayetteville, Ariz.) was transferred to a 0.5 mL eppendorf tube. Solid diaminoPEG₃₄₀₀ (approx. 5 mg) and EDC (approx. 3 mg) were added, and the reaction shaken at RT for 2 hr. The reaction was diluted in water and passed over a 30K MWCO spin filter unit, followed by 3×100 μL water washes. The product was retained on the membrane filter and stored at 4° C. in water for further use in FRET efficiency (FE) studies. A typical FRET efficiency experiment is described below for the non-polymer-coated PED-Qdots. Briefly, the PEG-QD was diluted 6000-fold from the stock solution and applied to a clean glass slide, which was then analyzed on a single-molecule fluorescence microscopy system. The system specifications were as follows: 457 nm laser at 1 mW; QuadView image splitter with the following dichroics: 570, 650; bandpass filters for the three channels of collected light: 555/40, 635/30, 670/40. dA12A1610 was added directly to the slide for a final concentration of ˜2.5 μM, and data collected at 10, 50, and 100 ms integration times for 500 frames. The data was run through our proprietary FRET analysis software (FRETAN) and the 50 ms set was used for FRET efficiency analysis. For the calculation, only anti-correlated FRET events were selected and analyzed manually (only a handful were discarded). The donor and acceptor intensities were corrected to account for the amount of fluorescence captured in the filters, then FRET efficiency calculated using the formula FE=AcInt/(Dint+AcInt). The average FE was found to be 0.74±0.19; the ASN was 3.4±1.3. For a system with similar expected Rₒ and a distance of 60 Å, a theoretical calculation gave a FE of 0.75, which matches quite well with our data. The above procedure was also used to measure FE between acceptor fluorophores and the other Qdot types shown in FIG. 15 and the table below. Infrequently, non-specific FRET interactions were detected in the presence of these acceptor labeled nucleotides. The FRET and acceptor signal-to-noise ratios for these signals are tabulated below, along with the distance from the center of the quantum dot to its surface, and the expected FRET between the Qdot and an acceptor at that distance. The data indicate that the non-specific interactions correspond to temporary binding of the acceptor to the surfaces of the various Qdots or, in the case of the enzyme on the Qdot, to the enzyme.

| | Distance, R | FRET | FRET | Acc. S/N |
|---|---|---|---|---|
| QD type | Å | Calculated | Measured | Measured |
| Coating, PEG, | 117 | 0.05 | 0.03 ± 0.01 | 3.4 ± 0.9 |
| enzyme | | | | |
| Coating, PEG | 97 | 0.14 | 0.17 ± 0.13 | 9 ± 5 |
| Coating, COOH | 77 | 0.4 | 0.26 ± 0.14 | 10 ± 4 |
| PEG | 50-60 | 0.75-0.92 | 0.74 ± 0.19 | 3.4 ± 1.3 |

To generate the data in the first row of the above table, a quantum dot was used having a radius R=117 Å, where R is the distance from the center of the quantum dot to the expected location of the acceptor. This quantum dot was coated with a cross-linked polymer coating, to which PEG (MW=2000) and then polymerase enzyme had been attached. The size is given by the manufacturer and the additional distance (20 Å) is estimated as the distance to the enzyme active site. Unlike the other quantum dots shown here, the enzyme-modified Qdots did not show detectable decreases in the donor signal that anti-correlate with the acceptor signal due to the small amount of energy transfer compared to fluctuations in the quantum dot signal. The second and third rows were generated using quantum dots having radius R measurements of 97 Å and 77 Å, respectively, with a cross-linked polymer coating like the first; the second also has a PEG layer like the first. Measured FRET values are in line with predictions for these distances. The fourth row was generated using a quantum dot (R=50-60 Å) coated with mercaptoundecanoic acid to which diamino-PEG (MW=3400) has been attached via EDC chemistry. The R distances were estimated from information from the manufacturer and an estimation of the PEG layer thickness. In general, quantum dots without a cross-linked polymer coating are less bright and less stable in water than those with the coating. This leads to lower acceptor signal-to-noise ratios in spite of the higher FRET values. Hence, a cross-linked polymer coating is important to maintain high signal-to-noise ratios; thus far it seems advisable to keep the coating in favor of quantum dot stability rather than discarding it in favor of higher FRET. Our data support our hypothesis that to increase the efficiency of FRET between a Qdot and an acceptor-labeled nucleotide, it is necessary to decrease the distance between the core of the Qdot and the active site of the polymerase. Optionally, the quantum dot surface may be modified using methods that maintain the desired fluorescent properties while also keeping the diameter relatively small. Two exemplary such methods include: (1) cross-linking (via click or other chemistry) a small PEG coating after it is assembled on the surface to prevent its disassociation from the dot in solution; and (2) using controlled silane polymerization to create a thin siloxane shell (1-5 nm) around the dot (Gerion, Pinaud et al., 2001; Zhu, 2007).

Alternatively, the donor fluorophore may comprise one or more carbon nanoparticles (Cdots). Although they are not as bright as nanocrystalline donors such as Qdots, it has been reported that surface passivation with diaminoPEG leads to a significant enhancement of fluorescence intensity (Sum et al., 2006). Because the Cdots are ˜1 nm in diameter, they may be an ideal alternative to the larger inorganic Qdots. The small size of these dots should lead to greatly increased FRET efficiency with an acceptor fluorophore. In one exemplary embodiment, a wide spectrum of fluorescent nanoparticles from recovered candle soot was generated according to the published procedure of Liu et al., 2007. According to the literature, these carbon-based nanoparticles exhibited excellent water solubility and more robust fluorescence relative to typical Qdots. At the single molecule level, the Cdots are not as bright as Qdots, but are more stable in aqueous solution and likely more amenable to chemical modification. To study the effect of surface modification on fluorescence as well as to install a handle for further functionalization, these Cdots were coupled with various diamines via EDC activation of the surface carboxylates. The conversion of the surface carboxylate to a surface amine was confirmed by a shift in the direction of electrophoretic mobility on an agarose gel. In the case of smaller diamines, the fluorescence intensity was decreased, and the emission shifted to lower wavelengths. However, coupling of diamino-PEG₃₄₀₀ led to a product with higher bulk fluorescence and no spectral shift.

In some embodiments, fluorophore emissions may be suitably modified using radiative decay engineering techniques, which involve modification of the fluorophore's spontaneous emission rate by various means, usually by placing the emitting species close to a metal particle or surface. Transitions related to species as diverse as nuclear magnetic moments (Purcell, 1946), DNA (Lakowicz, Shen et al., 2001) and organic fluorophores (Malicka, 2002) may be affected by nearby metal. It is even possible to suppress radiation by constructing structures of the appropriate dimension (Kleppner, 1981; Yablonovitch, 1987), and to increase the two-photon excitation rate (Gryczynski, 2002) by placing fluorophores near silver particles. For organic fluorophores undergoing single photon absorption, the decrease in fluorescence lifetime leads to a decrease in photobleaching because the fluorophore spends less time in the excited state (Lakowicz, Shen et al., 2002; Malicka, 2002). Likewise, the increase in the radiative decay rate relative to non-radiative decay causes an increase in quantum yield, with low-quantum yield fluorophores benefiting the most (Lakowicz, 2001; Lakowicz, Shen et al., 2002; Lakowicz, 2003). The FRET rate may be increased orders of magnitude close to a particle with sharp features and with resonance frequency near the molecular transition frequency (Gersten, 1984). In one experiment, such an increase lengthened the effective Rₒ by a factor of two (Malicka, Gryczynski et al., 2003); this may be relevant to a strategy involving quantum dots as donors, wherein the acceptor is located at a large distance from the center of the quantum dot. Typically, the donor-labeled polymerase will be precisely positioned at the correct distance from a metal, e.g., silver particle, so as to obtain the best possible signal enhancement. (See, for example, Zhang et al, 2007). It has been shown (Malicka, Gryczynski et al., 2003) that layers of BSA-biotin and avidin on top of silver island films can provide this positioning. The first layer of BSA-biotin/avidin positions a Cy3-labeled duplex at a distance that enhances the fluorescence by a factor of 11. A silver island film or silver colloid could similarly be coated with PEG of an appropriate length to optimize enhancement.

The enhancement of fluorescence occurs for all fluorophores within a certain distance from a metal particle, although some may be enhanced more or less depending on the fluorophore's excitation properties. However, the excitation distance dependence is stronger for the enhancement due to metal particles than for excitation due to TIR, with decay constants of ˜6 nm (Malicka, Gryczynski et al., 2003) and 80 nm, respectively. Thus there is an advantage to using metal particles to enhance the signal because the enhancement of a properly placed donor will be greater than that of the acceptors in solution (which are in the TIR field but are not close enough to be enhanced by the metal particle).

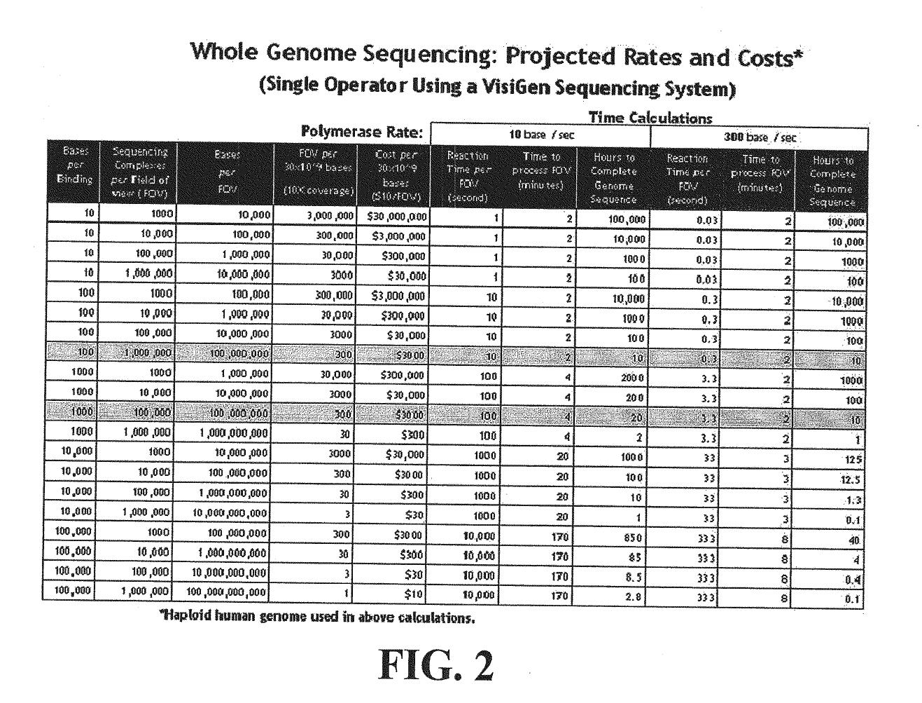

As illustrated in the table of FIG. 2, the sequencing methods of the present disclosure can be performed in a massively parallel manner, resulting in ultra-high sequence throughput and cost savings. There are a number of strategies to implement the disclosed sequencing applications in high-throughput format. In one exemplary embodiment, the polymerase enzyme may be immobilized in a closed system device, and primer, template and nucleotides delivered into the reaction chamber to initiate the reaction. Based on our current ability to resolve complexes using wide-field microscopy, CCD imagers containing 1M pixels in parallel for each fluorophore may be used image a field of view containing 50,000 distinct, arrayed sequencing complexes. The sequencing reactions can optionally be imaged for 30-60 seconds, after which the adjoining chamber (containing non-photobleached, donor-labeled polymerase) can be automatically repositioned into the field of view, and these reactions can be initiated and imaged. If this process is used to interrogate 100 fields of view within the closed-system device and one cycle of interrogation requires approximately 2 minutes (to move to the adjoining chamber, deliver reagents, focus, and image data), data will be collected from 5,000,000 complexes in a sequential and massively parallel fashion in less than 4 hours. Such techniques can optionally be used in conjunction with an imaging strategy that involves continuous data acquisition via moving stage detection. See, for example, Battulga et al., U.S. Ser. No. 11/781,166; Rea, U.S. Ser. No. 11/781,157 for description of exemplary imaging strategies.

The table shown in FIG. 2 illustrates the calculated effect of sequence throughput on sequencing cost for 10× coverage of the human genome, and the time (in hours) for completion as the indicated variables change (The variables include the number of bases detected per sequencing complex; the number of sequencing complexes per field of view (FOV); the number of FOV needed to collect 30 billion bases of sequence). The ‘Hours to Complete’ column was calculated by multiplying the reaction time/FOV by the number of FOVs required to determine 30×10⁹ bases. These numbers do not assume any down time and only account for the time to perform the sequencing reaction. The times required for DNA preparation and data analysis are not included.

In some embodiments, the amount of sequence information obtainable from a single sequencing run can be increased by employing long read lengths and/or increased rates of incorporation of detectably labeled nucleotides by the polymerase, in conjunction with a massively-parallel array of complexes, or a combination of the preceding strategies, respectively.

Also disclosed herein are methods for increasing acceptor signal during single-molecule FRET events. Such methods may optionally be used in conjunction with methods involving improved incorporation rates. Depending on the reaction steps that are accelerated to obtain the increased incorporation rate (10 vs 300 bases/sec), the donor-acceptor fluorophores may not be in close proximity long enough for the donor to transfer sufficient energy to produce detectable dynamic spFRET. The disclosed methods of increasing acceptor signal will improve detectability at the faster integration times needed to collect data at increased incorporation rates. In some embodiments, data are captured at 5-10 times faster than the incorporation rate so that the donor's return to pre-spFRET intensity is detected and can be used to delineate single incorporation signatures from sequentially incorporated nucleotides.

As illustrated in the table of FIG. 2, data quality necessitates high accuracy per reaction (>98%), and sufficient coverage of each base for accuracy and assembly purposes, such that a single-molecule approach may require deeper coverage than current sequencing methods. In some embodiments, sequencing coverage is 10-fold; optionally, the degree of coverage may be adjusted either upwards or downwards. The effect of collecting 100 vs. 1000 base reads from either 1,000,000 or 100,000 complexes, respectively, is highlighted in FIG. 2. In both cases, 10× coverage of a genome similar in size to the human is obtained from 300 FOV. 100-base read lengths are sufficient to assemble almost all regions of the genome, although there may be problems with repeated regions (Shendure, Mitra et al., 2004). As shown in FIG. 2, if 1,000,000 complexes are monitored in parallel, an incorporation rate as slow as 1 per second produces a sequencing rate of 1M bases/sec/machine.

In the disclosed sequencing applications, the spFRET event typically occurs from the time the acceptor fluorophore labeled nucleotide enters the active site of donor labeled DNA polymerase through the moment when the terminally labeled polyphosphate is released from the enzyme, which coincides with the chemistry step (bond cleavage and bond formation) during DNA synthesis. As discussed above, spFRET detectability can optionally be increased by prolonging the chemistry step through introduction of suitable mutations in the polymerase.

In addition to modifying the enzyme, another optional strategy to slow the chemistry step involves modifying nucleotides, especially at positions around the nucleotide alpha-phosphate (Dobrikov, Grady et al., 2003; Bakhtina, Lee et al., 2005). Disclosed herein are methods to detect a nucleotide incorporation event, comprising: conducting a nucleotide polymerase reaction in the presence of one or more detectably labeled nucleotides that have been modified to exhibit increased duration of association with the polymerase before, during or after a nucleotide incorporation event, which reaction results the production of a detectable signal before, after or during a nucleotide incorporation event; and detecting the detectable signal, thereby determining if a nucleotide incorporation event has occurred. Optionally, the detectable label of the nucleotide is a FRET acceptor, and/or the detectable signal is a FRET signal. Optionally, the methods further comprise the step of analyzing the signal to determine the identity of the nucleobase of the incorporated nucleotide.

Initial tests with [alpha]-P-borano-dGTP and [alpha]-P-thiol-dGTP show slightly slower reaction rates as compared to the natural counterpart (1.32- and 1.61-fold reduction, respectively; data not shown).

One component of some embodiments of the disclosed sequencing systems is the solid support on which the nucleotide polymerase reaction takes place. The sequencing reaction is typically accomplished with a polymerase/DNA complex immobilized on the solid support, glass or fused silica slide. In one exemplary embodiment, the solid support is biologically friendly for multiple components with very different physical properties. Since protein molecules are rather amphiphilic, nucleotides and DNA are negatively charged, and fluorophore labels are hydrophobic, this surface does not carry positive or negative charges, is hydrophilic, and has functional groups for the specific attachment of a polymerase or DNA duplex. Some exemplary surfaces used for the sequencing systems include Ni—NTA-HRP surface (for immobilization of His-tagged enzyme) or PEBN surface (for immobilization of biotinylated enzyme on and/or nucleic acid duplexes). Ni—NTA-HRP and PEG surfaces have high specificity for protein binding, but exhibit some level of background nucleotide binding under certain conditions. Another exemplary surface comprises a functionalized polyethylene glycol (PEG) layer to form a protein friendly surface. See, for example, Guo and Zhu, 2006. One exemplary approach to forming PEG surfaces involves the amino-silanization of glass slides, followed by a reaction with NHS—PEG, resulting in a surface that exhibits some level background binding to dye-labeled molecules (data not shown). Alternatively embodiments include carbohydrate surfaces with a possible addition of PEG chains, as well as replacement of adsorptive and ionic surfaces with hydrophilic non-ionic surfaces and generation of surfaces based on multi step, multi component modifications through hydroxy-silanization and/or carbohydrate coating (hyaluronic acid etc.), and/or surface PEGylation. For example, bis(hydroxy)-silane can be used to create hydrophilic surfaces that have organic hydroxyls available for chemical modification with, for example, functionalized phosphoramidites; hyaluronic acid may be added through adsorption to a glass or a positive layer, or alternatively by chemical binding. Alternatively, bi-functional PEG, along with a PEG-OH capping reagent, may be prepared and added to a silane-modified surface.

Advantages of the disclosed sequencing methods, systems and compositions include the ability to exploit the natural process of DNA replication in a way that enhances accuracy and minimally impacts efficiency. This approach involves engineering both polymerase and nucleotide triphosphates to act together as direct molecular sensors of DNA base identity in real-time.

One challenge of such sp-FRET based systems the ability to distinguish a γ-labeled nucleotide incorporation signal from either non-productive nucleotide binding or collisional FRET events. In some embodiments, this challenge may be addressed via fine-tuning our system to detect characteristics of the incorporation product (ie., labeled polyphosphate), and by training the software to distinguish non-productive interactions from incorporation events. For example, an incorporation event may be detected by monitoring approximately 50 attributes associated with spFRET, including the intensity and duration of donor and acceptor emission before, during and after nucleotide incorporation. These signals may be compared against characterized non-specific signals (background signals).

In some embodiments, to more selectively observe sequencing signals, extension reactions can be optimized to reduce non-productive binding and ‘background’ signal by 1) determining the lowest nucleotide concentration that supports desired enzyme activity, 2) identifying a polymerase that more efficiently binds the correct γ-nucleotide and slows the chemistry of its incorporation (producing a longer-lived signal), and 3) optimizing experimental conditions (temperature, buffer and co-factors) to improve the efficiency of γ-nucleotide associated spFRET as well as overall reaction efficiency.

A major strength of this technology is its highly parallel nature, which allows for increase of throughput. To illustrate this, the enzyme was immobilized in a closed system device, and primer, template and nucleotides are delivered into the reaction chamber to initiate the reaction. Based on the current threshold of resolution of complexes using wide-field microscopy, CCD imagers containing 1M pixels used in parallel for each wavelength can image a field of view containing 50,000 distinct sequencing complexes. The sequencing reactions will be imaged, after which the adjoining chamber will be automatically repositioned into the field of view, and these reactions will be initiated and imaged. If this process interrogates 100 chambers within the closed-system device and one cycle of interrogation requires approximately 2 minutes (to move to the adjoining chamber, deliver reagents, focus, and image data), data will be collected from 5,000,000 complexes in a sequential and massively parallel fashion in less than 4 hours.

A currently-used camera contains 512×512 pixels but if integration time is less than 25 msec a smaller area of the chip is scanned (at 25msec 360×360 pixels; 129,600 pixels). Using our current detection system equipped with the QuadView beam splitter, the maximum number of complexes that can be individually monitored with 1 pixel spacing between complexes is 2025 (129,600 total pixels/4 due to beam splitter/16 pixels per complex); the maximum number of complexes that can be individually followed with 2 pixels between complexes is 900 (129,600 total pixels/4 due to beam splitter/36 pixels per complex). However, because the complexes are (currently) randomly distributed on the surface, rather than arrayed in precise grids, 200-300 complexes per field of view are monitored. Using a random array of nano-sequencing machines, our limit of resolution is and will remain about 5% of chip capacity because each sequencing complex occupies 4 pixels and must be far enough away from its neighbors to distinguish separate nanomachines. Chip capacity of 1,000,000 pixels (using a 1Kx1K chip) could permit simultaneous monitoring of 50,000 complexes (using multiple cameras and ordered arrays). An ordered array could increase throughput.

Also disclosed herein are gamma-labeled nucleotides comprising a FRET acceptor operably linked to, or otherwise attached, to the terminal, gamma- or other non-persistent phosphate, as well as methods for synthesis of such nucleotides using triazole “click” chemistry. (Rostovtsev et al., 2002). “Click” based chemical reactions are typically modular, wide in scope, high yielding, create only inoffensive by-products (that can be removed without chromatography), are stereospecific, simple to perform and that require benign or easily removed solvent. Typically, the starting materials and reagents for ‘click’ reactions are also readily available. Several processes have been identified for the potential of click chemistry, including but not limited to nucleophilic ring opening reactions:epoxides, aziridines, aziridinium ions etc.; non-aldol carbonylchemistry: formation of ureas, oximes and hydrazones etc.; additions to carbon-carbon multiple bonds: especially oxidative addition, and Michael additions of Nu-H reactants; and cycloaddition reactions: especially 1,3-dipolar cycloaddition reactions, but also the Diels-Alder reaction. (See, e.g., Kolb et al., 2004; Rostovtsev et al., 2002; Diels et al., 1928; Holmes, 1948). Click chemistry can be used to prepare modified nucleotide libraries in large numbers and varieties from a single gamma-modified precursor. This highly efficient chemistry may also allow installation of highly complex structures and functions into modified nucleotides. Based on the specific desired synthesis, the appropriate chemistry can be chosen to meet the investigator's needs. Both diene-alkene click chemistry and acetylene-azide click chemistry offer several advantages in organic chemistry: high yield, no need of exclusion of oxygen and moisture, wide solvents compatibility including water, high orthogonal reactivity, etc. See, for example, (See, e.g., Kolb et al., 2004; Rostovtsev et al., 2002; Diels et al., 1928; Holmes, 1948).

Disclosed herein are methods for synthesizing a detectably labeled nucleotide, comprising: (a) introducing a first click group onto a nucleotide; (b) introducing a second click group capable of specifically reacting with the first click group onto a detectable label; and (c) reacting the nucleotide with the detectable label, thereby forming a detectably labeled nucleotide. In some embodiments, the first and second click groups are selected from the group consisting of: a terminal alkyne group, an azide group, a conjugated diene group, and a substituted alkene group. Optionally, the first click group can be introduced onto a phosphate group, nucleobase or sugar moiety of the nucleotide. In some embodiments, the first click group is introduced onto the terminal phosphate of the nucleotide.

Also disclosed herein are two different click chemistry-based methods of synthesizing labeled nucleotides using click chemistry. In one method, terminal alkyne groups groups (CH≡C—) were introduced onto the NTP terminal phosphate for click chemistry using a variety of linker-azide structures (see exemplary synthetic approach 1, below). In a second approach, an azide group (N₃—) is installed on to NTP terminal phosphate for click chemistry with a wide variety of linkers comprising a terminal alkyne group (see exemplary synthetic approach 2, below). Both produce new linking moieties comprising a triazole structure close to the terminal phosphate, to which suitable labels can then be attached.

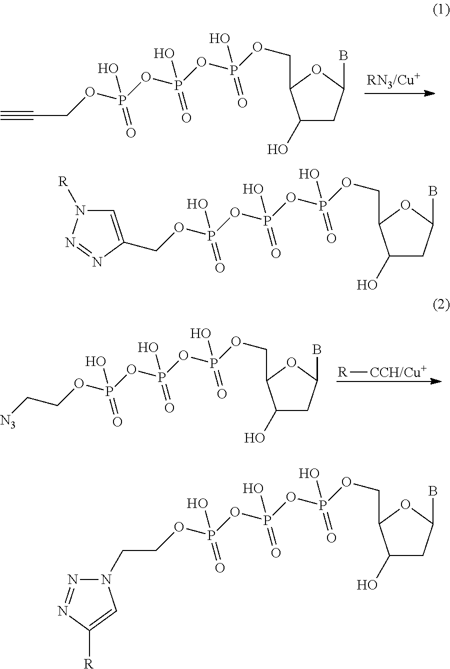

More generally, both terminal alkyne and azide functional groups can be introduced into favored NTP-linker structures at the linker termini and corresponding click chemistries can be performed (see exemplary synthetic approaches 3 and 4, below).

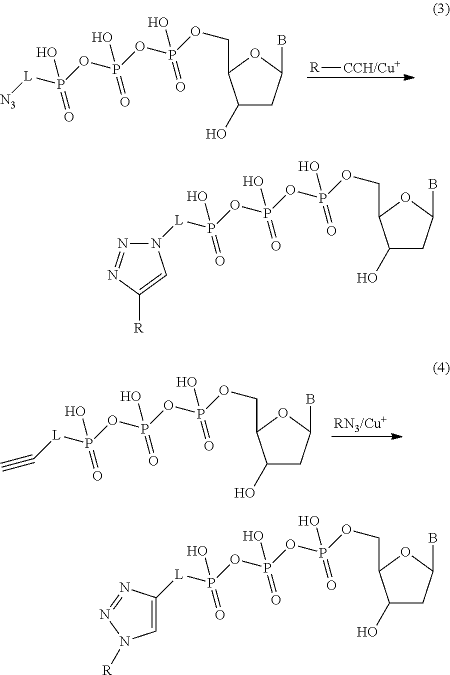

Such synthetic designs may be used to create large libraries of molecules using click chemistry. A variety of functional groups or their combinations can be incorporated into the final products from the linker with minimal protecting group chemistry. This will allow the freedom in tuning the molecular properties with charges, glycosylation, PEGs, etc.

Accordingly, disclosed herein is an exemplary method for click-based synthesis of a gamma-labeled nucleotide comprising: (a) introducing a terminal alkyne group onto the terminal phosphate of a nucleotide; (b) reacting the nucleotide with a first compound comprising an azide group and a linking group, thereby forming a nucleotide comprising a linking group attached to the terminal phosphate; and (c) reacting the linking group with a second compound comprising a detectable label, thereby forming a terminally labeled nucleotide. Optionally, the order of steps may be rearranged in any suitable order that results in the formation of a terminally labeled nucleotide, including the performance of step (c) prior to step (b). In some embodiments, the introducing step can further comprise replacing the leaving group of an alkyne-containing compound with the nucleotide to form a nucleotide comprising an alkyne group attached to the terminal phosphate.

In some embodiments, the first compound may selected from the group consisting of: azidoamine and an azide-containing linker. In one exemplary embodiment, the azide-containing linker has the formula CF₃CONH—CH₂CH₂—N₃.

In some embodiments, the first reacting step (b) is performed in the presence of one or more substances selected from the group comprising: Copper (Cu) and t-butanol.

In some embodiments, the second reacting step (c) is performed in the presence of sodium bicarbonate (NaHCO₃).

In an alternative embodiment, the method for synthesizing a terminally labeled nucleotide comprises: (a) introducing an azide group onto the terminal phosphate of a nucleotide; (b) reacting the nucleotide with a first compound comprising a terminal alkyne group and a linking group, thereby forming a nucleotide comprising a linking group attached to the terminal phosphate; and (c) reacting the linking group with a second compound comprising a detectable label, thereby forming a terminally labeled nucleotide. Optionally, the order of steps may be rearranged in any suitable order that results in the formation of a terminally labeled nucleotide, including the performance of step (c) prior to step (b).

Any suitable detectable label may be used in the disclosed nucleotide synthesis methods of the present disclosure. For example, the detectable label can optionally be selected from the group consisting of: fluorescent or fluorogenic labels, luminescent or luminogenic labels; chromogenic labels, electrochemical labels; mass tags; and radioactive labels. Typically, the detectable label of the above nucleotide synthesis method is a fluorescent label selected from the group consisting of: Alexa Fluor, fluorescein, Oregon Green, rhodol, rhodamine dyes, Tokyo Green, Texas Red, resorufin, ROX, pyrene, cyanine, coumarin, dansyl, BODIPY and derivatives thereof.

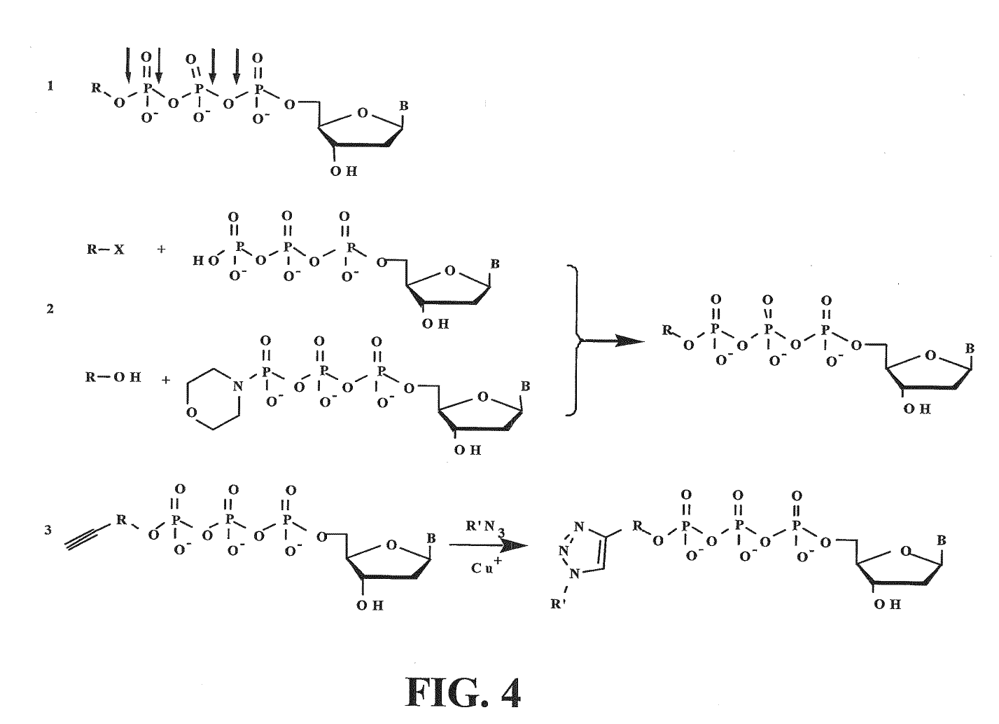

FIG. 4 outlines an exemplary synthetic method for synthesis of gamma-labeled nucleotides. In this method, the gamma-labeled nucleotide dA-1-Cy5 is accomplished by first making dA-1 (Compound 2 below). To achieve this, a mixture of dATP (7 mg, 12.7 mmol) and EDC (10 mg, 52 mmol) in MES buffer (0.1M, pH 5.7) was stirred at room temperature for 10 min. Ethylenediamine hydrochloride (7 mg, 53 mmol) was added and the mixture was stirred for an additional 2-3 hr. The pH was maintained at 5.7-5.8 throughout the reaction. The reaction mixture was lyophilized, resuspended in water (1 mL) and purified by HPLC (Amersham, Mono Q 5/5, followed by Supelco C18, TEAA/AcN). The product fractions were collected and lyophilized to a white powder, which was dissolved in water and the absorbance measured at 259 nm (72% yield). dA-1 (42 ml, 1 mmol) was then dissolved in NaHCO₃ buffer (1 M, pH 9.0, 70 ml) and Cy5-NHS (1 mg, 1.3 mmol, dissolved in 100 mL dry DMF) was added, and the reaction shaken overnight. The crude reaction mixture is lyophilized and run over a Sephadex G-25 column, followed by HPLC purification (C18, TEAA/AcN). The product (3) was lyophilized, resuspended in HEPES buffer (200 mL) and the absorbance measured at 646 nm (20% yield). The resulting product was characterized via digestion with phosphodiesterase I (PDE1). Specifically, the synthesis product (1.1 mM 1 ml) was treated with PDE1 at room temperature for 20 min and the digestion products were analyzed by thin layer chromatography (TLC). The products were identified as dAMP and PPi-1-Cy5 by comparison with authentic samples.

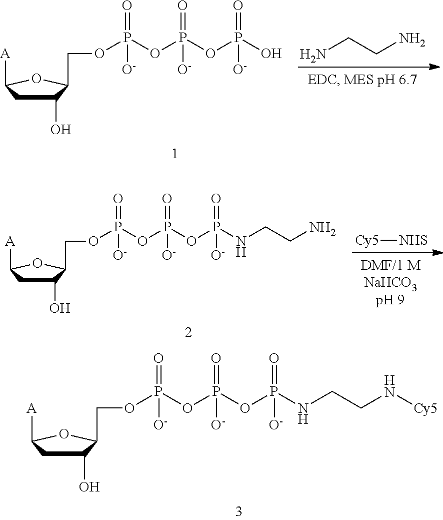

In another exemplary click-based embodiment, a nucleotide comprising an acetylene group was prepared using the reagents dATP, propargyl benzenesulfonate and DMF as depicted below:

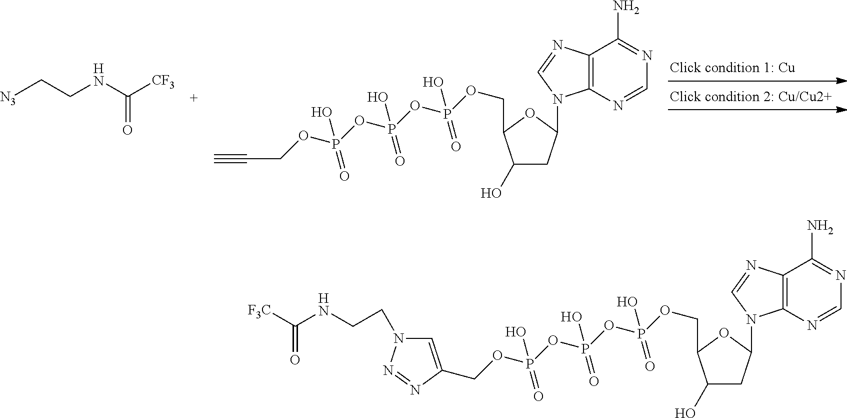

The azide was prepared from trifluoroacetyl β-iodoethylamine and sodium azide in DMSO and purified with silica flash column chromatography.

In an exemplary embodiment of the click-based methods disclosed herein, the nucleotide (40 nmol) and azide (120 nmol) were mixed in tert-butanol (25 uL)/water (35). A short copper wire (17 umol) was added to the mixture and the reaction vial was capped. The reaction was shaken on a shaker at r.t. for 24 hr and HPLC (SAX) showed complete conversion. The reaction was left on the shaker for another 24 hr before water (1.3 mL) was added. The mixture was taken to HPLC purification (SAX, TEAA) and afforded the desired product 35 nmol (88%). ESI Mass Spectrometry confirmed its structure (C₁₇H₂₃F₃N₉O₁₃P₃): experimental 711.4; calculated 711.03.

In another exemplary embodiment of click-based nucleotide synthesis, gamma-labeled nucleotides were synthesized using the same method described in the preceding paragraph, except that Cu₂SO4 (0.4 nmol) was added to the reaction. The product was analyzed with ESI Mass Spectrometry to give the same result 35 nmol (88%): C₁₇H₂₃F₃N₉O₁₃P₃: experimental 711.4; calculated 711.03. The results of the analysis indicate that the reaction is highly efficient at very low reactant concentrations (0.67 mM) and easily run on very small scales (40 nmol).

Such click-based techniques should allow installation of more complex structures into the linkers to offer more functions/desired properties than a spacer. It should be efficient in surface chemistry, immobilization chemistry, and dendritic chemistry as well in related research and applications.

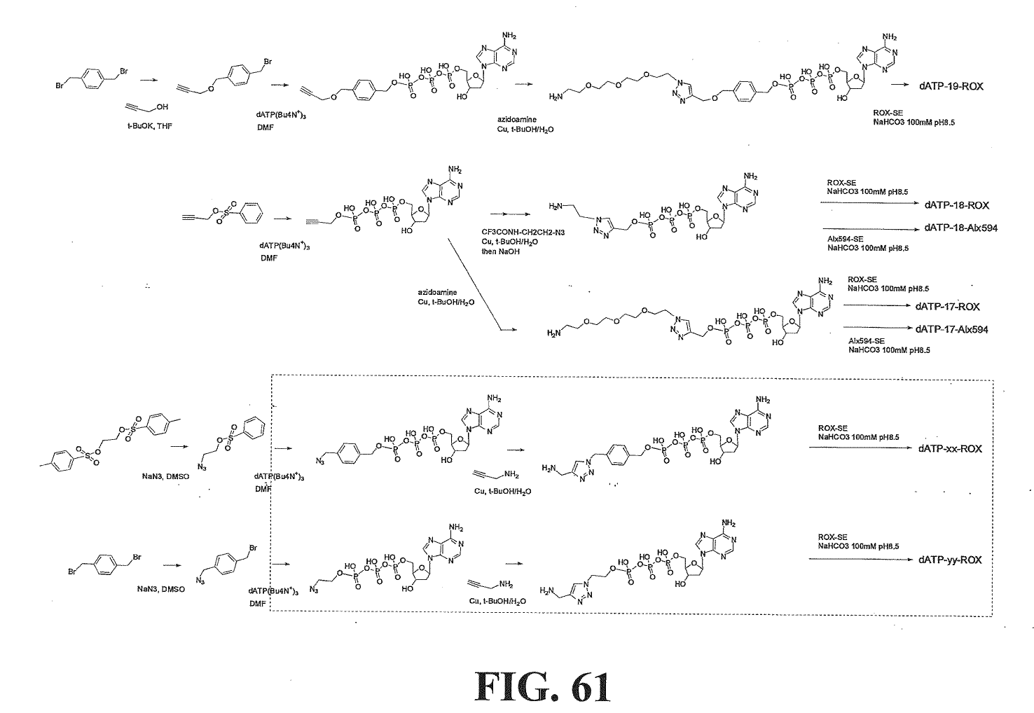

FIG. 61 illustrates some exemplary click synthetic methods according to the preceding paragraphs.

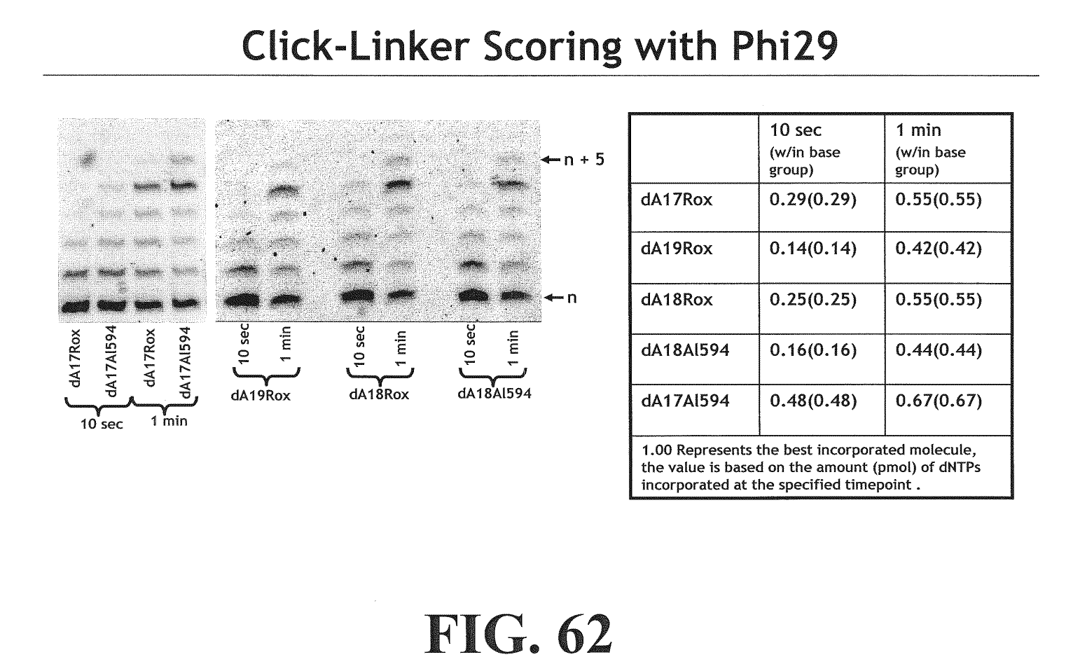

Referring now to FIG. 62, primer extension reactions using the click prepared γ-labeled nucleotides are shown (left), along with the numerical data scores. The scoring assay measures how well click-generated dNTPs are incorporated at a final concentration of 0.5 μM at either 10 seconds or 1 minute. This extension is on a 7 base homo-polymeric template. The final concentration of Phi29 polymerase and duplex is 330 nM and 100 nM, respectively. The reactions are separated on a 20% denaturing acrylamide gel. The data presented in FIG. 62 shows that the gamma-modified molecules made with click chemistry linkers are incorporated by Phi29 polymerase.

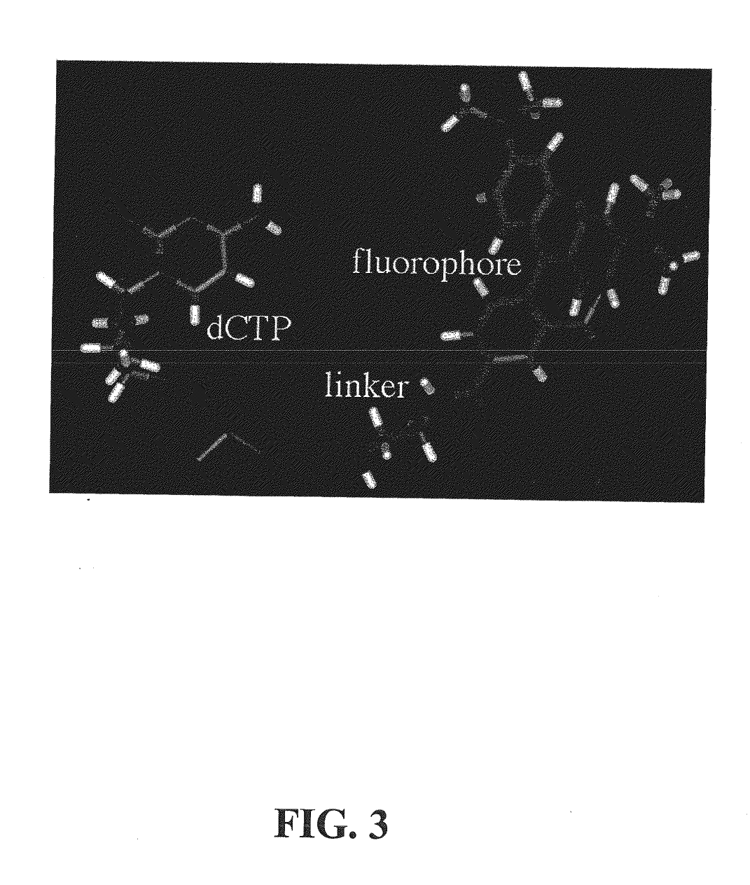

FIG. 3 depicts structures of an exemplary gamma-modified nucleotide according to the present disclosure; the nucleotide, linker and fluorophore components are indicated therein. As an example, the modified nucleotide is identified by its base (dCTP), linker type (#1), and fluorophore (TAMRA), and referred to as “dCTP-1-TAMRA” or “dC-1-T”.

Using such techniques, detectably labeled nucleotides may be synthesized using any suitable conditions that preserve the ability of the nucleotide to undergo polymerization by the polymerase and the ability to detect the label.

Optionally, the sequence applications disclosed herein may incorporate suitable methods of determining whether gamma- or terminally-labeled nucleotides can be efficiently incorporated by various polymerases using thin-layer chromatography (TLC) to separate intact nucleotide from labeled pyrophosphate or polyphosphate. Such methods allow, for example, the detection both of products of an incorporation event: (1) labeled pyrophosphate or polyphosphate via TLC analysis and (2) extended primer via gel electrophoresis. These dual assays provide a mechanism to (1) screen polymerases for the ability to utilize gamma or omega-modified nucleotides; (2) determine the incorporation efficiency for any given modified nucleotide; and (3) monitor the purity of labeled nucleotide stocks. The quantifiable detection range is on the order of 0.5-100 pmol.

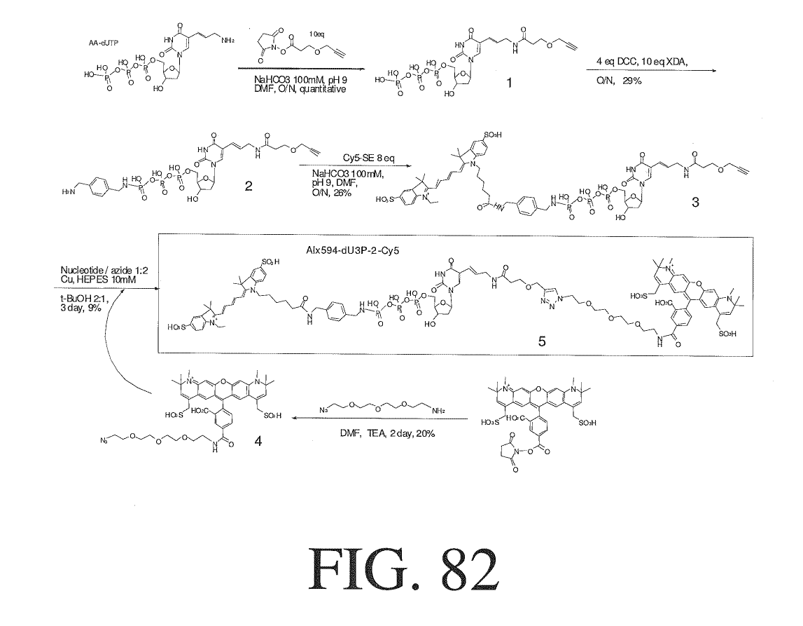

In some embodiments, sequence-specific incorporation of nucleotides labeled on the gamma or terminal phosphate may be accomplished using a dual-labeled nucleotide comprising two different labels: one “non-persistent” attached to a non-persistent portion of the nucleotide (such as the beta, gamma or terminal phosphate), and another “persistent” label attached to a portion of the nucleotide that becomes incorporated into the newly synthesized nucleic acid molecule, for example, the nucleobase. Such a dual labeled nucleotide will associate the intense and long-lived base-labeled nucleotide signal with the non-persistent signal of a γ-label of the nucleotide that is released from the nucleotide during or after incorporation by the polymerase. In some embodiments, the dual-labeled nucleotide contains an orange dye (ROX) on the base and a red dye (Cy5) on the γ-phosphate (see exemplary structure, below and FIG. 82). As the molecule approaches a donor-labeled polymerase we expect to detect Cy5 signal due to energy transfer from the donor through ROX to Cy5 (triple fluorophore FRET). Following incorporation, Cy5-PPᵢ will leave the proximity of the donor and the ROX signal will appear and persist. Traces may be examined to determine features associated with the Cy5 signal. If Rox and Cy5 quench each other, neither may be detected until Cy5 leaves, which is still informative.

Disclosed herein are compositions of dual labeled nucleotides, wherein a first detectable label is operably linked to the γ-phosphate and second detectable label is operably linked to the base, sugar or α-phosphate, and the first and second detectable labels do not significantly quench each other. The term “quenching” and its variants, as used herein, refer to any process which decreases the intensity of the detectable signal of a given substance. Quenching may occur through a range of mechanisms, such as spectral interference, excited state reactions, energy transfer, complex-formation and collisional quenching. Typically, the first and second detectable labels are covalently bonded to the γ-phosphate and the base, sugar or alpha phosphate, as the case may be. In some embodiments, the quenching effect of the first or second detectable label on the other label is less than 50%, less than 40%, less than 30%, less than 20% or less than 10%.

More particularly, provided herein are dual labeled nucleotide compositions, comprising a first detectable label operably linked to the terminal phosphate and a second detectable label operably linked to the nucleobase, wherein the first and second detectable labels do not significantly quench each other. More particularly, disclosed herein are nucleotide having the formula: D1-P—(P)ₙ-S-B-D2, wherein P is phosphate (P03) and derivatives thereof; n is 2 or greater; B is a nucleobase; S is an acyclic moiety, a carbocyclic moiety, or sugar moiety; D1 is a detectable label that is attached to the terminal phosphate; and D2 is a detectable label that is attached to nucleobase; and wherein D1 and D2 do not significantly quench each other.

In some embodiments, the dual labeled nucleotide comprises 2 or more phosphate groups. In some embodiments, the dual labeled nucleotide comprises 3, 4, 5 or more phosphate groups.

Optionally, D1 is attached to the terminal phosphate through a linker L1, or D2 is attached to the nucleobase through a linker L2, or both D1 and D2 are attached to the terminal phosphate and nucleobase through linkers L1 and L2, respectively. In some embodiments, D1 is attached to the terminal phosphate through a linker L1 and D2 is attached to the nucleobase through a linker L2.

In some embodiments, at least one of D1 and D2 are each selected from the group consisting of: fluorescent or fluorogenic labels, luminescent or luminogenic labels; chromogenic labels, electrochemical labels; mass tags; and radioactive labels. Optionally, at least one of D1 and D2 is a fluorescent label selected from the group consisting of: Alexa Fluor, fluorescein, Oregon Green, rhodol, rhodamine dyes, Tokyo Green, Texas Red, resorufin, ROX, pyrene, cyanine, coumarin, dansyl, BODIPY and derivatives thereof.

In some embodiments, the nucleobase is an adenine, guanine, cytosine or thymine.

In some embodiments, the sugar moiety of the nucleotide is selected from a group consisting of pentose and hexose sugars. Optionally, the sugar moiety of the nucleotide is selected from a group consisting of ribose, deoxyribose and derivatives thereof.

Typically but not necessarily the dual labeled nucleotide is capable of being incorporated onto the terminal 3′ OH of a synthesized DNA molecule by a polymerase. Optionally, the polymerase is a DNA polymerase, an RNA polymerase or a reverse transcriptase.

Optionally, at least one linker comprises a hydroxyl group, a sufhydryl group, an amino group, an azido group, an alkyne group, a haloalkyl group, a triazole group, or an amido group. In some embodiments, at least one linker contains a group suitable for forming a phosphate ester, a thiester, a phosphoramidate, an azide, an alkyne, or an alkyl phosphonate linkage between at least one detectable label and the nucleotide.

Optionally, at least one of D1 and D2 is a fluorogenic moiety whose fluorescence is enhanced after it is acted upon by an enzyme.

Optionally, D1 is Cy5 and D2 is Alexa Fluor 594.

In one exemplary embodiment, the dual labeled nucleotide has the structure:

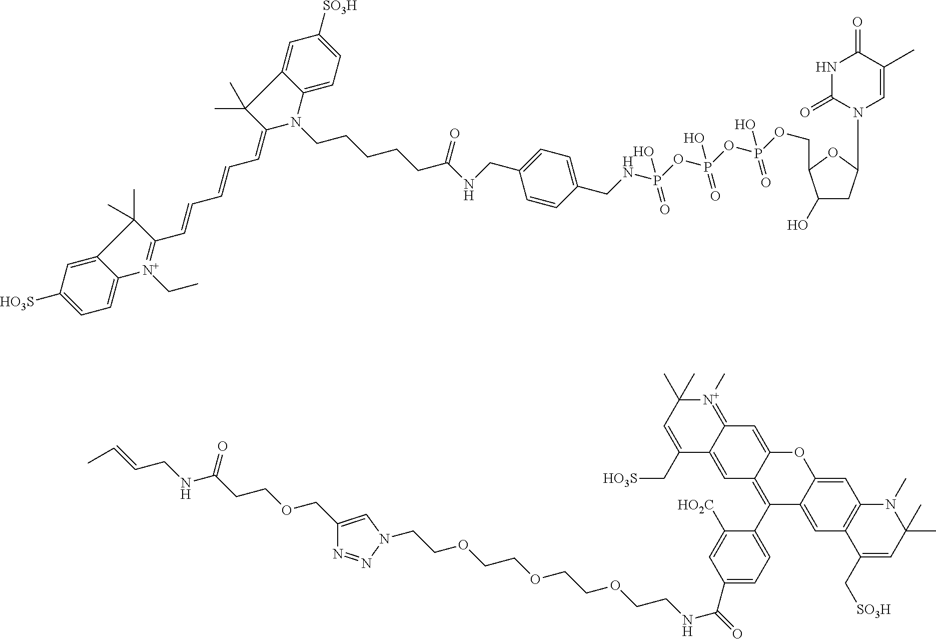

or alternatively has an equivalent structure wherein the sugar of the above structure is replaced by a different sugar, or the nucleobase by a different nucleobase.

In some embodiments, D1 is Alexa Fluor 647 and D2 is Alexa Fluor 680.

Also disclosed herein is a method for detecting a nucleotide incorporation event using a dual-labeled nucleotide, comprising: (a) conducting a nucleotide polymerase reaction in the presence of one or more dual labeled nucleotides, which reaction results the production of a detectable signal before, after or during a nucleotide incorporation event; and (b) detecting the detectable signal and thereby determining if a nucleotide incorporation event has occurred.

The present disclosure also provides methods for synthesis of dual labeled nucleotides using click chemistry. For example, disclosed herein is a method for synthesizing a dual labeled nucleotide, comprising: (a) introducing a terminal alkyne group onto the nucleobase of the nucleotide to form an alkynyl nucleotide; (b) attaching a first detectable label to the terminal phosphate of the nucleotide to form a labeled alkynyl nucleotide; and (c) reacting the terminal alkyne group of the nucleobase with a labeled azide compound comprising an azide group and a second detectable label, thereby forming a nucleotide comprising a first detectable label attached to the terminal phosphate and a second detectable label attached to the nucleobase.

In some embodiments, the azide group of the labeled azide compound reacts with the terminal alkyne group to form a triazole group linking the second detectable label to the nucleobase.

In some embodiments, the introducing step further comprises reacting a nucleotide comprising a terminal amine group attached to the nucleobase with a succinimide ester compound comprising a terminal alkyne group.

Optionally, the starting nucleotide is an amino allyl nucleotide.

In some embodiments, the amino allyl nucleotide has the structure:

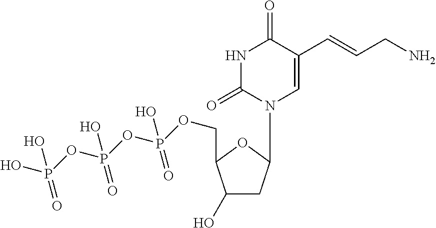

or alternatively has an equivalent structure wherein the sugar of the above structure is replaced by a different sugar, or the nucleobase by a different nucleobase.

In some embodiments, the alkynyl nucleotide has the structure:

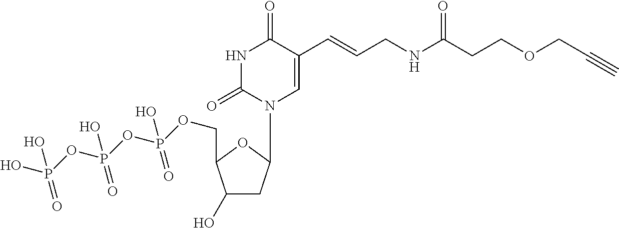

or alternatively has an equivalent structure wherein the sugar of the above structure is replaced by a different sugar, or the nucleobase by a different nucleobase.

In some embodiments the attaching step further comprises reacting the alkynyl nucleotide with a linking compound comprising a reactive group to form a reactive nucleotide; and reacting the reactive nucleotide with a compound comprising the first detectable label to form the labeled alkynyl nucleotide.

In some embodiments, the reactive group may include an amino, thio or carboxyl group.

Optionally, the linking compound is a diamine linker.

Optionally, the diamine linker can be selected from the group consisting of: xylene diamine (XDA), 2,4,6-trimethylphenylene diamine (TMPDA), and 3,5-diaminobenzoic acid (DBA).

In some embodiments, the step of forming a reactive nucleotide is performed in the presence of dicyclohexylcarboimide.

Optionally, the reactive nucleotide may comprise a reactive group that is attached to the terminal phosphate via a phosphoester (P—O) bond, a phosphoramide (P—N) bond, a phosphothio bond (P—S), or a phospho-carbon bond (P—C).

In some embodiments, the reactive nucleotide has the structure:

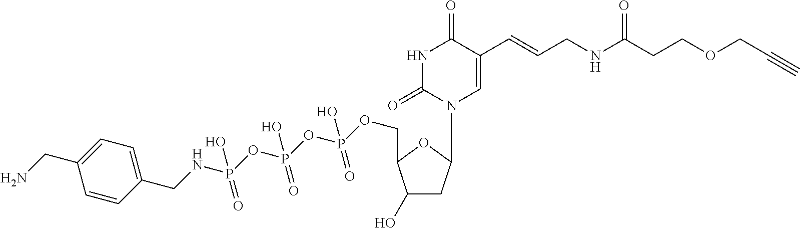

or alternatively has an equivalent structure wherein the sugar of the above structure is replaced by a different sugar, or the nucleobase by a different nucleobase.

Optionally, the compound comprising the first detectable label further comprises a succinimide ester.

In some embodiments, the succinimide ester reacts with an amino group on the terminal phosphate of the nucleotide.

Optionally, the labeled azide compound can be formed by reacting a compound comprising the second detectable label and a succinimide ester group with a compound comprising an azide group and an amino group.

In some embodiments, the first detectable label D1 is active, and the second detectable label D2 is active or non-active.

Also disclosed herein is an alternative method for synthesizing a dual labeled nucleotide, comprising: (a) introducing a terminal azide group onto the nucleobase of the nucleotide to form a nucleotide azide; (b) attaching a first detectable label to the terminal phosphate of the nucleotide to form a labeled nucleotide azide; and (c) reacting the azide group of the nucleobase with a labeled alkyne compound comprising a terminal alkyne group and a second detectable label, thereby forming a nucleotide comprising a first detectable label attached to the terminal phosphate and a second detectable label attached to the nucleobase.

In some embodiments, the azide group of the labeled nucleotide azide reacts with the terminal alkyne group to form a triazole group linking the second detectable label to the nucleobase.

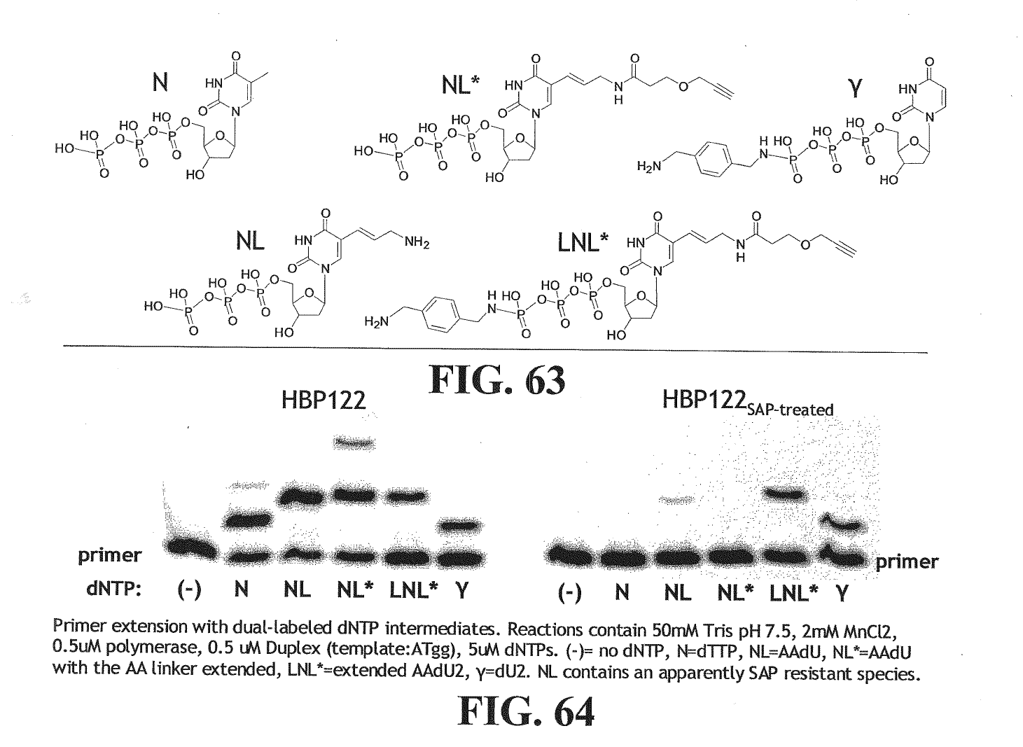

FIG. 63 depicts a set of nucleotides to illustrate the construction of a dual labeled nucleotide according to the present disclosure, where the γ-phosphate label is a fluorescent label and the base label can be fluorescent or not and can be designed to change the incorporation dynamics of the nucleotide. Referring to FIG. 64, extension data for the nucleotides of FIG. 63 using two Phi29 variant polymerases of this disclosure are shown. Primer extension was performed using dual-labeled dNTP intermediates of FIG. 63. Each reaction contained 50 mM Tris pH 7.5, 2 mM MnCl₂, 0.5 μM Phi-29 polymerase mutant S388G (also referred to as HBP122) and 0.5 μM duplex (template:ATgg), 5 μM dNTPs (−)=no dNTP; NL=AAdU; NL*=AAdU with the AA linker extended; LNL*=extended AAdU2; γ=dU2. NL contains an apparently SAP resistant species.

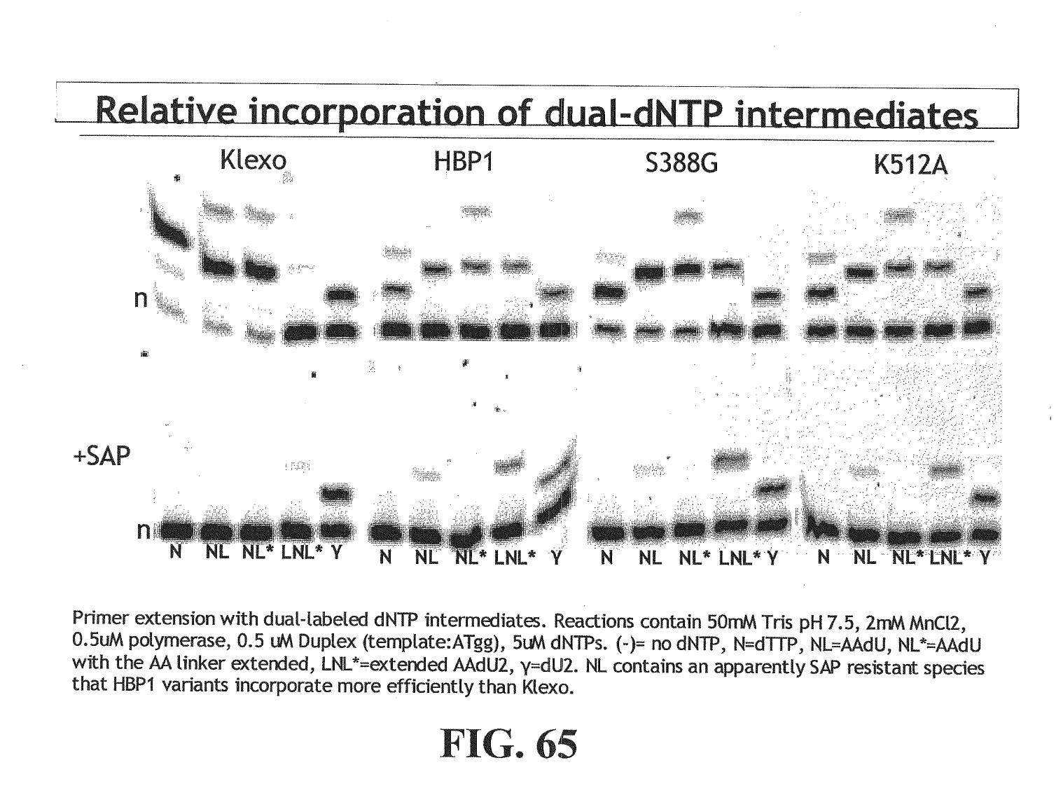

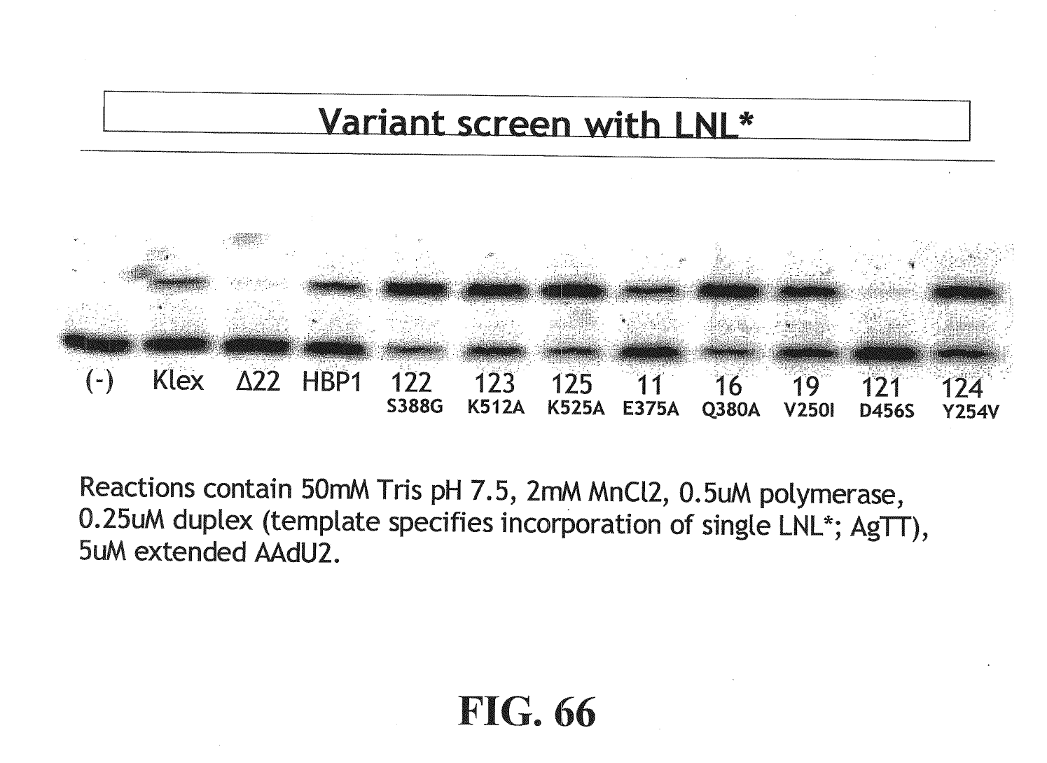

Referring to FIG. 65, extension data for the nucleotides of FIG. 63 using Klenow, Phi29 and two other Phi29 variant polymerases of this disclosure are shown. Referring to FIG. 66, extension data for the nucleotides of FIG. 63 using Klenow, Phi29 and two other Phi29 variant polymerases of this disclosure are shown.

An exemplary dual labeled nucleotide is depicted in FIG. 82. This nucleotide, named A1x594-dU3P-2-Cy5 (Compound 5 of FIG. 82), comprises an Alexa Fluor 594 dye label attached to the nucleobase of the nucleotide and a Cy5 dye label attached to the terminal phosphate. FIG. 82 also illustrates an exemplary method of synthesis for this dual labeled nucleotide. In the first step, Nucleotide 5-Aminoallyl-2′-deoxyuridine-5′-Triphosphate (AA-dUTP, 14 umol, TriLink N-2049) was dissolved in NaHCO₃ buffer (1M, pH 9.0, 75 μL) and diluted to 375 uL with water. Propargyl-dPEG™1-NHS ester (135 μmol, Quanta BioDesign Catalog #10511) was dissolved in 307 uL of dimethylformamide (DMF) and the resulting solution was added to the above AA-dUTP solution. The mixture was set on vortex overnight at room temperature, resulting in the formation of Compound 1. The reaction mixture was subjected to high performance liquid chromatography (Waters 1525 binary HPLC pump, 2996 photodiode array detector, Empower software) with passage through a semi-preparative Waters C18 column, followed by elution of the product (Compound 1) with an appropriate gradient of triethylammonium acetate buffer (TEAA, 100 mM) and methanol (MeOH). After lyophilization the product was obtained at quantitative yield (14 umol).

Compound 2 was then prepared from Compound 1. Briefly, 9 umol of Compound 1 was conjugated with diamine linker 2 using published dicyclohexylcarboimide (DCC) chemistry in the presence of the diamine linker XDA (Knorre, FEBS Letters 1976, 105). Purification was achieved through HPLC (Waters Protein-Pak strong anion exchange column, SAX) followed by elution with an appropriate gradient of water and triethylammonium bicarbonate (1M). The yield of Compound 2 was 2.6 umol (29%).

Compound 3 was then prepared from Compound 2. Briefly, 340 nmol of Compound 2 was labeled with 2720 nmol of Cy5 succinimidyl ester (Cy5-SE, GE Healthcare, PA15100) in essentially the same manner as for Compound 1 preparation. The reaction mixture was first treated with Sephadex G25 column chromatography to remove bulk Cy5 fluorophore. The first eluting fraction was taken through HPLC (SAX, TEAB 1M/H₂O) and HPLC (C18, TEAA100 mM/MeOH) to offer 88 umol of the desired product (26%). It was shown to be a substrate for the enzyme phosphodiesterase 1 (PDE1).

Compound 4 was prepared by reaction of 1 μmol of Alexa594-SE (Invitrogen #A20004) with 3. 1 μmol of 11-Azido-3,6,9-trioxaundecan-l-amine, the azido-amine (Aldrich #17758) in 20 μL of DMF in the presence of triethylamine (TEA, 36 μmol) for 60 hours at room temperature. Purification with HPLC (GE Healthcare Mono Q 17-0506-01, TEAB1M/H₂O)) gave 140 nmol of the desired product, Compound 4 (14%). The product was treated with amine scavenging resin, Methylisocyanate polystyrene HL 200-400 mesh, 2 E DVB (Novobiochem 01-64-0169) to remove any residual amine.

In the final step, Compound 5 (Alx594-dU3P-2-Cy5) was prepared by reacting Compound 4 with Compound 2. Briefly, Compound 5 was prepared by two Click reactions with the following materials: Cy5-nucleotide 2 (9 nmol), Alx594-azide 4 (19 nmol), 10 mM pH 8.5 HEPES/t-BuOH (2:1). Two different copper sources were used: Copper powder (46 umol, Aldrich 266086) and five pieces of Copper wire (17 umol each, 85 umol total, Aldrich 326429). It was monitored using HPLC (SAX, TEAB 1M/H₂O) at 3 wavelengths: 260, 646, 590 nm. Both reactions were clean and efficient after O/N. The fractions 17 min and 18 min, possessing both absorptions, were assigned as the desired product (2.4 nmol, 13%).

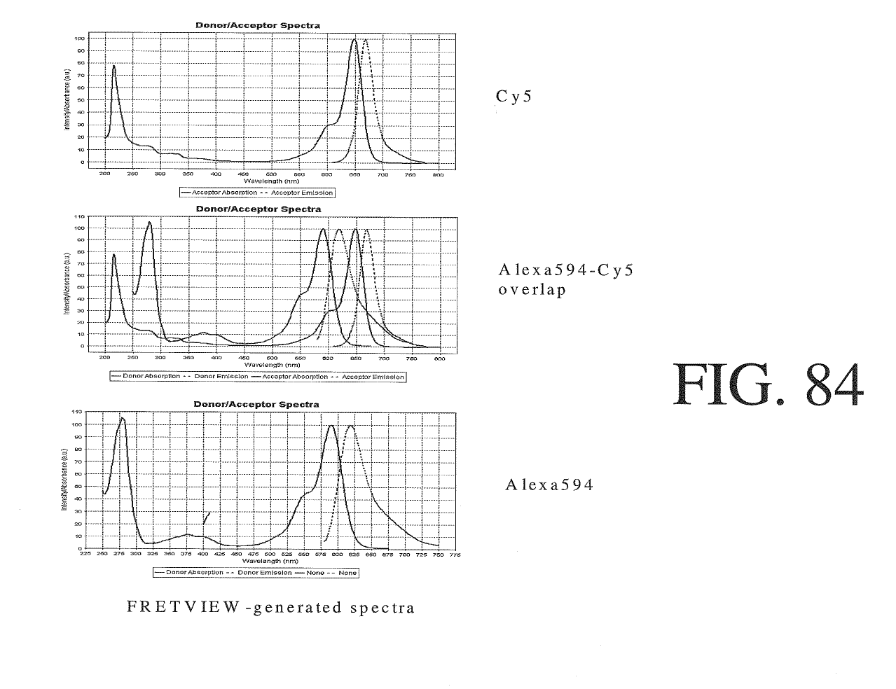

As illustrated in FIG. 84, the two labels of the dual labeled nucleotide Alx594-dU3P-2-Cy5 are well adapted to undergo FRET with each other because Alexa Fluor 594 emission is able to excite the Cy5 dye label. The top panel of FIG. 84 shows the absorption and emission spectra of Cy5 dye, the bottom shows those of Alexa594, and the middle panel shows the composite overlay of spectra of both labels. In the middle panel, the four peaks in the range of 500-750 nm are, from left to right: Alexa594 excitation, Alexa594 emission, Cy5 excitation, Cy5 emission, respectively. It can be seen that Alexa594 emission overlaps Cy5 excitation. Also it appears there is direct excitation of Cy5 above 550 nm.

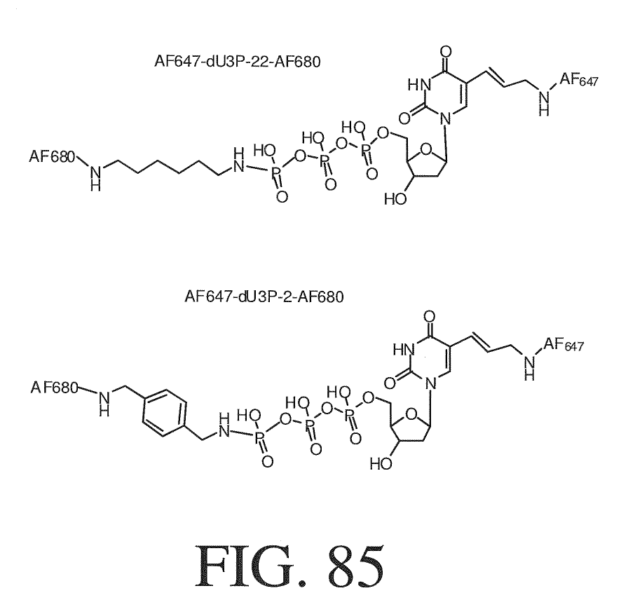

FIG. 85 depicts two more exemplary dual labeled nucleotide molecules, named AF647-dU3P-22-AF680 and AF647-dU3P-2-AF680, respectively. Both nucleotides comprises an Alexa Fluor 647 dye label operably linked to the nucleobase of the nucleotide, and an Alexa Fluor 680 dye label linked to the terminal phosphate.

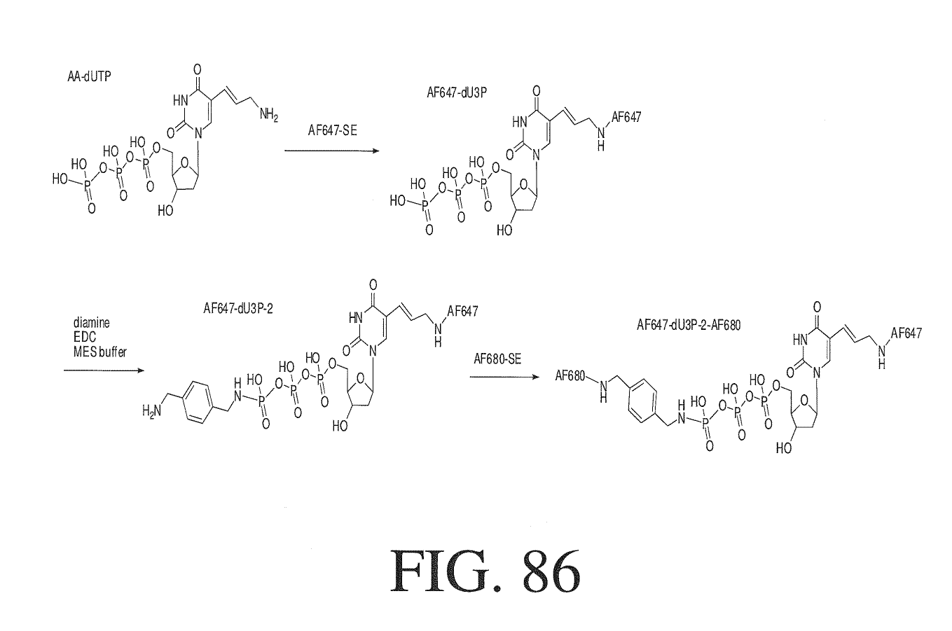

FIG. 86 depicts an exemplary method of synthesis for the dual labeled nucleotide AF647-dU3P-2-AF680. Briefly, the nucleotide AF647-dU3P was first prepared from AA-dUTP (4 umol) through reaction with Alexa647-SE (5eq, Invitrogen #A20006) in the presence of NaHCO₃ (pH 9.0, 100 mM) and DMF (2:1) following the vendor's recommended protocol. HPLC (SAX, TEAB/H₂O) purification gave 2.4 μmol of the product at a yield of 60%.

The nucleotide AF647-dU3P-2 was then prepared via EDC (50 eq) mediated coupling of the indicated diamine linker (25 eq) and Alx647-dUTP (480 nmol) in MES buffer (2-(N-morpholino)ethanesulfonic acid, 600 mM, pH5.8) overnight at room temperature. HPLC purification (SAX, TEAB/H₂O) yielded the product (230 nmol, 48%).

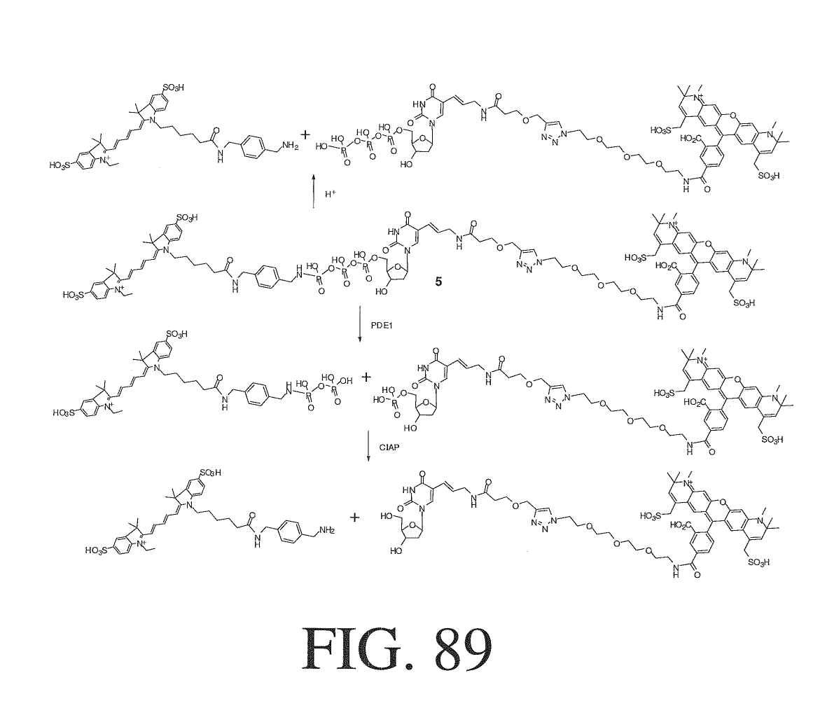

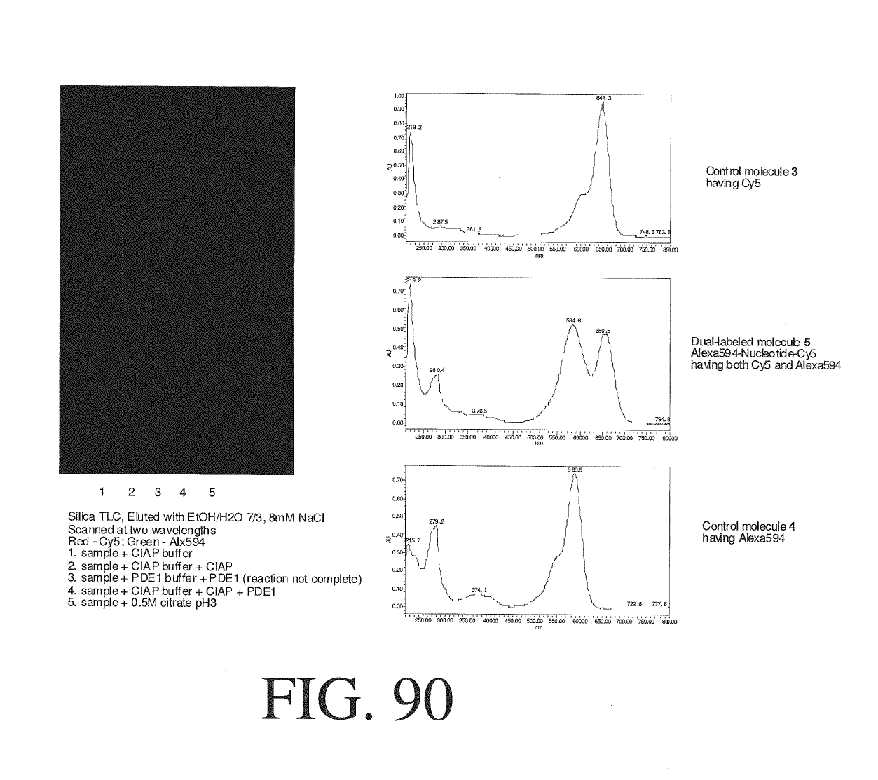

The nucleotide AF647-dU3P-2-AF680 was then prepared from the nucleotide AF647-dUTP-2 (51 nmol) and Alx680-SE (5 eq, Invitrogen #A20008) and HPLC (SAX, TEAB/H₂O). Purification yielded 25 nmol of the product (49%). Phosphatase assays using the enzymes CIAP, PDE1, and a CIAP/PDE1 mixture, as well as acid treatment (citrate acid, pH 3.0) were performed as for Compound 5 (see FIGS. 89-90 and associated description, below) in combination with PEI cellulose ion exchange TLC (TEAB 1M). The results were consistent with the assumed product (data not shown).

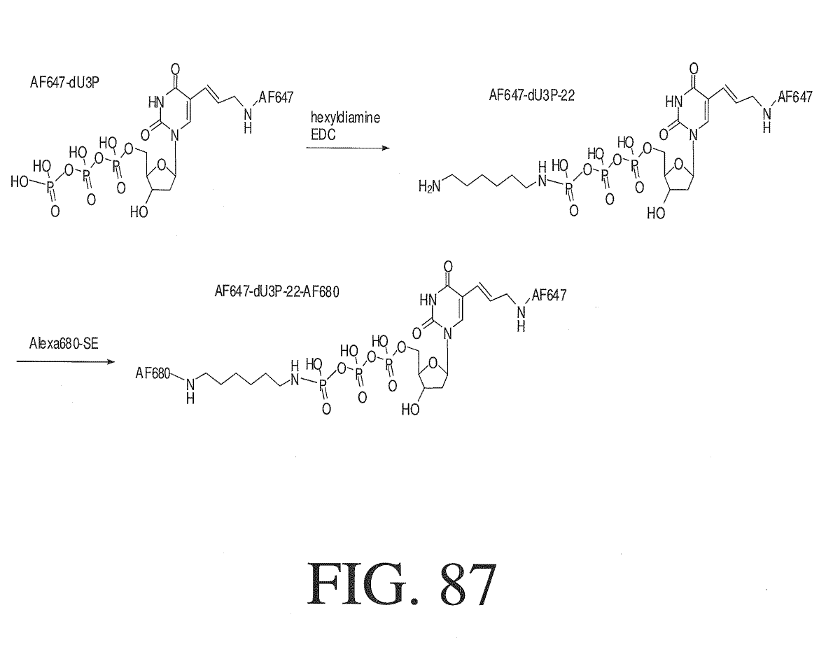

FIG. 87 depicts a very similar method of synthesis for the dual labeled nucleotide AF647-dU3P-22-AF680. Briefly, the nucleotide AF647-dU3P-22 was prepared via EDC-mediated coupling of diamine linker N—C6-N to Alx647-dUTP (640 nmol). This reaction yielded 95 nmol of the product, one CIAP-active product (85 nmol), and recovered Alx647-dUTP (232 nmol). Reaction setup and purification was similar to those used for the nucleotide AF647-dU3P-2. Next, the nucleotide AF647-dU3P-22-AF680 was prepared by labeling 42 nmol of nucleotide AF647-dU3P-22 with Alx680-SE (5 eq) to yield 16 nmol of product (38% yield). Reaction setup and purification was similar to those used for the nucleotide AF647-dU3P-2-AF680. Phosphatase assays using the enzymes CIAP, PDE1, and a CIAP/PDE1 mixture, as well as acid treatment (citrate acid, pH 3.0) were performed as for Compound 5 (see FIGS. 89-90 and associated description, below) in combination with PEI cellulose ion exchange TLC (TEAB 1M). The results were consistent with the assumed product (data not shown).

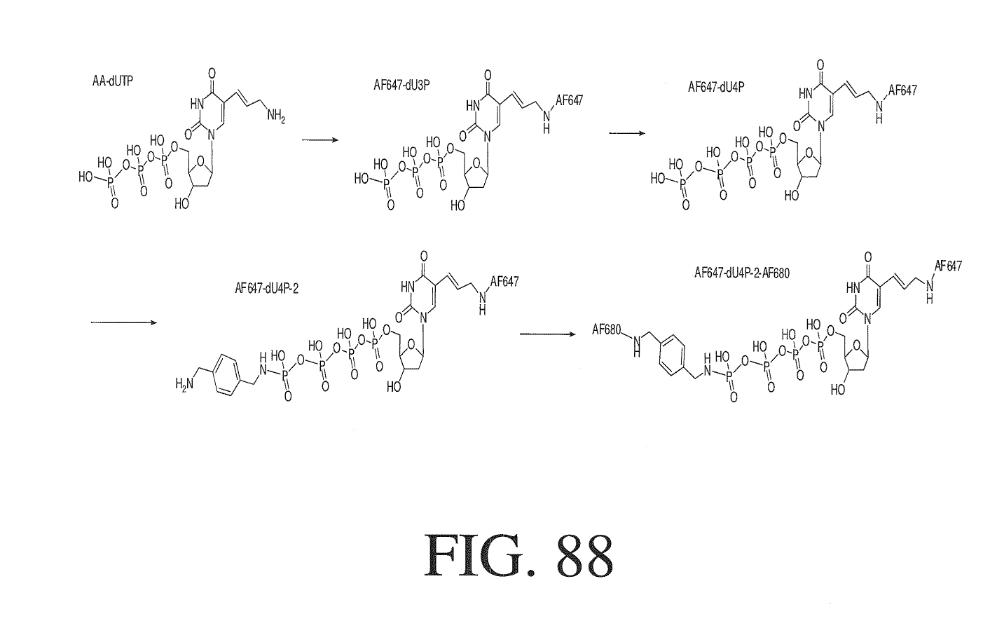

FIG. 88 depicts a very similar method of synthesis for the dual labeled nucleotide AF647-dU4P-2-AF680, which comprises a nucleotide tetraphosphate instead of a nucleotide triphosphate as in AF647-dU3P-2-AF680 and AF647-dU3P-22-AF680. To prepare this compound, the nucleotide AF647-dU4P was first prepared from Alx647-dU3P (1.2 umol) following the published procedure of Kumar et al, J. Am. Chem. Soc. 2005, 2394. The yield of AF647-dU4P product was 33%. Next, the nucleotide AF647-dU4P-2 was prepared from Alx647-dU4P (65 nmol) and diamine linker 2 using DCC chemistry similar to that used to synthesize AF647-dU3P-2 (see above), with a product yield of 65%. Phosphatases assays were run in combination with ion exchange TLC. These were consistent with assumed product.

Nucleotide AF647-dU4P-2-AF680 was prepared by labeling of AF647-dU4P-2 (21 nmol) with Alx680-SE similar to AF647-dU3P-2-AF680. The yield was 34%. Phosphatase assays using the enzymes CIAP, PDE1, and a CIAP/PDE1 mixture were performed as for Compound 5 (see FIGS. 89-90 and associated description, below) in combination with PEI cellulose ion exchange TLC (TEAB 1M). The results were consistent with the assumed product (data not shown).

As depicted in FIG. 89, chemical and enzymatic assays were designed for the characterization of the dual-labeled nucleotide Alx594-dU3P-2-Cy5 (Compound 5 of FIG. 82). Acid treatment should cleave the P—N bond as indicated; treatment with phosphodiesterase-1 (PDE1) should cleave the nucleotide between the α- and β-phosphates and separate the dyes into two molecules; treatment of the PDE-1 digestion products with calf intestine alkaline phosphate (CIAP) cleave the bonds between the remaining phosphates, yielding two neutral dye-containing molecules. However, the dual-labeled nucleotide 5 should be inert to CIAP digestion per se.

FIG. 90 illustrates the results of analysis of the dual-labeled nucleotide of Compound 5 (Alx594-dU3P-2-Cy5) using both enzymatic digestion followed by silica thin layer chromatography (TLC) (left) and fluorescence scanning (right). To generate the panel on the left, CIAP (Worthington #LS004228) and PDE1 (Worthington #LS003926) were used to treat Alx594-dU3P-2-Cy5 in the presence of enzyme buffer prepared as recommended by the buffer. Briefly, 2 uL of enzyme or citrate buffer was mixed with 0.3 uL of the nucleotide 5 (16 uM) followed by 0.3 uL of the enzyme or enzymes (CIAP 1 U/uL, PDE1 0.065 U/uL) or 0.3 uL of water (for acid hydrolysis). After 3 hours of incubation at room temperature the samples were subjected to TLC/scanning analysis as mentioned above. As shown in FIG. 90, left panel, the dual-labeled nucleotide is inert to CIAP (see lane 1 and lane 2). Digestion by both PDE1 and CIAP (lane 4) yielded a product that migrated much faster than the PDE-1 digestion product (lane 3), possibly because CIAP digestion effectively removed the PDE1 substrate compound from the reaction. Acid hydrolysis clearly showed the color separation as expected for the P—N bond cleavage.

In addition, the dual labeled nucleotide was subjected to fluorescence scanning As shown in FIG. 90, right panel, the UV spectra of control molecules 3 (Cy5) and 4 (Alexa Fluor 594) overlap with the spectra of the dual-labeled nucleotide 5, which exhibited the combined absorption peaks of Cy5 and Alexa594.

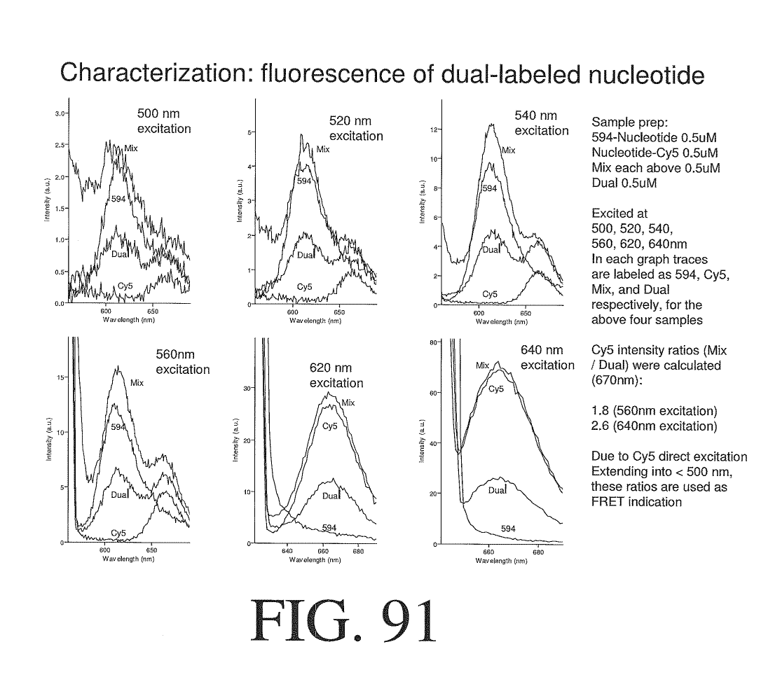

FIG. 91 shows a series of graphs depicting the result of spectral analysis of single- and dual-labeled nucleotide compounds, as well as a mixture of the two. Briefly, samples comprising 0.5 μM of single labeled nucleotides labeled with Alexa Fluor 594 and Cy5, respectively, as well as a sample comprising the dual-labeled nucleotide Alexa594-dU3P-2-Cy5 (Compound 5 of FIG. 82) and a sample comprising a mixture of the two single-labeled nucleotides. The four emission traces for the each of these four samples were measured and plotted for a series of different excitation wavelengths, each excitation wavelength yielding a separate plot. Due to Cy5 direct excitation, the ratios of Cy5 emission intensities at 670 nm were calculated for the Mixture and the Dual labeled nucleotides. At 560 nm excitation, where Cy5 is minimally excited, the Mix/Dual emission intensity ratio was 1.8. At 640 nm excitation where Cy5 is maximally excited, the ratio was 2.6, confirming the occurrence of FRET between Alexa594 and Cy5 in the dual labeled nucleotide.

Also disclosed herein are specially designed nucleotide structures, termed “star” nucleotides, and the use of these “star” nucleotides to reduce the signal background resulting from the presence of labeled nucleotides during real-time sequencing. Such labeled “star” molecules were designed to allow investigators to increase the nucleotide concentration without concomitantly increasing the fluorescent label concentration. The “star” nucleotides typically comprise multiple nucleotide moieties operably linked to otherwise attached to the same acceptor fluorophore, while maintaining close spacing between the acceptor and donor fluorophores during incorporation, as disclosed more fully in Wang et al., 60/891,029). One requirement for star molecules is that the acceptor fluorophore must not photobleach before the attached nucleotides are consumed in the sequencing reaction, thus requiring an optimal balance between the number of nucleotides attached to the acceptor fluorophore and the acceptor photobleaching.

More particularly, disclosed herein are dendrimer compounds comprising a branched molecular structure containing multiple instances of a first linking capable of attachment to a nucleotide. In some embodiments, the compound further comprises a single instance of a second linking group capable of attachment to a detectable label. Also disclosed herein are methods for synthesizing a branched and labeled nucleotide compound using a dendrimer compound, comprising: (a) attaching a single dye moiety to a branched dendrimer compound, and (b) attaching multiple nucleotides to the dendrimers. In some embodiments, the linking group is an amino group, azide group, terminal alkyne group, carboxyl, sulfhydryl or alkyl group.

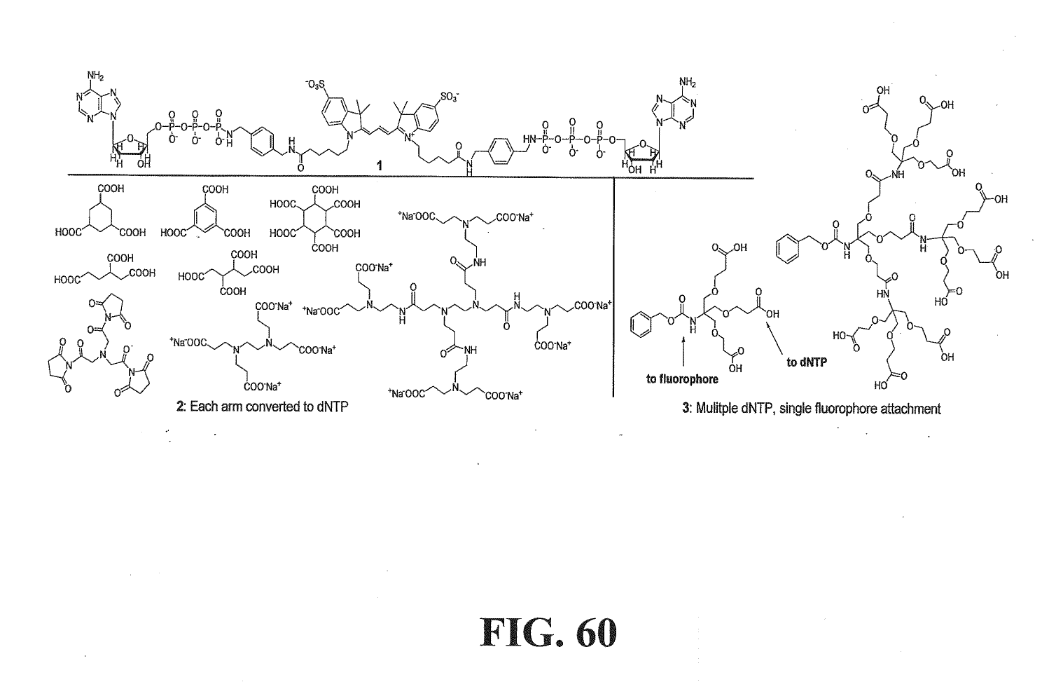

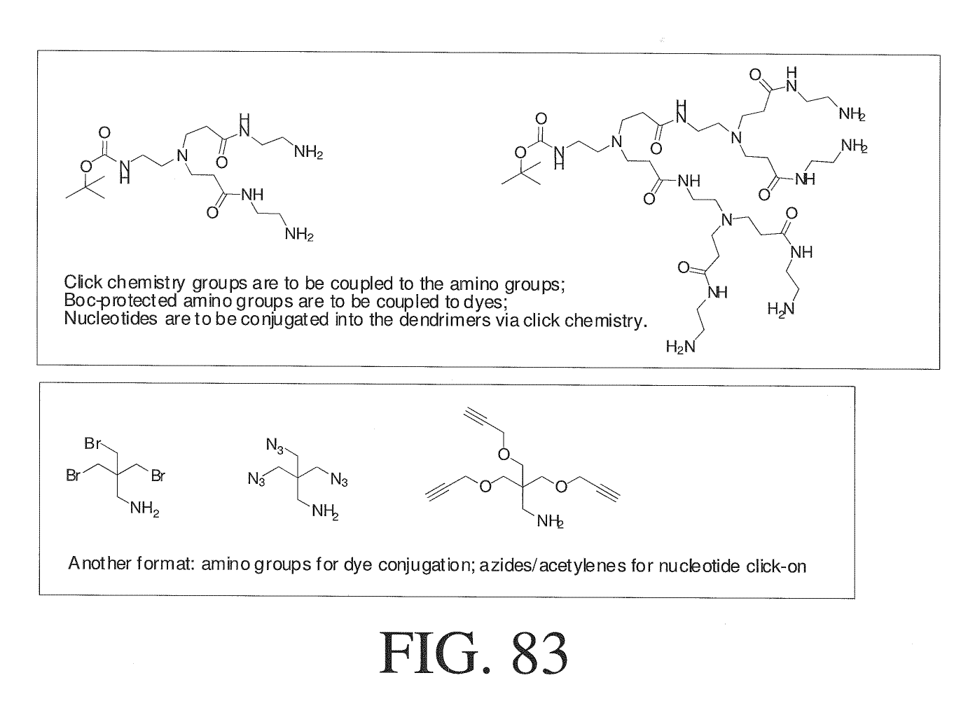

In one exemplary embodiment, star molecules are synthesized by attaching amino-terminated γ-modified nucleotides to cores of various shapes and sizes, either labeled or non-labeled. Commercially available bis-reactive dyes such as Cy3-bis NHS, Cy5-bis NHS, and Oyster645-bis NHS, were used to couple two γ-modified nucleotides in linear star molecules (FIG. 60, part (1)). Because both of the attached nucleotides were incorporated by our enzymes (determined via TLC), non-fluorescent star molecules that utilized commercially available cores of different shapes, flexibility, and number of branches were tested (FIG. 60, part (2)). These cores were modified with amino-terminated γ-modified nucleotides via amide bond formation using various coupling reagents, and the resulting star molecules are incorporated by polymerase enzymes (data not shown). Subsequently, star molecules were designed with asymmetrical dendritic cores that allow the attachment of multiple nucleotides to a single amino group, which can be coupled to any desired dye (FIG. 60, part (3)). Additional star molecules are also provided in FIG. 83.

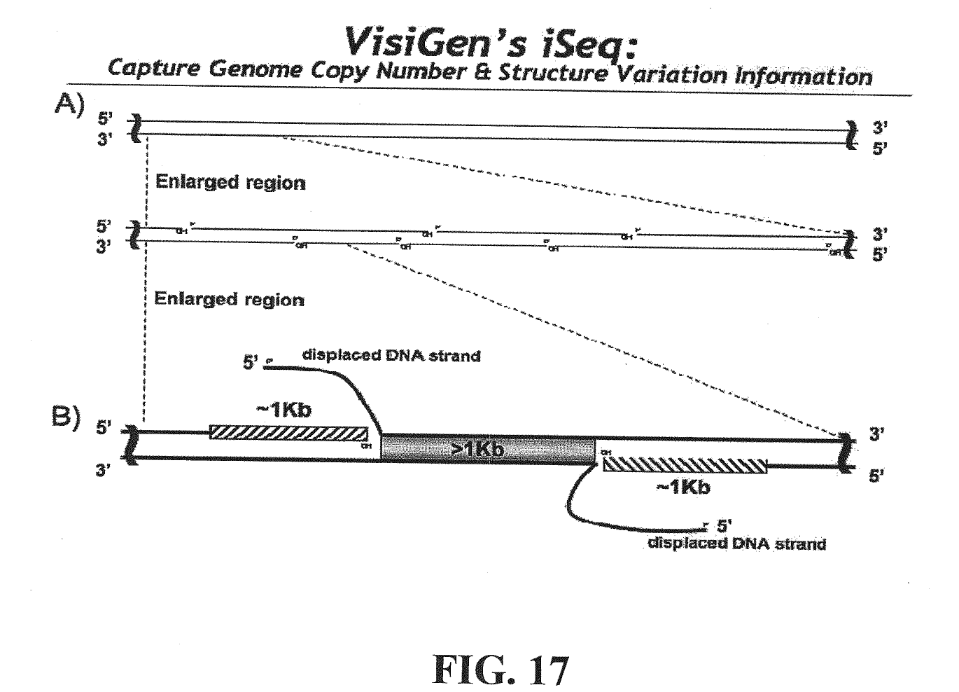

Optionally, the systems and methods disclosed herein can be adapted to incorporation intercalation sequencing, also termed ‘donor replacement sequencing’, a method wherein a nucleotide intercalating dye is used as the FRET donor, as described more fully in PCT Application No. PCT/US2008/080843, filed Oct. 22, 2008. As described in that application, nicks exposing 3′ hydroxyl termini can be introduced via enzymatic or chemical means approximately every 3-5 Kb along a DNA strand. The frequency of extendable 3′ termini can be characterized by incorporating a base-labeled nucleotide at the nick site in solution, immobilizing the strands on a single-molecule detection system, and visualizing the incorporated bases by either direct excitation of the acceptor or detection of FRET between a donor dye used to stain the DNA (i.e., SYBR Green I, YOYO-1 or similar intercalation or groove-binding dye) and the incorporated acceptor. FIG. 17 depicts an overview of intercalation based sequencing. After the nicking reaction is refined to obtain optimal spacing, the number of donor fluorophores that associate with the DNA will be optimized to identify a staining concentration that produces high FRET. Optimal spacing between a donor fluorophore and an acceptor on the incorporated nucleotide should be closer than the Rₒ of the donor-acceptor pair so that high FRET results, typically resulting in greater than 80% FRET. If too few fluorophores interact with the DNA, they will not be spaced closely enough to produce high FRET with the acceptor fluorophore. However, if too many donor fluorophores intercalate or bind the DNA, fluorophore quenching may occur.

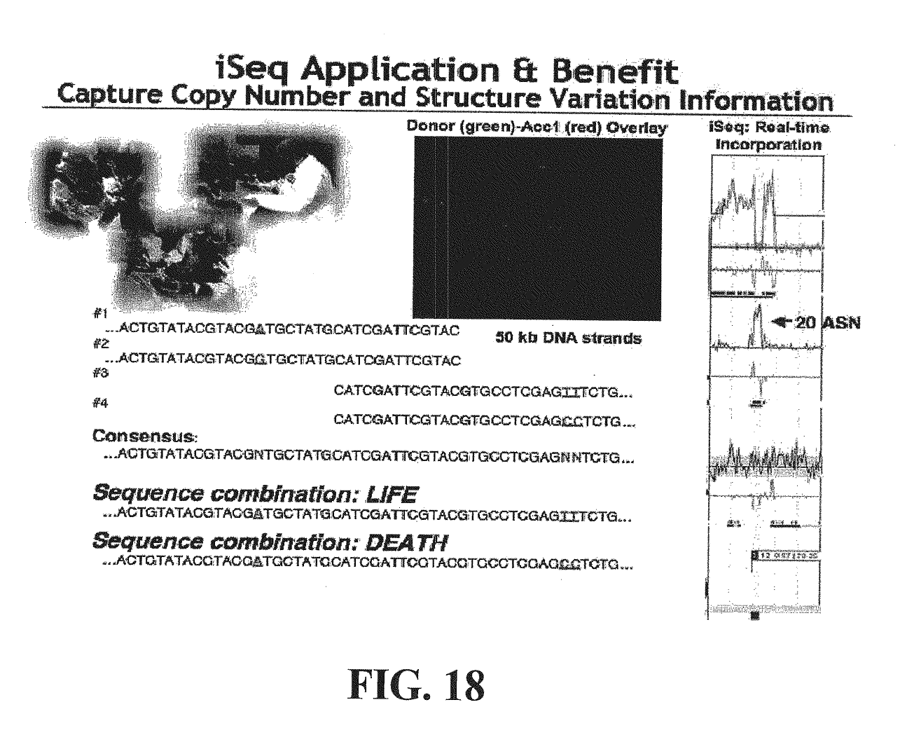

In some embodiments, extension buffer, DNA polymerase and fluorescently-labeled nucleotides can be added into the reaction chamber to initiate the sequencing reaction. The DNA polymerase in the sequencing solution will recognize and bind the exposed 3′ hydroxyl termini and initiate the DNA sequencing reaction. An acceptor-labeled nucleotide will enter the enzyme's active site and a high efficiency FRET event will result via energy transfer from donors located both 3′ and 5′ of the initiation site to the acceptor. Similar to the discussion regarding Qdots, the ability to detect a dip in donor intensity likely depends on a variety of conditions. Preliminary data detects donor dipping, as shown in FIG. 18, wherein real-time incorporation trace is provided at right showing 20 ASN with BL-nucleotide. Alternatively, if the acceptor signals are intense and well-defined it may not be problematic if donor dipping is not detected.

Following removal of the acceptor attached to the incorporated nucleotide by either enzymatic or photochemical cleavage, the next acceptor labeled nucleotide will enter the active site and produce the next high FRET event. This process will continue and the sequence 3′ of the nick site will be determined. Independent sequence information will be determined from the enzymatically accessible 3′ termini that are strategically spaced along the length of the DNA strand. Importantly, each sequencing complex along the strand provides sequence information about a region contained within the extended fragment and, further, each sequence read along the strand is both discrete and ordered.

The polymerase used in this immobilized DNA variation of the spFRET sequencing technology will possess either a strong strand displacement activity or 5′ to 3′ exonuclease activity to remove the downstream strand, thereby facilitating DNA synthesis. Using a highly processive polymerase (i.e., Phi29), the downstream strand will be displaced but, because the 5′ terminated strand cannot serve as a template in the absence of added primer, no secondary sequence information from this site will be detected. If an intercalating dye (i.e., SYBR Green I) is included in the reaction buffer as the donor fluorophore, a SYBR Green I fluorophore should effectively replenish and position a new donor when it inserts into the newly synthesized, double-stranded DNA. Dyes and dye concentrations will be chosen that optimize donor emission and maximize acceptor intensities. Additionally, certain combinations of DNA-binding donor dyes may produce higher intensity acceptor signals when paired with the spectrally-resolved acceptors used to determine base identity, and these donor dyes may need to be present in particular ratios to maximize these effects. Continuing with SYBR Green I as an example, dye in solution and dye interacting with the displaced single strand exhibits reduced fluorescence intensity, relative to dye bound to double-stranded DNA (Zipper, Brunner et al., 2004). As an additional confirmation of distance between sequencing sites, others have determined that integrated fluorescence intensity measurements coupled with quantile analysis provides an accurate measure for the amount of DNA (Li, Valouev et al., 2007), and the effect of this method on consensus sequence production will be examined.

Because the DNA will be attached to the surface at various points along its length, it will consist of a series of closed DNA domains. Optionally, a topoisomerase and/or a gyrase may be included to modulate the number of DNA supercoils that may be introduced during the sequencing reaction (Champoux, 2001). The need for inclusion of such enzymes will reflect both sequence read length and the degree to which the DNA is immobilized onto the surface. Longer read lengths and increased number of attachment sites between the DNA strand and the surface will more quickly increase the number or impact of helical windings generated during sequencing and, thus, these situations may benefit from inclusion of an enzyme that can maintain DNA supercoiling at levels that support efficient replication.

Advantages of donor replacement sequencing strategies include: (1) the production of discrete and ordered reads that will facilitate accurate genome assembly; (2) the ability to use a polymerase (i.e., Phi29 slowed chemistry variant) that is neither labeled nor immobilized: (3) potential to continuously optimize donor energy transfer capabilities by positioning a new donor at a distance that will produce a high FRET event, relative to the more upstream donor that may have photobleached or, as a result of nucleotide incorporation and enzyme translocation, become too distant from the acceptor-labeled nucleotide bound at the enzyme's active site to efficiently FRET, and (4) increasing acceptor signal (relative to interaction with a single donor fluorophore). For these reasons, donor replacement sequencing strategies will typically be used in parallel with the previously discussed labeled polymerase strategy.

Donor replacement sequencing of long DNA strands will facilitate the identification of genomic rearrangements and improve the assembly accuracy of chromosomal sequences (i.e., correctly identifying independent HIV genomes; associating sequence reads with the correct maternal/paternal chromosome). Production of haplotype information is especially important because it is shown to have more power than individual nucleotide variation in the context of association studies and in predicting disease risks (Stephens, Schneider et al., 2001; HapMap Project). The first diploid genome sequence of a single human demonstrates that maternal and paternal chromosomes are 99.5% similar when genetic variation due to insertion and deletion is taken into account (Levy, Sutton et al., 2007). The combination of longer read lengths and discrete, ordered reads will facilitate correct assembly of the maternal and paternal chromosome sequences. Currently, and for many fold more than $1000, very accurate and deep sequence coverage may allow this distinction to be made if the borders of the genomic breakpoints are identified. However, because donor replacement sequencing strategies will directly couple sequence information with mapping information, these genomic variations will be identified.

Also disclosed herein are mutant or variant polymerase proteins that exhibit improved or altered abilities to incorporate labeled nucleotides onto the terminal 3′OH of a newly synthesized nucleic acid molecule. In some embodiments, the mutant polymerase is a mutated, modified or engineered form of Taq DNA polymerase, Phi-29 DNA polymerase, Klenow polymerase or variants thereof. In some embodiments, the polymerase is a variant of a Phi29 polymerase. (For the protein sequence of wild type Phi-29 polymerase, see, for example, U.S. Pat. No. 5,198,543. In some embodiments, the isolated variant is a variant of a protein having the amino acid sequence of SEQ ID NO: 3, wherein the variant comprises an amino acid sequence that is at least 80% identical to SEQ ID NO: 3. Optionally, the variant further comprises one or more mutations selected from the group of mutations shown in Table 1, below. Optionally, the variant comprises one or more mutations selected from the group consisting of: V250A/E375Y, V250A/E375A/Q380A, V250A/E375C, V250A/E375Y, V250I/E375A/Q380A, V250I/E375C, V250A, V250I, E375A, E375C, E375Y, E375A/Q380A, Q380A, D456N, D456E, D456S, D458N, V250A/E375A/Q380A/D456E, E375Y/V250L, E375Y/V250P, E375Y/V250Q, E375Y/V250R, E375Y/V250Y, E375Y/V250F, E375YN2505, E375Y/V250C, E375Y/V250T, E375Y/V250K, E375Y/V250H, E375Y/V250N, E375Y/V250D, E375Y/V250G, E375Y/V250W, E375Y/S388G, E375Y/K512A, E375Y/K525A, Y254V/E375Y, K132A, K383A, K383R, K383P, K371A, K371T, Y254F, Y254V, Y254S, Y254V, Y254S, K379A, K525A, K135A, P255S, S388G, K512A, L384R, E486A, E486D, K478A, E375W, N387A, N387Y, V250A/E375W, D456N/D458N/L351P, Y254V/A377E, D456N/D458N, D169A, D12A/D66A/D169A, T151, N62D, C22S, C290S, C448S, C530S, C290S/C448S/C530S, C22S/C448S/C530S, C22S/C290S/C530S and C22S/C290S/C448S. Typically, the variant has polymerase activity, i.e., is an active polymerase. Optionally, the variant is operably linked to a FRET donor. In some embodiments, the FRET donor is capable of undergoing FRET with an acceptor attached to a nucleotide before, during or after the nucleotide is incorporated by the polymerase onto the terminal 3′OH of a synthesized DNA molecule. In some embodiments, the FRET donor is a nanocrystal. Typically, the variant comprises an amino acid sequence that is at least 85%, 90%, 95% or 99% identical to the amino acid sequence of SEQ ID NO: 3. Optionally, the variant exhibits altered ability to incorporate labeled nucleotides onto the terminal 3′OH of a newly synthesized nucleic acid molecule as compared to its wild-type counterpart.

In another embodiment, the polymerase is a variant of Taq DNA polymerase having a wild-type sequence as disclosed in Lawyer et al., (1989) and that comprises the mutation F647C. Typically, the variant has polymerase activity, i.e., is an active polymerase. Optionally, the variant is operably linked to a FRET donor. In some embodiments, the FRET donor is capable of undergoing FRET with an acceptor attached to a nucleotide before, during or after the nucleotide is incorporated by the polymerase onto the terminal 3′OH of a synthesized DNA molecule. In some embodiments, the FRET donor is a nanocrystal. Typically, the variant comprises an amino acid sequence that is at least 85%, 90%, 95% or 99% identical to the amino acid sequence of SEQ ID NO: 3. Optionally, the variant exhibits altered ability to incorporate labeled nucleotides onto the terminal 3′OH of a newly synthesized nucleic acid molecule as compared to its wild-type counterpart.

By the term “% identity” and its variants is meant that two peptide sequences, when optimally aligned, such as by the programs GAP or BESTFIT using default gap weights, share at least 65 percent sequence identity, preferably at least 80 or 90 percent sequence identity, more preferably at least 95 percent sequence identity or more (e.g., 99 percent sequence identity or higher). Preferably, residue positions which are not identical differ by conservative amino acid substitutions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89, 10915 (1989))

The term “mutant” and its variations, as used herein, refers to a polypeptide or combination of polypeptides characterized by an amino acid sequence that differs from the wild-type sequence(s) by the substitution of at least one amino acid residue of the wild-type sequence(s) with a different amino acid residue and/or by the addition and/or deletion of one or more amino acid residues to or from the wild-type sequence(s). The additions and/or deletions can be from an internal region of the wild-type sequence and/or at either or both of the N- or C-termini. A mutant antibodies or antibody fragments may have, but need not have neutralization activity. Typically, a mutant displays biological activity that is substantially similar to that of the wild-type Aβ peptide or antibody or antibody fragment. In some embodiments of a mutant protein, at least one amino acid residue from the wild-type sequence(s) is substituted with a different amino acid residue that has similar physical and chemical properties, i.e., an amino acid residue that is a member of the same class or category, as defined above. For example, a conservative mutant may be a polypeptide or combination of polypeptides that differs in amino acid sequence from the wild-type sequence(s) by the substitution of a specific aromatic Phe (F) residue with an aromatic Tyr (Y) or Trp (W) residue.

As used herein with respect to particular nucleic acid sequences, the term “variant” and its variants refers to those nucleic acids that encode substantially similar or essentially identical amino acid sequences, or where the nucleic acid does not encode an amino acid sequence, to substantially similar or essentially identical sequences.

As used herein with respect to particular nucleic acid sequences, the term “variant” and its variants refers to individual substitutions, deletions or additions to a nucleic acid, peptide, polypeptide, or protein sequence which alters, adds or deletes a single amino acid or a small percentage of amino acids in the encoded sequence.

Also disclosed herein are methods for determining a nucleotide sequence of a nucleic acid molecule, comprising: conducting a nucleic acid polymerase reaction in the presence of at least one detectably labeled nucleotide and any polymerase variant of the present disclosure, which reaction results the production of a detectable signal before, after or during a nucleotide incorporation event; detecting a time sequence of incorporation events and thereby determining the identity of individual nucleotides incorporated during the polymerase reaction, and thereby determining a nucleotide sequence of the nucleic acid molecule.

Optionally, the detectable signal is a FRET signal.

Optionally, the detectable label of the detectably-labeled nucleotide is a chromophore, fluorophore or luminophore.

In some embodiments, the detectable label of the detectably-labeled nucleotide can be a fluorophore selected from the group consisting of: xanthine dye, fluorescein, cyanine, rhodamine, coumarin, acridine, Texas Red dye, BODIPY, ALEXA, GFP, and a derivative or modification of any of the foregoing.

Optionally, the nucleic acid polymerase of the nucleic acid polymerase reaction can be an RNA polymerase, DNA polymerase or reverse transcriptase. In some embodiments, the DNA polymerase of the nucleic acid polymerase reaction is a Klenow fragment of DNA polymerase I, E. coli DNA polymerase I, T7 DNA polymerase, T4 DNA polymerase, Thermus acquaticus DNA polymerase, or Thermococcus litoralis DNA polymerase.

Optionally, the nucleic acid polymerase of the nucleic acid polymerase reaction can be operably linked to a Forster resonance energy transfer (FRET) donor.

In some embodiments, the FRET donor is a nanocrystal.

Optionally, the nanocrystal can be surrounded with a coating material. In some embodiments, the coating material may comprise imidazole, histidine or carnosine.

Optionally, the nanocrystal may comprise a core comprising a first semiconductor material and a capping later deposited on the core comprising a second semiconductor material.

In some embodiments, the nanocrystal emits light with a quantum yield of greater than about 10%, 50%, or 70%.

In some embodiments, the nanocrystal further comprises cadmium selenide (CdSe), cadmium sulfide (CdS), cadmium telluride (CdTe), or mixtures thereof.

Optionally, the nanocrystal is a doped metal oxide nanocrystal.

In some embodiments, the nucleic acid polymerase of the nucleic acid polymerase reaction is further contacted with a nucleotide primer.

In some embodiments, the nucleotide primer is extended by a plurality of nucleotides. Typically, the nucleotide primer is extended by at least 100, 250, 500 or 1000 nucleotides.

In some embodiments, the nucleotide primer comprises at least 10, 25 or 50 nucleotides.

In some embodiments, the detectably labeled nucleotide has three, four or more phosphates.

In some embodiments, the rate of nucleotide sequence determination of a single nucleic acid molecule is equal to or greater than 1, 10 or 100 bases per second.

In some embodiments, the error rate of nucleotide sequence determination is equal to or less than 10%, 5%, 3%, 1%, 0.1%, 0.01% and 0.001%.

Optionally, the nucleic acid molecule comprises chromosomal DNA. In some embodiments, the nucleic acid molecule comprises a complete and intact chromosome.

Also provided for herein is a method for determining the sequence of one or more additional nucleic acid molecules in parallel with determining the sequence of a first DNA molecule according to the methods provided herein.

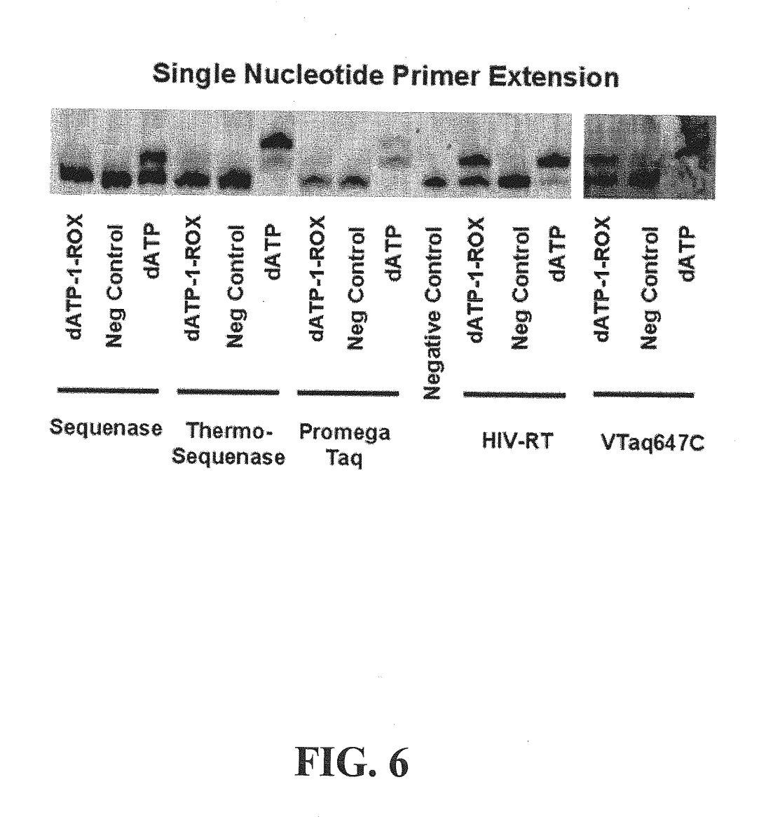

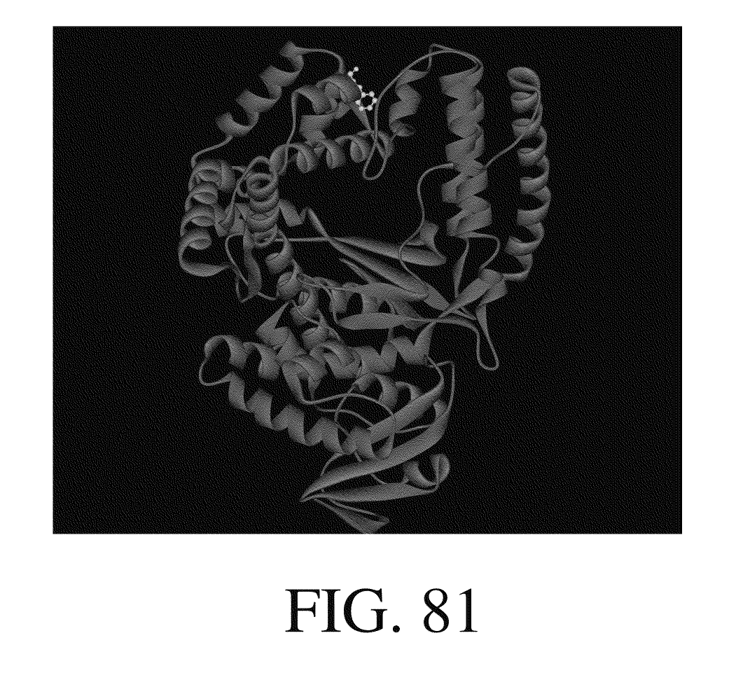

FIG. 6 depicts the results of experiments to assess the nucleotide incorporation efficiency of various DNA mutated and non-mutated polymerases (Sequenase; Thermo-Sequenase; wild-type Taq from Promega; HIV reverse transcriptase; and a mutant Taq polymerase comprising the mutation F647C) using both natural (unlabeled) dATP as well as the gamma-modified nucleotide dATP-1-Rox. Each reaction was conducted at the individual polymerase's optimal temperature with equimolar concentrations of enzymatically cleared gamma-labeled nucleotides and units of polymerase. As shown in FIG. 6, different polymerases reacted differently to γ-modified nucleotides. Primer extension reactions were performed in the presence of the nucleotide dATP-1-ROX or natural dATP. The ‘Neg Control’ is a control lacking both polymerase and nucleotide. The extension products were size separated using denaturing gel electrophoresis, and visualized on a Bio-Rad fx Imager. Incorporation of the gamma-labeled nucleotide results in the production of a resolvable extension duplex that indicates incorporation of a single dATP. The non-extended primer is the lower band; extension products are more slowly migrating. As shown in FIG. 6, the enzyme Sequenase, which incorporates natural dATP but not γ-dATP, could not find enough natural nucleotide in the cleared γ-dATP stock to produce detectable extension products. Furthermore, the engineered mutant Taq polymerase named “VTaq647C,” which comprises the mutation F647C, exhibits significantly improved incorporation of gamma-labeled nucleotides as compared to wild type Taq polymerase. FIG. 81 is a solid ribbon diagram generated in DS Viewer Pro of Taq DNA Polymerase. The residue F647 (highlighted in white and displayed in scaled ball and stick format) resides at the upper portion of the finger domain, and may potentially affect entry of the nucleotide into the nucleotide binding pocket.

As described in further detail herein, approximately 50 variants of Phi29 and ˜30 variants of Klenow have been engineered to incorporate γ-modified nucleotides, and many of these variants have been labeled with either an organic (i.e., Alexa488) or inorganic donor fluorophore (i.e., a quantum dot) (data not shown). Many of these polymerases incorporate 4-color, γ-labeled nucleotides into extended DNA strands, although at reduced efficiency (data not shown).

In addition to fluorescent modification, preliminary data (not shown) indicates that enzyme immobilization may affect its activity. Therefore, the ensemble activity of each labeled polymerase variant can optionally be tested following immobilization. In one embodiment, the relative amount of fluorescence from the donor-labeled enzyme is assayed with a plate reading fluorometer to determine the amount bound to the surface. Subsequently, primer, template and nucleotides are introduced into the well, extension is initiated, and reaction products are recovered and analyzed via polyacrylamide gel electrophoresis. An immobilized enzyme must exhibit >80% of its solution activity level to pass to the detection team for single molecule analysis, where it will be determined if any detected reduction is due to a decrease in the percent of active enzyme in the population or to a decrease in the activity of each enzyme.

Also disclosed herein are methods of increasing the detectability of the gamma incorporation signal through rationally design of an exemplary polymerase, to slow the chemistry step (i.e., bond cleavage and bond formation) of the incorporation reaction. For example, incorporation rates may be slowed by introducing mutations that increase the residence time of the γ-nucleotide in the polymerase's nucleotide binding pocket, thereby increasing the number of photons generated through spFRET and improving both event detection and color identification. The chemistry step was identified as the optimal step to increase acceptor signal because this step is associated with incorporation and will further distinguish a binding event from an incorporation event. In some embodiments, the polymerase is mutated in such a manner that: (1) the chemistry step is slowed without significantly reducing the overall extension efficiency; (2) the Kₘ for terminally labeled nucleotides is decreased (thereby reducing background); and (3) the labeled PPᵢ or polyphosphate product formed upon incorporation is efficiently released (to prevent reverse polymerization). In one embodiment, residues in and around the active site are mutated to accomplish these goals. Such mutagenesis-based approaches may be carried out in conjunction with efforts to identify additional candidate polymerases for mutagenesis or engineered. For example, additional candidates may be identified by screening viral (prokaryotic or eukaryotic) polymerases that undergo a lytic life cycle, grow in a host that lives at 4-50 C and preferably has a rapid replication time (if the host has a rapid replication time, the viral genome must replicate quickly to complete the process prior to host cell division; the polymerase may also be especially efficient at binding and incorporating nucleotides). Typically, the viral polymerase should be responsible for replicating a relatively long genome with minimal accessory proteins and have a relatively low error rate, although the nucleotide binding pocket may be somewhat flexible. Typically, the candidate polymerase should be able to replicate either a DNA or RNA genome. Such requirements should aid identification of a polymerase that uses terminal phosphate modified nucleotides, is processive, performs rapid-DNA-synthesis, exhibits high fidelity, and can be rapidly adapted to our sequencing system

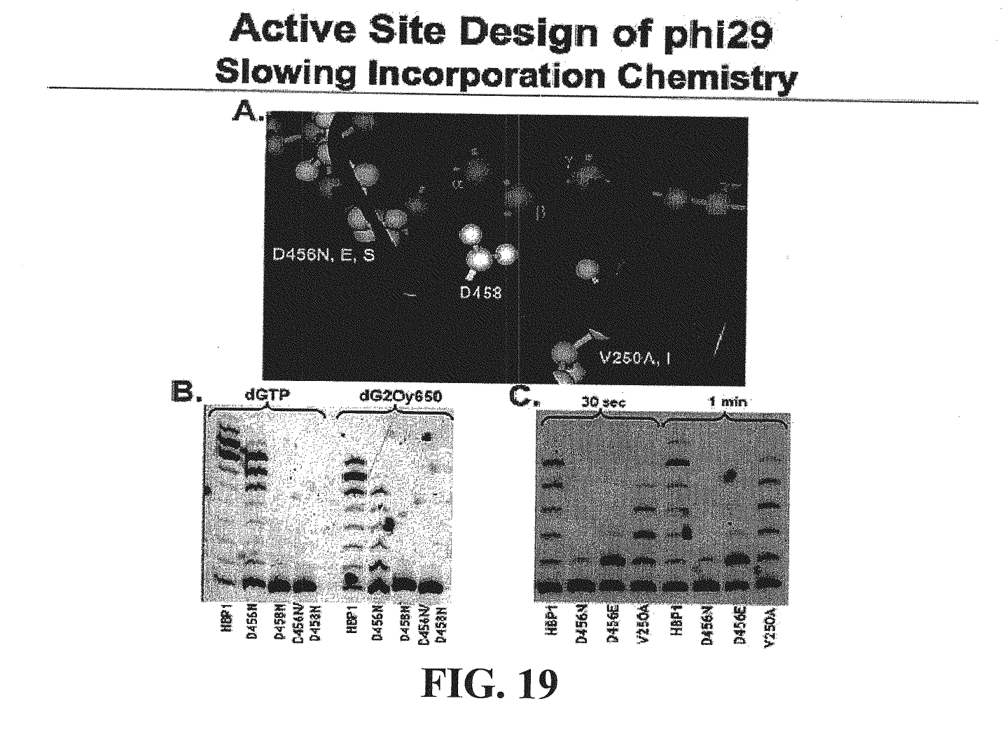

To illustrate the mutagenesis-based approaches of the present disclosure, the polymerase from Phi29 was selected and subjected to a mutagenesis study to obtain improved polymerase variants exhibiting enhanced ability to incorporated terminally-labeled nucleotides. FIG. 19A depicts the active site of Phi29 DNA polymerase. Selected amino acid residues are highlighted in white and yellow to indicate the crucial residue for catalysis and the residues targeted to reduce the rate of catalysis, respectively. To assess the effect of selected residues on incorporation rate and fidelity of replication, various mutations were introduced into a Phi29 exo(−) mutant polymerase. This exo(−) mutant polymerase comprises the protein encoded by SEQ. ID: 3, which comprises wild type Phi29 polymerase (SEQ. ID: 1) with mutations D12A and D66A. Selected residues of the exo(−) mutant were mutated using standard mutagenesis techniques to generate the polymerase mutants listed in Table 1, as described in further detail below. Each mutant was sequenced to confirm the presence of the mutation, purified and then assayed to determine the rate of incorporation and activity on a single-molecule detection system.

As shown in FIG. 19B, the aspartic acid at Phi29 position 458 is crucial to catalysis. When the aspartic acid at position 456 is mutated, the activity is reduced but still detectable. FIG. 19B also depicts the results of 7-base homopolymeric extension reactions, which were carried out for 5 minutes with 5 μM nucleotides. FIG. 19C depicts the results of extension by the Phi29 polymerase mutants D456N as well as D456E under more limiting conditions (1 μM γ-dGTP and 30 second to 1 minute extension time), illustrating that the mutations D456N and D456E have a significant impact on Phi29 extension. The mutation of V250 to an alanine while visibly slowing down extension still allows for at least 5 bases of extension within a minute.

Three residues that have been implicated in catalysis of nucleotide incorporation by phi29 DNA polymerase are D458, D456, and V250 (Berman, Kamtekar et al., 2007). D458 is essential for catalysis—its mutation abolishes enzyme activity (data is shown in FIG. 19B). Mutation of D456 to an asparagine resulted in somewhat reduced extension under permissive conditions, but significantly reduced extension at lower γ-nucleotide concentration and limited time. In one embodiment, D456 is mutated to glutamic acid, thus distorting the active site, but still allowing catalysis due to the presence of the functional group. The backbone of V250 additionally appears to play a role in catalysis; in some embodiments, the chemistry step will be slowed by mutating the V250 residue to an isoleucine or alanine in order to slow the chemistry step.

Using such methods, the polymerase enzyme's catalytic activities can be engineered to affect overall fidelity and processivity. In one embodiment, the polymerase will be maintained in solution, with sequence information being determined by the action of many independent enzymes, with each adding a γ-labeled nucleotide onto the immobilized primer-template (similar to donor replacement sequencing strategies, above).

In addition, modification of residues E375 and Q380 increases incorporation of γ-nucleotides (data not shown). Based on structural analysis, one possible mechanism for this effect may be that removing E375 allows γ-nucleotides better access to the active site via removing hindrance associated with the fluorophore on the γ-phosphate. In one embodiment, the E375A or C mutation will be combined with one or more mutations that facilitate terminally labeled nucleotide detection to ensure rapid binding of the nucleotides and rapid release of the labeled pyrophosphate or polyphosphate product after slowed catalysis, thereby preserving overall reaction efficiency.

The following table lists the Phi29 mutants prepared and/or tested for activity in gamma-labeled nucleotide incorporation assays and in single molecule detection systems. Each mutant was introduced into an already-mutated Phi29 polymerase termed Phi29 exo(−), which comprises the protein sequence of wild type Phi29 polymerase, as provided in SEQ. ID: 1, but additionally includes the mutations D12A and D66A, and exhibits reduced exonuclease activity as compared to its wild-type counterpart. The protein sequence of the Phi29 exo(−) mutant is provided in SEQ. ID: 3.

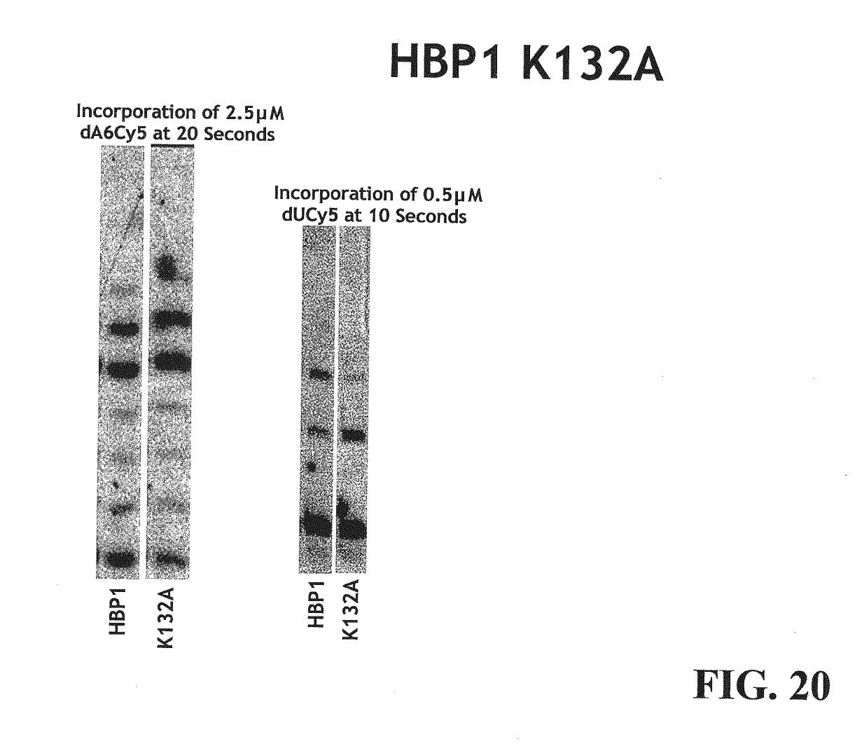

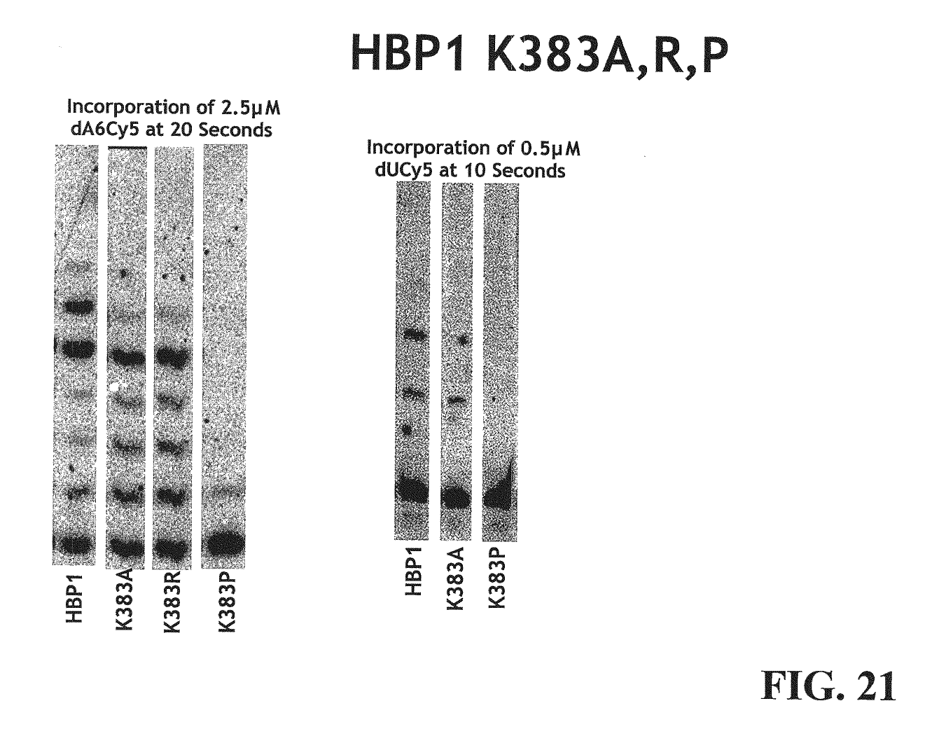

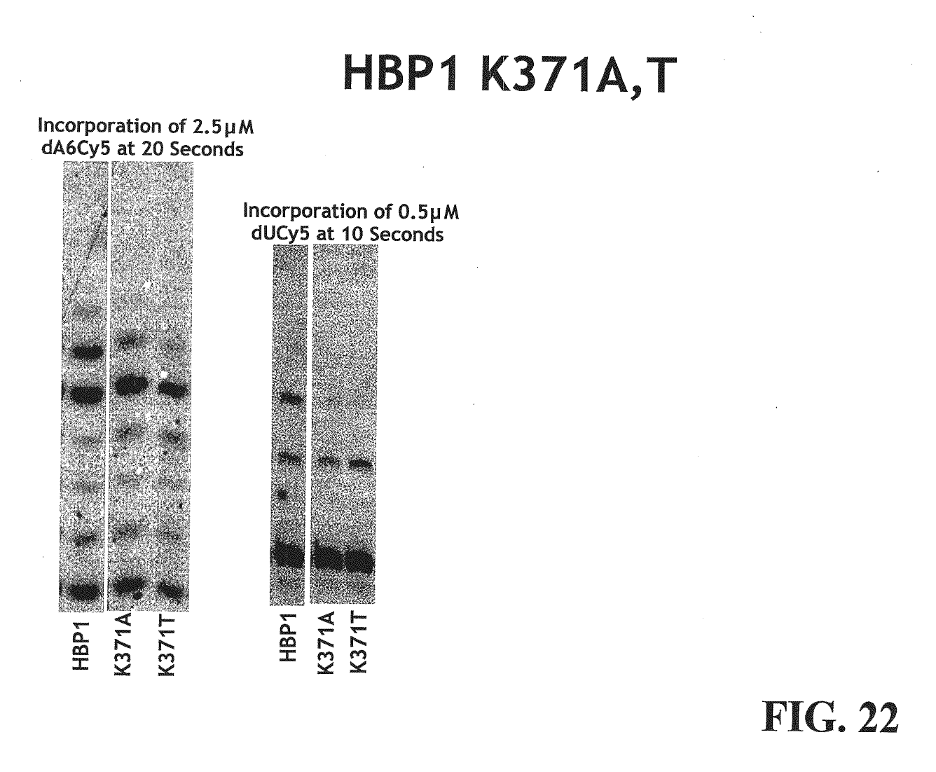

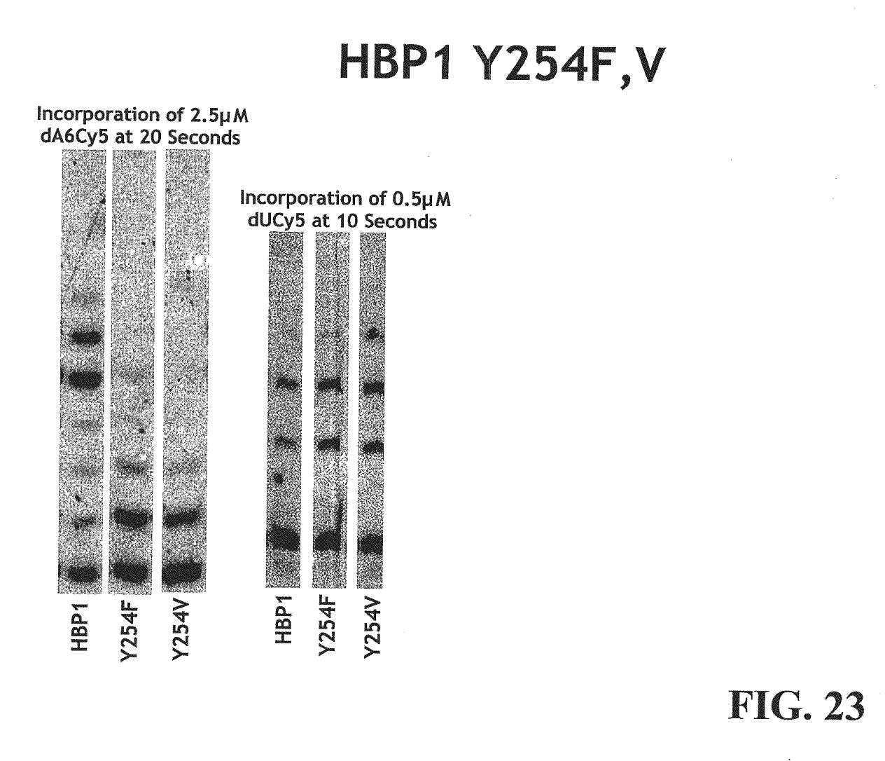

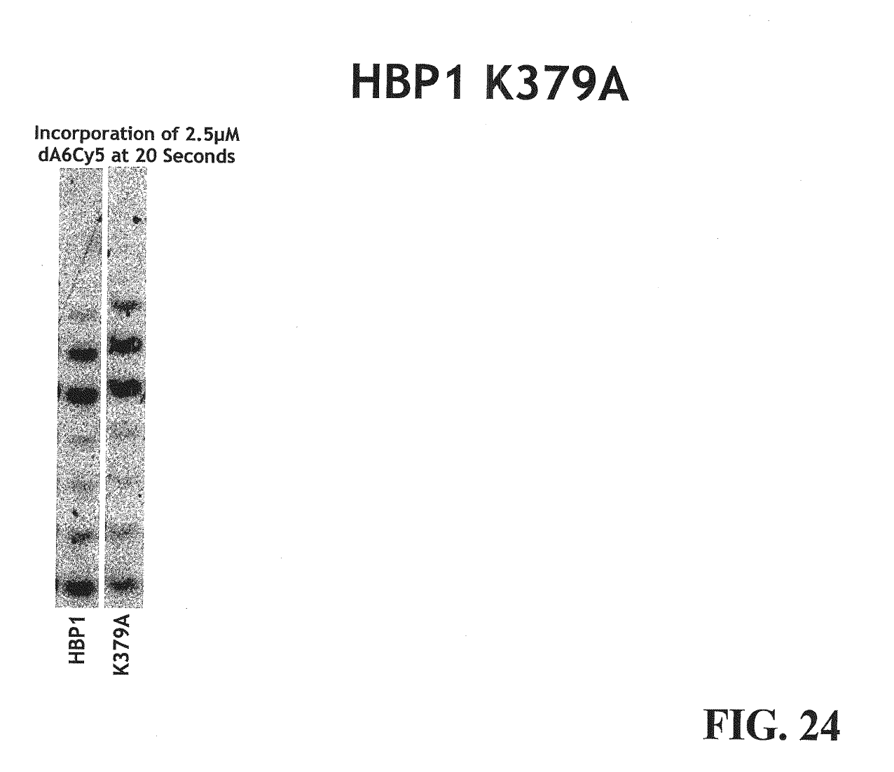

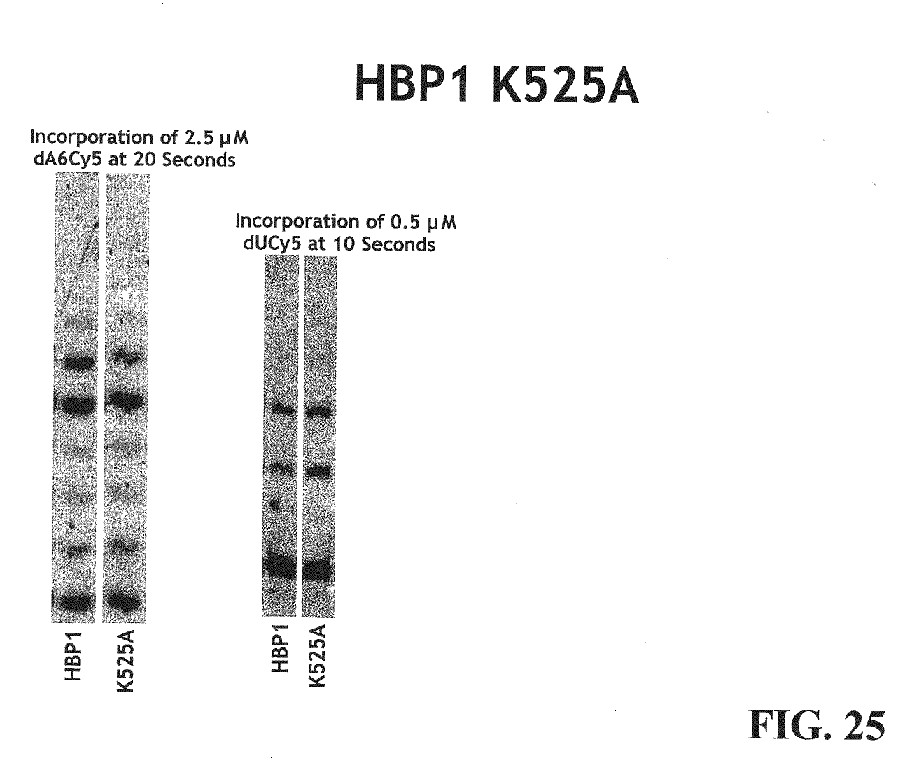

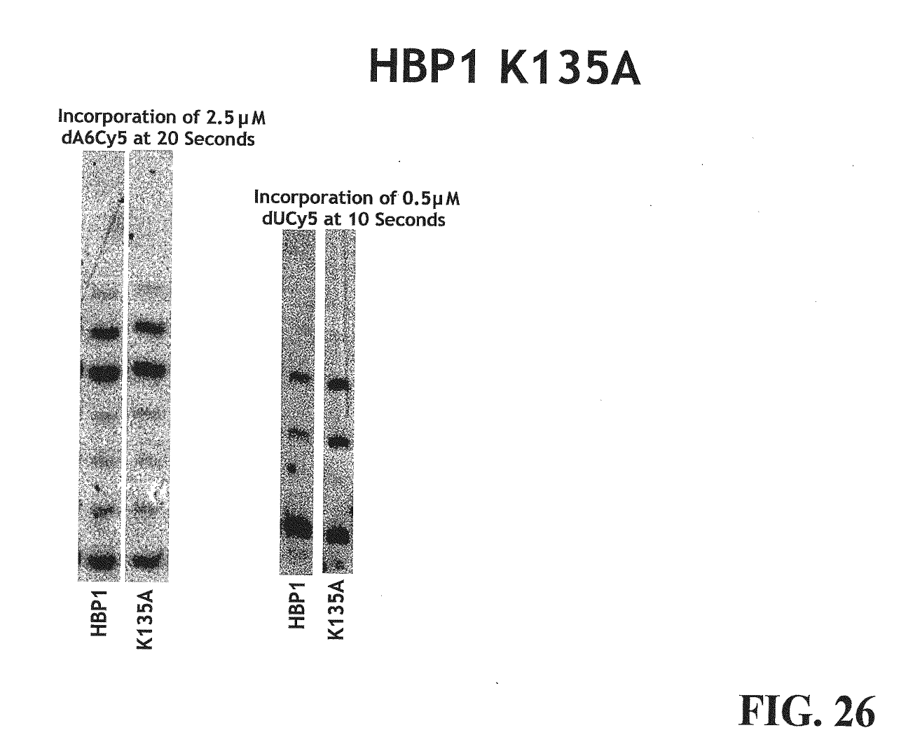

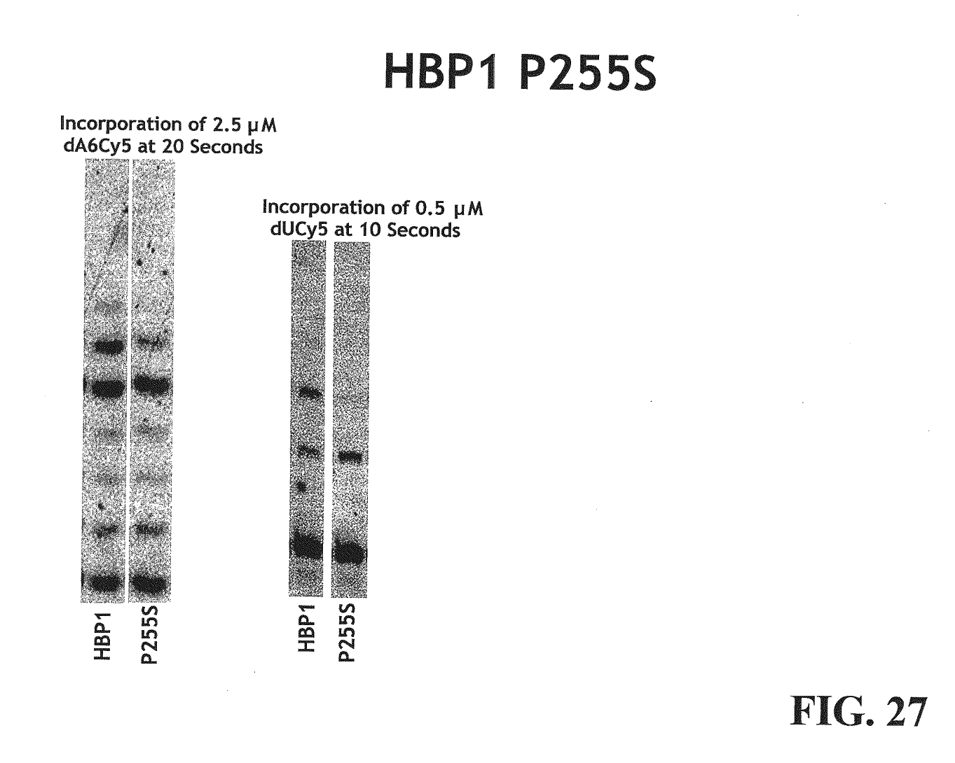

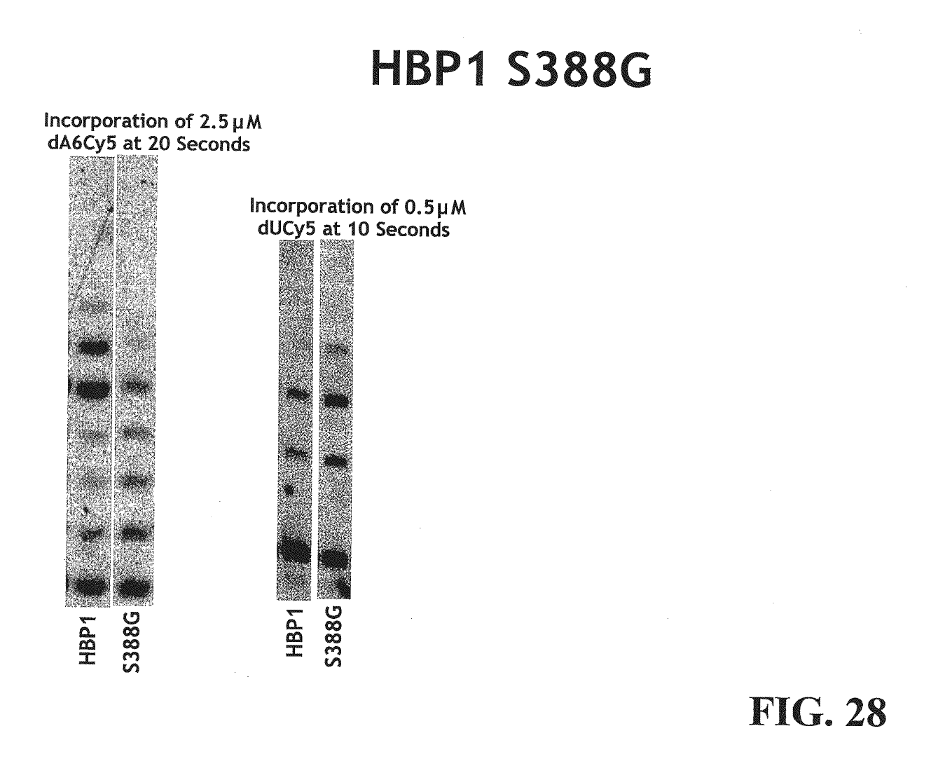

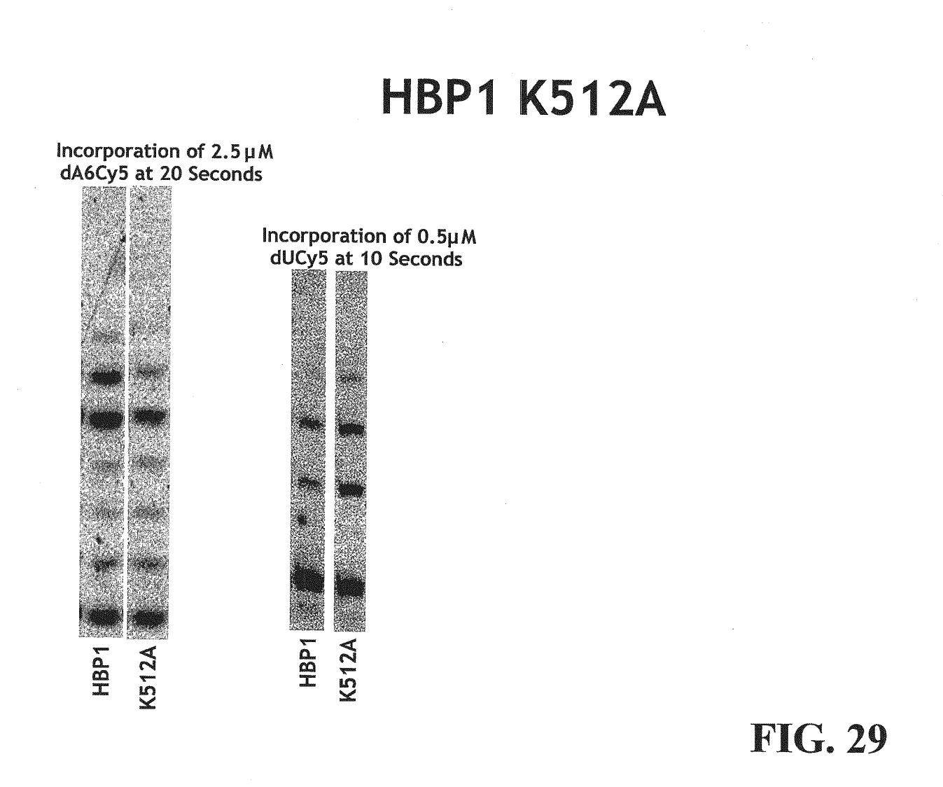

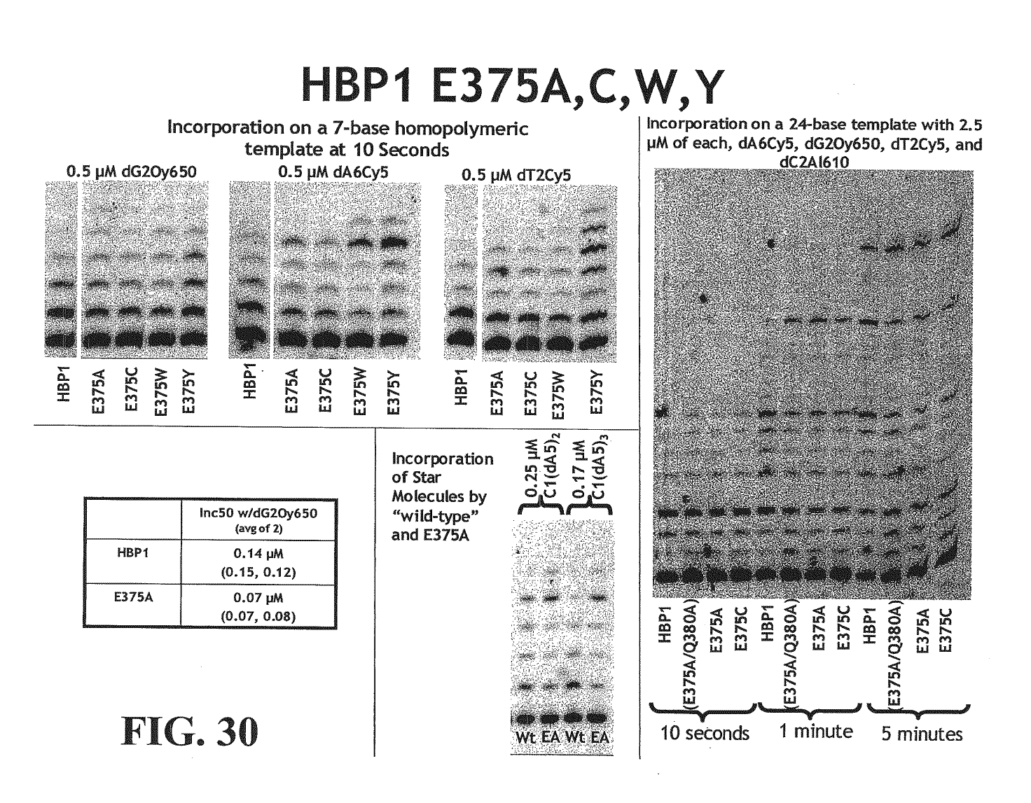

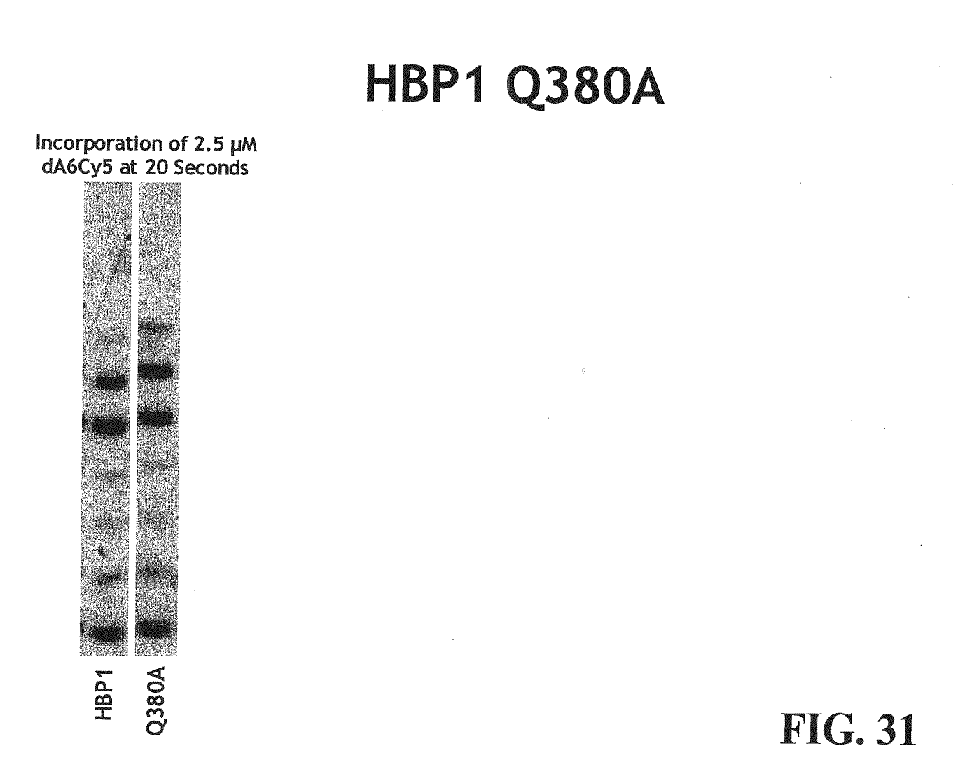

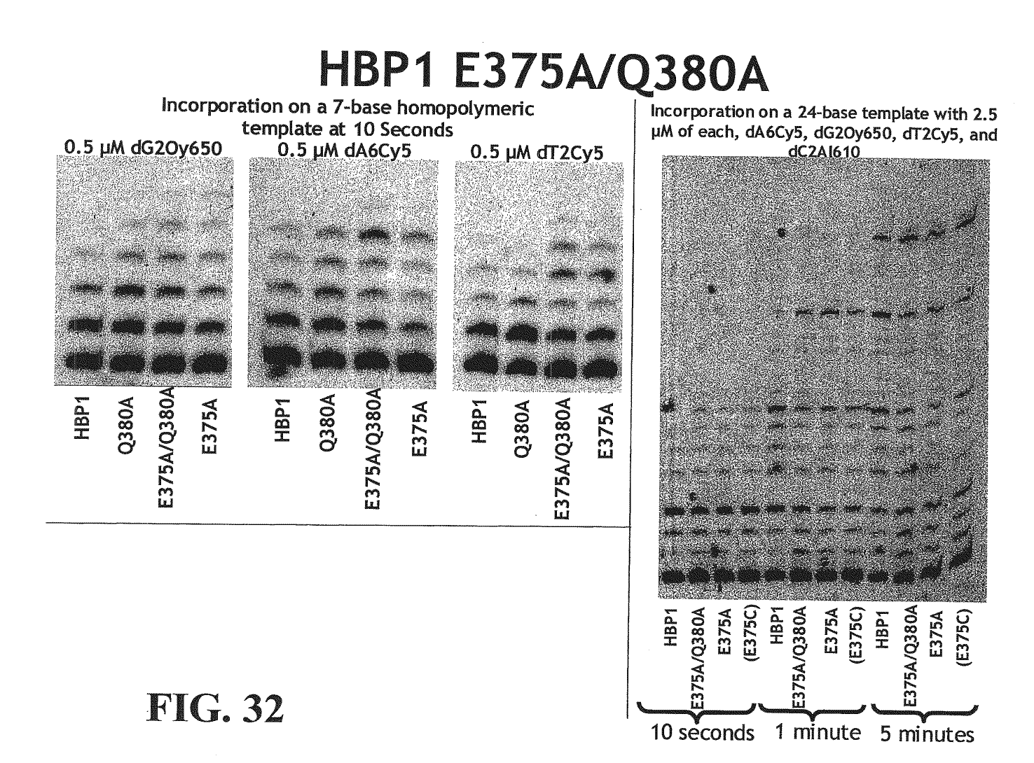

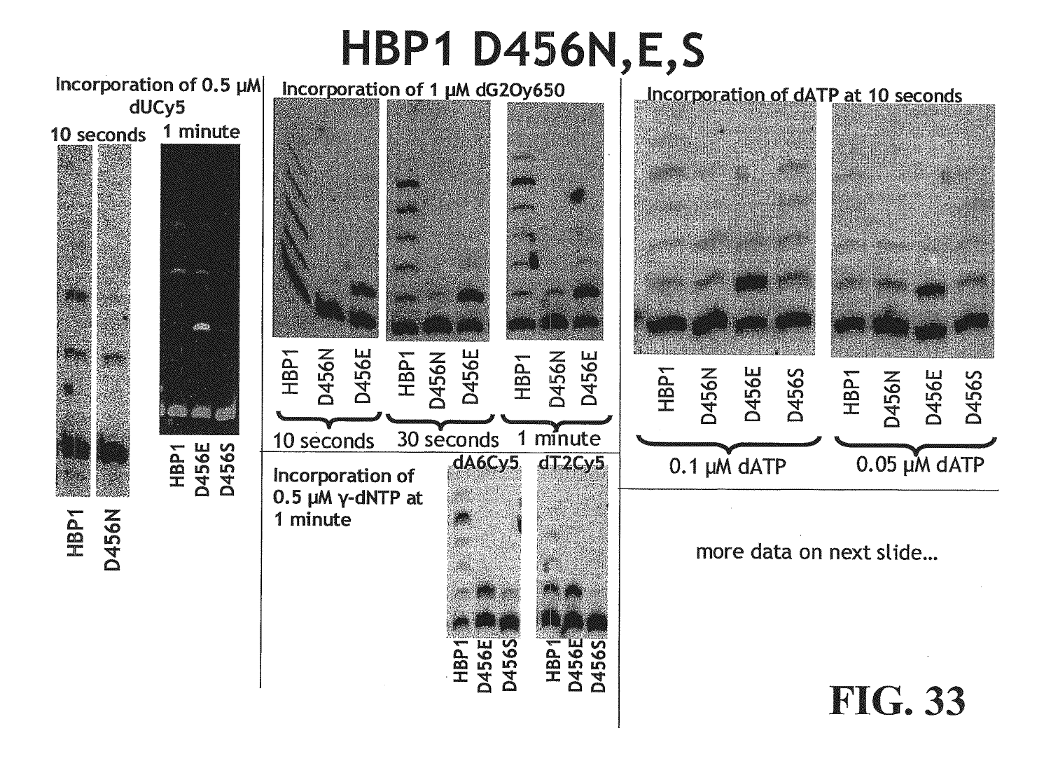

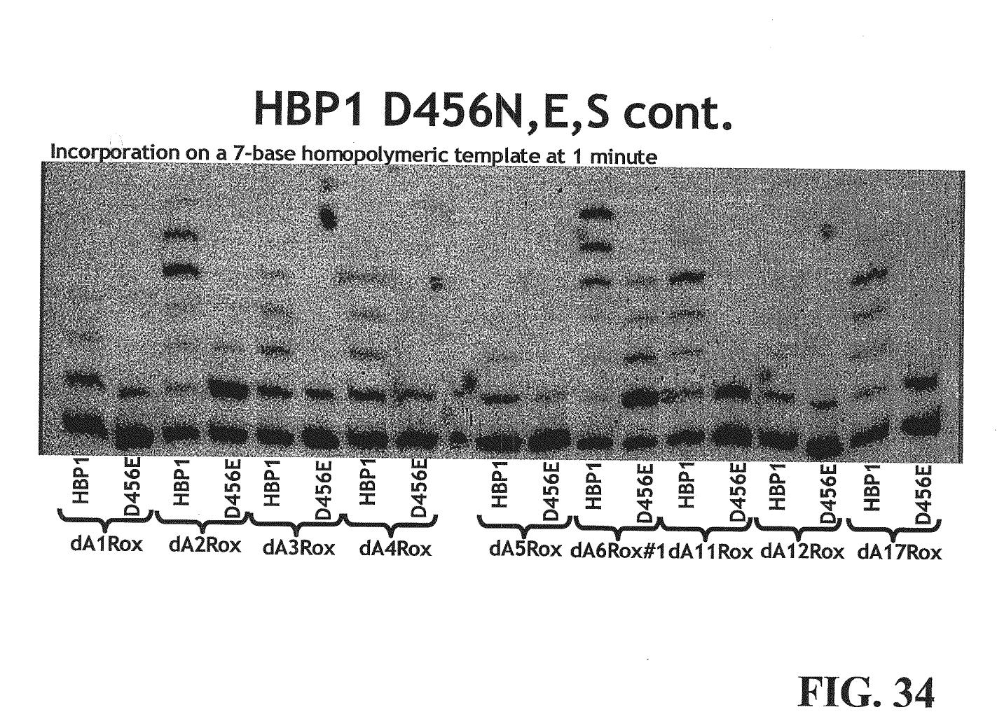

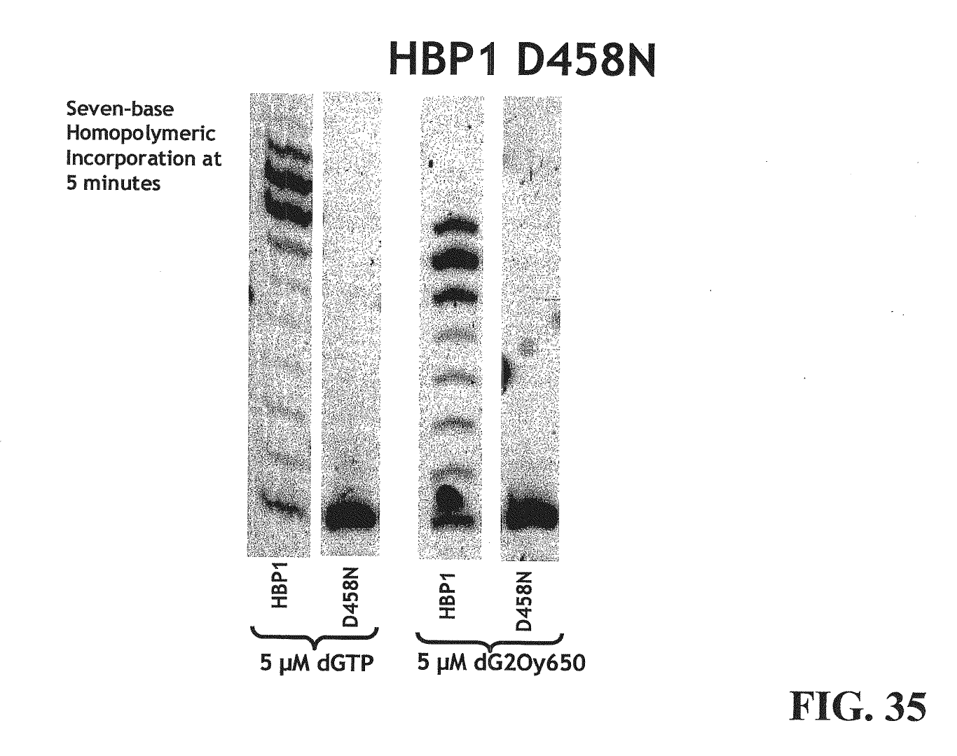

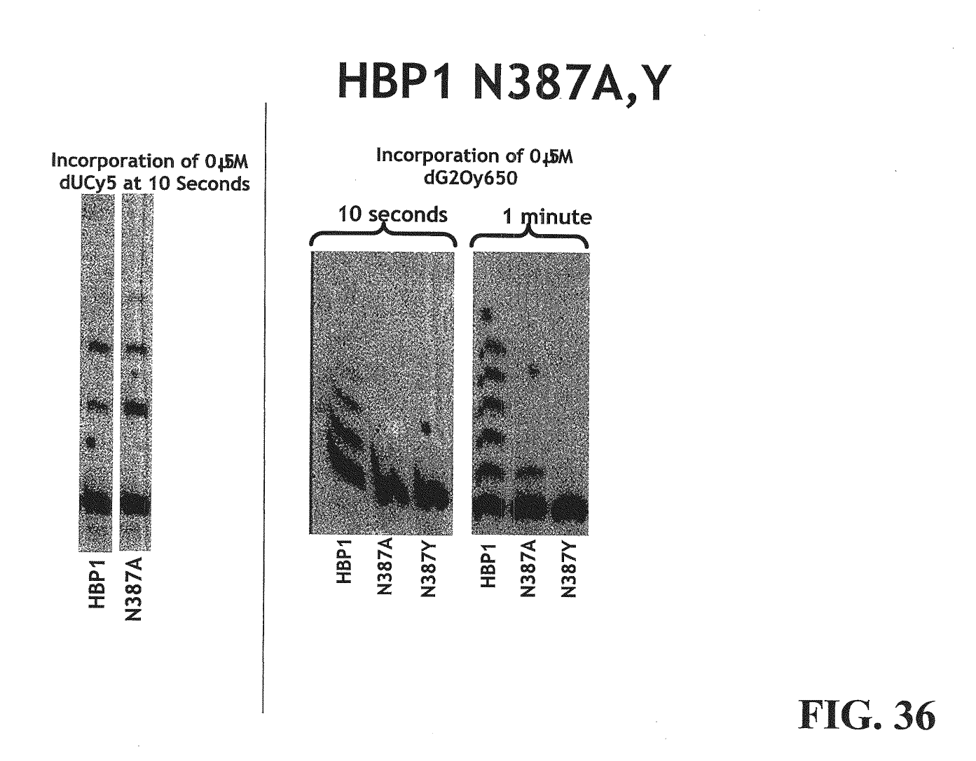

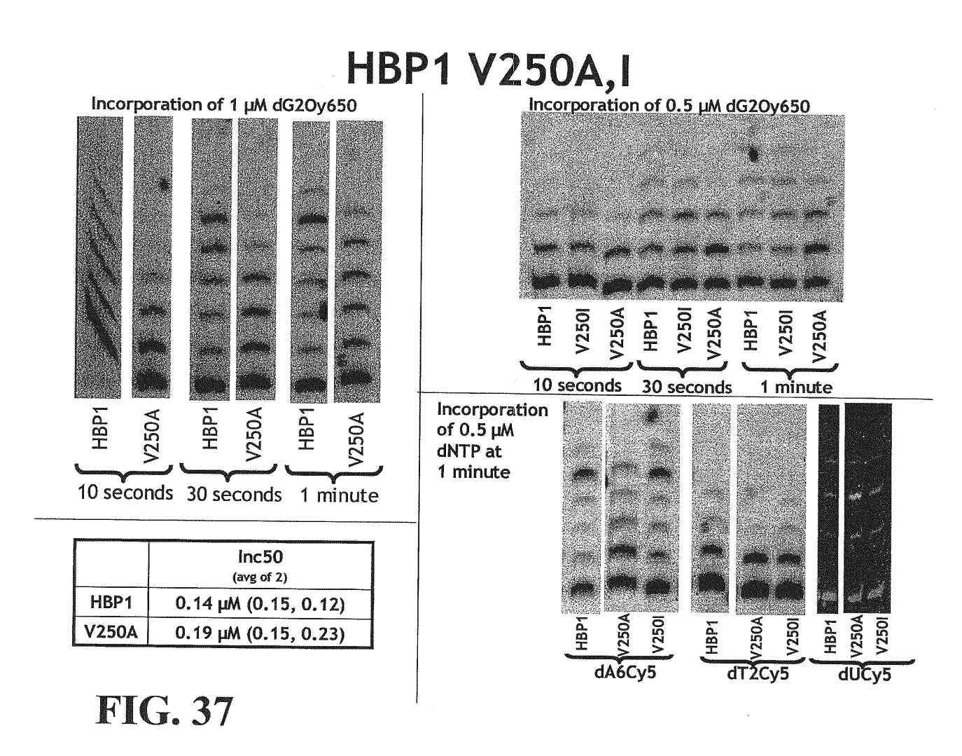

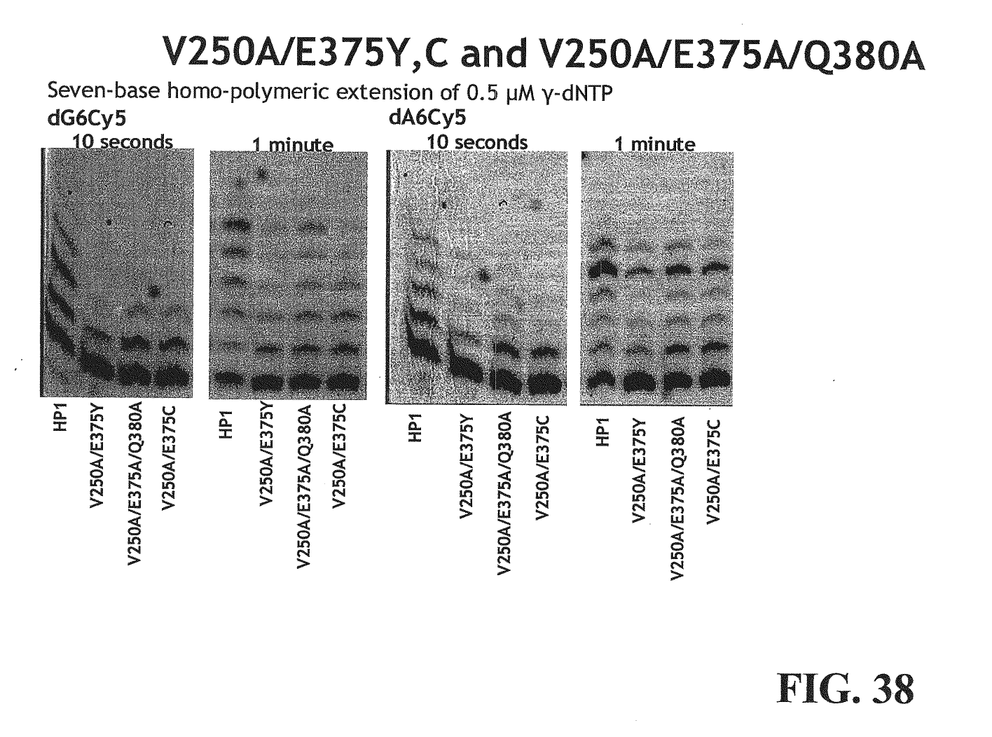

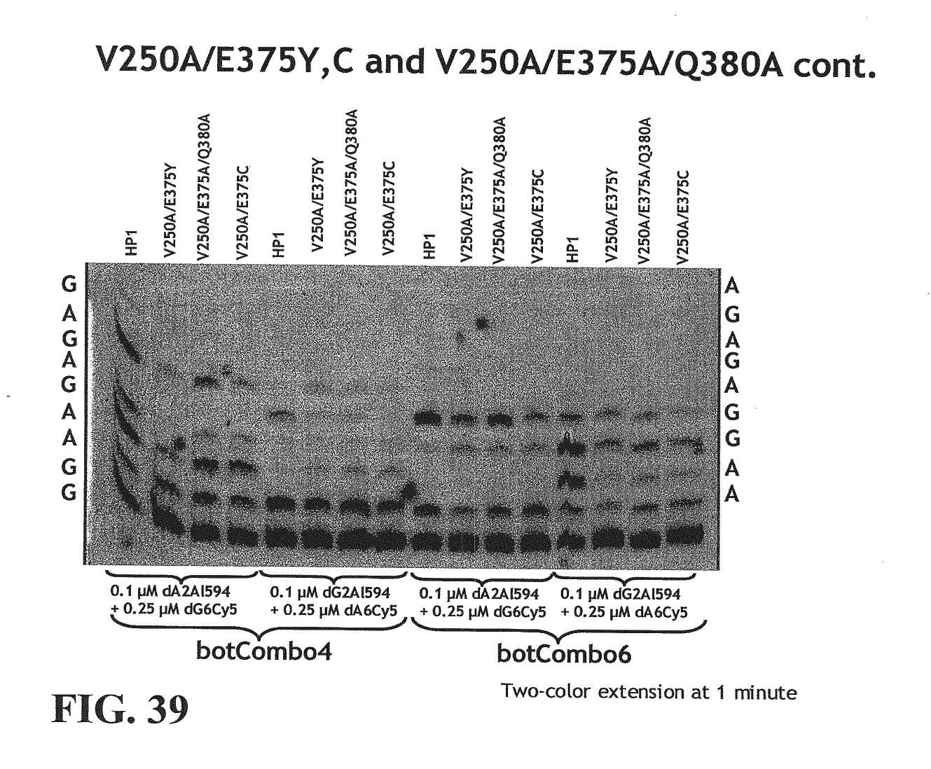

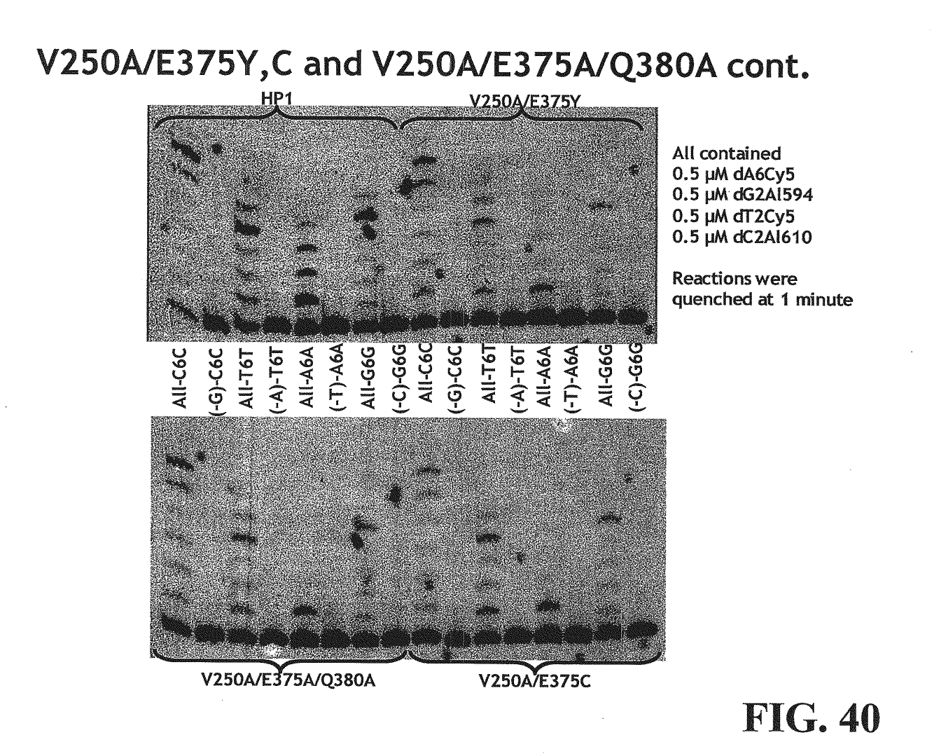

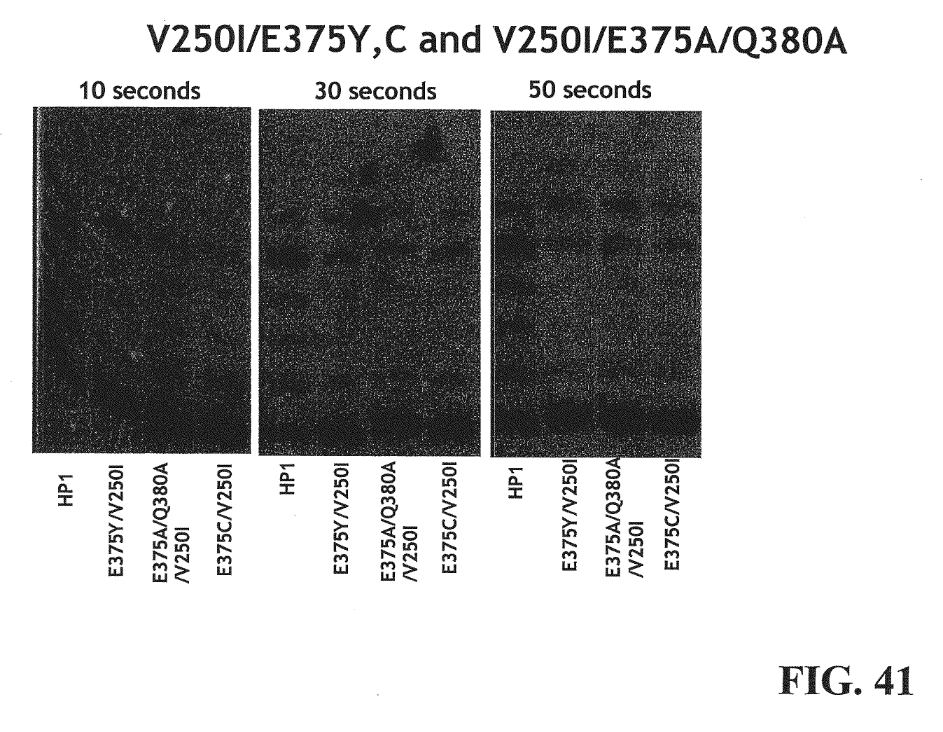

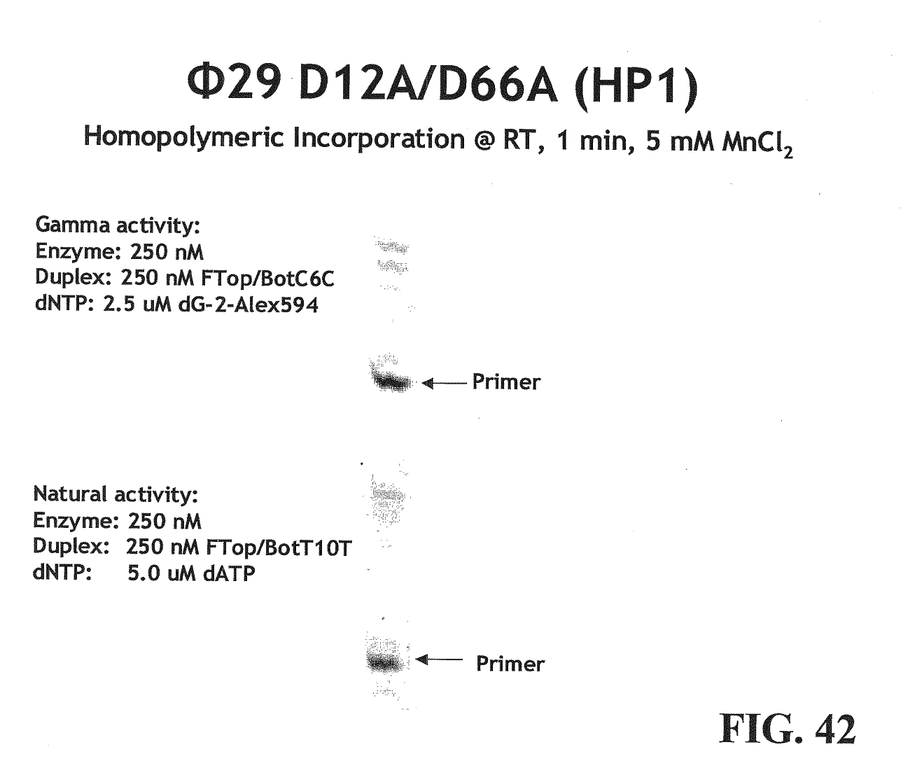

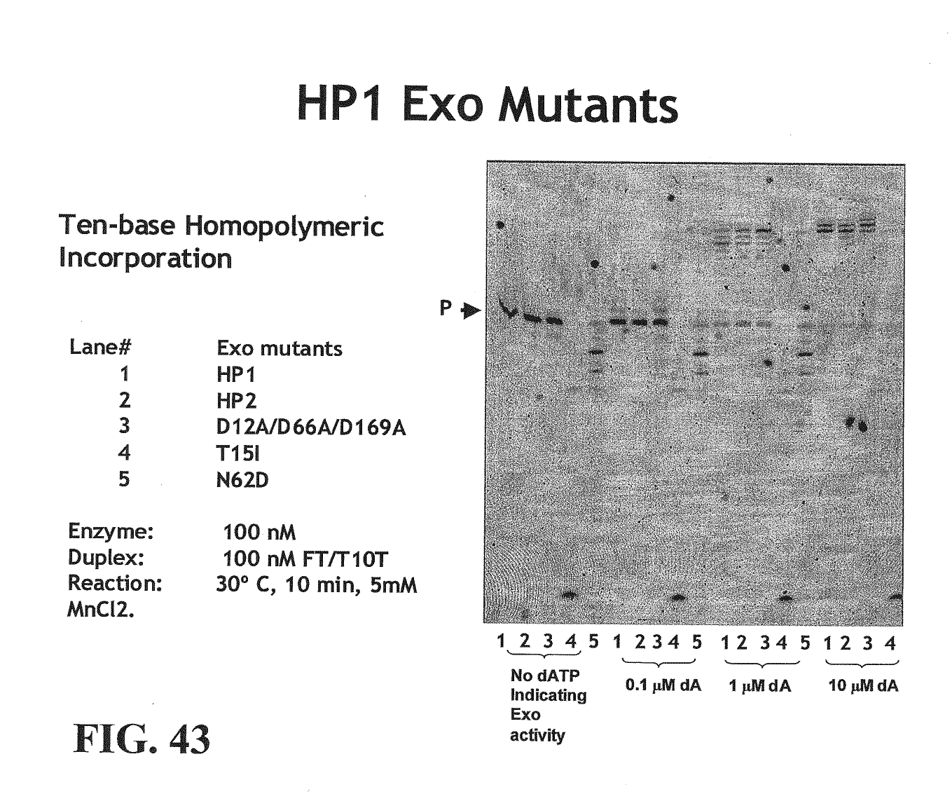

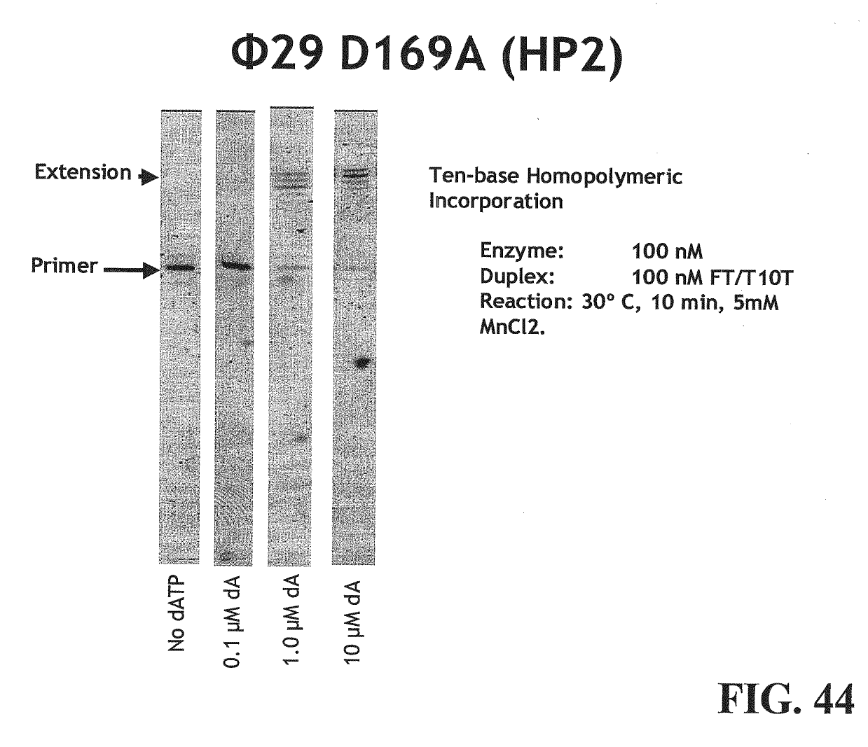

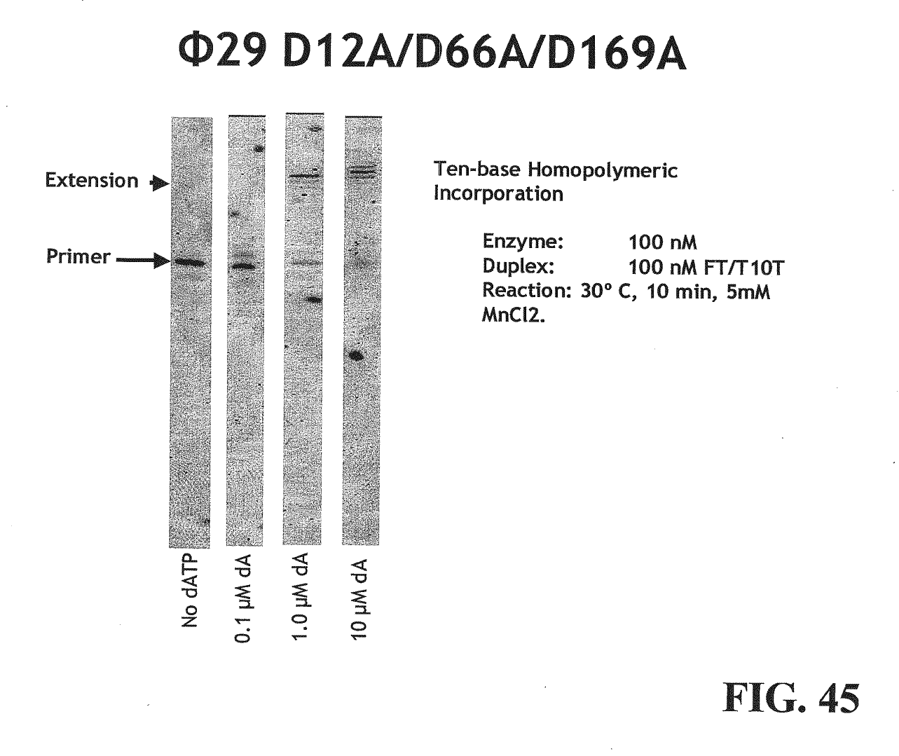

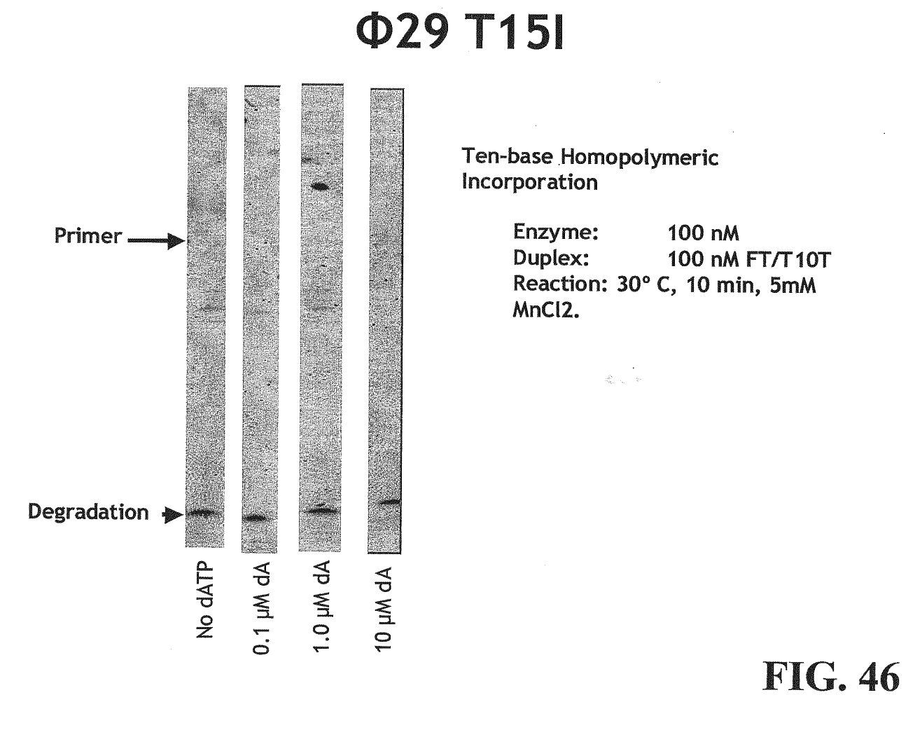

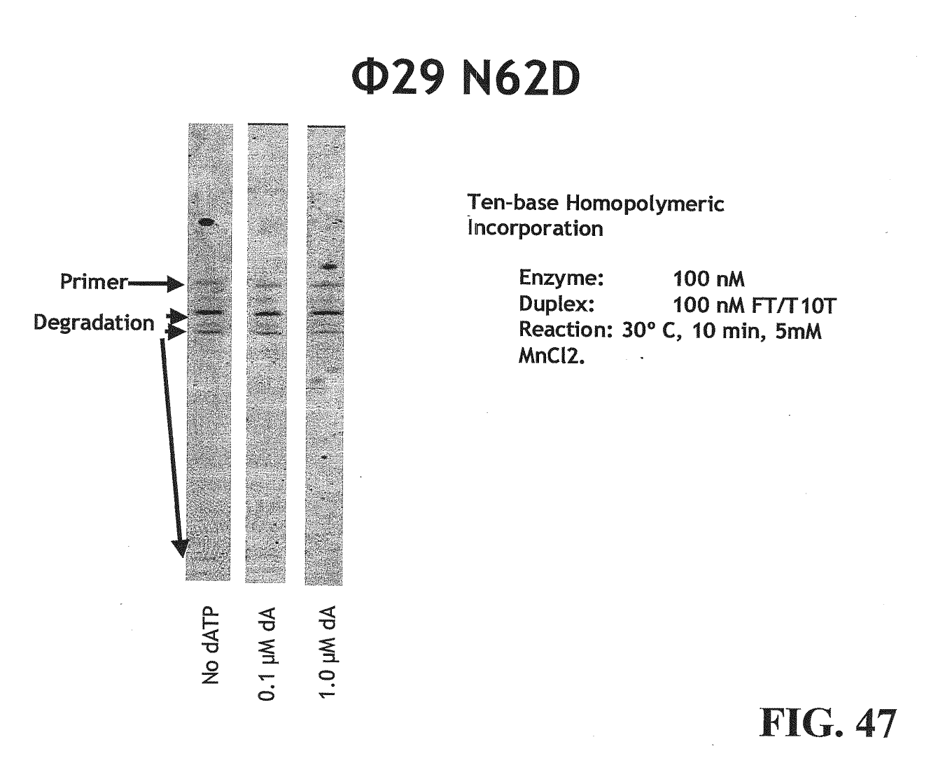

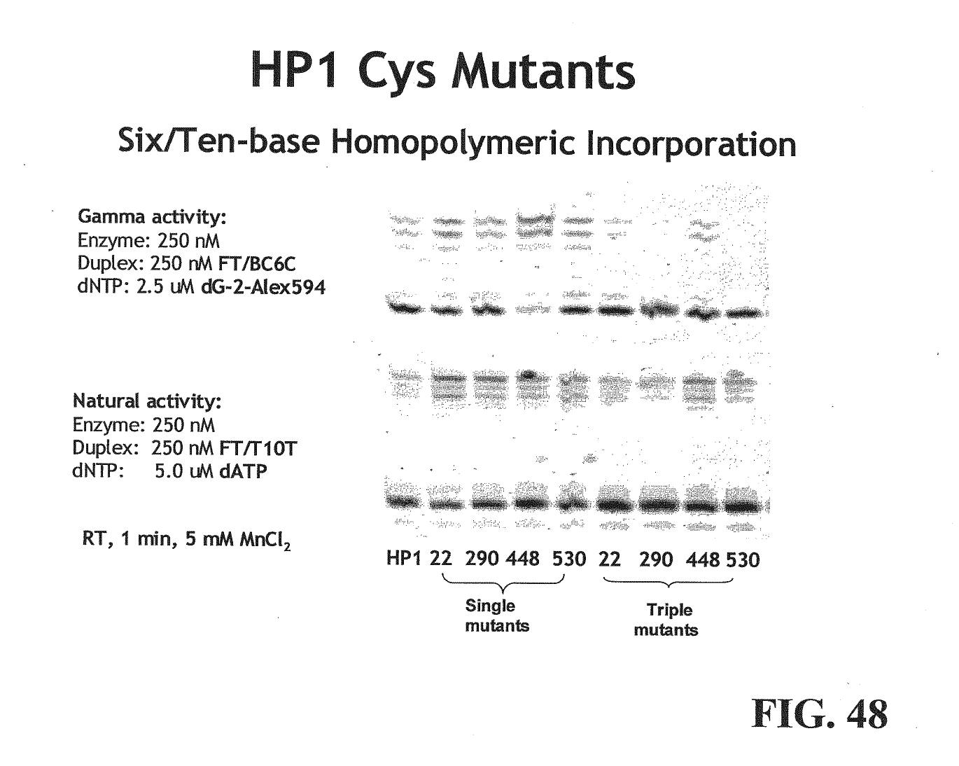

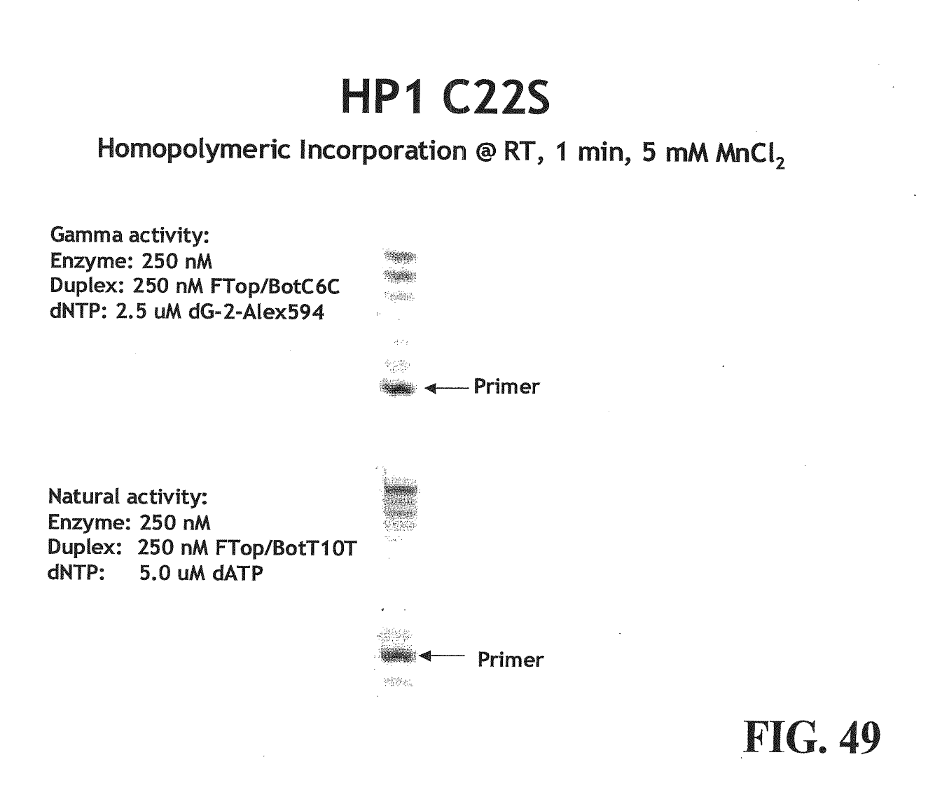

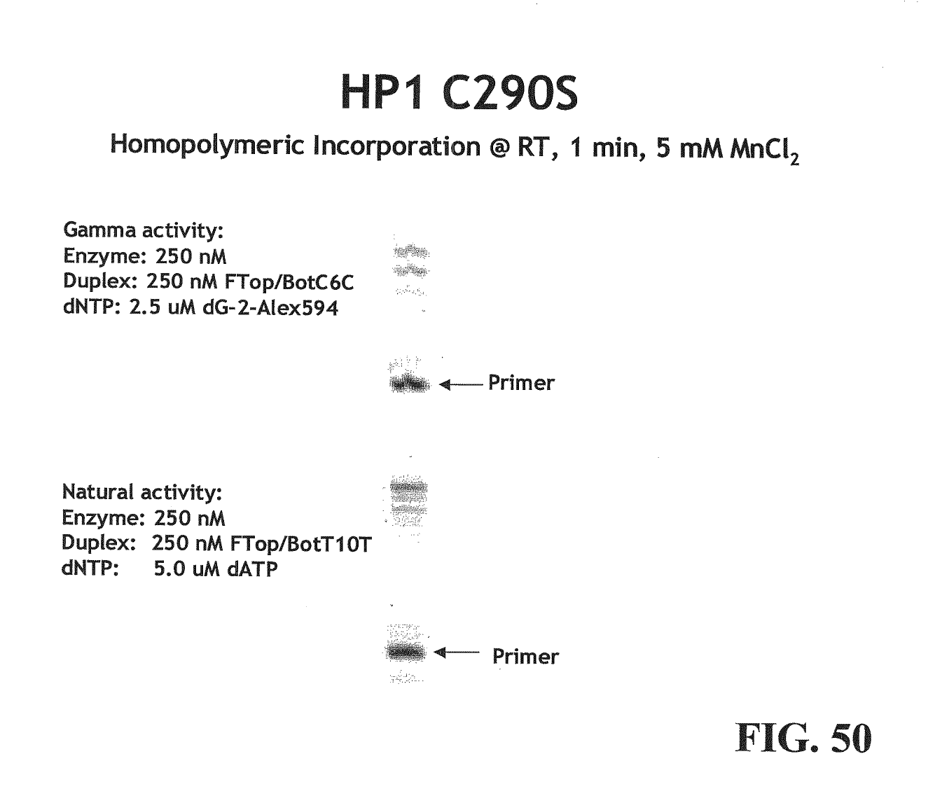

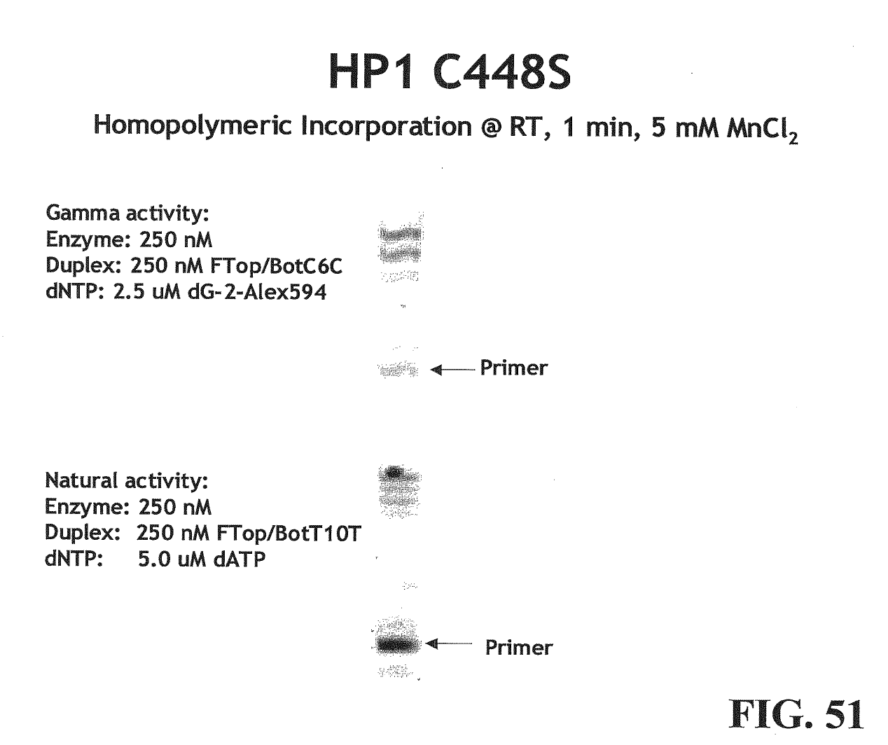

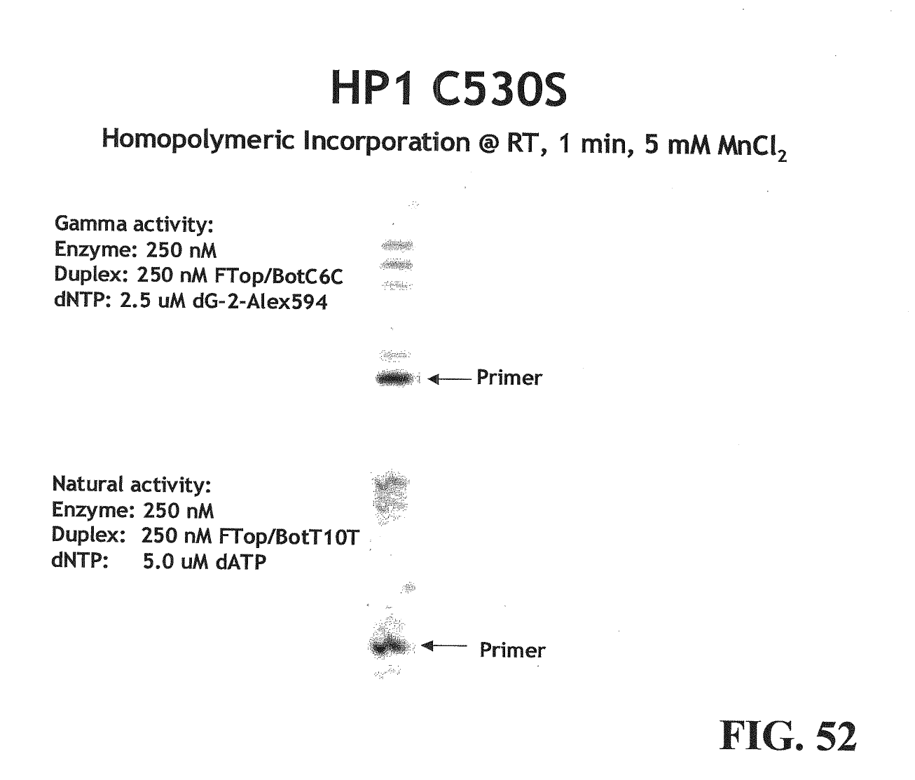

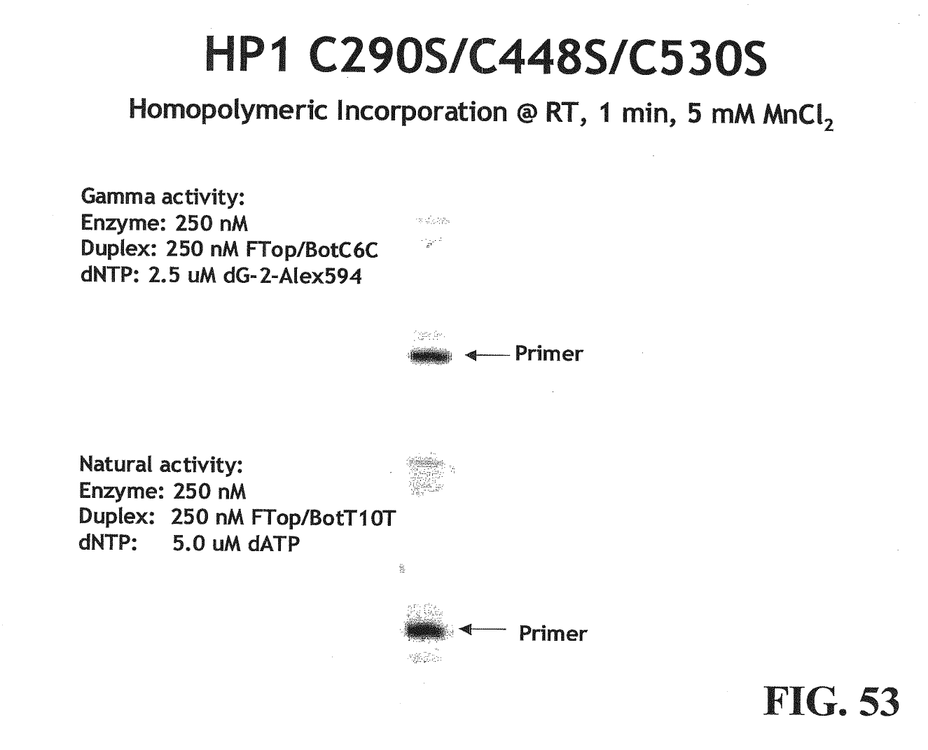

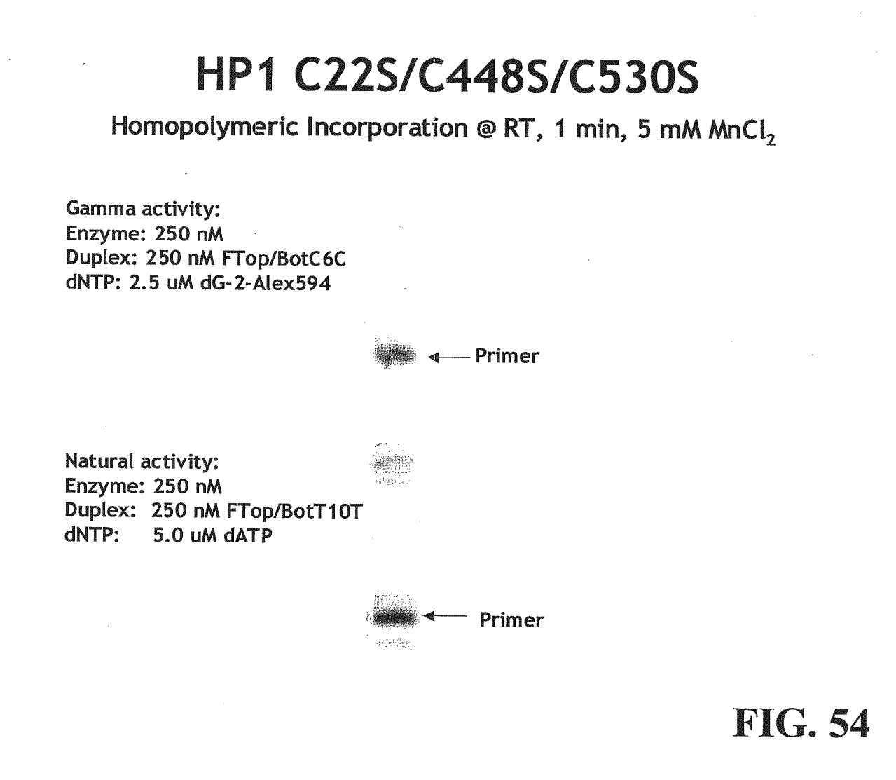

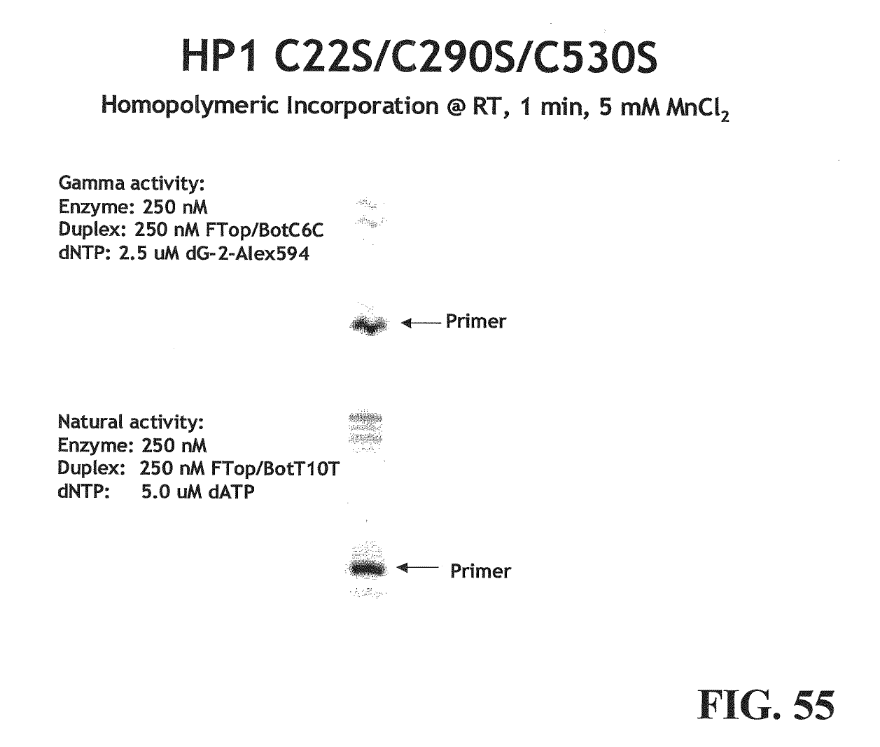

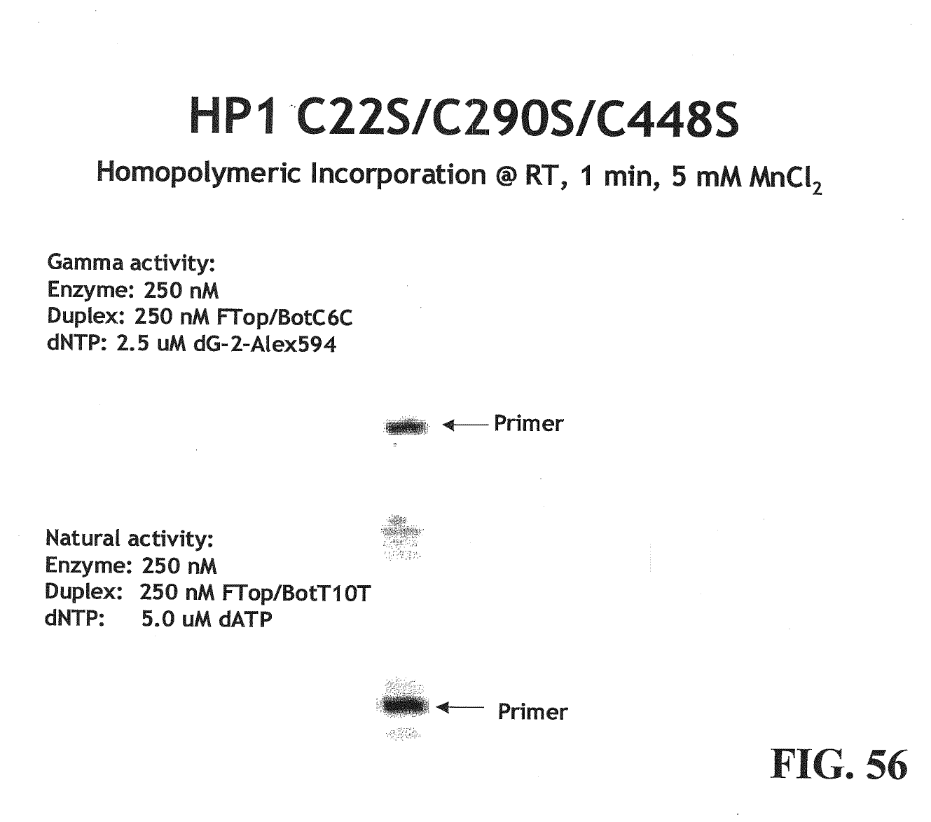

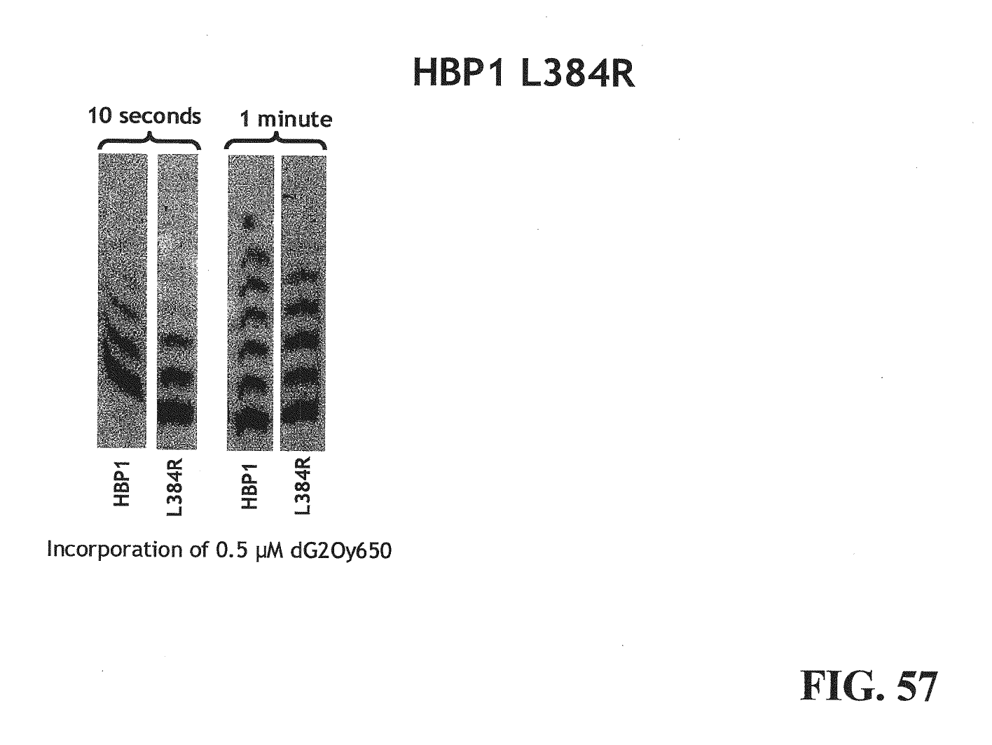

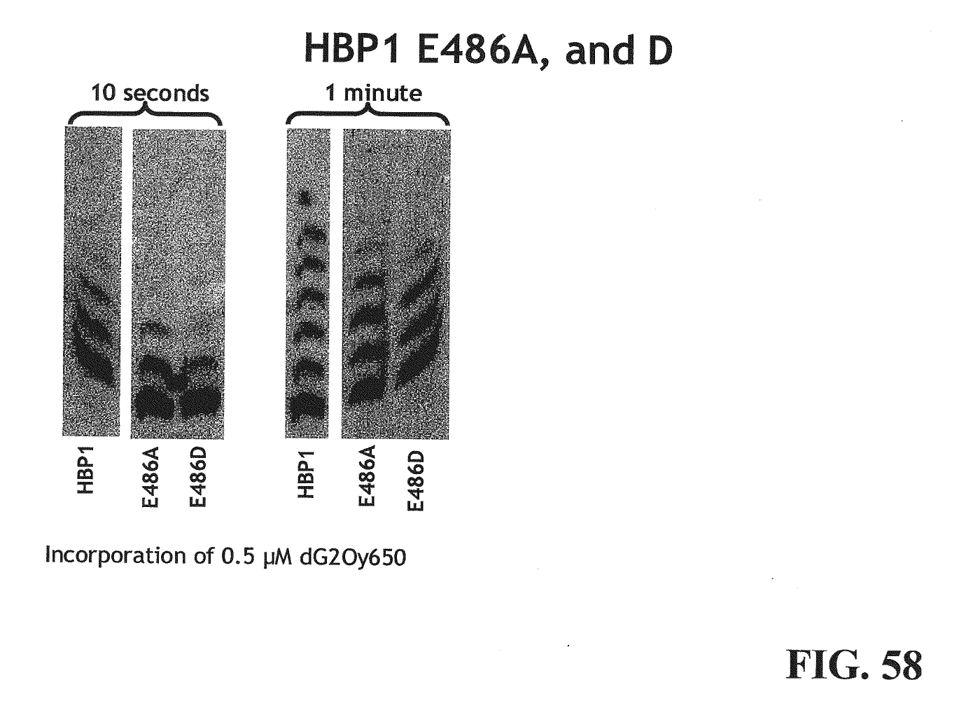

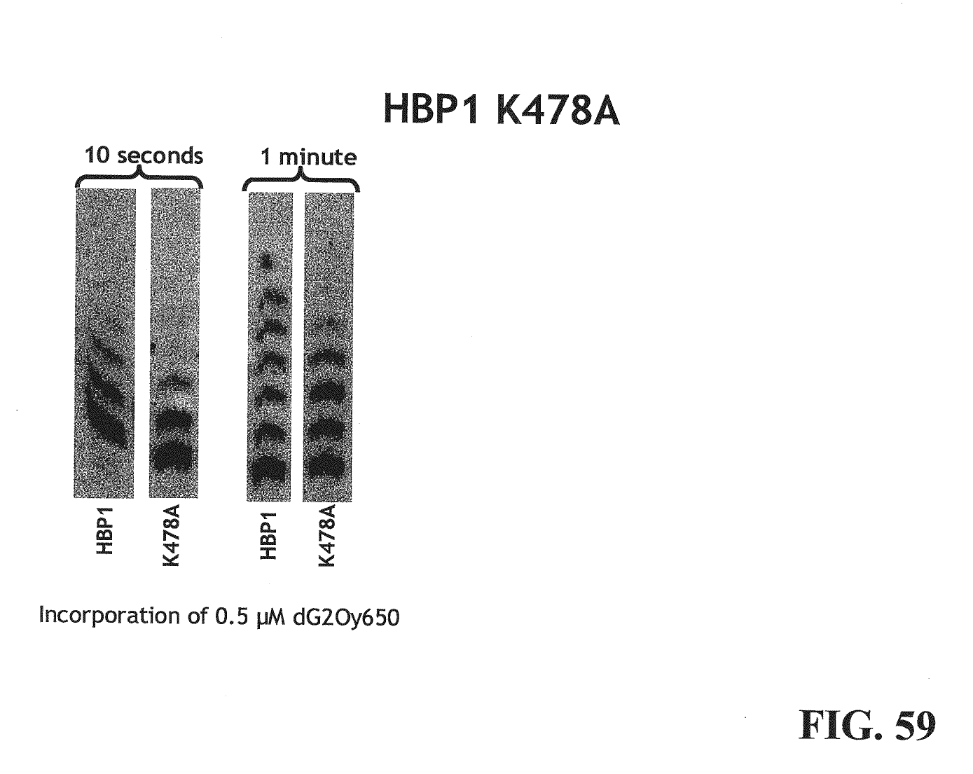

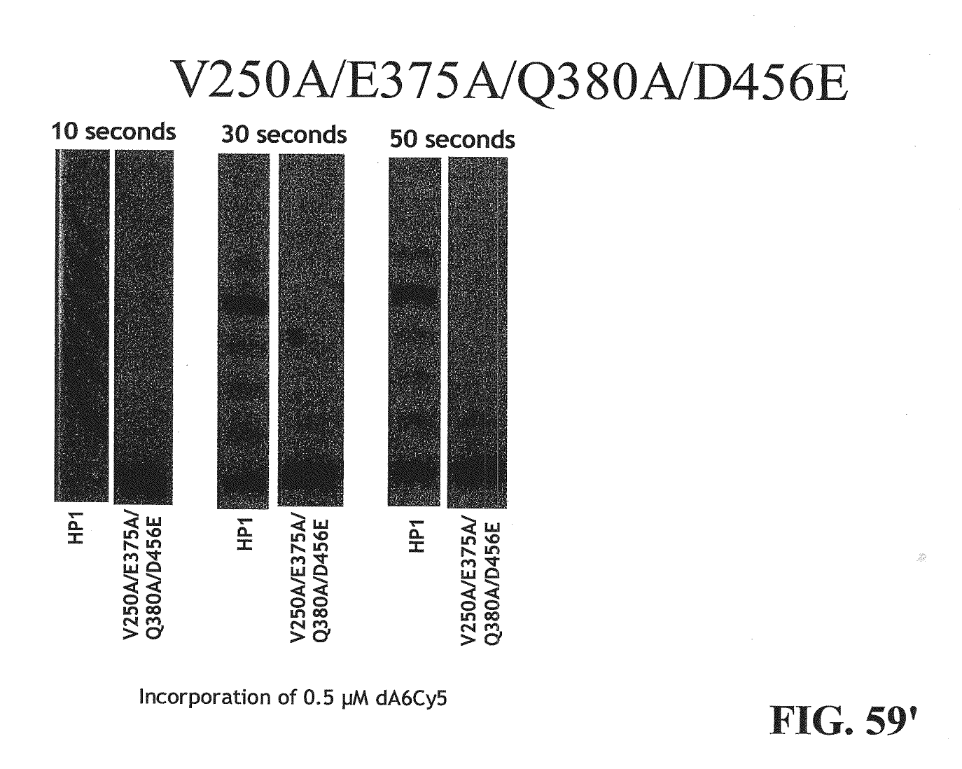

Table 1, below, summarizes the ability of each mutant Phi29 polymerase to incorporate gamma-labeled nucleotides (as indicated in the column entitled “Ensemble Extension Activity”), fidelity of replication (as indicated in the column entitled “Fidelity”) and for activity in a single-molecule detected system, as depicted in FIGS. 20-59′ and 69-76 and associated description (see below), and indicated in the last column, entitled “Activity on Detection System.” Each and every mutation introduced into a given Phi29 mutant protein is listed in the column entitled “Mutations”; mutations are designated by the original amino acid/its position/its replacement, e.g., V250A means that valine at position 250 was replaced by alanine by site specific mutagenesis. If two or more amino acid substitutions are made, then each is designated as above and separated by a “/”. The predicted effect of a given mutation on polymerase activity, based on structure-function analysis of Phi29 polymerase, is provided in the column entitled “Expected Effect.” The various Phi29 mutants generated via selective mutagenesis, along with any applicable results of extension studies, fidelity assays and single molecule detection studies using each mutant protein, are collectively summarized in Table 1, below. To perform the extension studies, 330 nM Phi29 mutant protein and 100 nM 5′ fluorescein-labeled primer:template duplex were co-incubated in a solution comprising 50 mM Tris (pH 7.0), 2 mM MnCl₂, 2 mM DTT, 0.1% Triton-X-100, 0.01% Tween-20. The reactions were initiated by addition of 0.5 μM or 5 μM of gamma-labeled dNTP, and quenched via addition of 30 mM EDTA at timepoints ranging from 10 seconds to 5 minutes. The reaction products were then separated on a 20% denaturing acrylamide gel and imaged on a Bio-Rad Molecular Imager FX. The intensity of each reaction product was quantified using Bio-Rad Quantity One software. FIGS. 20-59′ depict the results of extension studies conducted using specific Phi29 mutants.

To assay the replication fidelity of each mutant Phi29 polymerase protein, primer extensions were carried out in the same manner, except that an incorrect (i.e., non-complementary) gamma-labeled nucleotide was added to the extension mixture in lieu of the complementary nucleotide, and the amount of extended product following extension in presence of the incorrect nucleotide was analyzed.

TABLE 1
Phi29 Variants

| | | | Ensemble | | Activity on |
|---|---|---|---|---|---|
| | | | Extension | | Detection |
| Mutations | Expected Effect | Purified? | Activity | Fidelity | System |
| Parent Enzyme | 3′ to 5′ | yes | Benchmark | No mis- | Benchmark |
| Phi29exo(−) | exo-nuclease | | Activity | incorporation | Activity |
| D12A/D66A ^{¶} ^{#} | deficient | | | detected | |
| V250A/E375Y | *** | yes | Decreased rate | No mis- | 3x more signals. |
| | | | of γ-nucleotide | incorporation | More high ASB signals. |
| | | | incorporation | detected | More long duration |
| | | | | | signals. |
| V250A/E375A/ | *** | yes | Decreased rate | No mis- | 3x more signals. |
| Q380A | | | of γ-nucleotide | incorporation | More high ASB signals. |
| | | | incorporation | detected | More long duration |
| | | | | | signals. |
| | | | | | Signals detected |
| | | | | | earlier. |
| V250A/E375C | *** | yes | Decreased rate | No mis- | 3.5x more signals. |
| | | | of γ-nucleotide | incorporation | More long duration |
| | | | incorporation | detected | signals. |
| | | | | | Signals detected |
| | | | | | earlier. |
| V250I/E375Y | *** | yes | Decreased rate | pending | Pending |
| | | | of γ-nucleotide | | |
| | | | incorporation | | |
| V250I/E375A/ | *** | yes | Decreased rate | pending | Pending |
| Q380A | | | of γ-nucleotide | | |
| | | | incorporation | | |
| V250I/E375C | *** | yes | Decreased rate | pending | Pending |
| | | | of γ-nucleotide | | |
| | | | incorporation | | |
| V250A | ** | yes | Decreased rate | pending | Fewer signals. |
| | | | of γ-nucleotide | | |
| | | | incorporation | | |
| | | | Inc50 = [symbol] | | |
| | | | (Phi29 = 0.14) | | |
| V250I | ** | yes | Decreased rate | pending | More long duration |
| | | | of γ-nucleotide | | signals. |
| | | | incorporation | | |
| E375A | * | yes | More efficient | No mis- | 3x more signals. |
| | | | γ-nucleotide | incorporation | |
| | | | incorporation | detected | |
| | | | Inc50 = [symbol] | | |
| | | | (Phi29 = 0.14) | | |
| E375C | * | yes | More efficient | pending | Slightly higher |
| | | | γ-nucleotide | | # of signals. |
| | | | incorporation | | More high ASB signals. |
| | | | | | Signals detected |
| | | | | | earlier. |
| E375Y | * | yes | More efficient | pending | 3x more signals. |
| | | | γ-nucleotide | | More long duration |
| | | | incorporation | | signals. |
| E375A/Q380A | * | yes | More efficient | pending | 3.5x more signals. |
| | | | γ-nucleotide | | |
| | | | incorporation | | |
| Q380A | | yes | More efficient | No mis- | 2.5x more signals. |
| | | | γ-nucleotide | incorporation | |
| | | | incorporation | detected | |
| D458N | Create a | yes | No activity | No activity | Pending |
| | “dead” | | with γ- or | with γ- or | |
| | enzyme | | natural | natural | |
| | | | nucleotides | nucleotides | |
| D456N | ** | yes | Impaired | pending | Fewer signals. |
| | | | Incorporation | | |
| | | | of γ-nucleotides | | |
| D456E | ** | yes | Increased first | pending | Fewer signals. |
| | | | base extension, | | |
| | | | but impaired | | |
| | | | thereafter | | |
| D456S | ** | yes | Impaired | pending | Fewer signals. |
| | | | incorporation | | |
| | | | of γ-nucleotides | | |
| V250A/E375A/ | | yes | Impaired | pending | Pending |
| Q380A/D456E | | | incorporation | | |
| | | | of γ-nucleotides | | |
| E375Y/V250L | *** | yes | decreased | pending | Pending |
| E375Y/V250P | *** | yes | no activity | pending | Pending |
| E375Y/V250Q | *** | yes | decreased | pending | Pending |
| E375Y/V250R | *** | yes | no activity | pending | Pending |
| E375Y/V250Y | *** | yes | similar to | pending | Pending |
| | | | parent Phi29 | | |
| | | | exo(−) D12A/D66A | | |
| E375Y/V250F | | yes | similar to | pending | Pending |
| | | | parent Phi29 | | |
| | | | exo(−) D12A/D66A | | |
| E375Y/V250S | *** | yes | decreased | pending | Pending |
| E375Y/V250C | *** | yes | decreased | pending | Pending |
| E375Y/V250T | *** | yes | decreased | pending | Pending |
| E375Y/V250K | *** | yes | no activity | pending | Pending |
| E375Y/V250H | *** | yes | decreased rate | pending | Pending |
| E375Y/V250N | | yes | similar to | pending | Pending |
| | | | parent Phi29 | | |
| | | | exo(−) D12A/D66A | | |
| E375Y/V250D | *** | yes | no activity | pending | Pending |
| E375Y/V250G | *** | yes | no activity | pending | Pending |
| E375Y/V250W | *** | yes | no activity | pending | Pending |
| E375Y/S388G | ♦ | yes | pending | pending | Pending |
| E375Y/K512A | ♦ | yes | Pending | pending | Pending |
| E375Y/K525A | ♦ | yes | Pending | pending | Pending |
| Y254V/E375Y | ♦ | yes | Pending | pending | Pending |
| K132A | * | Yes | similar to | pending | Pending |
| | | | parent Phi29 | | |
| | | | exo(−) D12A/D66A | | |
| K383A | * | Yes | Slightly lower than | pending | Pending |
| | | | parentPhi29 exo(−) | | |
| | | | D12A/D66A | | |
| | | | in γ-nucleotide | | |
| | | | incorporation, but | | |
| | | | decreased activity with | | |
| | | | base-labeled nucleotides. | | |
| K383R | * | yes | Slightly lower than | pending | Pending |
| | | | parent Phi29 exo(−) | | |
| | | | D12A/D66A | | |
| | | | in γ-nucleotide | | |
| | | | incorporation | | |
| K383P | Create a | yes | Significantly | pending | Pending |
| | “dead” | | decreased γ-nucleotide | | |
| | enzyme | | incorporation | | |
| K371A | * | yes | Slightly lower than | pending | Pending |
| | | | parent Phi29 exo(−) | | |
| | | | D12A/D66A in | | |
| | | | γ-nucleotide | | |
| | | | incorporation, but | | |
| | | | decreased activity with | | |
| | | | base-labeled nucleotides. | | |
| K371T | * | yes | Slightly lower than | pending | Pending |
| | | | parent Phi29 exo(−) | | |
| | | | D12A/D66A in | | |
| | | | γ-nucleotide | | |
| | | | incorporation, but | | |
| | | | decreased activity with | | |
| | | | base-labeled nucleotides. | | |
| Y254F | * | yes | Decreased γ-nucleotide | pending | Pending |
| | | | incorporation, but increased | | |
| | | | activity with base-labeled | | |
| | | | nucleotides. | | |
| Y254V | * | yes | Decreased γ-nucleotide | pending | Pending |
| | | | incorporation, but increased | | |
| | | | activity with base-labeled | | |
| | | | nucleotides. | | |
| Y254S | * | pending | pending | pending | Pending |
| K379A | * | yes | Similar to parent Phi29 | pending | Pending |
| | | | exo(−) D12A/D66A | | |
| K525A | * | yes | Similar to parent Phi29 | pending | Pending |
| | | | exo(−) D12A/D66A in | | |
| | | | γ-nucleotide incorporation, | | |
| | | | but increased activity with | | |
| | | | base-labeled nucleotides. | | |
| K135A | * | yes | Similar to parent Phi29 | pending | Pending |
| | | | exo(−) D12A/D66A | | |
| P255S | * | yes | Similar to parent Phi29 | pending | Pending |
| | | | exo(−) D12A/D66A in | | |
| | | | γ-nucleotide incorporation, | | |
| | | | but decreased activity with | | |
| | | | base-labeled nucleotides. | | |
| S388G | * | yes | Decreased γ-nucleotide | pending | Pending |
| | | | incorporation but | | |
| | | | increased activity with | | |
| | | | base-labeled nucleotides. | | |
| K512A | * | yes | Slightly decreased γ- | pending | Pending |
| | | | nucleotide incorporation, | | |
| | | | but increased activity with | | |
| | | | base-labeled nucleotides. | | |
| L384R | * | yes | Slightly decreased γ- | pending | Pending |
| | | | nucleotide incorporation | | |
| E486A | * | yes | Slightly decreased γ- | pending | Pending |
| | | | nucleotide incorporation | | |
| E486D | * | yes | Decreased γ-nucleotide | pending | Pending |
| | | | incorporation | | |
| K478A | * | yes | Decreased γ-nucleotide | pending | Pending |
| | | | incorporation | | |
| E375W | * | yes | Increased γ-nucleotide | pending | Pending |
| | | | incorporation | | |
| N387A | * | yes | Significantly decreased γ- | pending | Fewer signals. |
| | | | nucleotide incorporation, | | |
| | | | but increased activity with | | |
| | | | base-labeled nucleotides. | | |
| N387Y | * | yes | Significantly decreased γ- | pending | Pending |
| | | | nucleotide incorporation | | |
| V250A/E375W | *** | pending | pending | pending | Pending |
| D456N/D458N/ | random | pending | pending | pending | Pending |
| L351P | | | | | |
| Y254V/A377E | random | Pending | pending | pending | Pending |
| D456N/D458N | Create a | pending | pending | pending | Pending |
| | Dead | | | | |
| | Enzyme | | | | |
| D12A/D66A ^{¶} ^{#} | Exo | yes | exo deficient | N/A | N/A |
| | minus | | | | |
| D169A ^{¶} | Exo | yes | exo deficient | N/A | N/A |
| | minus | | | | |
| D12A/D66A/ | Exo | yes | exo deficient | N/A | N/A |
| D169A | minus | | | | |
| T15I ^{§} | Exo | yes | Still has exo | N/A | N/A |
| | minus | | activity | | |
| N62D ^{§} | Exo | yes | Still has exo | N/A | N/A |
| | minus | | activity | | |
| C22S | Remain | yes | Active | N/A | N/A |
| | active | | | | |
| C290S | Remain | yes | Active | N/A | N/A |
| | active | | | | |
| C448S | Remain | yes | Active | N/A | N/A |
| | active | | | | |
| C530S | Remain | yes | Active | N/A | N/A |
| | active | | | | |
| C290S/C448S/ | Remain | yes | Slightly less | N/A | N/A |
| C530S | active | | active | | |
| C22S/C448S/ | Remain | yes | Slightly less | N/A | N/A |
| C530S | active | | active | | |
| C22S/C290S/ | Remain | yes | Slightly less | N/A | N/A |
| C530S | active | | active | | |
| C22S/C290S/ | Remain | yes | Slightly less | N/A | N/A |
| C448S | active | | active | | |

* Increase γ-nucleotide binding efficiency
** Decrease catalytic rate
*** Increase γ-nucleotide binding efficiency while decreasing catalytic rate to detect a longer lived γ-signal
♦ Optimization of dual-labeled nucleotideF incorporation
^{¶} Variants generated by total DNA synthesis
^{#} D12A/D66A served as template for mutagenesis of all above mutants except T15I and N62D
^{§} Mutagenesis based on wild type Φ29 DNA polymerase [symbol]
The Inc50 was done using dG2Oy650 in duplicate

### Phi29 Protein Sequence

The single letter protein sequence of Phi29 (SEQ. ID: 1) is given below:

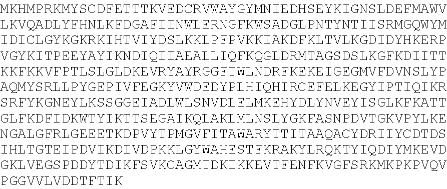

### Phi29 DNA Sequence

The DNA sequence encoding Phi29 (SEQ. ID: 2) is given below:

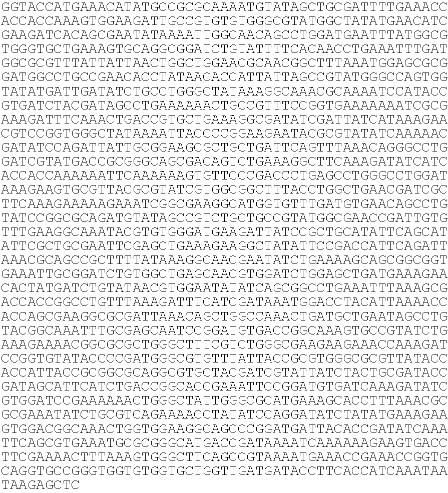

### Phi29 Exo(−) Protein Sequence

The single letter protein sequence of Phi29 exo(−) mutant polymerase, comprising the mutations D12A and D66A (SEQ. ID: 3) is given below:

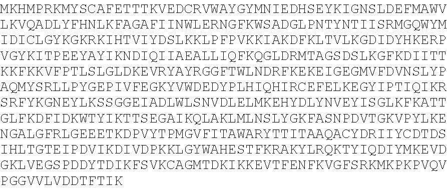

### Phi29 Exo(−)DNA Sequence

The nucleotide sequence of Phi29 exo(−) mutant polymerase, comprising the mutations D12A and D66A (SEQ. ID: 4) is given below:

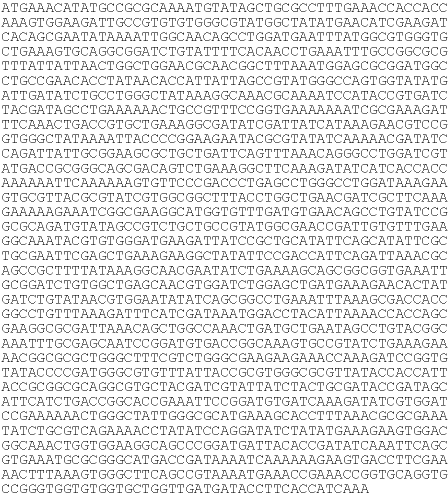

### Wild Type Taq Polymerase Protein Sequence

The protein sequence of Taq DNA polymerase (SEQ. ID: 5) is given below:

ALEEAPWPPPEGAFVGFVLSRKEPMWADLLALAAARGGRVHRAPEPYKAL
RDLKEARGLLAKDLSVLALREGLGLPPGDDPMLLAYLLDPSNTTPEGVAR
RYGGEWTEEAGERAALSERLFANLWGRLEGEERLLWLYREVERPLSAVLA
HMEATGVRLDVAYLRALSLEVAEEIARLEAEVFRLAGHPFNLNSRDQLER
VLFDELGLPAIGKTEKTGKRSTSAAVLEALREAHPIVEKILQYRELTKLK
STYIDPLPDLIHPRTGRLHTRFNQTATATGRLSSSDPNLQNIPVRTPLGQ
RIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAF
IERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAP
KERAEAVARLAKEVMEGVYPLAVPLEVEVGIGEDWLSAKE

### Wild Type Taq Polymerase DNA Sequence

The nucleotide sequence of Taq DNA polymerase (SEQ. ID: 6) is given below:

GCCCTGGAGGAGGCCCCCTGGCCCCCGCCGGAAGGGGCCTTCGTGGGCTT
TGTGCTTTCCCGCAAGGAGCCCATGTGGGCCGATCTTCTGGCCCTGGCCG
CCGCCAGGGGGGGCCGGGTCCACCGGGCCCCCGAGCCTTATAAAGCCCTC
AGGGACCTGAAGGAGGCGCGGGGGCTTCTCGCCAAAGACCTGAGCGTTCT
GGCCCTGAGGGAAGGCCTTGGCCTCCCGCCCGGCGACGACCCCATGCTCC
TCGCCTACCTCCTGGACCCTTCCAACACCACCCCCGAGGGGGTGGCCCGG
CGCTACGGCGGGGAGTGGACGGAGGAGGCGGGGGAGCGGGCCGCCCTTTC
CGAGAGGCTCTTCGCCAACCTGTGGGGGAGGCTTGAGGGGGAGGAGAGGC
TCCTTTGGCTTTACCGGGAGGTGGAGAGGCCCCTTTCCGCTGTCCTGGCC
CACATGGAGGCCACGGGGGTGCGCCTGGACGTGGCCTATCTCAGGGCCTT
GTCCCTGGAGGTGGCCGAGGAGATCGCCCGCCTCGAGGCCGAGGTCTTCC
GCCTGGCCGGCCACCCCTTCAACCTCAACTCCCGGGACCAGCTGGAAAGG
GTCCTCTTTGACGAGCTAGGGCTTCCCGCCATCGGCAAGACGGAGAAGAC
CGGCAAGCGCTCCACCAGCGCCGCCGTCCTGGAGGCCCTCCGCGAGGCCC
ACCCCATCGTGGAGAAGATCCTGCAGTACCGGGAGCTCACCAAGCTGAAG
AGCACCTACATTGACCCCTTGCCGGACCTCATCCACCCCAGGACGGGCCG
CCTCCACACCCGCTTCAACCAGACGGCCACGGCCACGGGCAGGCTAAGTA
GCTCCGATCCCAACCTCCAGAACATCCCCGTCCGCACCCCGCTTGGGCAG
AGGATCCGCCGGGCCTTCATCGCCGAGGAGGGGTGGCTATTGGTGGCCCT
GGACTATAGCCAGATAGAGCTCAGGGTGCTGGCCCACCTCTCCGGCGACG
AGAACCTGATCCGGGTCTTCCAGGAGGGGCGGGACATCCACACGGAGACC
GCCAGCTGGATGTTCGGCGTCCCCCGGGAGGCCGTGGACCCCCTGATGCG
CCGGGCGGCCAAGACCATCAACTTCGGGGTCCTCTACGGCATGTCGGCCC
ACCGCCTCTCCCAGGAGCTAGCCATCCCTTACGAGGAGGCCCAGGCCTTC
ATTGAGCGCTACTTTCAGAGCTTCCCCAAGGTGCGGGCCTGGATTGAGAA
GACCCTGGAGGAGGGCAGGAGGCGGGGGTACGTGGAGACCCTCTTCGGCC
GCCGCCGCTACGTGCCAGACCTAGAGGCCCGGGTGAAGAGCGTGCGGGAG
GCGGCCGAGCGCATGGCCTTCAACATGCCCGTCCAGGGCACCGCCGCCGA
CCTCATGAAGCTGGCTATGGTGAAGCTCTTCCCCAGGCTGGAGGAAATGG
GGGCCAGGATGCTCCTTCAGGTCCACGACGAGCTGGTCCTCGAGGCCCCA
AAAGAGAGGGCGGAGGCCGTGGCCCGGCTGGCCAAGGAGGTCATGGAGGG
GGTGTATCCCCTGGCCGTGCCCCTGGAGGTGGAGGTGGGGATAGGGGAGG
ACTGGCTCTCCGCCAAGGAGTGA

### Taq Polymerase (F647C Mutant) Protein Sequence

The protein sequence of Taq DNA polymerase comprising the highlighted mutation F647C (“VisiTaq” polymerase; SEQ. ID: 7) is given below:

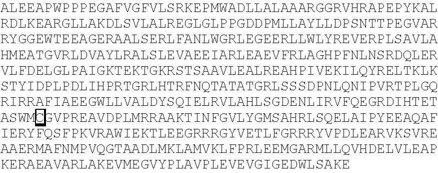

### Taq Polymerase (F647C Mutant) DNA Sequence

The nucleotide sequence of Taq DNA polymerase, comprising the mutated codon at position647 (SEQ. ID: 8) is given below:

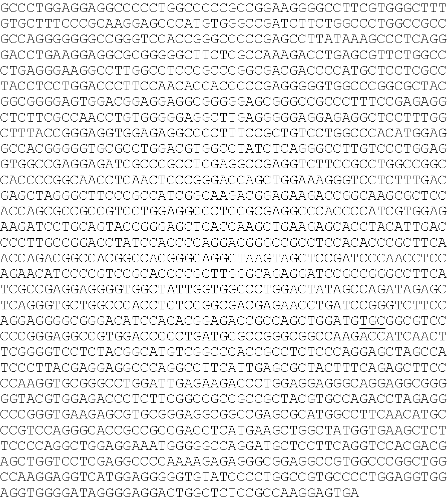

The following table list the primers and sequences of the Phi29 variants of Table 1:

TABLE 2
LIST OF PRIMERS AND ENCODING SEQUENCES

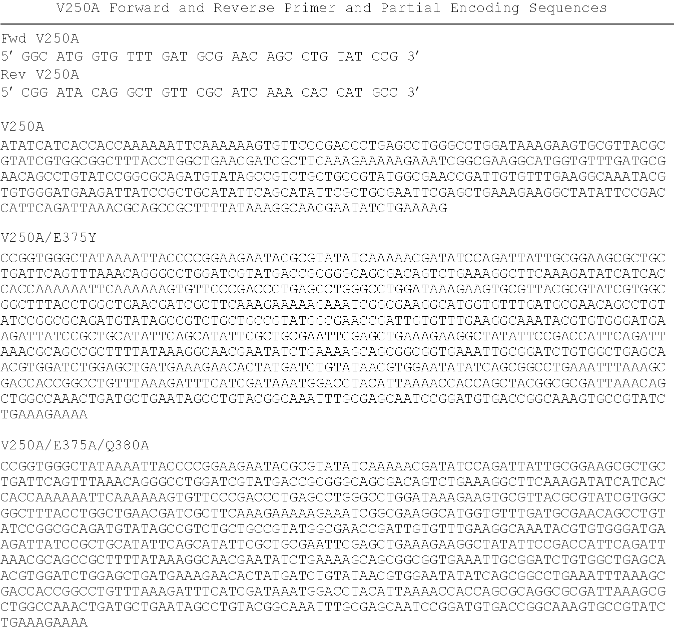

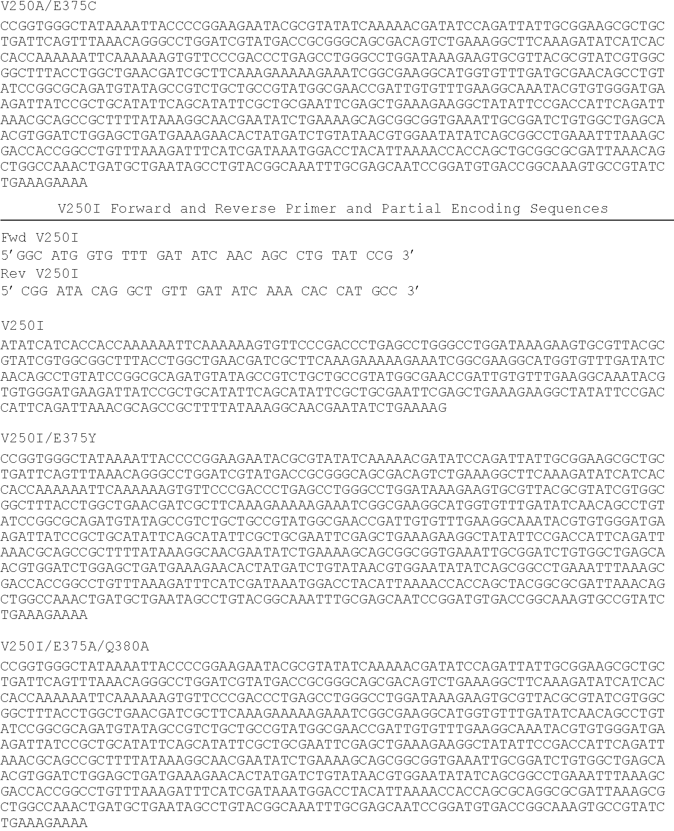

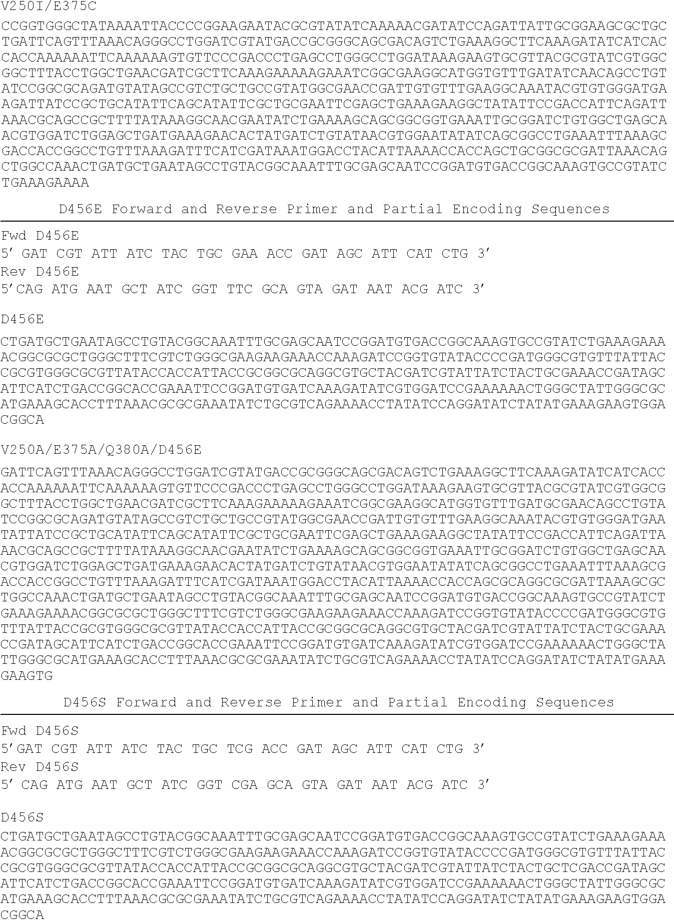

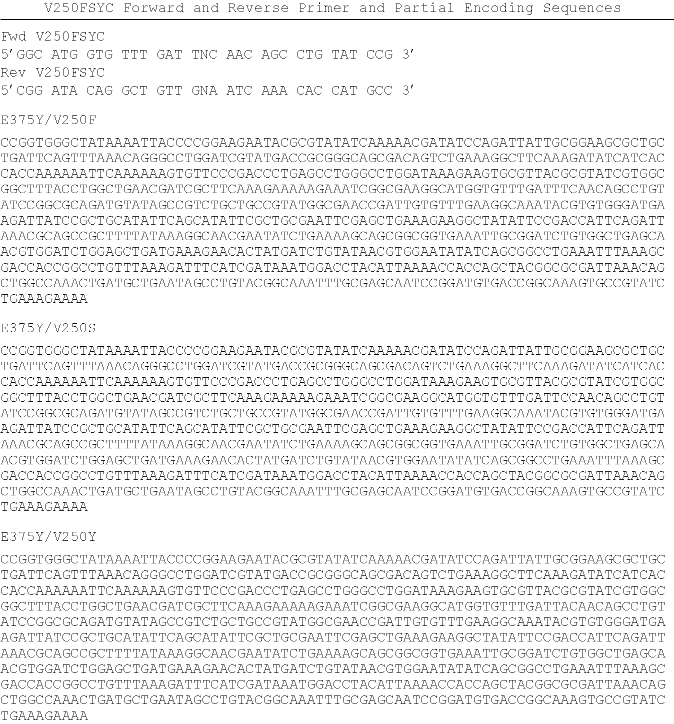

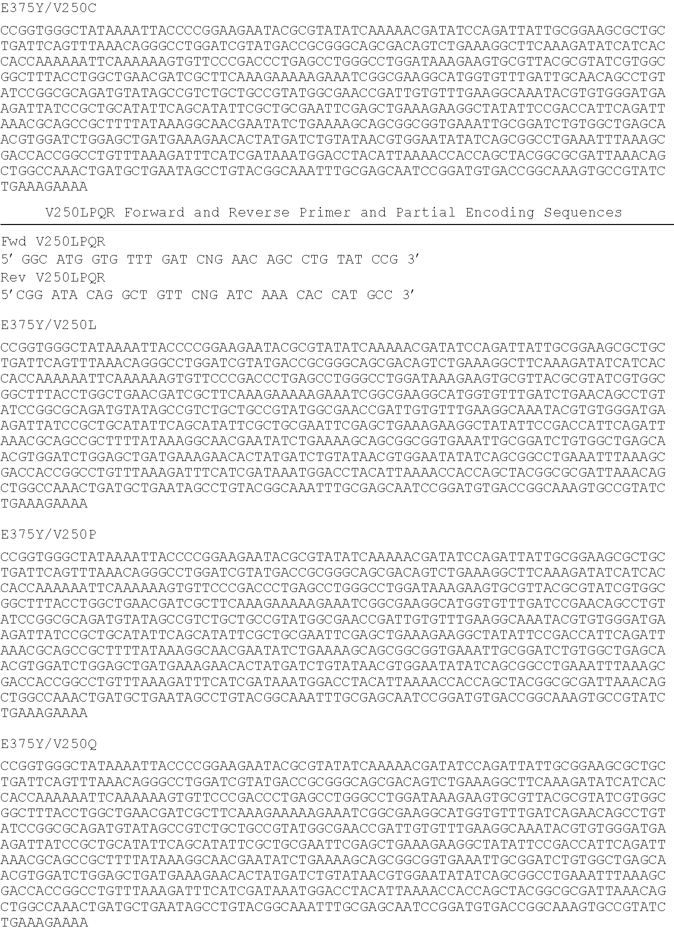

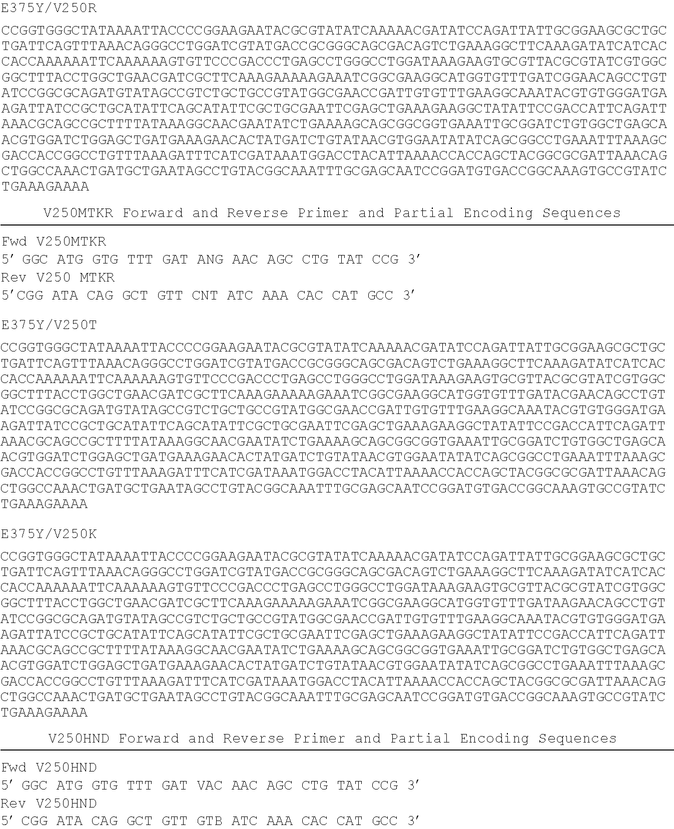

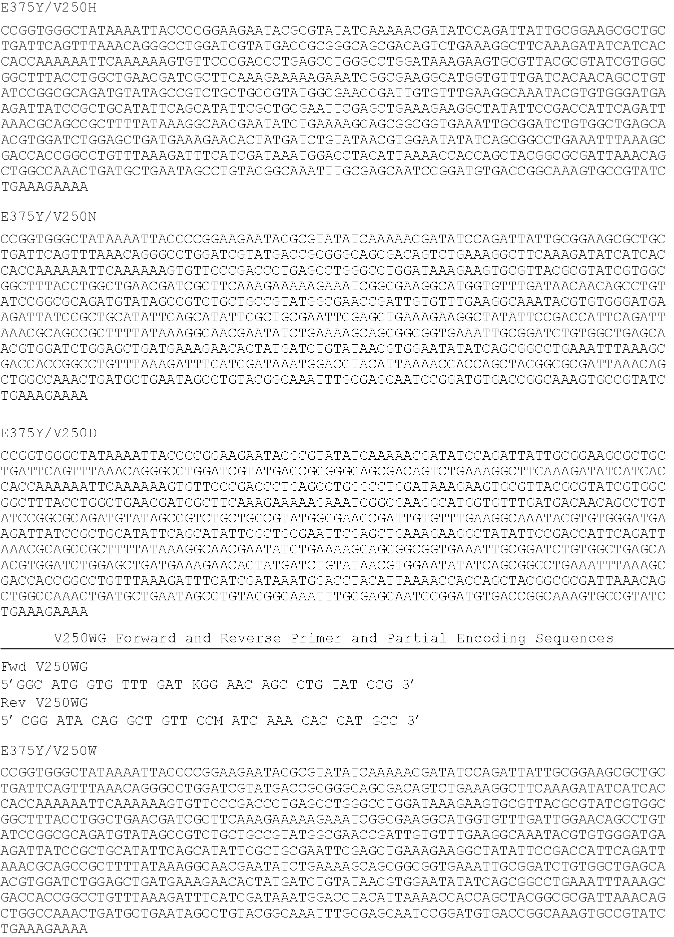

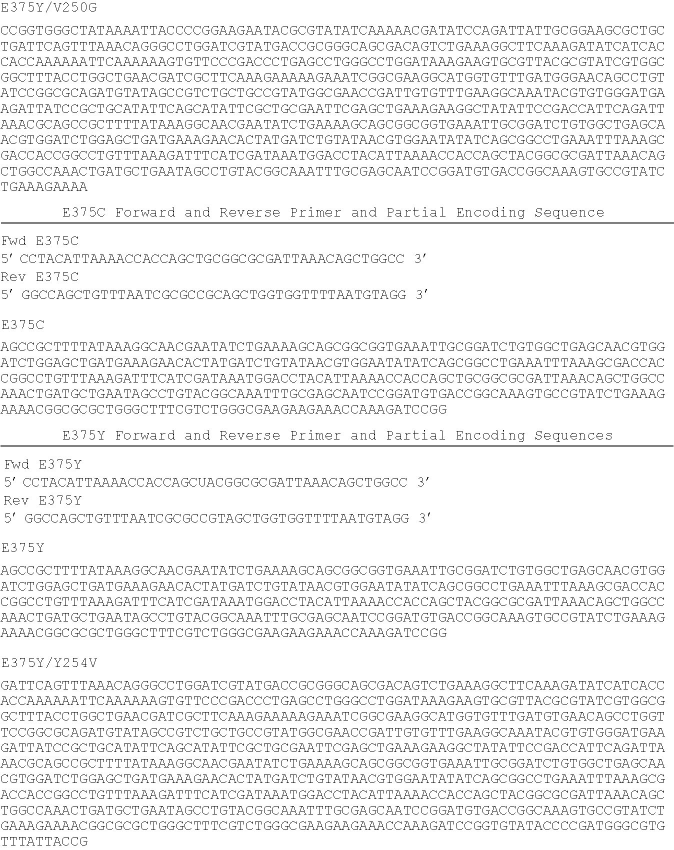

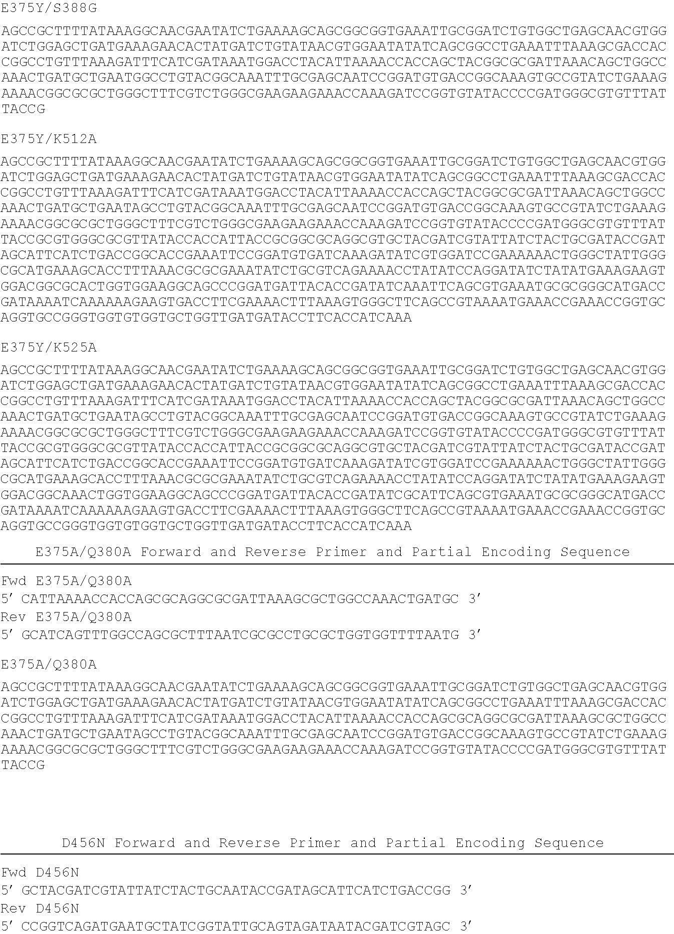

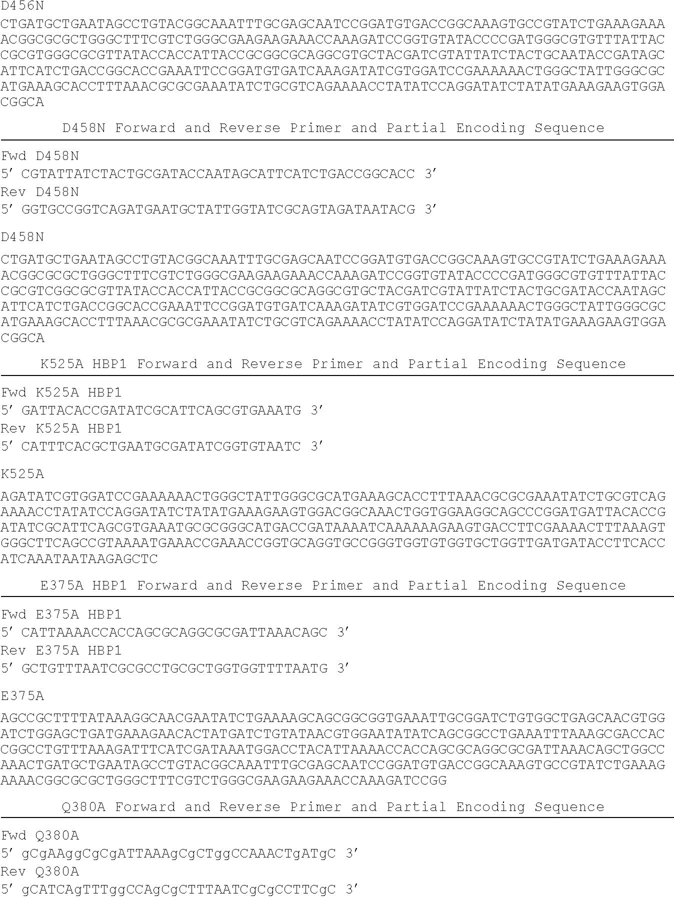

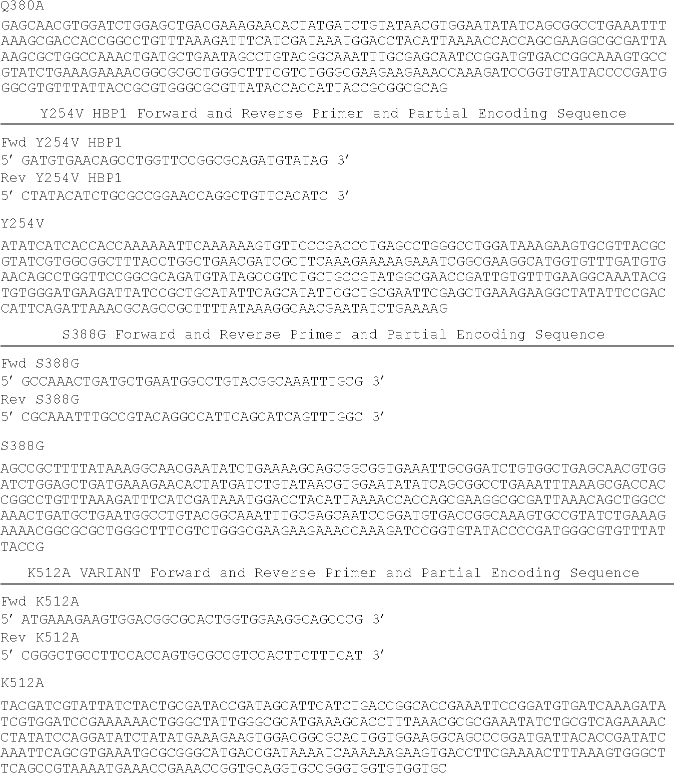

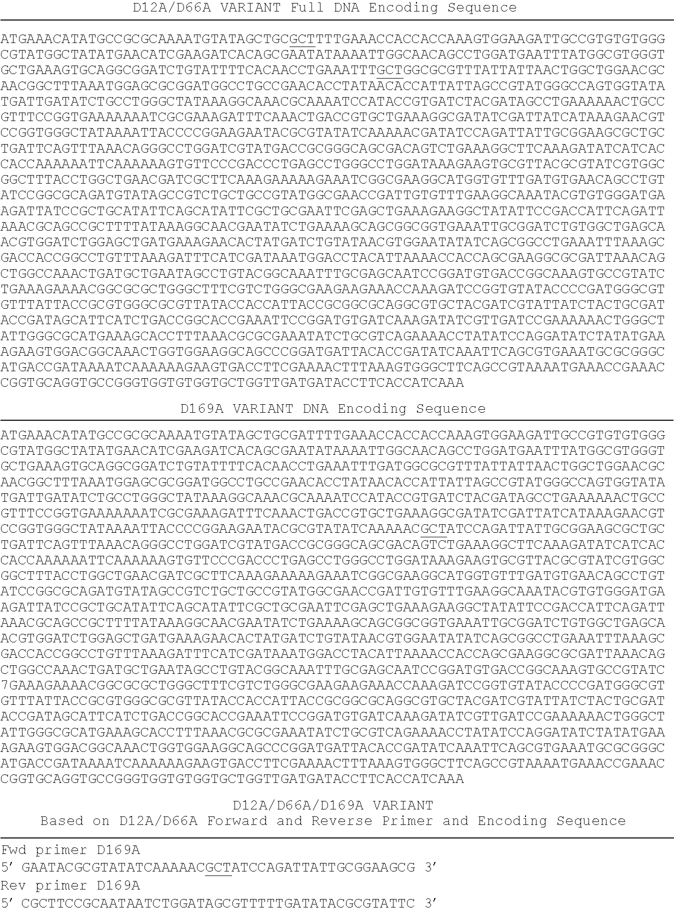

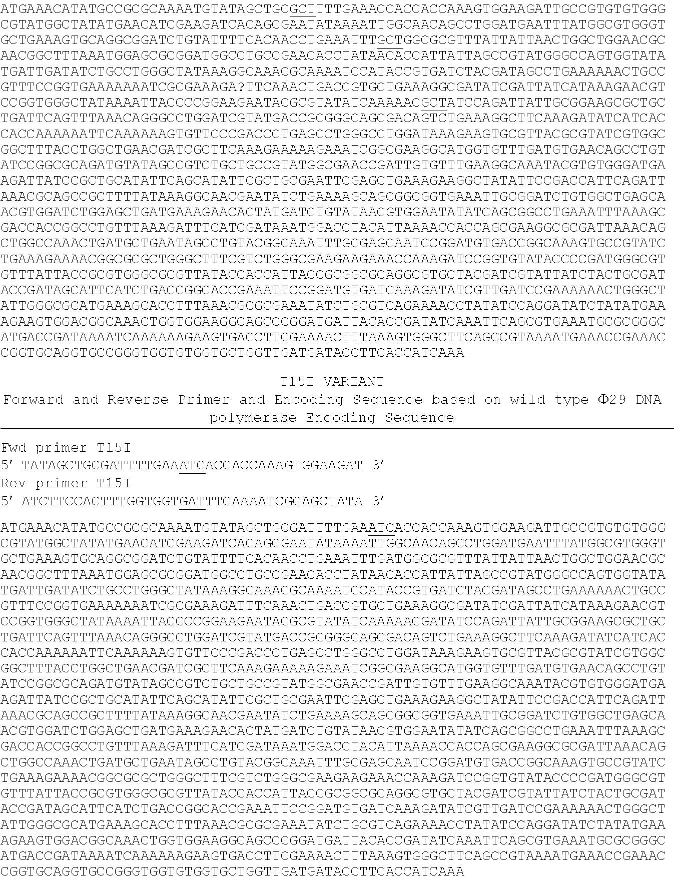

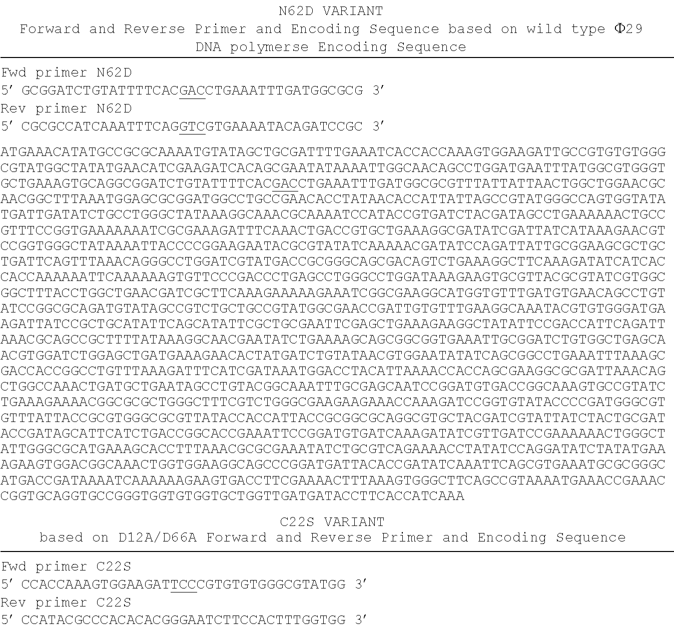

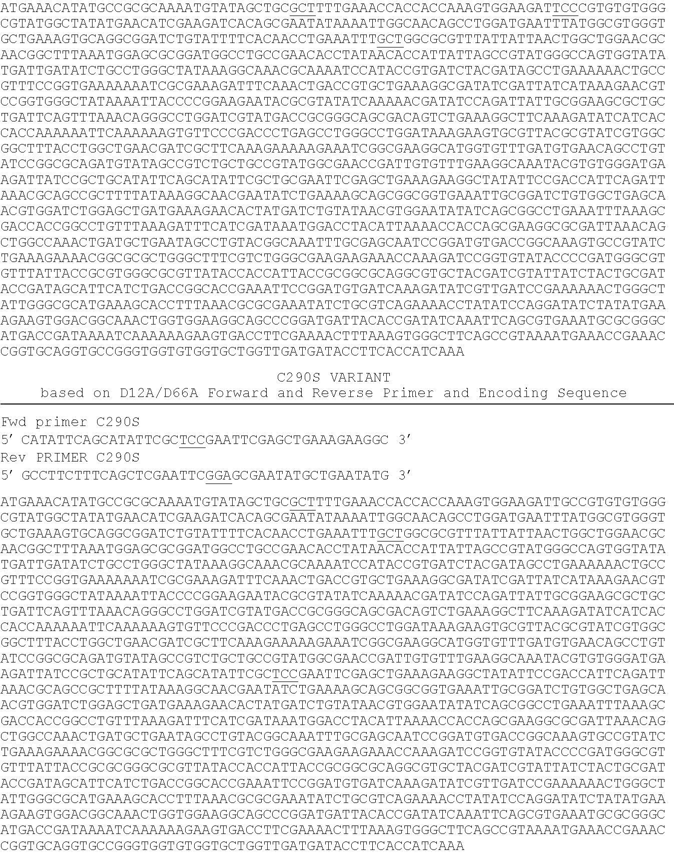

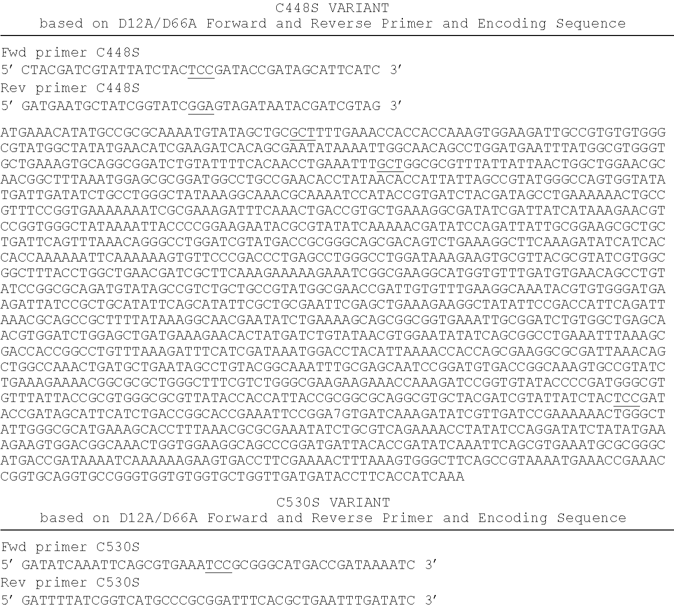

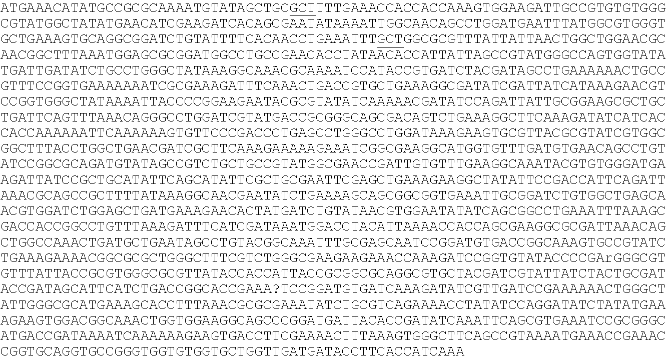

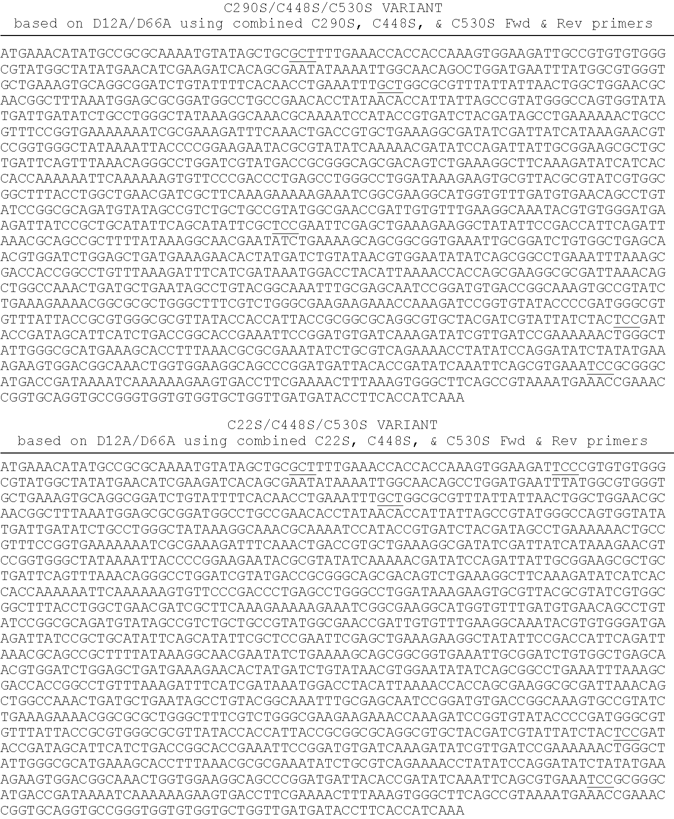

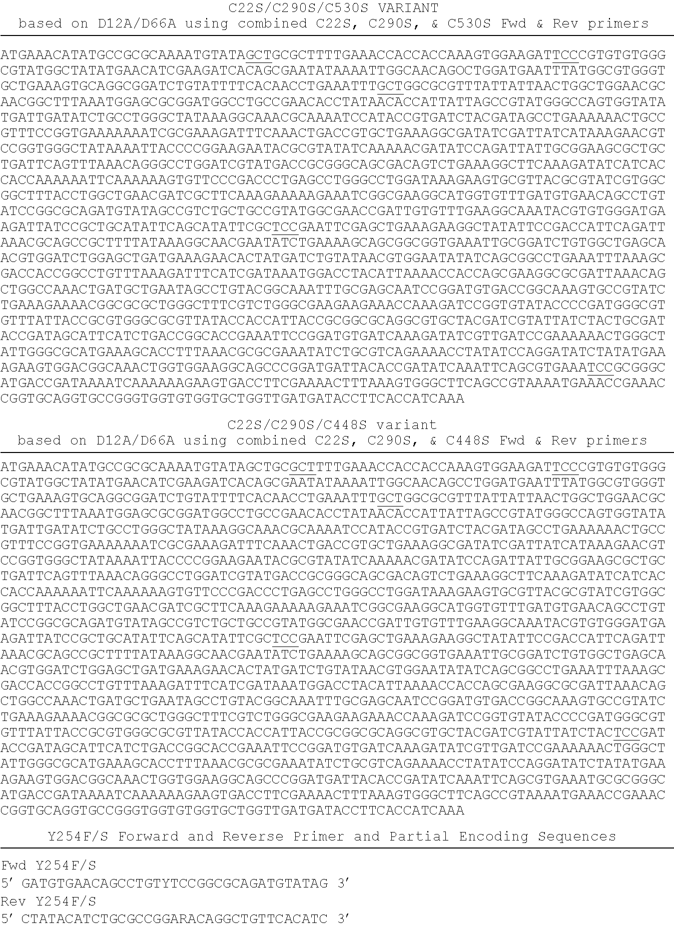

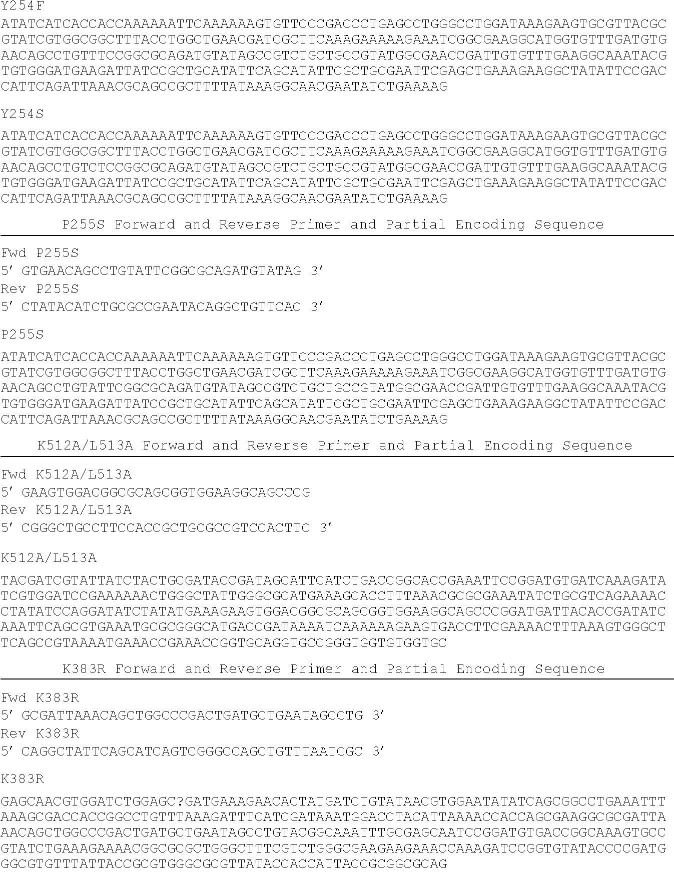

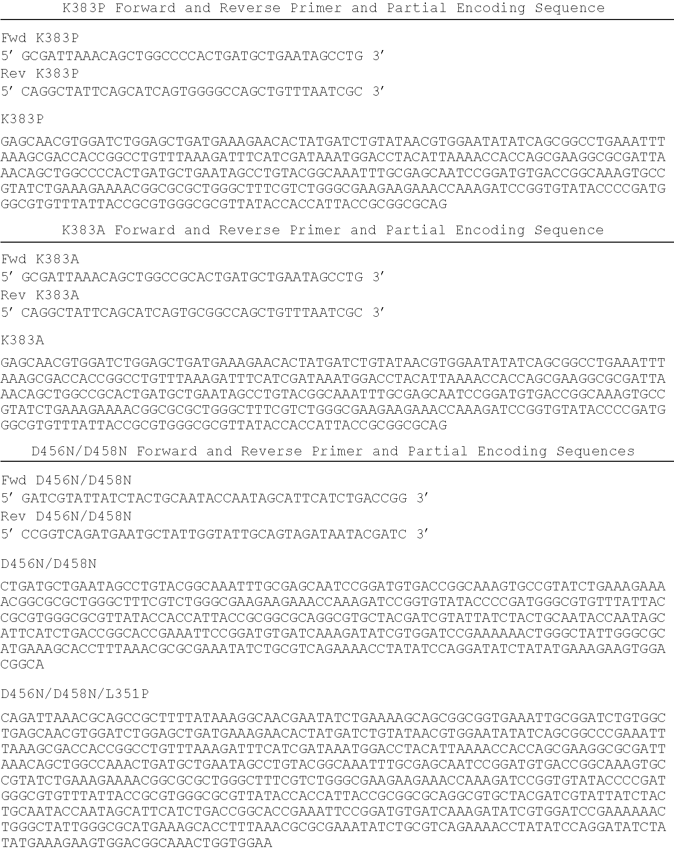

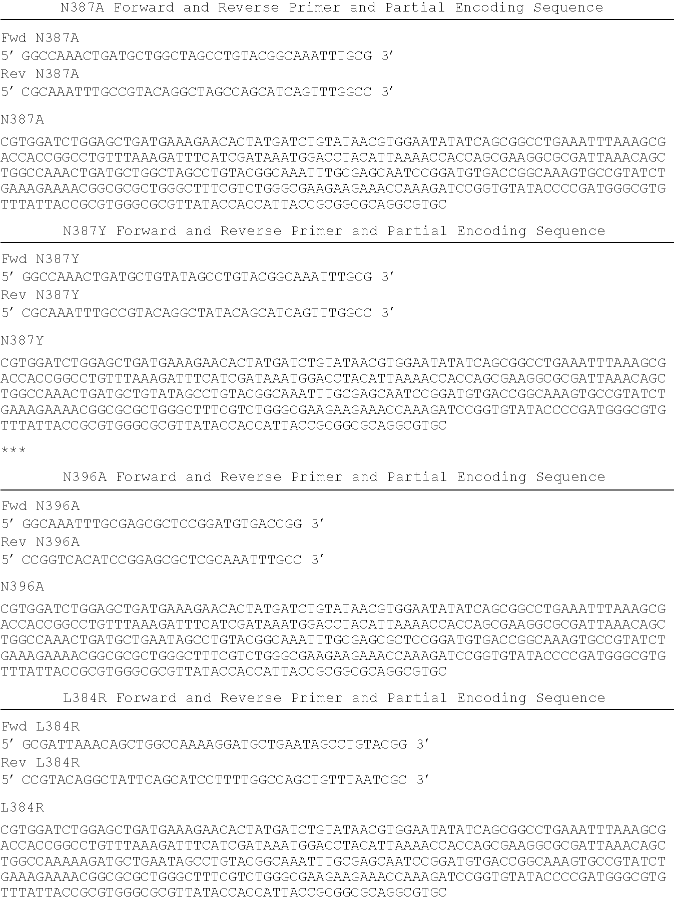

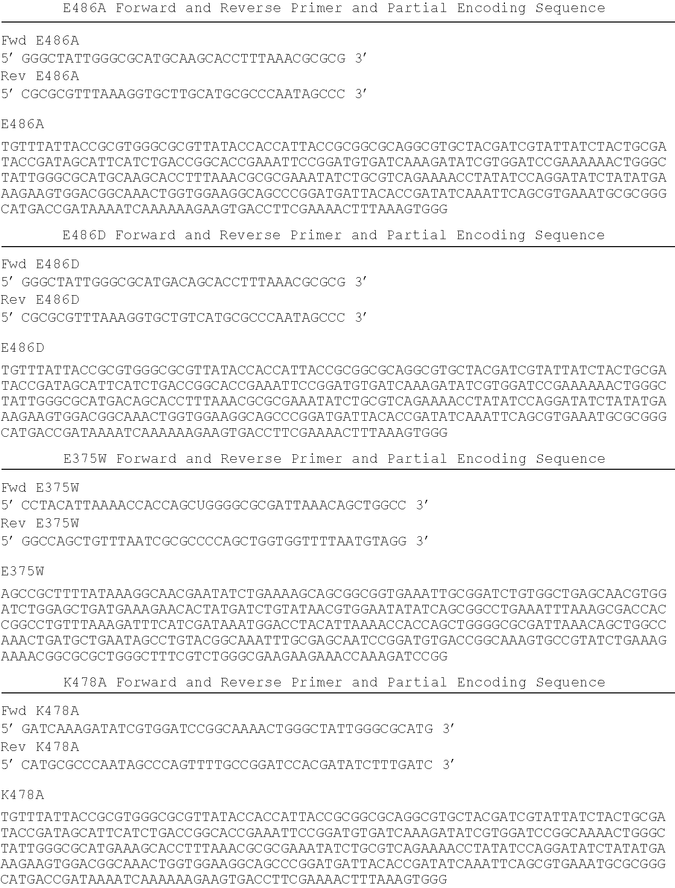

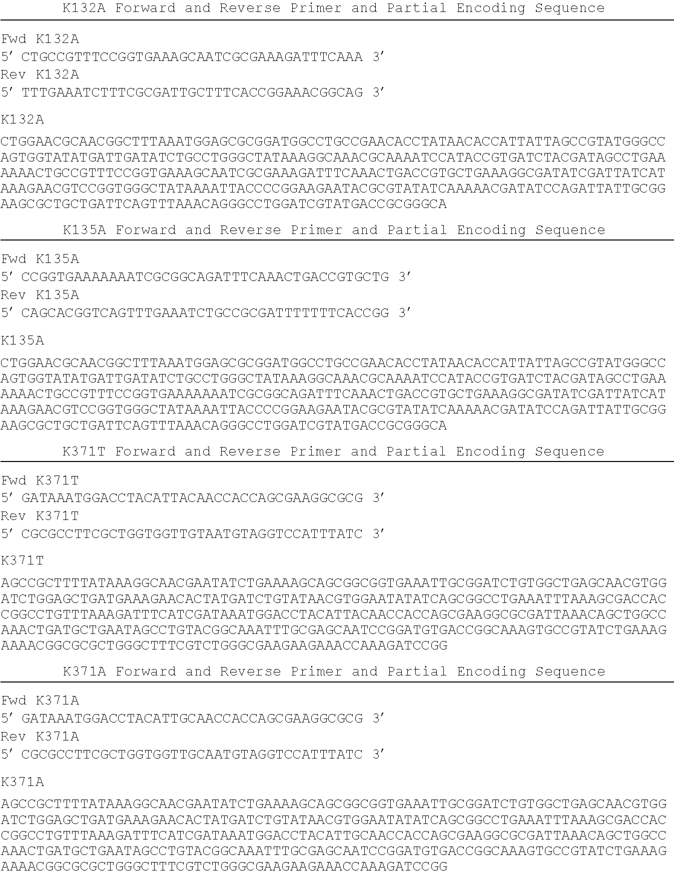

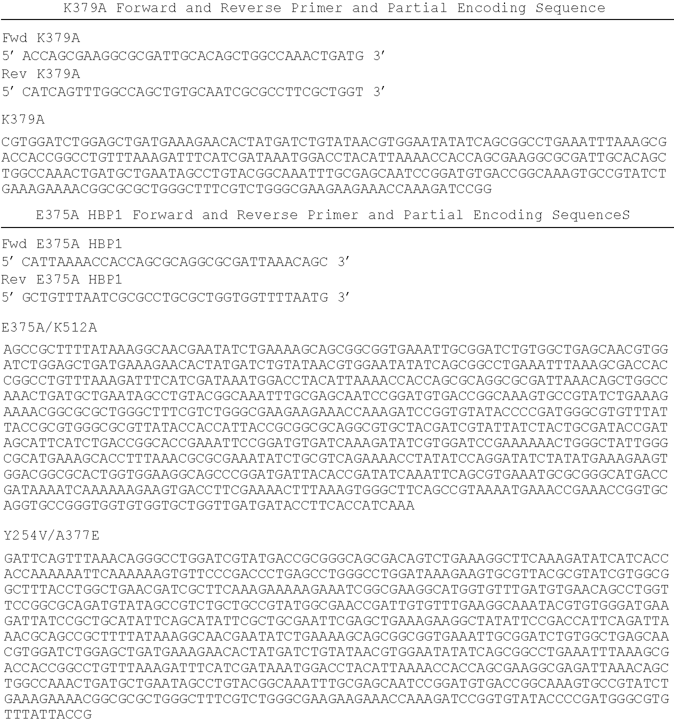

Referring now to FIG. 60, a set of molecular structures and starting compound for preparing fluorescent molecular structures capable of accommodating a plurality of γ-phosphate labeled nucleotides, where the nucleotides can be the same or different. In certain embodiments, the sequencing composition will include molecular structures having a plurality of each of the four types of nucleotides, a plurality having γ-phosphate labeled dATPs, a plurality having γ-phosphate labeled dTTPs, a plurality having γ-phosphate labeled dCTPs and a plurality having γ-phosphate labeled dGTPs. In other embodiments, each molecular structure will include two or more γ-phosphate labeled nucleotide types per structure. In other embodiments, each molecular structure will include at least one of each γ-phosphate labeled types.

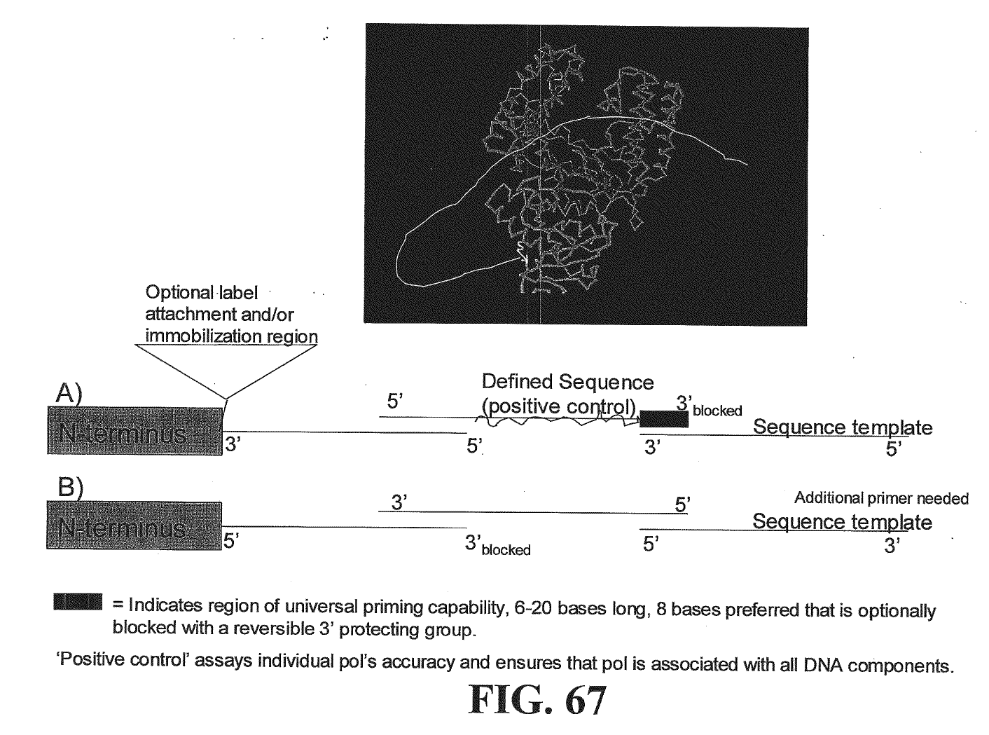

Also disclosed herein are methods and compositions relating to anchored duplex/polymerase compositions useful in FRET-based single molecule systems. Referring now to FIG. 67, Left) Green indicates the polymerase, here represented by 1KTQ. The white arrow and ‘S’ indicate the N-terminus of the polymerase. As indicated, the DNA is covalently attached to the N-terminus of the polymerase, as detailed further below. Some variations of the associations are illustrated below. In this technique, the duplex including the template is attached to the polymerase by a sufficient long attachment nucleotide sequence so that a bridging primer and duplex with a portion of the attachment sequence and to a template to be sequenced.

In anchored duplex A, the template 3′ end can be extended to the right to give a known sequence of the bridging primer to ensure detector integrity. Once detector integrity is shown, the 3′ end of the bridging primer can be deprotected and to begin template sequencing.

In anchored duplex B, after the template bridging duplex is formed, short primers can be duplexed to the template with extension occurring to the right from the 3′ end of the short primers.

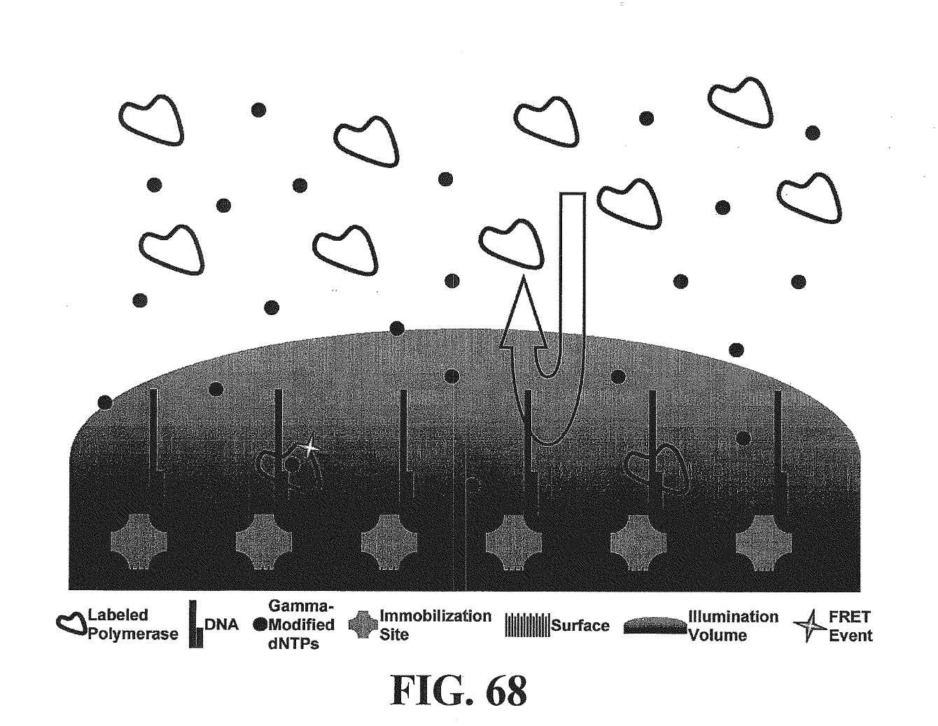

Another method to increase S/N ratio is to apply higher laser power to drive donor fluorescence. One problem with this approach is that it decreases the lifetime of the donor which in turn decreases the amount of time during which useful signals can be collected. Disclosed herein are methods to alleviate the problems encountered when using higher laser intensities by using unlabeled duplex attached to the surface with donor labeled polymerase and gamma-labeled nucleotides in solution as shown in FIG. 68. Such methods have at least three benefits: (1) the donor can be replenished by exchanging enzymes; (2) there is no concern of the duplex disassociating from the enzyme complex and (3) incorporation will only occur and be detected when a donor (enzyme) binds to the duplex. This is indicated by the detection of FRET signals between the donor-labeled polymerase and the acceptor-labeled gamma-nucleotides. For this method, the use of less processive polymerases is beneficial to the experimental setup because it allows for more rapid exchange of the donor and the donor is less likely to photo-bleach. Experiments are being carried out to determine the most appropriate enzyme for this method.

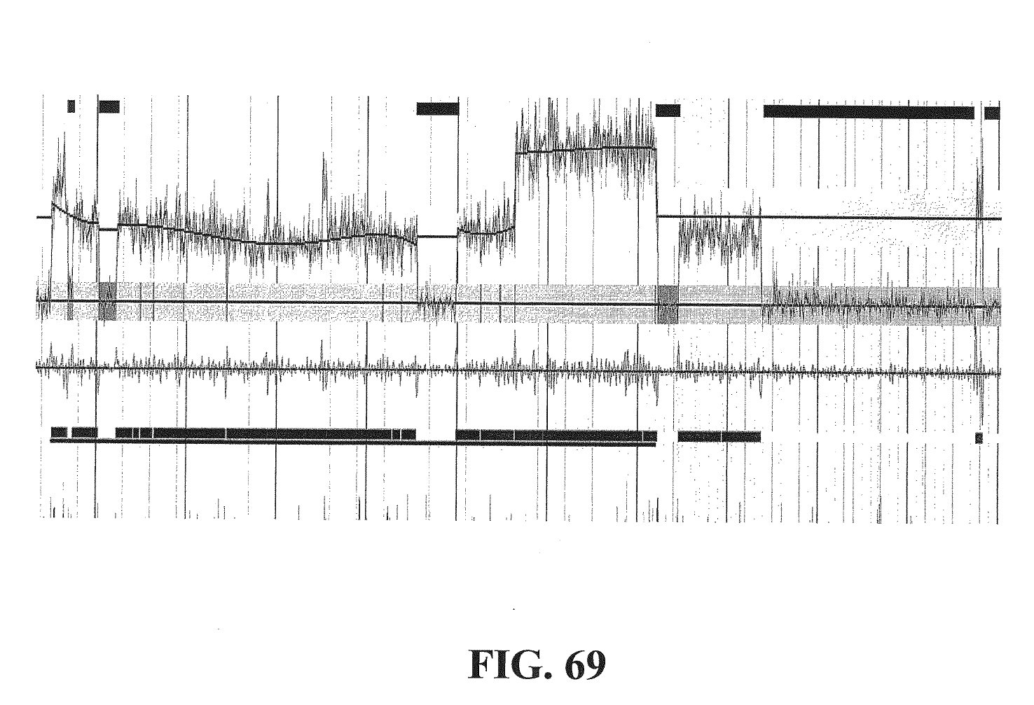

Disclosed disclosed herein are mutant polymerases having increased activity with gamma-modified nucleotides, as summarized in Table 1. Some of these mutants also exhibit decreased processivity. Each tested polymerase was analyzed with regard to donor duration and donor signal frequency over the collection time. The donor signals were assigned as segments of excited (digital unit), and dark (digital zero) depending on their intensities compared to the noise level. The excited donor segments are denoted by a horizontal dark green bar and the dark regions are denoted by horizontal black bars (figure below). The number of donor segments of the excited state was extracted for every donor in the field of view and attributes of these segments such as the duration, intensity and frequency are analyzed. A comparison of these attributes of donor segments was made between different polymerases binding to immobilized duplex on a surface as shown in FIG. 69.

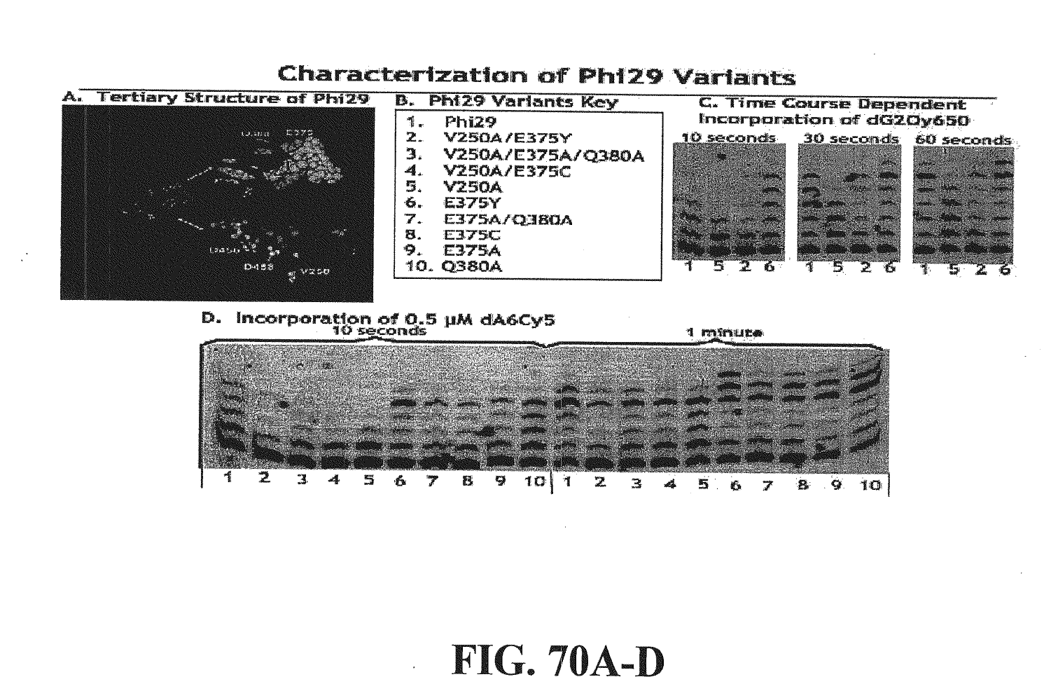

Optionally, the detectability of the gamma incorporation signal may be increased by engineering Phi29 DNA polymerase such that the chemistry step of the incorporation reaction is slowed. By increasing the time that the labeled-nucleotide remains in the active site, we will collect more photons, thereby improving both event detection and color identification. In some embodiments, this will be accomplished by combining amino acid mutations in the active site that hinder the chemistry of incorporation with amino acid mutations outside of the active site that allow for improved binding of nucleotides (FIG. 70A). Some of the present Phi29 variants are included in this disclosure.

In one exemplary embodiment, an alanine was substituted for the valine at position 250. As is seen in FIG. 70C (Variant 5) the rate of extension by this mutant polymerase is decreased relative to wild type. Although the rate of extension by V250 is decreased, the Inc50 (the concentration of nucleotide that allows for half of the primer to be extended one base in sixty seconds) remains relatively unaffected (Table 1), indicating that we have not negatively impacted γ-nucleotide binding by this variant enzyme. To compensate for the reduced extension we combined V250A with several mutations that increase binding efficiency of γ-modified nucleotides. The mutations were based on glutamic acid at position 375, which when mutated to an alanine allows for increased extension and a reduced Inc50 (Table 1). The most significant increase in extension is seen with the E375Y variant (FIG. 70C, Variant 6). As is seen in FIG. 70C, Variant 2, the extension of the V250A mutant is improved through introduction of the E375Y mutation. The mutation V250A was also combined with the mutations E375A/Q380A as well as E375C. FIG. 70D shows the ensemble extension activity of these various enzyme variants with dA6Cy5, which is one of the γ-nucleotides used in our sequencing system.

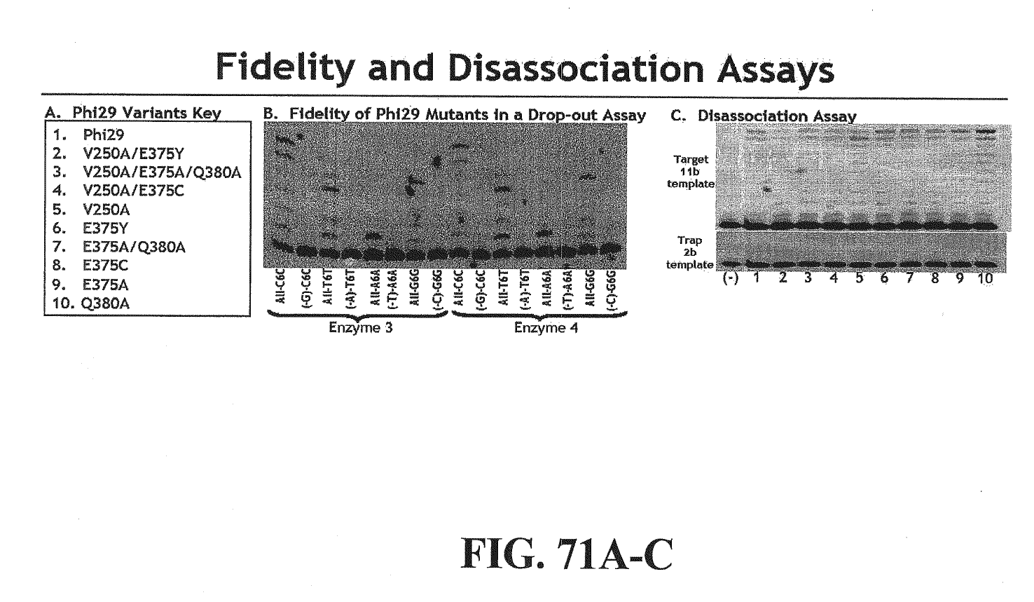

Although we hypothesize that V250A has slower extension due to the chemistry of incorporation being less efficient, until we can analyze the reaction in greater detail, such as via Stop-Flow analysis, we cannot rule out V250A having other effects on incorporation. However, we have been able to determine that the fidelity of the enzyme variants, V250A/E375Y (2), V250A/E375A/Q380A (3), and V250A/E375C (4), appears to remain intact (FIG. 71B, and data not shown). A reduction in processivity was also ruled out as a factor for the reduced extension by V250A via a dissociation assay in which the enzyme is bound to a fluorescein-labeled target duplex. Gamma-nucleotides are then added at the same time as Cy5 labeled trap duplex to initiate extension. The V250A variants make complete products without moving to the trap duplex indicating that they are moving processively, but more slowly than the Phi29 exo(−) enzyme (FIG. 71C).

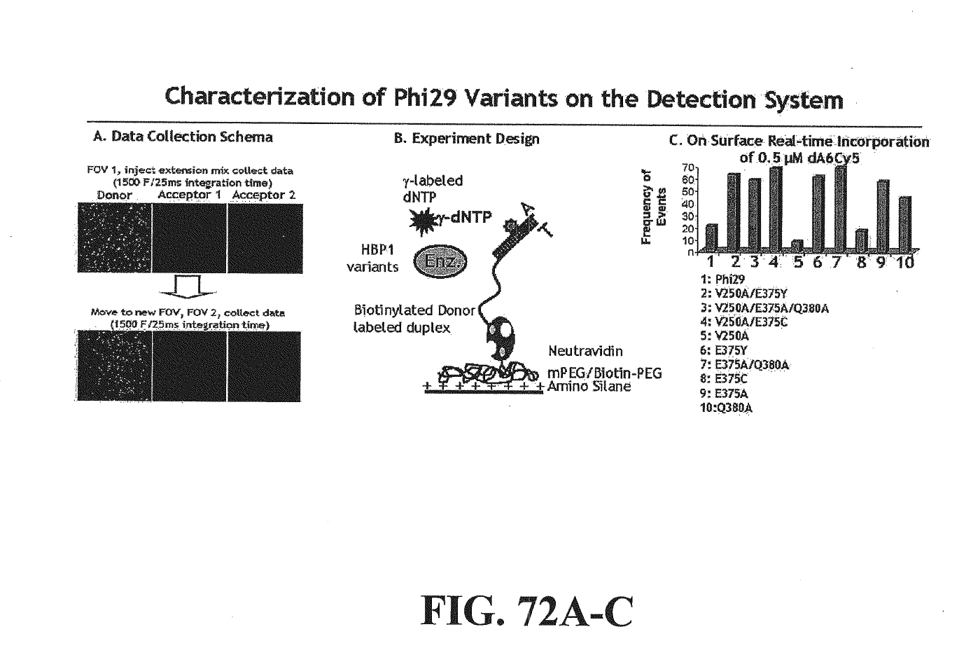

Real time “On surface” experiments were performed to characterize the activity of the Phi29 variants on an exemplary single molecule sequencing system (FIGS. 72A and B). Chambered slides were prepared to facilitate injection and multiple experiments data collection from several chambers using a single slide. The template specified incorporation of a single γ-labeled nucleotide. The samples were excited using a 488 nm Argon ion laser at 2.5 mW and the data were collected at 25 ms integration time. Each stream of data was collected for 1500 frames, consecutive streams were collected for ˜5 minutes by moving to new fields of views (FOVs). FRETAN software was used to obtain the donor and acceptor traces. Trace analysis revealed that several of the Phi29 variants (2, 3, 4, 6, 7, 9 and 10) show between 2.5 to 3 fold increases in the number of γ-signals detected, relative to the parent enzyme (FIG. 72C).

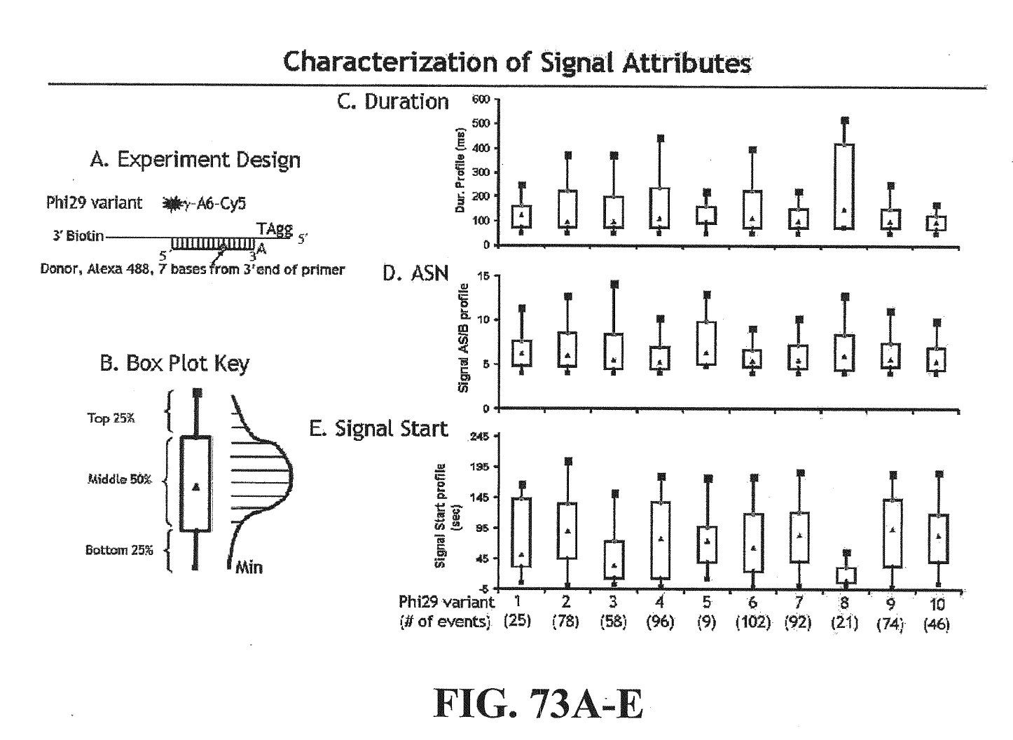

FIG. 73 depicts the results of preliminary analyses of a subset of ten different Phi29 variants (These variants are numbered 1 through 10 as per the list indicated in FIG. 72). The results are shown in box plots (FIG. 73). A box plot visualizes data without making assumptions of the underlying statistical distribution, nx is divided into 5 regions: minima, lowest quartile (25th percentile), median, upper quartile (75 percentile) and maxima. It graphically displays the data location and distribution at a glance, thus indicating symmetry and skew-ness in the data set.

After trace analysis, the selected signals (with acceptor signal over background greater than 4, i.e., ASN≧4) were characterized by examining attributes including duration, ASN and timing of signal appearance (i.e., start of signals). The variants that are of special interest include Variants 2 and 3. Both of these variants display higher frequency of events detected (FIG. 72C). Additionally they also have a population of molecules with longer duration and higher ASN (FIGS. 73C & D), both features facilitate detection of signals using the current donors and integration time for data collection. These results are especially encouraging because they reflect the time course ensemble PAGE analyses, which strongly suggest that these two variants are slowed in incorporation (FIG. 70D). Additionally, looking at the first appearance of signals associated with a particular donor data, variant 8 stands out among the enzymes tested, it displays a very early appearance of the g-signals, this result also corroborated with the ensemble PAGE analyses data where this variant displayed increased extension as well as a reduced Inc50.

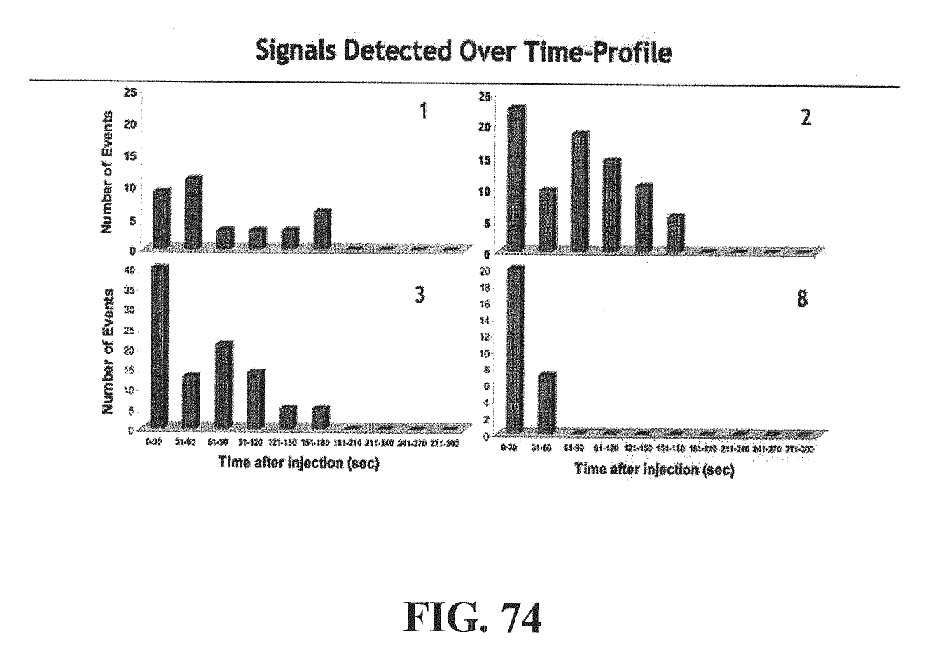

Example sets of signals detected (using Phi29 variants) over time are shown as bar graphs (FIG. 74). These data show when most of the signals are detected after injection. For variants 2 and 3, it appears that most of the signals are detected in the first 90 seconds and, as previously stated, most of the signals associated with variant 8 are detected within the first 30 seconds.

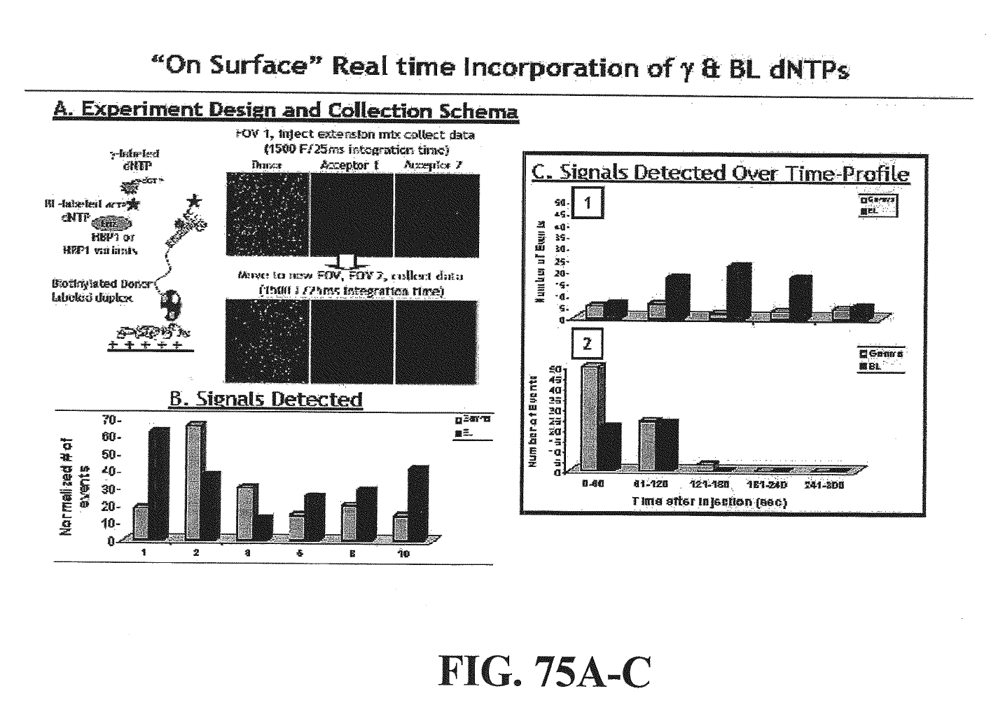

Similar experiments were performed using a template specifying incorporation of a single γ-labeled nucleotide followed by a base-labeled nucleotide and data were analyzed as described above. As observed in the single γ-labeled nucleotide incorporation results, in this single γ and base-labeled nucleotide incorporation data Variants 2 and 3 show a higher frequency of γ-signals detected (FIG. 75B). The appearance of the stable base-labeled signals indicates that the preceding γ-nucleotides were incorporated in these experiments.

Example sets of signals detected using Phi29 variants over time are shown (FIGS. 72-75). These data show when most of the signals (both γ and BL) are detected after injection, moving from one FOV to the next over a period of ˜5 minutes. First, considering the appearance of γ-signals for the parent enzyme (#1) a clear pattern is not discernable because few signals are detected. However, because Variant 2 has a higher frequency of events, it appears that most of the γ-signals are detected in the first 60 seconds. Next, considering the appearance of BL-signals, the signals for the parent enzyme (#1) are nicely distributed and follow a Gaussian distribution (peak in the 121-180 second bin), whereas Variant 2's BL-signals appear between 0-120 seconds—again indicating an earlier appearance of signals when Variant 2 is compared with the parent enzyme.

Collectively, these data indicate that several of the tested Phi-29 exo(−) mutants allow improved γ signal detection, presumably through slowing down the chemistry step involving cleavage of the bonds between the alpha and beta phosphate, and formation of a new bond between the alpha phosphate and the 3′OH of the polynucleotide strand. In addition, several variants appear to have overall activity remaining un-compromised, but at the same time facilitating detection of y signals by increasing duration and ASN. The data from these experiments are summarized in the last column of Table 1, entitled “Activity on Detection System.”

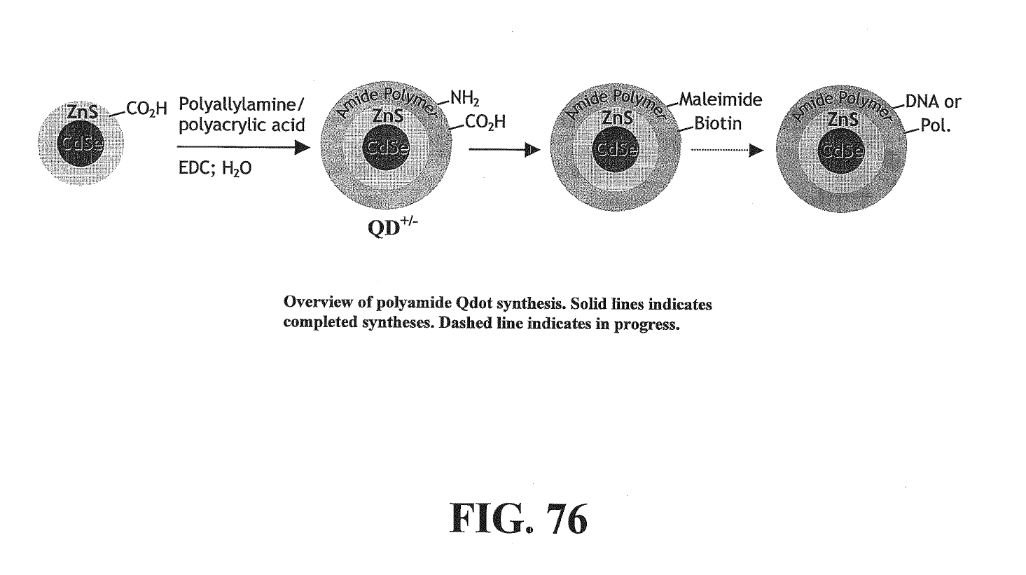

Also disclosed herein are methods to prepare chemically and optically stable, water soluble Qdots that can be further modified for protein or DNA attachment (FIG. 76). Although the literature contains reports of various coatings for Qdots (Medintz et al., 2005, Jiang et al., 2006, Smith et al., 2006, Zhu et al., 2007), these preparations have been difficult to reproduce, particularly those based on siloxane chemistry (this difficulty has also been reported by others; Zhu et al., 2007, Pinaud et al., 2004). Studies using our modified Qdots indicate that they may have a longer soluble shelf life in aqueous solution than the commercial starting material (data not shown).

Covalent attachment of Cy5 to a Qdot using NHS chemistry showed distinct FRET in bulk studies (data not shown).

In another embodiment, DNA and/or polymerase is attached to the surface of a Qdot using the following technique: the surface amines are reacted with the succinimidyl esters of various compounds to generate Qdots with a desired surface group (e.g. biotin or maleimide). DNA and/or polymerase can then be specifically attached using the new surface group. Importantly, the Qdot products of these reactions maintain both their water solubility and the optical properties of the starting material. In some embodiments, AFM and dynamic light scattering is used to characterize the size of these Qdots.

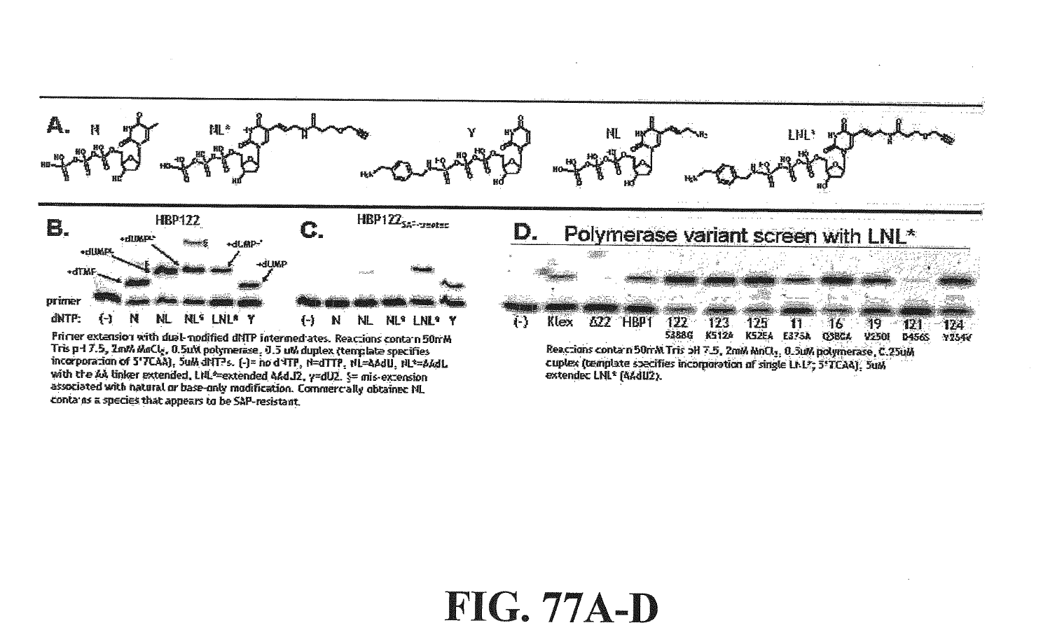

Also disclosed herein is an exemplary synthesis scheme to produce dual-labeled nucleotides that will be used to better characterize gamma-nucleotide incorporation signals. Intermediates in the dual labeled nucleotide synthesis pathways have been made and have been tested with several of the polymerase variants that improve incorporation of base-labeled (BL), γ-labeled or both BL- and γ-labeled nucleotides (discussed above; FIG. 77A). The five molecules are either directly assayed in primer extension assays (FIG. 77B) or treated with phosphatase to preferentially hydrolyze nucleotides that are not modified at the gamma-phosphate before being assayed (FIG. 77C). Note that the base-modified, ‘NL’, starting material (from TriLink Biotechnologies) contains a species that appears to be phosphatase resistant. Additionally, some mis-extension was detected in the lanes containing the natural nucleotide and the modified 5-aminoallyl extended linker nucleotide (the latter may result due to the presence of the extended linker; similar to Lacenere et al., 2006). Importantly, the presence of the γ-modification reduces mis-extension and several variants were identified that incorporate the dual-modified (but not yet labeled) synthesis intermediate, LNL* (FIG. 77D).

In some embodiments, use of a gamma-labeled and base-modified molecule in combination with an engineered polymerase further increases the time that the donor and acceptor are in close proximity to undergo efficient FRET, thereby improving the acceptor signal to noise ratio (ASN). Studies have identified a dual-modified nucleotide that is incorporated by several polymerase variants. In a typical embodiment, base-modification will be optimized to facilitate sequential nucleotide incorporations, typically without a requirement for removal of the (minor) modification.

All of the compositions and methods disclosed and/or claimed herein can be made and executed without undue experimentation in light of the present disclosure. While the compositions and methods of this invention have been described in terms of preferred embodiments, these embodiments are in no way intended to limit the scope of the claims, and it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references are cited in this application.
Bakhtina, M., S. Lee, et al. (2005). “Use of viscogens, nucleotidealphaS, and rhodium(III) as probes in stopped-flow experiments to obtain new evidence for the mechanism of catalysis by DNA polymerase beta.” Biochemistry 44(13): 5177-87.
Berman, A. J., S. Kamtekar, et al. (2007). “Structures of Phi29 DNA polymerase complexed with substrate: the mechanism of translocation in B-family polymerases.” Embo J 26(14): 3494-505.
Braslavsky, I., B. Hebert, et al. (2003). “Sequence information can be obtained from single DNA molecules.” Proc Natl Acad Sci USA 100(7): 3960-4.
Champoux, J. J. (2001). “DNA topoisomerases: structure, function, and mechanism.” Annu Rev Biochem 70: 369-413.
Clegg, R. M., A. I. Murchie, et al. (1993). “Observing the helical geometry of double-stranded DNA in solution by fluorescence resonance energy transfer.” Proc Natl Acad Sci USA 90(7): 2994-8.
Dale, R. E., J. Eisinger, et al. (1979). “The orientational freedom of molecular probes. The orientation factor in intramolecular energy transfer.” Biophys J 26(2): 161-93.
Diels, O.; Alder, K. (1928). “Synthesen in der hydroaromatischen Reihe”. Liebigs Annalen der Chemie 460 (1): 98-122.
Dobrikov, M. I., K. M. Grady, et al. (2003). “Introduction of the alpha-P-borano-group into deoxynucleoside triphosphates increases their selectivity to HIV-1 reverse transcriptase relative to DNA polymerases.” Nucleosides Nucleotides Nucleic Acids 22(3): 275-82.
Ewing, B. and P. Green (1998). “Base-calling of automated sequencer traces using phred. II. Error probabilities.” Genome Res 8(3): 186-94.
Ewing, B., L. Hillier, et al. (1998). “Base-calling of automated sequencer traces using phred. I. Accuracy assessment.” Genome Res 8(3): 175-85.
Föster, T. (1948). “Zwischenmolekulare Energiewanderung and Fluoreszenz.” Annalen der Physik 2: 55-75.
Gerion, D., F. Pinaud, et al. (2001). “Synthesis and Properties of Biocompatible Water-Soluble Silica-Coated CdSe/ZnS Semiconductor Quantum Dots.” J Phys Chem B 105(37): 8861-71.
Gersten, J. I. N., A. (1984). “Accelerated energy transfer between molecules near a solid particle.” Chem. Phys. Lett. 104: 31-7.
Gryczynski, I. M., J.; Shen, Y.; Gryczynski, Z.; Lakowicz, J. R. (2002). “Multiphoton excitation of fluorescence near metallic particles: enhanced and localized excitation.” J. Phys. Chem. B 106: 2191-5.
Guo, A. Z., X.-Y. (2006). The critical role of surface chemistry in protein microarrays. Functional protein microarrays: Pathways to discovery. P. Predki, CRC press. 4: 53-72.
Ha, T., I. Rasnik, et al. (2002). “Initiation and re-initiation of DNA unwinding by the Escherichia coli Rep helicase.” Nature 419(6907): 638-41.
Hoard, D. E. and D. G. Ott (1965). “Conversion of Mono- and Oligodeoxyribonucleotides to 5-Triphosphates.” J Am Chem Soc 87: 1785-8.
Holmes, H. L. Org. React. 1948, 4, 60-173.
Jiang, W.; Mardyani, S.; Fischer, H.; and Chan, W. C. W. “Design and Characterization of Lysine Cross-Linked Mercapto-Acid Biocompatible Quantum Dots” Chem. Mater., 2006, 18, 872-878.
Kartalov, E. P., M. A. Unger, et al. (2003). “Polyelectrolyte surface interface for single-molecule fluorescence studies of DNA polymerase.” Biotechniques 34(3): 505-10.
Kleppner, D. (1981). “Inhibited spontaneous emission.” Phys Rev Lett 47: 233-6.
Knorre, D. G., V. A. Kurbatov, et al. (1976). “General method for the synthesis of ATP gamma-derivatives.” FEBS Lett 70(1): 105-8.
Lacenere, C. J.; Garg, N. K.; Stoltz, B. M.; Quake, S. R. “Effects of a Modified Dye-labeled Nucleotide Spacer Arm on Incorporation by Thermophilic DNA Polymerases” Nucleosides, Nucleotides, and Nucleic Acids, 2006, 25:9-15.
H. C. Kolb, M. G. Finn and K. B. Sharpless, Angew. Chem., Int. Ed., 2001, 40, 2004.

Lakowicz, J. R. (2001). “Radiative decay engineering: biophysical and biomedical applications.” Anal Biochem 298(1): 1-24.
Lakowicz, J. R., B. Shen, et al. (2001). “Intrinsic fluorescence from DNA can be enhanced by metallic particles.” Biochem Biophys Res Commun 286(5): 875-9.
Lakowicz, J. R., Y. Shen, et al. (2002). “Radiative decay engineering. 2. Effects of Silver Island films on fluorescence intensity, lifetimes, and resonance energy transfer.” Anal Biochem 301(2): 261-77.
Lakowicz, J. R. M., J.; Gryczynski, I.; Gryczynski, Z.; Geddes, C. D. (2003). “Radiative decay engineering: the role of photonic mode density in biotechnology.” J. Phys. D: Appl. Phys. 36: R240-9.
Lawyer et al. (1989) “Isolation, characterization, and expression in Escherichia coli of the DNA polymerase gene from Thermus aquaticus” J. Biol. Chem. 264(11): 6427-6437
Levy, S., G. Sutton, et al. (2007). “The Diploid Genome Sequence of an Individual Human.” PLoS Biol 5(10): e254.
Li, H., A. Valouev, et al. (2007). “A quantile method for sizing optical maps.” J Comput Biol 14(3): 255-66.
Liu, H., T. Ye, et al. (2007). “Fluorescent Carbon Nanoparticles Derived from Candle Soot.” Angew Chem Int Ed Engl 46(34): 6473-6475.
Malicka, J., I. Gryczynski, et al. (2003). “Increased resonance energy transfer between fluorophores bound to DNA in proximity to metallic silver particles.” Anal Biochem 315(2): 160-9.
Malicka, J. G., I.; Fang, J.; Kusba, J.; Lakowicz, J.R. (2002). “Photostability of Cy3 and Cy5-labeled DNA in the presence of metallic silver particles.” J. Fluorescence 12: 439-47.
Malicka, J. G., I.; Gryczynski, Z.; Lakowicz, J. R. (2003). “Effects of fluorophore-to-silver distance on the emission of cyanine-dye labeled oligonucleotides.” Biochem. 315: 57-66.
Medintz, I.; Uyeda, H. T.; Goldman, E. R.; and Mattoussi, H. “Quantum dot bioconjugates for imaging, labelling and sensing” Nature Mater., 2005, 4, 435-446.
Moffatt, J. G. (1964). “A general synthesis of nucleoside 5′-triphosphates.” Canadian Journal of Chemistry 42: 599.
Osborne, M. A., C. L. Barnes, et al. (2001). “Probing DNA surface attachment and local environment using single molecule spectroscopy.” J. Phys. Chem. B 105: 3120-6.
Pinaud, F.; King, F.; Moore, H-P.; and Weiss, S. “Bioactivation and Cell Targeting of Semiconductor CdSe/ZnS Nanocrystals with Phytochelatin-Related Peptides” J. Am. Chem. Soc., 2004, 126, 6115-5123.
Purcell, E. M. (1946). “Spontaneous emission probabilities at radio frequencies.” Phys Rev 69: 681.
Rostovtsev, V. V., L. G. Green, et al. (2002). “A stepwise huisgen cycloaddition process: copper(I)-catalyzed regioselective “ligation” of azides and terminal alkynes.” Angew Chem Int Ed Engl 41(14): 2596-9.
Selvin, P. R. (2000). “The renaissance of fluorescence resonance energy transfer.” Nat Struct Biol 7(9): 730-4.
Shendure, J., R. D. Mitra, et al. (2004). “Advanced sequencing technologies: methods and goals.” Nat Rev Genet 5(5): 335-44.
Smith, A. M.; Duan, H.; Rhyner, M. N.; Ruan, G.; and Nie, S. “A systematic examination of surface coatings on the optical and chemical properties of semiconductor quantum dots” Phys. Chem. Chem. Phys., 2006, 8, 3895-3903
Stephens, J. C., J. A. Schneider, et al. (2001) Haplotype Variation and Linkage Disequilibrium in 313 Human Genes.” Science 293: 489-493.
Stryer, L. (1978). “Fluorescence energy transfer as a spectroscopic ruler.” Annu Rev Biochem 47: 819-46.
Stryer, L. and R. P. Haugland (1967). “Energy transfer: a spectroscopic ruler.” Proc Natl Acad Sci USA 58(2): 719-26.
Sun, Y.-P. Z., B.; Lin, Y.; Wang, W.; Fernando, K. A. S.; Pathak, P.; Meziani, M. J.; Harruff, B. A.; Wang, X.; Wang, H.; Luo, P. G.; Yang, H.; Kose, M. E.; Chen, B.; Veca, M.; Xie, S-Y. (2006). “Quantum-sized carbon dots for bright and colorful photoluminescence.” J Am Chem Soc 128: 7756-7.
Waterston, R. H., K. Lindblad-Toh, et al. (2002). “Initial sequencing and comparative analysis of the mouse genome.” Nature 420(6915): 520-62.
Weiss, S. (2000). “Measuring conformational dynamics of biomolecules by single molecule fluorescence spectroscopy.” Nat Struct Biol 7(9): 724-9.
Yablonovitch, E. (1987). “Inhibited spontaneous emission in solid-state physics and electronics.” Phys Rev Lett 58(20): 2059-2062.
Zhang, J., Fu, Y., Lakowicz, J. R., (2007) “Enhanced Forster Resonance Energy Transfer (FRET) on a Single Metal Particle” J. Phys. Chem. C 111: 50-56
Zhu, M.-Q. C., E.; Sun, J.; Drezek, R. A. (2007). “Surface modification and functionalization of semiconductor quantum dots through reactive coating of silanes in toluene.” Journal of Materials Chemistry 17: 800-5.
Zipper, H., H. Brunner, et al. (2004). “Investigations on DNA intercalation and surface binding by SYBR Green I, its structure determination and methodological implications.” Nucleic Acids Res 32(12): e103.

1.-51. (canceled)

52. An isolated variant of Phi-29 polymerase comprising the amino acid sequence shown in SEQ ID NO: 3, wherein the variant comprises an amino acid sequence that is at least 80% identical to SEQ ID NO: 3, and wherein the variant further comprises one or more mutations selected from the group consisting of:
V250A/E375Y, V250A/E375A/Q380A, V250A/E375C, V250A/E375Y, V250I/E375A/Q380A, V250I/E375C, V250A, V250I, E375A, E375C, E375Y, E375A/Q380A, Q380A, D456N, D456E, D456S, D458N, V250A/E375A/Q380A/D456E, E375Y/V250L, E375Y/V250P, E375Y/V250Q, E375Y/V250R, E375Y/V250Y, E375Y/V250F, E375Y/V250S, E375Y/V250C, E375Y/V250T, E375Y/V250K, E375Y/V250H, E375Y/V250N, E375Y/V250D, E375Y/V250G, E375Y/V250W, E375Y/S388G, E375Y/K512A, E375Y/K525A, Y254V/E375Y, K132A, K383A, K383R, K383P, K371A, K371T, Y254F, Y254V, Y254S, Y254V, Y254S, K379A, K525A, K135A, P255S, S388G, K512A, L384R, E486A, E486D, K478A, E375W, N387A, N387Y, V250A/E375W, D456N/D458N/L351P, Y254V/A377E, D456N/D458N, D169A, D12A/D66A/D169A, T15I, N62D, C22S, C290S, C448S, C530S, C290S/C448S/C530S, C22S/C448S/C530S, C22S/C290S/C530S and C22S/C290S/C448S.

53. An isolated variant of Phi-29 polymerase that comprises one or more mutations selected from the group consisting of: V250A/E375Y, V250A/E375A/Q380A, V250A/E375C, V250A/E375Y, V250I/E375A/Q380A, V250I/E375C, V250A, V250I, E375A, E375C, E375Y, E375A′Q380A, Q380A, D456N, D456E, D456S, D458N, V250A/E375A/Q380A/D456E, E375Y/V250L, E375Y/V250P, E375Y/V250Q, E375Y/V250R, E375Y/V250Y, E375Y/V250F, E375Y/V250S, E375Y/V250C, E375Y/V250T, E375Y/V250K, E375Y/V250H, E375Y/V250N, E375Y/V250D, E375Y/V250G, E375Y/V250W, E375Y/S388G, E375Y/K512A, E375Y/K525A, Y254V/E375Y, K132A, K383A, K383R, K383P, K371A, K371T, Y254F, Y254V, Y254S, Y254V, Y254S, K379A, K525A, K135A, P255S, S388G, K512A, L384R, E486A, E486D, K478A, E375W, N387A, N387Y, V250A/E375W, D456N/D458N/L351P, Y254V/A377E, D456N/D458N, D169A, D12A/D66A/D169A, T15I, N62D, C22S, C290S, C448S, C530S, C290S/C448S/C530S, C22S/C448S/C530S, C22S/C290S/C530S and C22S/C290S/C448S.

54. The isolated variant of claim 53, wherein the variant comprises an amino acid sequence that is at least 80% identical to the amino acid sequence of SEQ ID NO: 3.

55. The variant of claim 54, wherein the protein is operably linked to a FRET donor.

56. The protein of claim 55, wherein the FRET donor is a nanocrystal.

57. The variant of claim 56, wherein the FRET donor is capable of undergoing FRET with an acceptor attached to a nucleotide before, during or after the nucleotide is incorporated by the polymerase onto the terminal 3′OH of a synthesized DNA molecule.

58.-64. (canceled)

65. A method for detecting one or more nucleotide incorporation events, comprising: conducting a nucleotide polymerase reaction in the presence of one or more detectably labeled nucleotides and a mutant polymerase according to claim 52, which reaction results the production of a detectable signal before, after or during a nucleotide incorporation event; and detecting the detectable signal, thereby determining if a nucleotide incorporation event has occurred.

66. The method of claim 65, further comprising the step of analyzing the signal to determine the identity of the nucleobase of the incorporated nucleotide.

67. The method of claim 66, wherein the detectable signal is a FRET signal.
